SEQUENCE LISTING (452 sequences)

SEQ ID NO: 1 - protein, 11 aa, synthetic construct
  source 1..11: note = B17B01 HCDR1
GYSITSGYFWN

SEQ ID NO: 2 - protein, 9 aa, synthetic construct
  source 1..9: note = B17B01 HCDR2
YISYDGSNN

SEQ ID NO: 3 - protein, 11 aa, synthetic construct
  source 1..11: note = B17B01 HCDR3
PSPGTGYAVDY

SEQ ID NO: 4 - protein, 16 aa, synthetic construct
  source 1..16: note = B17B01 LCDR1
RSSQSLVHSNGNTYLH

SEQ ID NO: 5 - protein, 7 aa, synthetic construct
  source 1..7: note = B17B01 LCDR2
KVSNRFS

SEQ ID NO: 6 - protein, 9 aa, synthetic construct
  source 1..9: note = B17B01 LCDR3
SQSTHVPFT

SEQ ID NO: 7 - protein, 444 aa, synthetic construct
  source 1..444: note = B17B01 Heavy Chain
NVQLQESGPGLVKPSQSLSLTCSVAGYSITSGYFWNWIRQFPGNKLEWMGYISYDGSNNYNPSLKNRISITRDTSKNQFFLKLNSVTTEDTATYYCASPSPGTGYAVDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTYGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 8 - protein, 219 aa, synthetic construct
  source 1..219: note = B17B01 Light Chain
NVVMTQTPLSLPVSLGDQASISCRSSQSLVHSNGNTYLHWYLQKPGQSPKFLIYKVSNRFSGVPDRFSGGGSGTEFTLKISRVEAEDLGVYFCSQSTHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - protein, 447 aa, synthetic construct
  source 1..447: note = B17B1 Heavy Chain
NVQLQESGPGLVKPSQSLSLTCSVAGYSITSGYFWNWIRQFPGNKLEWMGYISYDGSNNYNPSLKNRISITRDTSKNQFFLKLNSVTTEDTATYYCASPSPGTGYAVDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 10 - protein, 219 aa, synthetic construct; identical to SEQ ID NO: 8
  source 1..219: note = B17B1 Light Chain

SEQ ID NO: 11 - protein, 447 aa, synthetic construct
  source 1..447: note = B17B2 Heavy Chain
DVQLKESGPGLVKPSQSLSVTCSVTGYSITSGYYWNWYRQFPGNKLEWMGYISYDGSNNYNPSLKNRISITRDTSKNQILLKLTYVTTEDTATYYCTRPSPGTGYAVDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 12 - protein, 219 aa, synthetic construct
  source 1..219: note = B17B2 Light Chain
DIVMTQSPDSLAVSLGERATINCRSSQSLVHSNGNTYLHWYQQKPGQPPKLLIYKVSNRFSGVPDRFSGSGSGTDFTLTISSLQAEDVAVYYCSQSTHVPFTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - protein, 447 aa, synthetic construct
  source 1..447: note = B17B21 Heavy Chain
QVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 14 - protein, 219 aa, synthetic construct
  source 1..219: note = B17B21 Light Chain
DIQMTQSPSSLSASVGDRVTITCRSSQSLVHSNGNTYLHWYQQKPGKAPKFLIYKVSNRFSGVPSRFSGSGSGTDFTLTISSLQPEDFATYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 445 aa, synthetic construct
  source 1..445: note = I3RB217 Heavy Chain
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 215 aa, synthetic construct
  source 1..215: note = I3RB217 Light Chain
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 447 aa, synthetic construct
  source 1..447: note = Null Heavy Chain
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 218 aa, synthetic construct
  source 1..218: note = Null Light Chain
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 119 aa, synthetic construct
  source 1..119: note = B17H3
EVQLLESGGGLVQPGGSLRLSCAASGYSITSGYFWNWVRQAPGKGLEWVSYISYDGSNNYADSVKGRFTISRDNSKNTLYLQMNSLRAEDTAVYYCAKPSPGTGYAVDYWGQGTLVTVS

SEQ ID NO: 20 - protein, 120 aa, synthetic construct
  source 1..120: note = B17H4
EVQLLESGGGLVQPGGSLRLSCAASGYSITSGYFWNWVRQAPGKGLEWVSYISYDGSNNYADSVKGRFTISRDNSKNTLYLQMNSLRAEDTAVYYCASPSPGTGYAVDYWGQGTLVTVSS

SEQ ID NO: 21 - protein, 120 aa, synthetic construct
  source 1..120: note = B17H5
QVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSS

SEQ ID NO: 22 - protein, 112 aa, synthetic construct
  source 1..112: note = B17L3
DIQMTQSPSSLSASVGDRVTITCRSSQSLVHSNGNTYLHWYQQKPGKAPKLLIYKVSNRFSGVPSRFSGSGSGTDFTLTISSLQPEDFATYYCSQSTHVPFTFGQGTKLEIK

SEQ ID NO: 23 - protein, 112 aa, synthetic construct
  source 1..112: note = B17L4
DIQMTQSPSSLSASVGDRVTITCRSSQSLVHSNGNTYLHWYQQKPGKAPKFLIYKVSNRFSGVPSRFSGSGSGTDFTLTISSLQPEDFATYYCSQSTHVPFTFGQGTKLEIK

SEQ ID NO: 24 - protein, 112 aa, synthetic construct
  source 1..112: note = B17L5
DVVMTQSPLSLPVTLGQPASISCRSSQSLVHSNGNTYLHWFQQRPGQSPRFLIYKVSNRFSGVPDRFSGSGSGTDFTLKISRVEAEDVGVYYCSQSTHVPFTFGQGTKLEIK

SEQ ID NO: 25 - protein, 120 aa, synthetic construct
  source 1..120: note = B17H1
NVQLQESGPGLVKPSQSLSLTCSVAGYSITSGYFWNWIRQFPGNKLEWMGYISYDGSNNYNPSLKNRISITRDTSKNQFFLKLNSVTTEDTATYYCASPSPGTGYAVDYWGQGTSVTVSS

SEQ ID NO: 26 - protein, 112 aa, synthetic construct
  source 1..112: note = B17L1
NVVMTQTPLSLPVSLGDQASISCRSSQSLVHSNGNTYLHWYLQKPGQSPKFLIYKVSNRFSGVPDRFSGGGSGTEFTLKISRVEAEDLGVYFCSQSTHVPFTFGSGTKLEIK

SEQ ID NO: 27 - protein, 493 aa, synthetic construct
  source 1..493: note = TCR-Vbeta17_Fc fusion
MAWVWTLLFLMAAAQSIQAVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSRSSYEQYFGPGTRLTVTEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 28 - protein, 719 aa, synthetic construct
  source 1..719: note = B17B21 half antibody
MAWVWTLLFLMAAAQSIQADIQMTQSPSSLSASVGDRVTITCRSSQSLVHSNGNTYLHWYQQKPGKAPKFLIYKVSNRFSGVPSRFSGSGSGTDFTLTISSLQPEDFATYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSEGKSSGSGSESKSTEGKSSGSGSESKSTGGSQVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 29 - DNA, 4320 bp, synthetic construct
  source 1..4320: mol_type = other DNA; note = B17B21 half antibody
atggcctgggtgtggaccctgctgttcctgatggccgccgcccagagcatccaggccgacatccagatgacccagagcccaagcagcctgagcgccagcgtgggcgaccgcgtgaccatcacctgccgcagcagccagagcctggtgcacagcaacggcaacacctacctgcactggtaccagcagaagccaggcaaggccccaaagttcctgatctacaaggtgagcaaccgcttcagcggcgtgccaagccgcttcagcggcagcggcagcggcaccgacttcaccctgaccatcagcagcctgcagccagaggacttcgccacctactactgcagccagagcacccacgtgccattcaccttcggccagggcaccaagctggagatcaagcgcaccgtggccgccccaagcgtgttcatcttcccaccaagcgacgagcagctgaagagcggcaccgccagcgtggtgtgcctgctgaacaacttctacccacgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagcccagtgaccaagagcttcaaccgcggcgagtgcggcggcagcgagggcaagagcagcggcagcggcagcgagagcaagagcaccgagggcaagagcagcggcagcggcagcgagagcaagagcaccggcggcagccaggtgcagctgcaggagagcggcccaggcctggtgaagccaagcgagaccctgagcctgacctgcaccgtgagcggctacagcatcaccagcggctacttctggaactggatccgccagccaccaggcaagggcctggagtggatcggctacatcagctacgacggcagcaacaactacaacccaagcctgaagagccgcgtgaccatcagccgcgacaccagcaagaaccagttcagcctgaagctgagcagcgtgaccgccgccgacaccgccgtgtactactgcgccagcccaagcccaggcaccggctacgccgtggactactggggccagggcaccctggtgaccgtgagcagcgccagcaccaagggcccaagcgtgttcccactggccccatgcagccgcagcaccagcgagagcaccgccgccctgggctgcctggtgaaggactacttcccagagccagtgaccgtgagctggaacagcggcgccctgaccagcggcgtgcacaccttcccagccgtgctgcagagcagcggcctgtacagcctgagcagcgtggtgaccgtgccaagcagcagcctgggcaccaagacctacacctgcaacgtggaccacaagccaagcaacaccaaggtggacaagcgcgtggagagcaagtacggcccaccatgcccaccatgcccagccccagaggccgccggcggcccaagcgtgttcctgttcccaccaaagccaaaggacaccctgatgatcagccgcaccccagaggtgacctgcgtggtggtggacgtgagccaggaggacccagaggtgcagttcaactggtacgtggacggcgtggaggtgcacaacgccaagaccaagccacgcgaggagcagttcaacagcacctaccgcgtggtgagcgtgctgaccgtgctgcaccaggactggctgaacggcaaggagtacaagtgcaaggtgagcaacaagggcctgccaagcagcatcgagaagaccatcagcaaggccaagggccagccacgcgagccacaggtgtacaccctgccaccaagccaggaggagatgaccaagaaccaggtgagcctgagctgcgccgtgaagggcttctacccaagcgacatcgccgtggagtgggagagcaacggccagccagagaacaactacaagaccaccccaccagtgctggacagcgacggcagcttcttcctggtgagccgcctgaccgtggacaagagccgctggcaggagggcaacgtgttcagctgcagcgtgatgcacgaggccctgcacaaccgcttcacccagaagagcctgagcctgagcctgggcaagatggcctgggtgtggaccctgctgttcctgatggccgccgcccagagcatccaggccgacatccagatgacccagagcccaagcagcctgagcgccagcgtgggcgaccgcgtgaccatcacctgccgcagcagccagagcctggtgcacagcaacggcaacacctacctgcactggtaccagcagaagccaggcaaggccccaaagttcctgatctacaaggtgagcaaccgcttcagcggcgtgccaagccgcttcagcggcagcggcagcggcaccgacttcaccctgaccatcagcagcctgcagccagaggacttcgccacctactactgcagccagagcacccacgtgccattcaccttcggccagggcaccaagctggagatcaagcgcaccgtggccgccccaagcgtgttcatcttcccaccaagcgacgagcagctgaagagcggcaccgccagcgtggtgtgcctgctgaacaacttctacccacgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagcccagtgaccaagagcttcaaccgcggcgagtgcggcggcagcgagggcaagagcagcggcagcggcagcgagagcaagagcaccgagggcaagagcagcggcagcggcagcgagagcaagagcaccggcggcagccaggtgcagctgcaggagagcggcccaggcctggtgaagccaagcgagaccctgagcctgacctgcaccgtgagcggctacagcatcaccagcggctacttctggaactggatccgccagccaccaggcaagggcctggagtggatcggctacatcagctacgacggcagcaacaactacaacccaagcctgaagagccgcgtgaccatcagccgcgacaccagcaagaaccagttcagcctgaagctgagcagcgtgaccgccgccgacaccgccgtgtactactgcgccagcccaagcccaggcaccggctacgccgtggactactggggccagggcaccctggtgaccgtgagcagcgccagcaccaagggcccaagcgtgttcccactggccccatgcagccgcagcaccagcgagagcaccgccgccctgggctgcctggtgaaggactacttcccagagccagtgaccgtgagctggaacagcggcgccctgaccagcggcgtgcacaccttcccagccgtgctgcagagcagcggcctgtacagcctgagcagcgtggtgaccgtgccaagcagcagcctgggcaccaagacctacacctgcaacgtggaccacaagccaagcaacaccaaggtggacaagcgcgtggagagcaagtacggcccaccatgcccaccatgcccagccccagaggccgccggcggcccaagcgtgttcctgttcccaccaaagccaaaggacaccctgatgatcagccgcaccccagaggtgacctgcgtggtggtggacgtgagccaggaggacccagaggtgcagttcaactggtacgtggacggcgtggaggtgcacaacgccaagaccaagccacgcgaggagcagttcaacagcacctaccgcgtggtgagcgtgctgaccgtgctgcaccaggactggctgaacggcaaggagtacaagtgcaaggtgagcaacaagggcctgccaagcagcatcgagaagaccatcagcaaggccaagggccagccacgcgagccacaggtgtacaccctgccaccaagccaggaggagatgaccaagaaccaggtgagcctgagctgcgccgtgaagggcttctacccaagcgacatcgccgtggagtgggagagcaacggccagccagagaacaactacaagaccaccccaccagtgctggacagcgacggcagcttcttcctggtgagccgcctgaccgtggacaagagccgctggcaggagggcaacgtgttcagctgcagcgtgatgcacgaggccctgcacaaccgcttcacccagaagagcctgagcctgagcctgggcaagtgatag

SEQ ID NO: 30 - protein, 713 aa, synthetic construct
  source 1..713: note = I3RB217 half antibody
MAWVWTLLFLMAAAQSIQAEIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSEGKSSGSGSESKSTEGKSSGSGSESKSTGGSEVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - DNA, 2139 bp, synthetic construct
  source 1..2139: mol_type = other DNA; note = I3RB217 half antibody
atggcctgggtgtggaccctgctgttcctgatggccgccgcccagagcatccaggccgagatcgtgctgacccagagcccaggcaccctgagcctgagcccaggcgagcgcgccaccctgagctgccgcgccagccagagcgtgagcagcagctacctggcctggtaccagcagaagccaggccaggccccacgcctgctgatctacggcgccagcagccgcgccaccggcatcccagaccgcttcagcggcagcggcagcggcaccgacttcaccctgaccatcagccgcctggagccagaggacttcgccgtgtactactgccagcaggactacggcttcccatggaccttcggccagggcaccaaggtggagatcaagcgcaccgtggccgccccaagcgtgttcatcttcccaccaagcgacgagcagctgaagagcggcaccgccagcgtggtgtgcctgctgaacaacttctacccacgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagcccagtgaccaagagcttcaaccgcggcgagtgcggcggcagcgagggcaagagcagcggcagcggcagcgagagcaagagcaccgagggcaagagcagcggcagcggcagcgagagcaagagcaccggcggcagcgaggtgcagctggtgcagagcggcgccgaggtgaagaagccaggcgagagcctgaagatcagctgcaagggcagcggctacagcttcaccagctactggatcagctgggtgcgccagatgccaggcaagggcctggagtggatgggcatcatcgacccaagcgacagcgacacccgctacagcccaagcttccagggccaggtgaccatcagcgccgacaagagcatcagcaccgcctacctgcagtggagcagcctgaaggccagcgacaccgccatgtactactgcgcccgcggcgacggcagcaccgacctggactactggggccagggcaccctggtgaccgtgagcagcgccagcaccaagggcccaagcgtgttcccactggccccatgcagccgcagcaccagcgagagcaccgccgccctgggctgcctggtgaaggactacttcccagagccagtgaccgtgagctggaacagcggcgccctgaccagcggcgtgcacaccttcccagccgtgctgcagagcagcggcctgtacagcctgagcagcgtggtgaccgtgccaagcagcagcctgggcaccaagacctacacctgcaacgtggaccacaagccaagcaacaccaaggtggacaagcgcgtggagagcaagtacggcccaccatgcccaccatgcccagccccagaggccgccggcggcccaagcgtgttcctgttcccaccaaagccaaaggacaccctgatgatcagccgcaccccagaggtgacctgcgtggtggtggacgtgagccaggaggacccagaggtgcagttcaactggtacgtggacggcgtggaggtgcacaacgccaagaccaagccacgcgaggagcagttcaacagcacctaccgcgtggtgagcgtgctgaccgtgctgcaccaggactggctgaacggcaaggagtacaagtgcaaggtgagcaacaagggcctgccaagcagcatcgagaagaccatcagcaaggccaagggccagccacgcgagccacaggtgtacaccctgccaccaagccaggaggagatgaccaagaaccaggtgagcctgtggtgcctggtgaagggcttctacccaagcgacatcgccgtggagtgggagagcaacggccagccagagaacaactacaagaccaccccaccagtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaag

SEQ ID NO: 32 - protein, 713 aa, synthetic construct; identical to SEQ ID NO: 30
  source 1..713: note = B23B49 half antibody

SEQ ID NO: 33 - DNA, 2154 bp, synthetic construct
  source 1..2154: mol_type = other DNA; note = B23B49 half antibody
atggcctgggtgtggaccctgctgttcctgatggccgccgcccagagcatccaggccgacatcgtgatgacccagagcccagacagcctggccgtgagcctgggcgagcgcgccaccatcaactgccgcgccagccagagcgtggactacaacggcatcagctacatgcactggtaccagcagaagccaggccagccaccaaagctgctgatctacgccgccagcaacccagagagcggcgtgccagaccgcttcagcggcagcggcagcggcaccgacttcaccctgaccatcagcagcctgcaggccgaggacgtggccgtgtactactgccagcagatcatcgaggacccatggaccttcggccagggcaccaaggtggagatcaagcgcaccgtggccgccccaagcgtgttcatcttcccaccaagcgacgagcagctgaagagcggcaccgccagcgtggtgtgcctgctgaacaacttctacccacgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagcccagtgaccaagagcttcaaccgcggcgagtgcggcggcagcgagggcaagagcagcggcagcggcagcgagagcaagagcaccgagggcaagagcagcggcagcggcagcgagagcaagagcaccggcggcagccagatcaccctgaaggagagcggcccaaccctggtgaagccaacccagaccctgaccctgacctgcaccttcagcggcttcagcctgagcaccagcggcatgggcgtgagctggatccgccagccaccaggcaaggccctggagtggctggcccacatctactgggacgacgacaagcgctacaacccaagcctgaagagccgcctgaccatcaccaaggacaccagcaagaaccaggtggtgctgaccatgaccaacatggacccagtggacaccgccacctactactgcgcccgcctgtacggcttcacctacggcttcgcctactggggccagggcaccctggtgaccgtgagcagcgccagcaccaagggcccaagcgtgttcccactggccccatgcagccgcagcaccagcgagagcaccgccgccctgggctgcctggtgaaggactacttcccagagccagtgaccgtgagctggaacagcggcgccctgaccagcggcgtgcacaccttcccagccgtgctgcagagcagcggcctgtacagcctgagcagcgtggtgaccgtgccaagcagcagcctgggcaccaagacctacacctgcaacgtggaccacaagccaagcaacaccaaggtggacaagcgcgtggagagcaagtacggcccaccatgcccaccatgcccagccccagaggccgccggcggcccaagcgtgttcctgttcccaccaaagccaaaggacaccctgatgatcagccgcaccccagaggtgacctgcgtggtggtggacgtgagccaggaggacccagaggtgcagttcaactggtacgtggacggcgtggaggtgcacaacgccaagaccaagccacgcgaggagcagttcaacagcacctaccgcgtggtgagcgtgctgaccgtgctgcaccaggactggctgaacggcaaggagtacaagtgcaaggtgagcaacaagggcctgccaagcagcatcgagaagaccatcagcaaggccaagggccagccacgcgagccacaggtgtacaccctgccaccaagccaggaggagatgaccaagaaccaggtgagcctgtggtgcctggtgaagggcttctacccaagcgacatcgccgtggagtgggagagcaacggccagccagagaacaactacaagaccaccccaccagtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaag

SEQ ID NO: 34 - protein, 5 aa, synthetic construct
  source 1..5: note = I3RB217 HCDR1
SYWIS

SEQ ID NO: 35 - protein, 17 aa, synthetic construct
  source 1..17: note = I3RB217 HCDR2
IIDPSDSDTRYSPSFQG

SEQ ID NO: 36 - protein, 9 aa, synthetic construct
  source 1..9: note = I3RB217 HCDR3
GDGSTDLDY

SEQ ID NO: 37 - protein, 11 aa, synthetic construct
  source 1..11: note = I3RB217 LCDR1
RASQSVSSSYL

SEQ ID NO: 38 - protein, 7 aa, synthetic construct
  source 1..7: note = I3RB217 LCDR2
GASSRAT

SEQ ID NO: 39 - protein, 9 aa, synthetic construct
  source 1..9: note = I3RB217 LCDR3
QQDYGFPWT

SEQ ID NO: 40 - protein, 118 aa, synthetic construct
  source 1..118: note = I3RB217 HC
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSS

SEQ ID NO: 41 - protein, 108 aa, synthetic construct
  source 1..108: note = I3RB217 LC
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIK

SEQ ID NO: 42 - protein, 11 aa, synthetic construct
  source 1..11: note = IGHJ1*01 HC
WGQGTLVTVSS

SEQ ID NO: 43 - protein, 10 aa, synthetic construct
  source 1..10: note = IGKJ2*01 LC
FGQGTKLEIK

SEQ ID NO: 44 - protein, 449 aa, synthetic construct
  source 1..449: note = Va10.2_Fc fusion
MAWVWTLLFLMAAAQSIQAQLLEQSPQFLSIQEGENLTVYCNSSSVFSSLQWYRQEPGEGPVLLVTVVTGGEVKKLKRLTFQFGDARKDSSLHITAAQPGDTGLYLCAGAGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 45 - protein, 11 aa, synthetic construct
GYSITSGYFWN

SEQ ID NO: 46 - protein, 9 aa, synthetic construct
YISYDGSNN

SEQ ID NO: 47 - protein, 11 aa, synthetic construct
PSPGTGYAVDY

SEQ ID NO: 48 - protein, 6 aa, synthetic construct
SGYFWN

SEQ ID NO: 49 - protein, 16 aa, synthetic construct
YISYDGSNNYNPSLKS

SEQ ID NO: 50 - protein, 11 aa, synthetic construct
PSPGTGYAVDY

SEQ ID NO: 51 - protein, 8 aa, synthetic construct
GYSITSGY

SEQ ID NO: 52 - protein, 5 aa, synthetic construct
SYDGS

SEQ ID NO: 53 - protein, 11 aa, synthetic construct
PSPGTGYAVDY

SEQ ID NO: 54 - protein, 9 aa, synthetic construct
GYSITSGYF

SEQ ID NO: 55 - protein, 7 aa, synthetic construct
ISYDGSN

SEQ ID NO: 56 - protein, 13 aa, synthetic construct
ASPSPGTGYAVDY

SEQ ID NO: 57 - protein, 7 aa, synthetic construct
TSGYFWN

SEQ ID NO: 58 - protein, 12 aa, synthetic construct
WIGYISYDGSNN

SEQ ID NO: 59 - protein, 12 aa, synthetic construct
ASPSPGTGYAVD

SEQ ID NO: 60 - protein, 16 aa, synthetic construct
RSSQSLVHSSGNTYLH

SEQ ID NO: 61 - protein, 7 aa, synthetic construct
KVSNRFS

SEQ ID NO: 62 - protein, 9 aa, synthetic construct
SQSTHVPFT

SEQ ID NO: 63 - protein, 16 aa, synthetic construct
RSSQSLVHSSGNTYLH

SEQ ID NO: 64 - protein, 7 aa, synthetic construct
KVSNRFS

SEQ ID NO: 65 - protein, 9 aa, synthetic construct
SQSTHVPFT

SEQ ID NO: 66 - protein, 16 aa, synthetic construct
RSSQSLVHSSGNTYLH

SEQ ID NO: 67 - protein, 7 aa, synthetic construct
KVSNRFS

SEQ ID NO: 68 - protein, 9 aa, synthetic construct
SQSTHVPFT

SEQ ID NO: 69 - protein, 11 aa, synthetic construct
QSLVHSSGNTY

SEQ ID NO: 70 - skipped

SEQ ID NO: 71 - protein, 9 aa, synthetic construct
SQSTHVPFT

SEQ ID NO: 72 - protein, 12 aa, synthetic construct
VHSSGNTYLHWY

SEQ ID NO: 73 - protein, 10 aa, synthetic construct
FLIYKVSNRF

SEQ ID NO: 74 - protein, 8 aa, synthetic construct
SQSTHVPF

SEQ ID NO: 75 - protein, 120 aa, synthetic construct
QVQLQESGPGLVKPSETLSLTCTVSGYSITSGYFWNWIRQPPGCGLEWIGYISYDGSNNYNPSLKSRVTISRDTSKNQFSLKLSSVTAADTAVYYCASPSPGTGYAVDYWGQGTLVTVSS

SEQ ID NO: 76 - protein, 112 aa, synthetic construct
DIQMTQSPSSLSASVGDRVTITCRSSQSLVHSSGNTYLHWYQQKPGKAPKFLIYKVSNRFSGVPSRFSGSGSGTDFTLTISSLQPEDFATYYCSQSTHVPFTFGCGTKLEIK

SEQ ID NO: 77 - protein, 11 aa, synthetic construct
GYSITSGYFWN

SEQ ID NO: 78 - protein, 9 aa, synthetic construct
YISYDGSNN

SEQ ID NO: 79 - protein, 11 aa, synthetic construct
PSPGTGYAVDY

SEQ ID NO: 80 - protein, 6 aa, synthetic construct
SGYFWN

SEQ ID NO: 81 - protein, 16 aa, synthetic construct
YISYDGSNNYNPSLKS

SEQ ID NO: 82 - protein, 11 aa, synthetic construct
PSPGTGYAVDY

SEQ ID NO: 83 - protein, 8 aa, synthetic construct
GYSITSGY

SEQ ID NO: 84 - protein, 5 aa, synthetic construct
SYDGS

SEQ ID NO: 85 - protein, 11 aa, synthetic construct
PSPGTGYAVDY

SEQ ID NO: 86 - protein, 9 aa, synthetic construct
GYSITSGYF

SEQ ID NO: 87 - protein, 7 aa, synthetic construct
ISYDGSN

SEQ ID NO: 88 - protein, 13 aa, synthetic construct
ASPSPGTGYAVDY

SEQ ID NO: 89 - protein, 7 aa, synthetic construct
TSGYFWN

SEQ ID NO: 90 - protein, 12 aa, synthetic construct
WIGYISYDGSNN

SEQ ID NO: 91 - protein, 12 aa, synthetic construct
ASPSPGTGYAVD

SEQ ID NO: 92 - protein, 16 aa, synthetic construct
RSSQSLVHSSGNTYLH

SEQ ID NO: 93 - protein, 7 aa, synthetic construct
KVSNRFS

SEQ ID NO: 94 - protein, 9 aa, synthetic construct
SQSTHVPFT

SEQ ID NO: 95 - protein, 16 aa, synthetic construct
RSSQSLVHSSGNTYLH

SEQ ID NO: 96 - protein, 7 aa, synthetic construct
KVSNRFS

SEQ ID NO: 97 - protein, 9 aa, synthetic construct
SQSTHVPFT

SEQ ID NO: 98 - protein, 16 aa, synthetic construct
RSSQSLVHSSGNTYLH

SEQ ID NO: 99 - protein, 7 aa, synthetic construct
KVSNRFS

SEQ ID NO: 100 - protein, 9 aa, synthetic construct
SQSTHVPFT

SEQ ID NO: 101 - protein, 11 aa, synthetic construct
QSLVHSSGNTY

SEQ ID NO: 102 - skipped

SEQ ID NO: 103 - protein, 9 aa, synthetic construct
SQSTHVPFT

SEQ ID NO: 104 - protein, 12 aa, synthetic construct
VHSSGNTYLHWY

SEQ ID NO: 105 - protein, 10 aa, synthetic construct
FLIYKVSNRF

SEQ ID NO: 106 - protein, 8 aa, synthetic construct
SQSTHVPF

SEQ ID NO: 107 - protein, 120 aa, synthetic construct; identical to SEQ ID NO: 21

SEQ ID NO: 108 - protein, 112 aa, synthetic construct
DIQMTQSPSSLSASVGDRVTITCRSSQSLVHSSGNTYLHWYQQKPGKAPKFLIYKVSNRFSGVPSRFSGSGSGTDFTLTISSLQPEDFATYYCSQSTHVPFTFGQGTKLEIK

SEQ ID NO: 109 - protein, 11 aa, synthetic construct
GYSITSGYFWN

SEQ ID NO: 110 - protein, 9 aa, synthetic construct
YISYDGSNN

SEQ ID NO: 111 - protein, 11 aa, synthetic construct
PSPGTGYAVDY

SEQ ID NO: 112 - protein, 6 aa, synthetic construct
SGYFWN

SEQ ID NO: 113 - protein, 16 aa, synthetic construct
YISYDGSNNYNPSLKS

SEQ ID NO: 114 - protein, 11 aa, synthetic construct
PSPGTGYAVDY

SEQ ID NO: 115 - protein, 8 aa, synthetic construct
GYSITSGY

SEQ ID NO: 116 - protein, 5 aa, synthetic construct
SYDGS

SEQ ID NO: 117 - protein, 11 aa, synthetic construct
PSPGTGYAVDY

SEQ ID NO: 118 - protein, 9 aa, synthetic construct
GYSITSGYF

SEQ ID NO: 119 - protein, 7 aa, synthetic construct
ISYDGSN

SEQ ID NO: 120 - protein, 13 aa, synthetic construct
ASPSPGTGYAVDY

SEQ ID NO: 121 - protein, 7 aa, synthetic construct
TSGYFWN

SEQ ID NO: 122 - protein, 12 aa, synthetic construct
WIGYISYDGSNN

SEQ ID NO: 123 - protein, 12 aa, synthetic construct
ASPSPGTGYAVD

SEQ ID NO: 124 - protein, 16 aa, synthetic construct
RSSQSLVHSSGNTYLH

SEQ ID NO: 125 - protein, 7 aa, synthetic construct
KVSNRFS

SEQ ID NO: 126 - protein, 9 aa, synthetic construct
SQSTHVPFT

SEQ ID NO: 127 - protein, 16 aa, synthetic construct
RSSQSLVHSSGNTYLH

SEQ ID NO: 128 - protein, 7 aa, synthetic construct
KVSNRFS

SEQ ID NO: 129 - protein, 9 aa, synthetic construct
SQSTHVPFT

SEQ ID NO: 130 - protein, 16 aa, synthetic construct
RSSQSLVHSSGNTYLH

SEQ ID NO: 131 - protein, 7 aa, synthetic construct
KVSNRFS

SEQ ID NO: 132 - protein, 9 aa, synthetic construct
SQSTHVPFT

SEQ ID NO: 133 - protein, 11 aa, synthetic construct
QSLVHSSGNTY

SEQ ID NO: 134 - skipped

SEQ ID NO: 135 - protein, 9 aa, synthetic construct
SQSTHVPFT

SEQ ID NO: 136 - protein, 12 aa, synthetic construct
VHSSGNTYLHWY

SEQ ID NO: 137 - protein, 10 aa, synthetic construct
FLIYKVSNRF

SEQ ID NO: 138 - protein, 8 aa, synthetic construct
SQSTHVPF

SEQ ID NO: 139 - protein, 120 aa, synthetic construct
QVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGCGTLVTVSS

SEQ ID NO: 140 - protein, 112 aa, synthetic construct
DIQMTQSPSSLSASVGDRVTITCRSSQSLVHSSGNTYLHWYQQKPGCAPKFLIYKVSNRFSGVPSRFSGSGSGTDFTLTISSLQPEDFATYYCSQSTHVPFTFGQGTKLEIK

SEQ ID NO: 141 - protein, 11 aa, synthetic construct
GYSITSGYFWN

SEQ ID NO: 142 - protein, 9 aa, synthetic construct
YISYDGSNN

SEQ ID NO: 143 - protein, 11 aa, synthetic construct
PSPGTGYAVDY

SEQ ID NO: 144 - protein, 6 aa, synthetic construct
SGYFWN

SEQ ID NO: 145 - protein, 16 aa, synthetic construct
YISYDGSNNYNPSLKS

SEQ ID NO: 146 - protein, 11 aa, synthetic construct
PSPGTGYAVDY

SEQ ID NO: 147 - protein, 8 aa, synthetic construct
GYSITSGY

SEQ ID NO: 148 - protein, 5 aa, synthetic construct
SYDGS

SEQ ID NO: 149 - protein, 11 aa, synthetic construct
PSPGTGYAVDY

SEQ ID NO: 150 - protein, 9 aa, synthetic construct
GYSITSGYF

SEQ ID NO: 151 - protein, 7 aa, synthetic construct
ISYDGSN

SEQ ID NO: 152 - protein, 13 aa, synthetic construct
ASPSPGTGYAVDY

SEQ ID NO: 153 - protein, 7 aa, synthetic construct
TSGYFWN

SEQ ID NO: 154 - protein, 12 aa, synthetic construct
WMGYISYDGSNN

SEQ ID NO: 155 - protein, 12 aa, synthetic construct
ASPSPGTGYAVD

SEQ ID NO: 156 - protein, 16 aa, synthetic construct
RSSQSLVHSNRNTYLH

SEQ ID NO: 157 - protein, 7 aa, synthetic construct
KVSNRFS

SEQ ID NO: 158 - protein, 9 aa, synthetic construct
SQSTHVPFT

SEQ ID NO: 159 - protein, 16 aa, synthetic construct
RSSQSLVHSNRNTYLH

SEQ ID NO: 160 - protein, 7 aa, synthetic construct
KVSNRFS

SEQ ID NO: 161 - protein, 9 aa, synthetic construct
SQSTHVPFT

SEQ ID NO: 162 - protein, 16 aa, synthetic construct
RSSQSLVHSNRNTYLH

SEQ ID NO: 163 - protein, 7 aa, synthetic construct
KVSNRFS

SEQ ID NO: 164 - protein, 9 aa, synthetic construct
SQSTHVPFT

SEQ ID NO: 165 - protein, 11 aa, synthetic construct
QSLVHSNRNTY

SEQ ID NO: 166 - skipped

SEQ ID NO: 167 - protein, 9 aa, synthetic construct
SQSTHVPFT

SEQ ID NO: 168 - protein, 12 aa, synthetic construct
VHSNRNTYLHWY

SEQ ID NO: 169 - protein, 10 aa, synthetic construct
LLIYKVSNRF

SEQ ID NO: 170 - protein, 8 aa, synthetic construct
SQSTHVPF

SEQ ID NO: 171 - protein, 120 aa, synthetic construct
QVQLQESGPGLVKPSETLSLTCTVSGYSITSGYFWNWIRQFPGKGLEWMGYISYDGSNNYNPSLKSRITISRDTSKNQFSLKLSSVTAADTAVYYCASPSPGTGYAVDYWGQGTTVTVSS

SEQ ID NO: 172 - protein, 112 aa, synthetic construct
DIQMTQSPSSLSASVGDRVTITCRSSQSLVHSNRNTYLHWYQQKPGKAPKLLIYKVSNRFSGVPSRFSGSGSGTDFTLTISSLQPEDFATYYCSQSTHVPFTFGQGTKLEIK

SEQ ID NO: 173 - protein, 11 aa, synthetic construct
GYSITSGYFWN

SEQ ID NO: 174 - protein, 9 aa, synthetic construct
YISYDGSNN

SEQ ID NO: 175 - protein, 11 aa, synthetic construct
PSPGTGYAVDY

SEQ ID NO: 176 - protein, 6 aa, synthetic construct
SGYFWN

SEQ ID NO: 177 - protein, 16 aa, synthetic construct
YISYDGSNNYNPSLKS

SEQ ID NO: 178 - protein, 11 aa, synthetic construct
PSPGTGYAVDY

SEQ ID NO: 179 - protein, 8 aa, synthetic construct
GYSITSGY

SEQ ID NO: 180 - protein, 5 aa, synthetic construct
SYDGS

SEQ ID NO: 181 - protein, 11 aa, synthetic construct
PSPGTGYAVDY

SEQ ID NO: 182 - protein, 9 aa, synthetic construct
GYSITSGYF

SEQ ID NO: 183 - protein, 7 aa, synthetic construct
ISYDGSN

SEQ ID NO: 184 - protein, 13 aa, synthetic construct
ASPSPGTGYAVDY

SEQ ID NO: 185 - protein, 7 aa, synthetic construct
TSGYFWN

SEQ ID NO: 186 - protein, 12 aa, synthetic construct
WMGYISYDGSNN

SEQ ID NO: 187 - protein, 12 aa, synthetic construct
ASPSPGTGYAVD

SEQ ID NO: 188 - protein, 16 aa, synthetic construct
RSSQSLVHSNRNTYLH

SEQ ID NO: 189 - protein, 7 aa, synthetic construct
KVSNRFS

SEQ ID NO: 190 - protein, 9 aa, synthetic construct
SQSTHVPFT

SEQ ID NO: 191 - protein, 16 aa, synthetic construct
RSSQSLVHSNRNTYLH

SEQ ID NO: 192 - protein, 7 aa, synthetic construct
KVSNRFS

SEQ ID NO: 193 - protein, 9 aa, synthetic construct
SQSTHVPFT

SEQ ID NO: 194 - protein, 16 aa, synthetic construct
RSSQSLVHSNRNTYLH

SEQ ID NO: 195 - protein, 7 aa, synthetic construct
KVSNRFS

SEQ ID NO: 196 - protein, 9 aa, synthetic construct
SQSTHVPFT

SEQ ID NO: 197 - protein, 11 aa, synthetic construct
QSLVHSNRNTY

SEQ ID NO: 198 - skipped

SEQ ID NO: 199 - protein, 9 aa, synthetic construct
SQSTHVPFT

SEQ ID NO: 200 - protein, 12 aa, synthetic construct
VHSNRNTYLHWY

SEQ ID NO: 201 - protein, 10 aa, synthetic construct
LLIYKVSNRF

SEQ ID NO: 202 - protein, 8 aa, synthetic construct
SQSTHVPF

SEQ ID NO: 203 - protein, 120 aa, synthetic construct
QVQLQESGPGLVKPSETLSLTCTVSGYSITSGYFWNWIRQFPGKGLEWMGYISYDGSNNYNPSLKSRITISRDTSKNQFSLKLSSVTAADTAVYYCASPSPGTGYAVDYWGCGTTVTVSS

SEQ ID NO: 204 - protein, 112 aa, synthetic construct
DIQMTQSPSSLSASVGDRVTITCRSSQSLVHSNRNTYLHWYQQKPGCAPKLLIYKVSNRFSGVPSRFSGSGSGTDFTLTISSLQPEDFATYYCSQSTHVPFTFGQGTKLEIK

SEQ ID NO: 205 - protein, 11 aa, synthetic construct
GYSITSGYFWN

SEQ ID NO: 206 - protein, 9 aa, synthetic construct
YISYDGSNN

SEQ ID NO: 207 - protein, 11 aa, synthetic construct
PSPGTGYAVDY

SEQ ID NO: 208 - protein, 6 aa, synthetic construct
SGYFWN

SEQ ID NO: 209 - protein, 16 aa, synthetic construct
YISYDGSNNYNPSLKS

SEQ ID NO: 210 - protein, 11 aa, synthetic construct
PSPGTGYAVDY

SEQ ID NO: 211 - protein, 8 aa, synthetic construct
GYSITSGY

SEQ ID NO: 212 - protein, 5 aa, synthetic construct
SYDGS

SEQ ID NO: 213 - protein, 11 aa, synthetic construct
PSPGTGYAVDY

SEQ ID NO: 214 - protein, 9 aa, synthetic construct
GYSITSGYF

SEQ ID NO: 215 - protein, 7 aa, synthetic construct
ISYDGSN

SEQ ID NO: 216 - protein, 13 aa, synthetic construct
ASPSPGTGYAVDY

SEQ ID NO: 217 - protein, 7 aa, synthetic construct
TSGYFWN

SEQ ID NO: 218 - protein, 12 aa, synthetic construct
WMGYISYDGSNN

SEQ ID NO: 219 - protein, 12 aa, synthetic construct
ASPSPGTGYAVD

SEQ ID NO: 220 - protein, 16 aa, synthetic construct
RSSQSLVHSNRNTYLH

SEQ ID NO: 221 - protein, 7 aa, synthetic construct
KVSNRFS

SEQ ID NO: 222 - protein, 9 aa, synthetic construct
SQSTHVPFT

SEQ ID NO: 223 - protein, 16 aa, synthetic construct
RSSQSLVHSNRNTYLH

SEQ ID NO: 224 - protein, 7 aa, synthetic construct
KVSNRFS

SEQ ID NO: 225 - protein, 9 aa, synthetic construct
SQSTHVPFT

SEQ ID NO: 226 - protein, 16 aa, synthetic construct
RSSQSLVHSNRNTYLH

SEQ ID NO: 227 - protein, 7 aa, synthetic construct
KVSNRFS

SEQ ID NO: 228 - protein, 9 aa, synthetic construct
SQSTHVPFT

SEQ ID NO: 229 - protein, 11 aa, synthetic construct
QSLVHSNRNTY

SEQ ID NO: 230 - skipped

SEQ ID NO: 231 - protein, 9 aa, synthetic construct
SQSTHVPFT

SEQ ID NO: 232 - protein, 12 aa, synthetic construct
VHSNRNTYLHWY

SEQ ID NO: 233 - protein, 10 aa, synthetic construct
LLIYKVSNRF

SEQ ID NO: 234 - protein, 8 aa, synthetic construct
SQSTHVPF

SEQ ID NO: 235 - protein, 120 aa, synthetic construct
QVQLQESGPGLVKPSETLSLTCTVSGYSITSGYFWNWIRQFPGCGLEWMGYISYDGSNNYNPSLKSRITISRDTSKNQFSLKLSSVTAADTAVYYCASPSPGTGYAVDYWGQGTTVTVSS

SEQ ID NO: 236 - protein, 112 aa, synthetic construct
DIQMTQSPSSLSASVGDRVTITCRSSQSLVHSNRNTYLHWYQQKPGKAPKLLIYKVSNRFSGVPSRFSGSGSGTDFTLTISSLQPEDFATYYCSQSTHVPFTFGCGTKLEIK

SEQ ID NO: 237 - protein, 10 aa, synthetic construct
GGSISSYYWS

SEQ ID NO: 238 - protein, 9 aa, synthetic construct
YIYYSGSTN

SEQ ID NO: 239 - protein, 17 aa, synthetic construct
TTIFGVVTPNFYYGMDV

SEQ ID NO: 240 - protein, 5 aa, synthetic construct
SYYWS

SEQ ID NO: 241 - protein, 16 aa, synthetic construct
YIYYSGSTNYNPSLKS

SEQ ID NO: 242 - protein, 17 aa, synthetic construct
TTIFGVVTPNFYYGMDV

SEQ ID NO: 243 - protein, 7 aa, synthetic construct
GGSISSY

SEQ ID NO: 244 - protein, 5 aa, synthetic construct
YYSGS

SEQ ID NO: 245 - protein, 17 aa, synthetic construct
TTIFGVVTPNFYYGMDV

SEQ ID NO: 246 - protein, 8 aa, synthetic construct
GGSISSYY

SEQ ID NO: 247 - protein, 7 aa, synthetic construct
IYYSGST

SEQ ID NO: 248 - protein, 19 aa, synthetic construct
AGTTIFGVVTPNFYYGMDV

SEQ ID NO: 249 - protein, 6 aa, synthetic construct
SSYYWS

SEQ ID NO: 250 - protein, 12 aa, synthetic construct
WIGYIYYSGSTN

SEQ ID NO: 251 - protein, 18 aa, synthetic construct
AGTTIFGVVTPNFYYGMD

SEQ ID NO: 252 - protein, 11 aa, synthetic construct
RASQGISSYLA

SEQ ID NO: 253 - protein, 7 aa, synthetic construct
AASTLQS

SEQ ID NO: 254 - protein, 9 aa, synthetic construct
QQLNSYPLT

SEQ ID NO: 255 - protein, 11 aa, synthetic construct
RASQGISSYLA

SEQ ID NO: 256 - protein, 7 aa, synthetic construct
AASTLQS

SEQ ID NO: 257 - protein, 9 aa, synthetic construct
QQLNSYPLT

SEQ ID NO: 258 - protein, 11 aa, synthetic construct
RASQGISSYLA

SEQ ID NO: 259 - protein, 7 aa, synthetic construct
AASTLQS

SEQ ID NO: 260 - protein, 9 aa, synthetic construct
QQLNSYPLT

SEQ ID NO: 261 - protein, 6 aa, synthetic construct
QGISSY

SEQ ID NO: 262 - skipped

SEQ ID NO: 263 - protein, 9 aa, synthetic construct
QQLNSYPLT

SEQ ID NO: 264 - protein, 7 aa, synthetic construct
SSYLAWY

SEQ ID NO: 265 - protein, 10 aa, synthetic construct
FLIYAASTLQ

SEQ ID NO: 266 - protein, 8 aa, synthetic construct
QQLNSYPL

SEQ ID NO: 267 - protein, 125 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSS

SEQ ID NO: 268 - protein, 107 aa, synthetic construct
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIK

SEQ ID NO: 269 - protein, 10 aa, synthetic construct
GFTFSSYAMS

SEQ ID NO: 270 - protein, 10 aa, synthetic construct
AISGGIGSTY

SEQ ID NO: 271 - protein, 12 aa, synthetic construct
DAVGATPYYFDY

SEQ ID NO: 272 - protein, 5 aa, synthetic construct
SYAMS

SEQ ID NO: 273 - protein, 17 aa, synthetic construct
AISGGIGSTYYADSVKG

SEQ ID NO: 274 - protein, 12 aa, synthetic construct
DAVGATPYYFDY

SEQ ID NO: 275 - protein, 7 aa, synthetic construct
GFTFSSY

SEQ ID NO: 276 - protein, 6 aa, synthetic construct
SGGIGS

SEQ ID NO: 277 - protein, 12 aa, synthetic construct
DAVGATPYYFDY

SEQ ID NO: 278 - protein, 8 aa, synthetic construct
GFTFSSYA

SEQ ID NO: 279 - protein, 8 aa, synthetic construct
ISGGIGST

SEQ ID NO: 280 - protein, 14 aa, synthetic construct
AKDAVGATPYYFDY

SEQ ID NO: 281 - protein, 6 aa, synthetic construct
SSYAMS

SEQ ID NO: 282 - protein, 13 aa, synthetic construct
WVSAISGGIGSTY

SEQ ID NO: 283 - protein, 13 aa, synthetic construct
AKDAVGATPYYFD

SEQ ID NO: 284 - protein, 13 aa, synthetic construct
SGSSSNIGINYVS

SEQ ID NO: 285 - protein, 7 aa, synthetic construct
DNNKRPS

SEQ ID NO: 286 - protein, 11 aa, synthetic construct
GTWDSSLSAVV

SEQ ID NO: 287 - protein, 13 aa, synthetic construct
SGSSSNIGINYVS

SEQ ID NO: 288 - protein, 7 aa, synthetic construct
DNNKRPS

SEQ ID NO: 289 - protein, 11 aa, synthetic construct
GTWDSSLSAVV

SEQ ID NO: 290 - protein, 13 aa, synthetic construct
SGSSSNIGINYVS

SEQ ID NO: 291 - protein, 7 aa, synthetic construct
DNNKRPS

SEQ ID NO: 292 - protein, 11 aa, synthetic construct
GTWDSSLSAVV

SEQ ID NO: 293 - protein, 8 aa, synthetic construct
SSNIGINY

SEQ ID NO: 294 - skipped

SEQ ID NO: 295 - protein, 11 aa, synthetic construct
GTWDSSLSAVV

SEQ ID NO: 296 - protein, 9 aa, synthetic construct
IGINYVSWY

SEQ ID NO: 297 - protein, 10 aa, synthetic construct
LLIYDNNKRP

SEQ ID NO: 298 - protein, 10 aa, synthetic construct
GTWDSSLSAV

SEQ ID NO: 299 - protein, 121 aa, synthetic construct
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGQGTLVTVSS

SEQ ID NO: 300 - protein, 110 aa, synthetic construct
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVL

SEQ ID NO: 301 - protein, 10 aa, synthetic construct
GFTFSSYAMS

SEQ ID NO: 302 - protein, 10 aa, synthetic construct
AISGSGGSTY

SEQ ID NO: 303 - protein, 14 aa, synthetic construct
DEGYSSGHYYGMDV

SEQ ID NO: 304 - protein, 5 aa, synthetic construct
SYAMS

SEQ ID NO: 305 - protein, 17 aa, synthetic construct
AISGSGGSTYYADSVKG

SEQ ID NO: 306 - protein, 14 aa, synthetic construct
DEGYSSGHYYGMDV

SEQ ID NO: 307 - protein, 7 aa, synthetic construct
GFTFSSY

SEQ ID NO: 308 - protein, 6 aa, synthetic construct
SGSGGS

SEQ ID NO: 309 - protein, 14 aa, synthetic construct
DEGYSSGHYYGMDV

SEQ ID NO: 310 - protein, 8 aa, synthetic construct
GFTFSSYA

SEQ ID NO: 311 - protein, 8 aa, synthetic construct
ISGSGGST

SEQ ID NO: 312 - protein, 16 aa, synthetic construct
AKDEGYSSGHYYGMDV

SEQ ID NO: 313 - protein, 6 aa, synthetic construct
SSYAMS

SEQ ID NO: 314 - protein, 13 aa, synthetic construct
WVSAISGSGGSTY

SEQ ID NO: 315 - protein, 15 aa, synthetic construct
AKDEGYSSGHYYGMD

SEQ ID NO: 316 - protein, 12 aa, synthetic construct
RASQSISSSFLT

SEQ ID NO: 317 - protein, 7 aa, synthetic construct
GASSRAT

SEQ ID NO: 318 - protein, 10 aa, synthetic construct
QHYGSSPMYT

SEQ ID NO: 319 - protein, 12 aa, synthetic construct
RASQSISSSFLT

SEQ ID NO: 320 - protein, 7 aa, synthetic construct
GASSRAT

SEQ ID NO: 321 - protein, 10 aa, synthetic construct
QHYGSSPMYT

SEQ ID NO: 322 - protein, 12 aa, synthetic construct
RASQSISSSFLT

SEQ ID NO: 323 - protein, 7 aa, synthetic construct
GASSRAT

SEQ ID NO: 324 - protein, 10 aa, synthetic construct
QHYGSSPMYT

SEQ ID NO: 325 - protein, 7 aa, synthetic construct
QSISSSF

SEQ ID NO: 326 - skipped

SEQ ID NO: 327 - protein, 10 aa, synthetic construct
QHYGSSPMYT

SEQ ID NO: 328 - protein, 8 aa, synthetic construct
SSSFLTWY

SEQ ID NO: 329 - protein, 10 aa, synthetic construct
LLIYGASSRA

SEQ ID NO: 330 - protein, 9 aa, synthetic construct
QHYGSSPMY

SEQ ID NO: 331 - protein, 123 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSS

SEQ ID NO: 332 - protein, 109 aa, synthetic construct
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIK

SEQ ID NO: 333 - protein, 10 aa, synthetic construct
GFTFSSYAMS

SEQ ID NO: 334 - protein, 10 aa, synthetic construct
AISGGIGSTY

SEQ ID NO: 335 - protein, 12 aa, synthetic construct
DGVGATPYYFDY

SEQ ID NO: 336 - protein, 5 aa, synthetic construct
SYAMS

SEQ ID NO: 337 - protein, 17 aa, synthetic construct
AISGGIGSTYYADSVKG

SEQ ID NO: 338 - protein, 12 aa, synthetic construct
DGVGATPYYFDY

SEQ ID NO: 339 - protein, 7 aa, synthetic construct
GFTFSSY

SEQ ID NO: 340 - protein, 6 aa, synthetic construct
SGGIGS

SEQ ID NO: 341 - protein, 12 aa, synthetic construct
DGVGATPYYFDY

SEQ ID NO: 342 - protein, 8 aa, synthetic construct
GFTFSSYA

SEQ ID NO: 343 - protein, 8 aa, synthetic construct
ISGGIGST

SEQ ID NO: 344 - protein, 14 aa, synthetic construct
AKDGVGATPYYFDY

SEQ ID NO: 345 - protein, 6 aa, synthetic construct
SSYAMS

SEQ ID NO: 346 - protein, 13 aa, synthetic construct
WVSAISGGIGSTY

SEQ ID NO: 347 - protein, 13 aa, synthetic construct
AKDGVGATPYYFD

SEQ ID NO: 348 - protein, 13 aa, synthetic construct
SGSSSNIGINYVS

SEQ ID NO: 349 - protein, 7 aa, synthetic construct
DNNKRPS

SEQ ID NO: 350 - protein, 11 aa, synthetic construct
GTWDSSLSAVV

SEQ ID NO: 351 - protein, 13 aa, synthetic construct
SGSSSNIGINYVS

SEQ ID NO: 352 - protein, 7 aa, synthetic construct
DNNKRPS

SEQ ID NO: 353 - protein, 11 aa, synthetic construct
GTWDSSLSAVV

SEQ ID NO: 354 - protein, 13 aa, synthetic construct
SGSSSNIGINYVS

SEQ ID NO: 355 - protein, 7 aa, synthetic construct
DNNKRPS

SEQ ID NO: 356 - protein, 11 aa, synthetic construct
GTWDSSLSAVV

SEQ ID NO: 357 - protein, 8 aa, synthetic construct
SSNIGINY

SEQ ID NO: 358 - skipped

SEQ ID NO: 359 - protein, 11 aa, synthetic construct
GTWDSSLSAVV

SEQ ID NO: 360 - protein, 9 aa, synthetic construct
IGINYVSWY

SEQ ID NO: 361 - protein, 10 aa, synthetic construct
LLIYDNNKRP

SEQ ID NO: 362 - protein, 10 aa, synthetic construct
GTWDSSLSAV

SEQ ID NO: 363 - protein, 121 aa, synthetic construct
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDGVGATPYYFDYWGQGTLVTVSS

SEQ ID NO: 364 - protein, 110 aa, synthetic construct; identical to SEQ ID NO: 300

SEQ ID NO: 365 - protein, 10 aa, synthetic construct
GFTFSSYAMS

SEQ ID NO: 366 - protein, 10 aa, synthetic construct
AISGGIGSTY

SEQ ID NO: 367 - protein, 12 aa, synthetic construct
DAVGATPYYFDY

SEQ ID NO: 368 - protein, 5 aa, synthetic construct
SYAMS

SEQ ID NO: 369 - protein, 17 aa, synthetic construct
AISGGIGSTYYADSVKG

SEQ ID NO: 370 - protein, 12 aa, synthetic construct
DAVGATPYYFDY

SEQ ID NO: 371 - protein, 7 aa, synthetic construct
GFTFSSY

SEQ ID NO: 372 - protein, 6 aa, synthetic construct
SGGIGS

SEQ ID NO: 373 - protein, 12 aa, synthetic construct
DAVGATPYYFDY

SEQ ID NO: 374 - protein, 8 aa, synthetic construct
GFTFSSYA

SEQ ID NO: 375 - protein, 8 aa, synthetic construct
ISGGIGST

SEQ ID NO: 376 - protein, 14 aa, synthetic construct
AKDAVGATPYYFDY

SEQ ID NO: 377 - protein, 6 aa, synthetic construct
SSYAMS

SEQ ID NO: 378 - protein, 13 aa, synthetic construct
WVSAISGGIGSTY

SEQ ID NO: 379 - protein, 13 aa, synthetic construct
AKDAVGATPYYFD

SEQ ID NO: 380 - protein, 13 aa, synthetic construct
SGSSSNIGINYVS

SEQ ID NO: 381 - protein, 7 aa, synthetic construct
DNNKRPS

SEQ ID NO: 382 - protein, 11 aa, synthetic construct
GTWDSSLSAVV

SEQ ID NO: 383 - protein, 13 aa, synthetic construct
SGSSSNIGINYVS

SEQ ID NO: 384 - protein, 7 aa, synthetic construct
DNNKRPS

SEQ ID NO: 385 - protein, 11 aa, synthetic construct
GTWDSSLSAVV

SEQ ID NO: 386 - protein, 13 aa, synthetic construct
SGSSSNIGINYVS

SEQ ID NO: 387 - protein, 7 aa, synthetic construct
DNNKRPS

SEQ ID NO: 388 - protein, 11 aa, synthetic construct
GTWDSSLSAVV

SEQ ID NO: 389 - protein, 8 aa, synthetic construct
SSNIGINY

SEQ ID NO: 390 - skipped

SEQ ID NO: 391 - protein, 11 aa, synthetic construct
GTWDSSLSAVV

SEQ ID NO: 392 - protein, 9 aa, synthetic construct
IGINYVSWY

SEQ ID NO: 393 - protein, 10 aa, synthetic construct
LLIYDNNKRP

SEQ ID NO: 394 - protein, 10 aa, synthetic construct
GTWDSSLSAV

SEQ ID NO: 395 - protein, 121 aa, synthetic construct
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGCGTLVTVSS

SEQ ID NO: 396 - protein, 110 aa, synthetic construct
QSVLTQPPSVSAAPGQKVTISCSGSSSNIGINYVSWYQQLPGCAPKLLIYDNNKRPSGIPDRFSGSKSGTSATLGITGLQTGDEADYYCGTWDSSLSAVVFGGGTKLTVL

SEQ ID NO: 397 - protein, 12 aa, synthetic construct
GASISSPSYYWG

SEQ ID NO: 398 - protein, 9 aa, synthetic construct
SIFYSGSSY

SEQ ID NO: 399 - protein, 15 aa, synthetic construct
QSGVSGWYGAEYFQH

SEQ ID NO: 400 - protein, 7 aa, synthetic construct
SPSYYWG

SEQ ID NO: 401 - protein, 16 aa, synthetic construct
SIFYSGSSYYNPSLKS

SEQ ID NO: 402 - protein, 15 aa, synthetic construct
QSGVSGWYGAEYFQH

SEQ ID NO: 403 - protein, 9 aa, synthetic construct
GASISSPSY

SEQ ID NO: 404 - protein, 5 aa, synthetic construct
FYSGS

SEQ ID NO: 405 - protein, 15 aa, synthetic construct
QSGVSGWYGAEYFQH

SEQ ID NO: 406 - protein, 10 aa, synthetic construct
GASISSPSYY

SEQ ID NO: 407 - protein, 7 aa, synthetic construct
IFYSGSS

SEQ ID NO: 408 - protein, 17 aa, synthetic construct
ASQSGVSGWYGAEYFQH

SEQ ID NO: 409 - protein, 8 aa, synthetic construct
SSPSYYWG

SEQ ID NO: 410 - protein, 12 aa, synthetic construct
WIGSIFYSGSSY

SEQ ID NO: 411 - protein, 16 aa, synthetic construct
ASQSGVSGWYGAEYFQ

SEQ ID NO: 412 - protein, 11 aa, synthetic construct
RASQSISSWLA

SEQ ID NO: 413 - protein, 7 aa, synthetic construct
KASSLES

SEQ ID NO: 414 - protein, 9 aa, synthetic construct
QQYNSYSRT

SEQ ID NO: 415 - protein, 11 aa, synthetic construct
RASQSISSWLA

SEQ ID NO: 416 - protein, 7 aa, synthetic construct
KASSLES

SEQ ID NO: 417 - protein, 9 aa, synthetic construct
QQYNSYSRT

SEQ ID NO: 418 - protein, 11 aa, synthetic construct
RASQSISSWLA

SEQ ID NO: 419 - protein, 7 aa, synthetic construct
KASSLES

SEQ ID NO: 420 - protein, 9 aa, synthetic construct
QQYNSYSRT

SEQ ID NO: 421 - protein, 6 aa, synthetic construct
QSISSW

SEQ ID NO: 422 - skipped

SEQ ID NO: 423 - protein, 9 aa, synthetic construct
QQYNSYSRT

SEQ ID NO: 424 - protein, 7 aa, synthetic construct
SSWLAWY

SEQ ID NO: 425 - protein, 10 aa, synthetic construct
LLIYKASSLE

SEQ ID NO: 426 - protein, 8 aa, synthetic construct
QQYNSYSR

SEQ ID NO: 427 - protein, 125 aa, synthetic construct
QLQLQESGPGLVKPSETLSLTCTVSGASISSPSYYWGWIRQPPGKGLEWIGSIFYSGSSYYNPSLKSRVIMSVDTSKNQFSLKLSSVTAADTALYYCASQSGVSGWYGAEYFQHWGCGTLVTVSS

SEQ ID NO: 428 - protein, 107 aa, synthetic construct
DIQMTQSPSTLSASVGDRVTITCRASQSISSWLAWYQQKPGCAPKLLIYKASSLESGVPSRFSGSGSGTEFTLTISSLQPDDFATYYCQQYNSYSRTFGQGTKVEIK

SEQ ID NO: 429 - protein, 484 aa, synthetic construct
DIQMTQSPSSLSASVGDRVTITCRSSQSLVHSSGNTYLHWYQQKPGKAPKFLIYKVSNRFSGVPSRFSGSGSGTDFTLTISSLQPEDFATYYCSQSTHVPFTFGQGTKLEIKGGSEGKSSGSGSESKSTGGSQVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 430 - protein, 451 aa, synthetic construct
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDGVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 431 - protein, 216 aa, synthetic construct
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 432 - protein, 484 aa, synthetic construct
DIQMTQSPSSLSASVGDRVTITCRSSQSLVHSNRNTYLHWYQQKPGKAPKLLIYKVSNRFSGVPSRFSGSGSGTDFTLTISSLQPEDFATYYCSQSTHVPFTFGQGTKLEIKGGSEGKSSGSGSESKSTGGSQVQLQESGPGLVKPSETLSLTCTVSGYSITSGYFWNWIRQFPGKGLEWMGYISYDGSNNYNPSLKSRITISRDTSKNQFSLKLSSVTAADTAVYYCASPSPGTGYAVDYWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 433 - protein, 453 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNRFTQKSLSLSPGK

SEQ ID NO: 434 - protein, 216 aa, synthetic construct
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 435 - protein, 482 aa, synthetic construct
DIQMTQSPSSLSASVGDRVTITCRSSQSLVHSSGNTYLHWYQQKPGCAPKFLIYKVSNRFSGVPSRFSGSGSGTDFTLTISSLQPEDFATYYCSQSTHVPFTFGQGTKLEIKGGGSGGSGGCPPCGGSGGQVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 436 - protein, 455 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 437 - protein, 214 aa, synthetic construct
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 438 - protein, 450 aa, synthetic construct
QVQLQESGPGLVKPSETLSLTCTVSGYSITSGYFWNWIRQFPGKGLEWMGYISYDGSNNYNPSLKSRITISRDTSKNQFSLKLSSVTAADTAVYYCASPSPGTGYAVDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 439 - protein, 219 aa, synthetic construct
DIQMTQSPSSLSASVGDRVTITCRSSQSLVHSNRNTYLHWYQQKPGKAPKLLIYKVSNRFSGVPSRFSGSGSGTDFTLTISSLQPEDFATYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 440 - protein, 482 aa, synthetic construct
DIQMTQSPSTLSASVGDRVTITCRASQSISSWLAWYQQKPGCAPKLLIYKASSLESGVPSRFSGSGSGTEFTLTISSLQPDDFATYYCQQYNSYSRTFGQGTKVEIKGGGSGGSGGCPPCGGSGGQLQLQESGPGLVKPSETLSLTCTVSGASISSPSYYWGWIRQPPGKGLEWIGSIFYSGSSYYNPSLKSRVIMSVDTSKNQFSLKLSSVTAADTALYYCASQSGVSGWYGAEYFQHWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 441 - protein, 482 aa, synthetic construct
DIQMTQSPSSLSASVGDRVTITCRSSQSLVHSNRNTYLHWYQQKPGCAPKLLIYKVSNRFSGVPSRFSGSGSGTDFTLTISSLQPEDFATYYCSQSTHVPFTFGQGTKLEIKGGGSGGSGGCPPCGGSGGQVQLQESGPGLVKPSETLSLTCTVSGYSITSGYFWNWIRQFPGKGLEWMGYISYDGSNNYNPSLKSRITISRDTSKNQFSLKLSSVTAADTAVYYCASPSPGTGYAVDYWGCGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 442 - protein, 451 aa, synthetic construct
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 443 - protein, 216 aa, synthetic construct; identical to SEQ ID NO: 431

SEQ ID NO: 444 - protein, 450 aa, synthetic construct
QVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 445 - protein, 219 aa, synthetic construct
DIQMTQSPSSLSASVGDRVTITCRSSQSLVHSSGNTYLHWYQQKPGKAPKFLIYKVSNRFSGVPSRFSGSGSGTDFTLTISSLQPEDFATYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 446 - protein, 481 aa, synthetic construct
QSVLTQPPSVSAAPGQKVTISCSGSSSNIGINYVSWYQQLPGCAPKLLIYDNNKRPSGIPDRFSGSKSGTSATLGITGLQTGDEADYYCGTWDSSLSAVVFGGGTKLTVLGGGSGGSGGCPPCGGSGGEVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 447 - protein, 482 aa, synthetic construct
QVQLQESGPGLVKPSETLSLTCTVSGYSITSGYFWNWIRQPPGCGLEWIGYISYDGSNNYNPSLKSRVTISRDTSKNQFSLKLSSVTAADTAVYYCASPSPGTGYAVDYWGQGTLVTVSSGGGSGGSGGCPPCGGSGGDIQMTQSPSSLSASVGDRVTITCRSSQSLVHSSGNTYLHWYQQKPGKAPKFLIYKVSNRFSGVPSRFSGSGSGTDFTLTISSLQPEDFATYYCSQSTHVPFTFGCGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 448 - protein, 451 aa, synthetic construct; identical to SEQ ID NO: 442

SEQ ID NO: 449 - protein, 216 aa, synthetic construct; identical to SEQ ID NO: 431

SEQ ID NO: 450 - protein, 482 aa, synthetic construct; identical to SEQ ID NO: 447

SEQ ID NO: 451 - protein, 451 aa, synthetic construct; identical to SEQ ID NO: 442

SEQ ID NO: 452 - protein, 216 aa, synthetic construct; identical to SEQ ID NO: 431

(19) United States
(12) Patent Application Publication
(10) Pub. No.: US 2026/0234252 A1
(43) Pub. Date: Aug. 13, 2026

(54) ANTI-V BETA 17/ANTI-CD123 BISPECIFIC ANTIBODIES

(71) Applicant: Janssen Biotech, Inc., Horsham, PA (US)

(72) Inventors: Rajkumar GANESAN, Blue Bell, PA (US); Iqbal S. GREWAL, Newtown, PA (US); Manuel Alejandro SEPULVEDA, West Windsor, NJ (US)

(21) Appl. No.: 19/155,208

(22) PCT Filed: Feb. 9, 2024
(86) PCT No.: PCT/IB2024/051202

§ 371 Date: Aug. 8, 2025

Related U.S. Application Data
(60) Provisional application No. 63/484,143, filed on Feb. 9, 2023.
(60) Provisional application No. 63/534,373, filed on Aug. 24, 2023.

(51) Int. Cl. C07K 16/28; C07K 16/30; C07K 16/40; C12N 15/63

(52) U.S. Cl. CPC C07K 16/2809; C07K 16/2866; C07K 16/2878; C07K 16/3069; C07K 16/40; C12N 15/63; C07K 2317/14; C07K 2317/24; C07K 2317/31; C07K 2317/565; C07K 2317/567; C07K 2317/92

(57) ABSTRACT

Anti-Vβ 17 antibodies or antigen-binding fragments thereof are described. Also described are nucleic acids encoding the antibodies, compositions comprising the antibodies, methods of producing the antibodies, and methods of using the antibodies.

### CROSS-REFERENCE

This application claims the benefit of U.S. Provisional Patent Application No. 63/484,143, filed Feb. 9, 2023, and U.S. Provisional Patent Application No. 63/391,676, filed Jul. 22, 2022, and U.S. Provisional Patent Application No. 63/534,373, filed Aug. 24, 2023, the contents of which are incorporated herein by reference in their entireties.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### FIELD OF THE INVENTION

This invention relates to monoclonal anti-Vβ17 antibodies, nucleic acids and expression vectors encoding the antibodies, recombinant cells containing the vectors, and compositions comprising the antibodies. Methods of making the antibodies, and methods of using the antibodies to kill cancer cells, are also provided.

### REFERENCE TO SEQUENCE LISTING SUBMITTED ELECTRONICALLY

This application contains a sequence listing, which is submitted electronically in XML file format with a file name “PRD4260WOPCT1 Sequence Listing” and a creation date of Jan. 25, 2024, and having a size of 420,374 bytes. The sequence listing submitted electronically is part of the specification and is herein incorporated by reference in its entirety.

### BACKGROUND OF THE INVENTION

Cytotoxic T cells (e.g., CD8+ T cells) can be utilized to directly kill cancer cells. Finding a way to direct cytotoxic T cells to a cancer cell could lead to the killing of such cells and an inhibition of cancer cell propagation. It has been demonstrated that cytotoxic T cells can be activated against cancer cells expressing cancer-associated antigens, by bring said cytotoxic T cells into close proximity to the cancer cells for an extended period of time using a bispecific antibody that binds both the cytotoxic T cell and the cancer cell. A variety of potential complications to this approach of killing cancer cells exist, such as selecting T cell and cancer cell antigens that mediate T cell activation, selecting parental antibodies that will have adequate affinity to mediate binding in the context of a bispecific antibody, and choosing a cancer cell antigen that will activate T cells to act specifically against cancer cells, rather than elicit nonspecific T cell activation. These complications are only compounded in the context of attempting to activate T cells to destroy cancer cells in an animal subject.

### BRIEF SUMMARY OF THE INVENTION

Provided herein are antibodies capable of binding Vβ17, an antigen associated with T cells. Also provided herein are antibodies capable of binding Vβ17 and a second antigen associated with cancer cells, where the second antigen is selected from the group consisting of CD123, kallikrein related peptidase 2 (KLK2), prostate-specific membrane antigen (PSMA), and B-cell maturation antigen (BCMA). Cytotoxic T cells express T cell receptors that consist of α- and β-chains, such as Vβ17. It is hypothesized that a bispecific antibody binding to Vβ17 and a cancer-associated antigen, such as CD123, KLK2, PSMA, or BMCA, may direct a cytotoxic T cell to an antigen-expressing cancer cell. Utilizing a bispecific antibody of this sort to recruit, or redirect, the cytotoxic T cell to an antigen-expressing cancer cell and could allow the T cell to kill the cancer cell.

In an aspect, the present disclosure relates to isolated bispecific antibodies or antigen-binding fragments thereof that bind to any one of Vβ17 and CD123; Vβ17 and KLK2; Vβ17 and PSMA; and Vβ17 and BCMA.

Provided herein are isolated Vβ17 bispecific antibodies or antigen-binding fragments thereof. The isolated Vβ17 bispecific antibody or antigen-binding fragment thereof comprises:
a. a first heavy chain (HC1);
b. a second heavy chain (HC2);
c. a first light chain (LC1); and
d. a second light chain (LC2),
   wherein HC1 is associated with LC1 and HC2 is associated with LC2, and wherein HC1 comprises a heavy chain complementarity determining region 1 (HCDR1), HCDR2, and HCDR3 comprising the amino acid sequences of SEQ ID NO:45, SEQ ID NO:46, and SEQ ID NO:47, respectively, and LC1 comprises a light chain complementarity determining region 1 (LCDR1), LCDR2, and LCDR3 comprising the amino acid sequences of SEQ ID NO:60, SEQ ID NO:61, and SEQ ID NO:62, respectively, to form a binding site for a first antigen, and wherein HC2 and LC2 form a binding site for a second antigen. In certain embodiments, the binding site for the first antigen binds to a Vβ17 on the surface of a CD8+ or CD4+ T cell. In certain embodiments, the binding site for the second antigen binds to a tumor antigen present on the surface of a cancer cell. In an aspect, the HCDR1, HCDR2, and HCDR3 comprise amino acid sequences of SEQ ID NOs: 48, 49, and 50, respectively, and the LCDR1, LCDR2, and LCDR3 comprise amino acid sequences of SEQ ID NO: 63, 64, and 65, respectively. In an aspect, the HCDR1, HCDR2, and HCDR3 comprise amino acid sequences of SEQ ID NOs: 51, 52, and 53, respectively, and the LCDR1, LCDR2, and LCDR3 comprise amino acid sequences of SEQ ID NO: 66, 67, and 68, respectively. In an aspect, the HCDR1, HCDR2, and HCDR3 comprise amino acid sequences of SEQ ID NOs: 54, 55, and 56, respectively, and the LCDR1, LCDR2, and LCDR3 comprise amino acid sequences of SEQ ID NO: 69, KVS, and SEQ ID NO: 71, respectively. In an aspect, the HCDR1, HCDR2, and HCDR3 comprise amino acid sequences of SEQ ID NOs: 57, 58, and 59, respectively, and the LCDR1, LCDR2, and LCDR3 comprise amino acid sequences of SEQ ID NOs: 72, 73, and 74, respectively. In an aspect, the HCDR1, HCDR2, and HCDR3 comprise amino acid sequences of SEQ ID NOs: 77, 78, and 79, respectively, and the LCDR1, LCDR2, and LCDR3 comprise amino acid sequences of SEQ ID NOs: 92, 93, and 94, respectively. In an aspect, the HCDR1, HCDR2, and HCDR3 comprise amino acid sequences of SEQ ID NOs: 80, 81, and 82, respectively, and the LCDR1, LCDR2, and LCDR3 comprise amino acid sequences of SEQ ID NOs: 95, 96, and 97, respectively. In an aspect, the HCDR1, HCDR2, and HCDR3 comprise amino acid sequences of SEQ ID NOs: 83, 84, and 85, respectively, and the LCDR1, LCDR2, and LCDR3 comprise amino acid sequences of SEQ ID NOs: 98, 99, and 100, respectively. In an aspect, the HCDR1, HCDR2, and HCDR3 comprise amino acid sequences of SEQ ID NOs: 86, 87, and 88, respectively, and the LCDR1, LCDR2, and LCDR3 comprise amino acid sequences of SEQ ID NOs: 101, KVS, and SEQ ID NO: 103, respectively. In an aspect, the HCDR1, HCDR2, and HCDR3 comprise amino acid sequences of SEQ ID NOs: 89, 90, and 91, respectively, and the LCDR1, LCDR2, and LCDR3 comprise amino acid sequences of SEQ ID NOs: 104, 105, and 106, respectively. In an aspect, the HCDR1, HCDR2, and HCDR3 comprise amino acid sequences of SEQ ID NOs: 109, 110, and 111, respectively, and the LCDR1, LCDR2, and LCDR3 comprise amino acid sequences of SEQ ID NOs: 124, 125, and 126, respectively. In an aspect, the HCDR1, HCDR2, and HCDR3 comprise amino acid sequences of SEQ ID NOs: 112, 113, and 114, respectively, and the LCDR1, LCDR2, and LCDR3 comprise amino acid sequences of SEQ ID NOs: 127, 128, and 129, respectively. In an aspect, the HCDR1, HCDR2, and HCDR3 comprise amino acid sequences of SEQ ID NOs: 115, 116, and 117, respectively, and the LCDR1, LCDR2, and LCDR3 comprise amino acid sequences of SEQ ID NOs: 130, 131, and 132, respectively. In an aspect, the HCDR1, HCDR2, and HCDR3 comprise amino acid sequences of SEQ ID NOs: 118, 119, and 120, respectively, and the LCDR1, LCDR2, and LCDR3 comprise amino acid sequences of SEQ ID NO: 133, KVS, and SEQ ID NO: 135, respectively. In an aspect, the HCDR1, HCDR2, and HCDR3 comprise amino acid sequences of SEQ ID NOs: 121, 122, and 123, respectively, and the LCDR1, LCDR2, and LCDR3 comprise amino acid sequences of SEQ ID NOs: 136, 137, and 138, respectively. In an aspect, the HCDR1, HCDR2, and HCDR3 comprise amino acid sequences of SEQ ID NOs: 141, 142, and 143, respectively, and the LCDR1, LCDR2, and LCDR3 comprise amino acid sequences of SEQ ID NOs: 156, 157, and 158, respectively. In an aspect, the HCDR1, HCDR2, and HCDR3 comprise amino acid sequences of SEQ ID NOs: 144, 145, and 146, respectively, and the LCDR1, LCDR2, and LCDR3 comprise amino acid sequences of SEQ ID NOs: 159, 160, and 161, respectively. In an aspect, the HCDR1, HCDR2, and HCDR3 comprise amino acid sequences of SEQ ID NOs: 147, 148, and 149, respectively, and the LCDR1, LCDR2, and LCDR3 comprise amino acid sequences of SEQ ID NOs: 162, 163, and 164, respectively. In an aspect, the HCDR1, HCDR2, and HCDR3 comprise amino acid sequences of SEQ ID NOs: 150, 151, and 152, respectively, and the LCDR1, LCDR2, and LCDR3 comprise amino acid sequences of SEQ ID NO: 165, KVS, and SEQ ID NO: 167, respectively. In an aspect, the HCDR1, HCDR2, and HCDR3 comprise amino acid sequences of SEQ ID NOs: 153, 154, and 155, respectively, and the LCDR1, LCDR2, and LCDR3 comprise amino acid sequences of SEQ ID NOs: 168, 169, and 170, respectively. In an aspect, the HCDR1, HCDR2, and HCDR3 comprise amino acid sequences of SEQ ID NOs: 173, 174, and 175, respectively, and the LCDR1, LCDR2, and LCDR3 comprise amino acid sequences of SEQ ID NOs: 188, 189, and 190, respectively. In an aspect, the HCDR1, HCDR2, and HCDR3 comprise amino acid sequences of SEQ ID NOs: 176, 177, and 178, respectively, and the LCDR1, LCDR2, and LCDR3 comprise amino acid sequences of SEQ ID NOs: 191, 192, and 193, respectively. In an aspect, the HCDR1, HCDR2, and HCDR3 comprise amino acid sequences of SEQ ID NOs: 179, 180, and 181, respectively, and the LCDR1, LCDR2, and LCDR3 comprise amino acid sequences of SEQ ID NOs: 194, 195, and 196, respectively. In an aspect, the HCDR1, HCDR2, and HCDR3 comprise amino acid sequences of SEQ ID NOs: 182, 183, and 184, respectively, and the LCDR1, LCDR2, and LCDR3 comprise amino acid sequences of SEQ ID NOs: 197, KVS, and SEQ ID NO: 199, respectively. In an aspect, the HCDR1, HCDR2, and HCDR3 comprise amino acid sequences of SEQ ID NOs: 185, 186, and 187, respectively, and the LCDR1, LCDR2, and LCDR3 comprise amino acid sequences of SEQ ID NOs: 200, 201, and 202, respectively. In an aspect, the HCDR1, HCDR2, and HCDR3 comprise amino acid sequences of SEQ ID NOs: 205, 206, and 207, respectively, and the LCDR1, LCDR2, and LCDR3 comprise amino acid sequences of SEQ ID NOs: 220, 221, and 222, respectively. In an aspect, the HCDR1, HCDR2, and HCDR3 comprise amino acid sequences of SEQ ID NOs: 208, 209, and 210, respectively, and the LCDR1, LCDR2, and LCDR3 comprise amino acid sequences of SEQ ID NOs: 223, 224, and 225, respectively. In an aspect, the HCDR1, HCDR2, and HCDR3 comprise amino acid sequences of SEQ ID NOs: 211, 212, and 213, respectively, and the LCDR1, LCDR2, and LCDR3 comprise amino acid sequences of SEQ ID NOs: 226, 227, and 228, respectively. In an aspect, the HCDR1, HCDR2, and HCDR3 comprise amino acid sequences of SEQ ID NOs: 214, 215, and 216, respectively, and the LCDR1, LCDR2, and LCDR3 comprise amino acid sequences of SEQ ID NO: 229, KVS, and SEQ ID NO: 231, respectively. In an aspect, the HCDR1, HCDR2, and HCDR3 comprise amino acid sequences of SEQ ID NOs: 217, 218, and 219, respectively, and the LCDR1, LCDR2, and LCDR3 comprise amino acid sequences of SEQ ID NO: 232, 233, and 234, respectively.

In certain embodiments, the binding of the bispecific antibody to Vβ17 present on the surface of the CD8+ or CD4+ T cell and the binding of the tumor antigen present on the surface of the cancer cells results in the killing of the cancer cell.

In an aspect, the isolated Vβ17 bispecific antibodies or antigen-binding fragments thereof are isolated anti-Vβ17/anti-CD123 bispecific antibodies or antigen-binding fragments thereof. In an aspect, the isolated Vβ17 bispecific antibodies or antigen-binding fragments thereof are isolated anti-Vβ17/anti-KLK2 bispecific antibodies or antigen-binding fragments thereof. In an aspect, the isolated Vβ17 bispecific antibodies or antigen-binding fragments thereof are isolated anti-Vβ17/anti-PSMA bispecific antibodies or antigen-binding fragments thereof. In an aspect, the isolated Vβ17 bispecific antibodies or antigen-binding fragments thereof are isolated anti-Vβ17/anti-BCMA bispecific antibodies or antigen-binding fragments thereof. In certain aspects, HC2 and LC2 bind to CD123. In certain aspects, HC2 and LC2 bind to KLK2. In certain aspects, HC2 and LC2 bind to PSMA. In certain aspects, HC2 and LC2 bind to BCMA. In an aspect, the HC2 comprises a heavy chain complementarity determining region 1 (HCDR1-2), a heavy chain complementarity determining region 2 (HCDR2-2), and a heavy chain complementarity determining region 3 (HCDR3-2) comprising the amino acid sequences of SEQ ID NO:34, SEQ ID NO:35, and SEQ ID NO:36, respectively, and the LC2 comprises a light chain complementarity determining region 1 (LCDR1-2), a light chain complementarity determining region 2 (LCDR2-2), and a light chain complementarity determining region 3 (LCDR3-2) comprising the amino acid sequences of SEQ ID NO:37, SEQ ID NO:38, and SEQ ID NO: 39, respectively. In an aspect, the HCDR1-2, HCDR2-2, and HCDR3-2 comprise amino acid sequences of SEQ ID NOs: 237, 238, and 239, respectively, and the LCDR1-2, LCDR2-2, and LCDR3-2 comprise amino acid sequences of SEQ ID NOs: 252, 253, and 254, respectively. In an aspect, the HCDR1-2, HCDR2-2, and HCDR3-2 comprise amino acid sequences of SEQ ID NOs: 240, 241, and 242, respectively, and the LCDR1-2, LCDR2-2, and LCDR3-2 comprise amino acid sequences of SEQ ID NOs: 255, 256, and 257, respectively. In an aspect, the HCDR1-2, HCDR2-2, and HCDR3-2 comprise amino acid sequences of SEQ ID NOs: 243, 244, and 245, respectively, and the LCDR1-2, LCDR2-2, and LCDR3-2 comprise amino acid sequences of SEQ ID NOs: 258, 259, and 260, respectively. In an aspect, the HCDR1-2, HCDR2-2, and HCDR3-2 comprise amino acid sequences of SEQ ID NOs: 246, 247, and 248, respectively, and the LCDR1-2, LCDR2-2, and LCDR3-2 comprise amino acid sequences of SEQ ID NO: 261, AAS, and SEQ ID NO: 263, respectively. In an aspect, the HCDR1-2, HCDR2-2, and HCDR3-2 comprise amino acid sequences of SEQ ID NOs: 249, 250, and 251, respectively, and the LCDR1-2, LCDR2-2, and LCDR3-2 comprise amino acid sequences of SEQ ID NOs: 264, 265, and 266, respectively. In an aspect, the HCDR1-2, HCDR2-2, and HCDR3-2 comprise amino acid sequences of SEQ ID NOs: 269, 270, and 271, respectively, and the LCDR1-2, LCDR2-2, and LCDR3-2 comprise amino acid sequences of SEQ ID NOs: 284, 285, and 286, respectively. In an aspect, the HCDR1-2, HCDR2-2, and HCDR3-2 comprise amino acid sequences of SEQ ID NOs: 272, 273, and 274, respectively, and the LCDR1-2, LCDR2-2, and LCDR3-2 comprise amino acid sequences of SEQ ID NOs: 287, 288, and 289, respectively. In an aspect, the HCDR1-2, HCDR2-2, and HCDR3-2 comprise amino acid sequences of SEQ ID NOs: 275, 276, and 277, respectively, and the LCDR1-2, LCDR2-2, and LCDR3-2 comprise amino acid sequences of SEQ ID NOs: 290, 291, and 292, respectively. In an aspect, the HCDR1-2, HCDR2-2, and HCDR3-2 comprise amino acid sequences of SEQ ID NOs: 278, 279, and 280, respectively, and the LCDR1-2, LCDR2-2, and LCDR3-2 comprise amino acid sequences of SEQ ID NO: 293, DNN, and SEQ ID NO: 295, respectively. In an aspect, the HCDR1-2, HCDR2-2, and HCDR3-2 comprise amino acid sequences of SEQ ID NOs: 281, 282, and 283, respectively, and the LCDR1-2, LCDR2-2, and LCDR3-2 comprise amino acid sequences of SEQ ID NOs: 296, 297, and 298, respectively. In an aspect, the HCDR1-2, HCDR2-2, and HCDR3-2 comprise amino acid sequences of SEQ ID NOs: 301, 302, and 303, respectively, and the LCDR1-2, LCDR2-2, and LCDR3-2 comprise amino acid sequences of SEQ ID NOs: 316, 317, and 318, respectively. In an aspect, the HCDR1-2, HCDR2-2, and HCDR3-2 comprise amino acid sequences of SEQ ID NOs: 304, 305, and 306, respectively, and the LCDR1-2, LCDR2-2, and LCDR3-2 comprise amino acid sequences of SEQ ID NOs: 319, 320, and 321, respectively. In an aspect, the HCDR1-2, HCDR2-2, and HCDR3-2 comprise amino acid sequences of SEQ ID NOs: 307, 308, and 309, respectively, and the LCDR1-2, LCDR2-2, and LCDR3-2 comprise amino acid sequences of SEQ ID NOs: 322, 323, and 324, respectively. In an aspect, the HCDR1-2, HCDR2-2, and HCDR3-2 comprise amino acid sequences of SEQ ID NOs: 310, 311, and 312, respectively, and the LCDR1-2, LCDR2-2, and LCDR3-2 comprise amino acid sequences of SEQ ID NO: 325, GAS, and SEQ ID NO: 327, respectively. In an aspect, the HCDR1-2, HCDR2-2, and HCDR3-2 comprise amino acid sequences of SEQ ID NOs: 313, 314, and 315, respectively, and the LCDR1-2, LCDR2-2, and LCDR3-2 comprise amino acid sequences of SEQ ID NOs: 328, 329, and 330, respectively. In an aspect, the HCDR1-2, HCDR2-2, and HCDR3-2 comprise amino acid sequences of SEQ ID NOs: 333, 334, and 335, respectively, and the LCDR1-2, LCDR2-2, and LCDR3-2 comprise amino acid sequences of SEQ ID NOs: 348, 349, and 350, respectively. In an aspect, the HCDR1-2, HCDR2-2, and HCDR3-2 comprise amino acid sequences of SEQ ID NOs: 336, 337, and 338, respectively, and the LCDR1-2, LCDR2-2, and LCDR3-2 comprise amino acid sequences of SEQ ID NOs: 351, 352, and 353, respectively. In an aspect, the HCDR1-2, HCDR2-2, and HCDR3-2 comprise amino acid sequences of SEQ ID NOs: 339, 340, and 341, respectively, and the LCDR1-2, LCDR2-2, and LCDR3-2 comprise amino acid sequences of SEQ ID NOs: 354, 355, and 356, respectively. In an aspect, the HCDR1-2, HCDR2-2, and HCDR3-2 comprise amino acid sequences of SEQ ID NOs: 342, 343, and 344, respectively, and the LCDR1-2, LCDR2-2, and LCDR3-2 comprise amino acid sequences of SEQ ID NO: 357, DNN, and SEQ ID NO: 359, respectively. In an aspect, the HCDR1-2, HCDR2-2, and HCDR3-2 comprise amino acid sequences of SEQ ID NOs: 345, 346, and 347, respectively, and the LCDR1-2, LCDR2-2, and LCDR3-2 comprise amino acid sequences of SEQ ID NOs: 360, 361, and 362, respectively. In an aspect, the HCDR1-2, HCDR2-2, and HCDR3-2 comprise amino acid sequences of SEQ ID NOs: 365, 366, and 367, respectively, and the LCDR1-2, LCDR2-2, and LCDR3-2 comprise amino acid sequences of SEQ ID NOs: 380, 381, and 382, respectively. In an aspect, the HCDR1-2, HCDR2-2, and HCDR3-2 comprise amino acid sequences of SEQ ID NOs: 368, 369, and 370, respectively, and the LCDR1-2, LCDR2-2, and LCDR3-2 comprise amino acid sequences of SEQ ID NOs: 383, 384, and 385, respectively. In an aspect, the HCDR1-2, HCDR2-2, and HCDR3-2 comprise amino acid sequences of SEQ ID NOs: 371, 372, and 373, respectively, and the LCDR1-2, LCDR2-2, and LCDR3-2 comprise amino acid sequences of SEQ ID NOs: 386, 387, and 388, respectively. In an aspect, the HCDR1-2, HCDR2-2, and HCDR3-2 comprise amino acid sequences of SEQ ID NOs: 374, 375, and 376, respectively, and the LCDR1-2, LCDR2-2, and LCDR3-2 comprise amino acid sequences of SEQ ID NO: 389, DNN, and SEQ ID NO: 391, respectively. In an aspect, the HCDR1-2, HCDR2-2, and HCDR3-2 comprise amino acid sequences of SEQ ID NOs: 377, 378, and 379, respectively, and the LCDR1-2, LCDR2-2, and LCDR3-2 comprise amino acid sequences of SEQ ID NOs: 392, 393, and 394, respectively. In an aspect, the HCDR1-2, HCDR2-2, and HCDR3-2 comprise amino acid sequences of SEQ ID NOs: 397, 398, and 399, respectively, and the LCDR1-2, LCDR2-2, and LCDR3-2 comprise amino acid sequences of SEQ ID NOs: 412, 413, and 414, respectively. In an aspect, the HCDR1-2, HCDR2-2, and HCDR3-2 comprise amino acid sequences of SEQ ID NOs: 400, 401, and 402, respectively, and the LCDR1-2, LCDR2-2, and LCDR3-2 comprise amino acid sequences of SEQ ID NOs: 415, 416, and 417, respectively. In an aspect, the HCDR1-2, HCDR2-2, and HCDR3-2 comprise amino acid sequences of SEQ ID NOs: 403, 404, and 405, respectively, and the LCDR1-2, LCDR2-2, and LCDR3-2 comprise amino acid sequences of SEQ ID NOs: 418, 419, and 420, respectively. In an aspect, the HCDR1-2, HCDR2-2, and HCDR3-2 comprise amino acid sequences of SEQ ID NOs: 404, 405, and 406, respectively, and the LCDR1-2, LCDR2-2, and LCDR3-2 comprise amino acid sequences of SEQ ID NO: 421, KAS, and SEQ ID NO: 423, respectively. In an aspect, the HCDR1-2, HCDR2-2, and HCDR3-2 comprise amino acid sequences of SEQ ID NOs: 407, 408, and 409, respectively, and the LCDR1-2, LCDR2-2, and LCDR3-2 comprise amino acid sequences of SEQ ID NOs: 424, 425, and 426, respectively. In certain embodiments, the Vβ17 is on the surface of a CD8+ or CD4+ T cell. In certain embodiments, the CD123 is on the surface of a cancer cell. In certain embodiments, the KLK2 is on the surface of a cancer cell. In certain embodiments, the PSMA is on the surface of a cancer cell. In certain embodiments, the BCMA is on the surface of a cancer cell.

In certain embodiments, the bispecific antibody or antigen-binding fragment thereof is an IgG isotype, such as IgG4 or IgG1.

In certain embodiments, the bispecific antibody or antigen-binding fragment thereof induces CD8+ or CD4+ T-cell dependent cytotoxicity of a cancer cell in vitro with an EC₅₀ of less than about 0.2 picomolar (pM).

In certain embodiments, the anti-Vβ17/anti-CD123 bispecific antibodies or antigen-binding fragments thereof are chimeric, partially humanized, or fully humanized.

In certain embodiments, the anti-Vβ17/anti-KLK2 bispecific antibodies or antigen-binding fragments thereof are chimeric, partially humanized, or fully humanized.

In certain embodiments, the anti-Vβ17/anti-PSMA bispecific antibodies or antigen-binding fragments thereof are chimeric, partially humanized, or fully humanized.

In certain embodiments, the anti-Vβ17/anti-BCMA bispecific antibodies or antigen-binding fragments thereof are chimeric, partially humanized, or fully humanized.

Also provided are isolated humanized Vβ17 monoclonal antibodies or antigen-binding fragments thereof. In an aspect, the isolated humanized Vβ17 monoclonal antibody or antigen-binding fragment thereof can comprise an amino acid sequence with at least 95% identity to the amino acid sequence of SEQ ID NO:28. In a certain aspect, the isolated humanized Vβ17 monoclonal antibody or antigen-binding fragment thereof comprises an amino acid sequence of SEQ ID NO:28. In aspects, the isolated humanized Vβ17 monoclonal antibody or antigen-binding fragment thereof can comprise a heavy chain (HC) having at least 95% identity to an amino acid sequence selected from the group consisting of SEQ ID NOs: 429, 432, 435, 438, 441, 444, 447, and 450. In aspects, the isolated humanized Vβ17 monoclonal antibody or antigen-binding fragment thereof can comprise a heavy chain (HC) having an amino acid sequence selected from the group consisting of SEQ ID NOs: 429, 432, 435, 438, 441, 444, 447, and 450. In aspects, the isolated humanized Vβ17 monoclonal antibody or antigen-binding fragment thereof can comprise a light chain (LC) having at least 95% identity to an amino acid sequence selected from the group consisting of SEQ ID NOs: 431, 434, 437, 439, 443, 445, 449, and 452. In aspects, the isolated humanized Vβ17 monoclonal antibody or antigen-binding fragment thereof can comprise a light chain (LC) having an amino acid sequence selected from the group consisting of SEQ ID NOs: 431, 434, 437, 439, 443, 445, 449, and 452. In aspects, the isolated humanized Vβ17 monoclonal antibody or antigen-binding fragment thereof can comprise a heavy chain (HC) having at least 95% identity to an amino acid sequence selected from the group consisting of SEQ ID NOs: 429, 432, 435, 438, 441, 444, 447, and 450, and a light chain (LC) having at least 95% identity to an amino acid sequence selected from the group consisting of SEQ ID NOs: 431, 434, 437, 439, 443, 445, 449, and 452.

Also provided are isolated nucleic acids encoding the monoclonal antibodies or antigen-binding fragments thereof and the bispecific antibodies or antigen-binding fragments thereof disclosed herein.

Also provided are vectors comprising the isolated nucleic acids encoding the monoclonal antibodies or antigen-binding fragments thereof and the bispecific antibodies or antigen-binding fragments thereof disclosed herein.

Also provided are host cells comprising the vectors comprising the isolated nucleic acids disclosed herein.

Also provided are methods of directing a Vβ17-expressing CD8+ or CD4+ T cell to a cancer cell. In aspects, the methods comprise contacting a Vβ17-expressing CD8+ or CD4+ T cell with anti-Vβ17/anti-CD123 bispecific antibodies or antigen-binding fragments thereof disclosed herein, where contacting the Vβ17-expressing CD8+ or CD4+ T cell with the anti-Vβ17/anti-CD123 bispecific antibodies or antigen-binding fragments thereof can direct the Vβ17-expressing CD8+ or CD4+ T cell to a cancer cell. In aspects, the methods comprise contacting a Vβ17-expressing CD8+ or CD4+ T cell with anti-Vβ17/anti-KLK2 bispecific antibodies or antigen-binding fragments thereof disclosed herein, wherein contacting the Vβ17-expressing CD8+ or CD4+ T cell with the anti-Vβ17/anti-KLK2 bispecific antibodies or antigen-binding fragments thereof can direct the Vβ17-expressing CD8+ or CD4+ T cell to a cancer cell. In aspects, the methods comprise contacting a Vβ17-expressing CD8+ or CD4+ T cell with anti-Vβ17/anti-PSMA bispecific antibodies or antigen-binding fragments thereof disclosed herein, wherein contacting the Vβ17-expressing CD8+ or CD4+ T cell with the anti-Vβ17/anti-PSMA bispecific antibodies or antigen-binding fragments thereof can direct the Vβ17-expressing CD8+ or CD4+ T cell to a cancer cell. In aspects, the methods comprise contacting a Vβ17-expressing CD8+ or CD4+ T cell with anti-Vβ17/anti-BCMA bispecific antibodies or antigen-binding fragments thereof disclosed herein, wherein contacting the Vβ17-expressing CD8+ or CD4+ T cell with the anti-Vβ17/anti-BCMA bispecific antibodies or antigen-binding fragments thereof can direct the Vβ17-expressing CD8+ or CD4+ T cell to a cancer cell.

Also provided are methods for inhibiting growth or proliferation of cancer cells. The methods comprise contacting the cancer cells with the bispecific antibodies disclosed herein. Contacting the cancer cells with the described antibodies can, for example, inhibit the growth or proliferation of the cancer cells, or promote T cell mediated killing of the cancer cells.

Also provided are methods of producing the bispecific antibodies or antigen-binding fragments thereof disclosed herein. The methods comprise culturing a cell comprising a nucleic acid encoding one heavy and light chain pair of the bispecific antibody under conditions to produce the heavy and light chains or an antigen-binding fragment thereof, and recovering the heavy and light chains of the bispecific antibody or an antigen-binding fragment thereof from the cell or culture. Following collection of heavy and light chains for both arms of the bispecific antibody, the heavy and light chain pairs are mixed in conditions suitable to allow for self-assembly, after which the self-assembled bispecific antibodies are collected.

Also provided are methods of producing compositions comprising the bispecific antibodies or antigen-binding fragments disclosed herein, such as buffered compositions or purified compositions and the like. For example, the methods may comprise combining the bispecific antibody or antigen-binding fragment thereof with a buffer acceptable that is acceptable for storage and use of the bispecific antibody.

Also provided are kits comprising bispecific antibodies or antigen-binding fragments thereof disclosed herein and packaging for the same.

Also provided are antibodies, e.g., monospecific and multispecific, that comprise one or more the CDRs or CDR combinations as disclosed throughout.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a schematic demonstrating the binding of an anti-Vβ17/anti-tumor antigen bispecific antibody to recruit T-cells to a cancer cell and to induce cancer cell death.

FIGS. 2A-2B shows that Vβ17+ CD8+ T cells exist in healthy subjects and upon culture with M1 peptide these cells can be expanded in vitro. FIG. 2A shows FACS histograms of gated peripheral blood mononuclear cells (PBMCs) for CD8+ T cells expressing Vβ17 (Vβ17+) on the cells surface from healthy subjects. FIG. 2B shows HLA sub-type of various donors and presence of percent Vβ17+CD8+ T cells identified as day 0, and after in vitro expansion with M1 peptide for 14 days (Day 14).

FIG. 3 shows Vβ17+CD8+ T cells have hallmarks of killer cytotoxic cells. Bar graph indicates expression of CD107a, CD69, Granzyme B (Gzb) and Interferon-γ (IFNγ) on gated PBMCs for CD8+ T cells expressing Vβ17 (Vβ17+) on the cell surface at day 0 (no M1) and at day 14 after stimulation with M1 peptide (+M1).

FIG. 4 shows binding of VB11 [anti-Vβ17/anti-CD123] bispecific as well as VB13 [Vβ17 null control bispecific] antibodies to CD8+ T cells. Data presented from CD8+ T cells isolated from PBMCs from 3 different donors (D203517, HPU09381 and HPU08694). The table below each graph presents EC₅₀ values for binding in nanomolar (nM).

FIG. 5 shows binding of Vβ17 and CD123 bispecific (VB11) as well as Vβ17 null control bispecific (VB13) antibodies to AML cancer cell line. Data presented shows binding of bispecific antibodies to Kasumi3 AML cell line. The table below the graph presents EC₅₀ values for binding in nM.

FIG. 6 shows redirection of Vβ17+ T cells by bispecific antibodies that induce efficient killing of AML cancer cells. Data in the left graph shows killing of Kasumi3 cancer cells at an effector to target (E:T) ratio 0.5:1 and dose titration of bispecific antibodies. Data in the middle graph shows killing of Kasumi3 cancer cells at an E:T ratio 1:1 and dose titration of bispecific antibodies. Data in the right graph shows killing of Kasumi3 cancer cells at an E:T ratio 5:1 and dose titration of bispecific antibodies. The table below the graphs shows EC₅₀ values calculated from the above graphs given in picomolar (pM).

FIGS. 7A-7B show specific binding of an anti-Vβ17/anti-CD123 bispecific antibody (VB11) and a Vβ17 null bispecific antibody (VB13) to CD8+ T cells isolated from PBMCs. FIG. 7A shows FACS histograms of gated PBMCs for CD8+ T cells expressing Vβ17 (Vβ17+) on the cell surface from healthy subjects (left graph, Vβ17 non-depleted) and from PBMCs that were depleted of Vβ17+ T cells using negative selection (right graph, Vβ17 depleted). FIG. 7B shows specific binding of an anti-Vβ17/anti-CD123 bispecific antibody (VB11) and a Vβ17 null bispecific antibody (VB13) to CD8+ T cells from FIG. 7A. A dose response of bispecific antibodies is shown in the figure. The table below the graph shows EC₅₀ values for binding calculated from the above graph given in nM.

FIG. 8 shows specific recruitment of Vβ17 T cells by a Vβ17-bispecific antibody for killing of Kasumi3 cancer cells. Left figure shows killing of Kasumi3 AML cell line when effectors cells were isolated from PBMCs containing CD8+ T cells expressing Vβ17 (Vβ17+) on the cell surface (untouched CD8 T cells). Insert shows presence of 10.1% Vβ17+CD8 T cells in the effector cell population. Right figure shows killing of Kasumi3 AML cell line when effector CD8+ T cells were isolated from PBMCs, but Vβ17+ T cells were depleted by negative selection. Insert shows presence of a minor population (0.086%) Vβ17+CD8+ T cells in the effector cell population.

FIGS. 9A-9B show that there is no pan activation of T cells when using Vβ17 bispecific antibodies. FIG. 9A shows FACS plots of Vβ17+ and Vβ17-gated CD8+ T cells. When T cells were activated with Vβ17 bispecific antibody there was high level of upregulation of CD69 (62.5%) on Vβ17+ as compared to Vβ17-CD8+ T cells (1.80%). FIG. 9B shows a bar graph for upregulation of CD69 on Vβ17+ and Vβ17-gated CD8+ T cells when activated using Vβ17 bispecific antibody.

FIG. 10 shows that Vβ17+ T cells from HLA A2 negative donor are also effector killer cells and no pre-stimulation of Vβ17+ cell required. Efficient cytotoxicity mediated by Vβ17 bispecific antibody of Kasumi3 cancer cells is shown from PBMCs containing Vβ17+ T cells from HLA A2 negative donor (HPU 09381).

FIG. 11 shows VH and VL amino acid sequences for parent, LH spFv, and HL spFv versions of (1) a Vβ17 binder having an N33S mutation, and (2) a Vβ17 binder having a G34R mutation. Also shown are VH-CDR1, VH-CDR2, VH-CDR3, VL-CDR1, VL-CDR2, and VL-CDR3 sequences for the two Vβ17 binders.

### DETAILED DESCRIPTION OF THE INVENTION

Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

It must be noted that as used herein and in the appended claims, the singular forms “a,” “an,” and “the” include plural reference unless the context clearly dictates otherwise.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., anti-Vβ17/anti-CD123 bispecific antibodies and polynucleotides that encode them, Vβ17 polypeptides and Vβ17 polynucleotides that encode them, CD123 polypeptides and CD123 polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1997) Nucleic Acids Res. 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

### Antibodies

Described herein are isolated anti-Vβ17 bispecific antibodies or antigen-binding fragments thereof, nucleic acids and expression vectors encoding the antibodies, recombinant cells containing the vectors, and compositions comprising the antibodies. In an aspect, the invention relates to isolated anti-Vβ17/anti-CD123 bispecific antibodies or antigen-binding fragments thereof, nucleic acids and expression vectors encoding the antibodies, recombinant cells containing the vectors, and compositions comprising the bispecific antibodies. In an aspect, the invention relates to isolated anti-Vβ17/anti-KLK2 bispecific antibodies or antigen-binding fragments thereof, nucleic acids and expression vectors encoding the antibodies, recombinant cells containing the vectors, and compositions comprising the bispecific antibodies. In an aspect, the invention relates to isolated anti-Vβ17/anti-PSMA bispecific antibodies or antigen-binding fragments thereof, nucleic acids and expression vectors encoding the antibodies, recombinant cells containing the vectors, and compositions comprising the bispecific antibodies. In an aspect, the invention relates to isolated anti-Vβ17/anti-BCMA bispecific antibodies or antigen-binding fragments thereof, nucleic acids and expression vectors encoding the antibodies, recombinant cells containing the vectors, and compositions comprising the bispecific antibodies. Methods of making the antibodies, and methods of using the antibodies to treat diseases, including cancer, are also provided herein. The antibodies disclosed herein possess one or more desirable functional properties, including but not limited to high-affinity binding to Vβ17 and/or CD123, high specificity to Vβ17 and/or CD123, high-affinity binding to Vβ17 and/or KLK2, high specificity to Vβ17 and/or KLK2, high-affinity binding to Vβ17 and/or PSMA, high specificity to Vβ17 and/or PSMA, high-affinity binding to Vβ17 and/or BCMA, high specificity to Vβ17 and/or BCMA, and the ability to treat or prevent cancer when administered alone or in combination with other anti-cancer therapies.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgAQ1, IgA2, IgG1, IgG2, IgG3 and IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies disclosed herein include heavy and/or light chain constant regions from mouse or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to Vβ17 is substantially free of antibodies that do not bind to Vβ17; an isolated antibody that specifically binds to CD123 is substantially free of antibodies that do not bind to CD123; an isolated antibody that specifically binds to KLK2 is substantially free of antibodies that do not bind to KLK2; an isolated antibody that specifically binds to PSMA is substantially free of antibodies that do not bind to PSMA; and/or an isolated antibody that specifically binds to BCMA is substantially free of antibodies that do not bind to BCMA). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies disclosed herein can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv) 2, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′).

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin heavy and light chain pair which has binding specificity for a first epitope (e.g., an epitope on a Vβ17 antigen) and a second immunoglobulin heavy and light chain pair that has binding specificity for a second epitope (e.g., an epitope on a CD123 antigen, an epitope of a KLK2 antigen, an epitope of a PSMA antigen, or an epitope of a BCMA antigen). In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on Vβ17 and the second epitope is located on CD123. In an embodiment, the first epitope is located on Vβ17 and the second epitope is located on KLK2. In an embodiment, the first epitope is located on Vβ17 and the second epitope is located on PSMA. In an embodiment, the first epitope is located on Vβ17 and the second epitope is located on BCMA. In an embodiment, the first epitope is located on Vβ17 and the second epitope is located on PD-1, PD-L1, CTLA-4, EGFR, HER-2, CD19, CD20, CD3 and/or other tumor associated immune suppressors or surface antigens.

The term “half antibody” as used herein refers to one immunoglobulin heavy chain associated with one immunoglobulin light chain. An exemplary half-antibody is depicted in SEQ ID NO: 28. One skilled in the art will readily appreciate that a half-antibody can encompass a fragment thereof and can also have an antigen binding domain consisting of a single variable domain, e.g., originating from a camelidae.

As used herein, the term “Vβ17” refers to a T cell receptor, which is expressed in response to an immune response on a cytotoxic T cell. Vβ17-expressing CD8+ T cells are commonly produced in response to influenza A virus exposure in a subject. Vβ17-expressing CD8+ T cells provide great recall in response to influenza exposure in the subject. The term “Vβ17” includes any Vβ17 variant, isoform, and species homolog, which is naturally expressed by cells (including T cells) or can be expressed on cells transfected with genes or cDNA encoding the polypeptide. Unless noted, preferably the Vβ17 is a human Vβ17. A human Vβ17 amino acid sequence is provided by GenBank Accession Number AAB49730.1.

The term “CD123” refers to a molecule that is found on cells which helps transmit the signal of interleukin-3, a soluble cytokine that is important in the immune system. CD123 can also be referred to as the “interleukin-3 receptor.” The receptor belongs to the type I cytokine receptor family and is a heterodimer with a unique alpha chain paired with the common beta subunit (beta c or CD131). The CD123 receptor can be found on pluripotent progenitor cells and can induce tyrosine phosphorylation within the cell and promote proliferation and differentiation within hematopoietic cell lines. CD123 can also be expressed in acute myeloid leukemia (AML) subtypes. The term “CD123” includes any CD123 variant, isoform, and species homolog, which is naturally expressed by cells (including T cells) or can be expressed on cells transfected with genes or cDNA encoding those polypeptides. Unless noted, preferably the “CD123” is a human CD123. A human CD123 amino acid sequence is provided by GenBank Accession Number AY789109.1.

The term “KLK2” refers to kallikrein-related peptidase-2, a serine protease present in prostatic tissue and seminal fluid. KLK2 can also be referred to as hK2, or human glandular kallikrein 1. KLK2 is aberrantly expressed in a range of human cancer types, including prostate cancer. The term “KLK2” includes any KLK2 variant, isoform, and species homolog, which is naturally expressed by cells (including T cells) or can be expressed on cells transfected with genes or cDNA encoding those polypeptides. Unless noted, preferably the “KLK2” is a human KLK2. A human KLK2 amino acid sequence is provided by Accession Number P20151.

The term “PSMA” refers to prostate-specific membrane antigen. PMSA is encoded by FOLH1, is highly expressed in the prostate, and is upregulated in cancerous prostate cells compared to non-cancerous prostate cells. PMSA is also a molecular target for diagnosis and treatment of prostate cancer by positron emission tomography (PET) scanning. PSMA can also be referred to as glutamate carboxypeptidase II, N-acetyl-L-aspartyl-L-glutamate peptidase I (NAALADase I), or NAAG peptidase. The term “PSMA” includes any PSMA variant, isoform, and species homolog, which is naturally expressed by cells (including T cells) or can be expressed on cells transfected with genes or cDNA encoding those polypeptides. Unless noted, preferably the “PSMA” is a human PSMA. A human KLK2 amino acid sequence is provided by Accession Number NP_004467.1.

The term “BCMA” refers to B-cell maturation antigen, which is a cell surface receptor of the tumor necrosis factor receptor family that is preferentially expressed in mature B-lymphocytes. BCMA is also highly expressed in multiple myeloma. BCMA can also be referred to as BCM or tumor necrosis factor receptor superfamily member 17 (TNFRSF17). The term “BCMA” includes any BCMA variant, isoform, and species homolog, which is naturally expressed by cells (including T cells) or can be expressed on cells transfected with genes or cDNA encoding those polypeptides. Unless noted, preferably the “BCMA” is a human PSMA. A human BCMA amino acid sequence is provided by Accession Number Q02223.

As used herein, an antibody that “specifically binds to Vβ17” refers to an antibody that binds to a Vβ17, preferably a human Vβ17, with a K_{D} of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

As used herein, an antibody that “specifically binds to CD123” refers to an antibody that binds to a CD123, preferably a human CD123, with a K_{D} of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. As used herein, an antibody that “specifically binds to KLK2” refers to an antibody that binds to a KLK2, preferably a human KLK2, with a K_{D} of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. As used herein, an antibody that “specifically binds to PSMA” refers to an antibody that binds to a PSMA, preferably a human PSMA, with a K_{D} of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. As used herein, an antibody that “specifically binds to BCMA” refers to an antibody that binds to a BCMA, preferably a human BCMA, with a K_{D} of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

The smaller the value of the KD of an antibody, the higher affinity that the antibody binds to a target antigen.

In an aspect, the invention relates to an isolated Vβ17 bispecific antibody or antigen-binding fragment thereof comprising (a) a first heavy chain (HC1); (b) a second heavy chain (HC2); (c) a first light chain (LC1); and (d) a second light chain (LC2). The HC1 can be associated with the LC1 and the HC2 can be associated with LC2. The HC1 can comprise a heavy chain complementarity determining region 1 (HCDR1), HCDR2, and HCDR3 comprising the amino acid sequences of SEQ ID NO:45, SEQ ID NO:46, and SEQ ID NO:47, respectively, and LC1 can comprise a light chain complementarity determining region 1 (LCDR1), LCDR2, and LCDR3 comprising the amino acid sequences of SEQ ID NO:60, SEQ ID NO:61, and SEQ ID NO: 62, respectively. In an aspect, the HCDR1, HCDR2, and HCDR3 comprise amino acid sequences of SEQ ID NOs: 48, 49, and 50, respectively, and the LCDR1, LCDR2, and LCDR3 comprise amino acid sequences of SEQ ID NOs: 63, 64, and 65, respectively. In an aspect, the HCDR1, HCDR2, and HCDR3 comprise amino acid sequences of SEQ ID NOs: 51, 52, and 53, respectively, and the LCDR1, LCDR2, and LCDR3 comprise amino acid sequences of SEQ ID NOs: 66, 67, and 68, respectively. In an aspect, the HCDR1, HCDR2, and HCDR3 comprise amino acid sequences of SEQ ID NOs: 54, 55, and 56, respectively, and the LCDR1, LCDR2, and LCDR3 comprise amino acid sequences of SEQ ID NO: 69, KVS, and SEQ ID NO: 71, respectively. In an aspect, the HCDR1, HCDR2, and HCDR3 comprise amino acid sequences of SEQ ID NOs: 57, 58, and 59, respectively, and the LCDR1, LCDR2, and LCDR3 comprise amino acid sequences of SEQ ID NOs: 72, 73, and 74, respectively. In an aspect, the HCDR1, HCDR2, and HCDR3 comprise amino acid sequences of SEQ ID NOs: 77, 78, and 79, respectively, and the LCDR1, LCDR2, and LCDR3 comprise amino acid sequences of SEQ ID NOs: 92, 93, and 94, respectively. In an aspect, the HCDR1, HCDR2, and HCDR3 comprise amino acid sequences of SEQ ID NOs: 80, 81, and 82, respectively, and the LCDR1, LCDR2, and LCDR3 comprise amino acid sequences of SEQ ID NOs: 95, 96, and 97, respectively. In an aspect, the HCDR1, HCDR2, and HCDR3 comprise amino acid sequences of SEQ ID NOs: 83, 84, and 85, respectively, and the LCDR1, LCDR2, and LCDR3 comprise amino acid sequences of SEQ ID NOs: 98, 99, and 100, respectively. In an aspect, the HCDR1, HCDR2, and HCDR3 comprise amino acid sequences of SEQ ID NOs: 86, 87, and 88, respectively, and the LCDR1, LCDR2, and LCDR3 comprise amino acid sequences of SEQ ID NO: 101, KVS, and SEQ ID NO: 103, respectively. In an aspect, the HCDR1, HCDR2, and HCDR3 comprise amino acid sequences of SEQ ID NOs: 89, 90, and 91, respectively, and the LCDR1, LCDR2, and LCDR3 comprise amino acid sequences of SEQ ID NOs: 104, 105, and 106, respectively. In an aspect, the HCDR1, HCDR2, and HCDR3 comprise amino acid sequences of SEQ ID NOs: 109, 110, and 111, respectively, and the LCDR1, LCDR2, and LCDR3 comprise amino acid sequences of SEQ ID NOs: 124, 125, and 126, respectively. In an aspect, the HCDR1, HCDR2, and HCDR3 comprise amino acid sequences of SEQ ID NOs: 112, 113, and 114, respectively, and the LCDR1, LCDR2, and LCDR3 comprise amino acid sequences of SEQ ID NOs: 127, 128, and 129, respectively. In an aspect, the HCDR1, HCDR2, and HCDR3 comprise amino acid sequences of SEQ ID NOs: 115, 116, and 117, respectively, and the LCDR1, LCDR2, and LCDR3 comprise amino acid sequences of SEQ ID NOs: 130, 131, and 132, respectively. In an aspect, the HCDR1, HCDR2, and HCDR3 comprise amino acid sequences of SEQ ID NOs: 118, 119, and 120, respectively, and the LCDR1, LCDR2, and LCDR3 comprise amino acid sequences of SEQ ID NO: 133, KVS, and SEQ ID NO: 135, respectively. In an aspect, the HCDR1, HCDR2, and HCDR3 comprise amino acid sequences of SEQ ID NOs: 121, 122, and 123, respectively, and the LCDR1, LCDR2, and LCDR3 comprise amino acid sequences of SEQ ID NOs: 136, 137, and 138, respectively. In an aspect, the HCDR1, HCDR2, and HCDR3 comprise amino acid sequences of SEQ ID NOs: 141, 142, and 143, respectively, and the LCDR1, LCDR2, and LCDR3 comprise amino acid sequences of SEQ ID NOs: 156, 157, and 158, respectively. In an aspect, the HCDR1, HCDR2, and HCDR3 comprise amino acid sequences of SEQ ID NOs: 144, 145, and 146, respectively, and the LCDR1, LCDR2, and LCDR3 comprise amino acid sequences of SEQ ID NOs: 159, 160, and 161, respectively. In an aspect, the HCDR1, HCDR2, and HCDR3 comprise amino acid sequences of SEQ ID NOs: 147, 148, and 149, respectively, and the LCDR1, LCDR2, and LCDR3 comprise amino acid sequences of SEQ ID NOs: 162, 163, and 164, respectively. In an aspect, the HCDR1, HCDR2, and HCDR3 comprise amino acid sequences of SEQ ID NOs: 150, 151, and 152, respectively, and the LCDR1, LCDR2, and LCDR3 comprise amino acid sequences of SEQ ID NO: 165, KVS, and SEQ ID NO: 167, respectively. In an aspect, the HCDR1, HCDR2, and HCDR3 comprise amino acid sequences of SEQ ID NOs: 153, 154, and 155, respectively, and the LCDR1, LCDR2, and LCDR3 comprise amino acid sequences of SEQ ID NOs: 168, 169, and 170, respectively. In an aspect, the HCDR1, HCDR2, and HCDR3 comprise amino acid sequences of SEQ ID NOs: 173, 174, and 175, respectively, and the LCDR1, LCDR2, and LCDR3 comprise amino acid sequences of SEQ ID NOs: 188, 189, and 190, respectively. In an aspect, the HCDR1, HCDR2, and HCDR3 comprise amino acid sequences of SEQ ID NOs: 176, 177, and 178, respectively, and the LCDR1, LCDR2, and LCDR3 comprise amino acid sequences of SEQ ID NOs: 191, 192, and 193, respectively. In an aspect, the HCDR1, HCDR2, and HCDR3 comprise amino acid sequences of SEQ ID NOs: 179, 180, and 181, respectively, and the LCDR1, LCDR2, and LCDR3 comprise amino acid sequences of SEQ ID NOs: 194, 195, and 196, respectively. In an aspect, the HCDR1, HCDR2, and HCDR3 comprise amino acid sequences of SEQ ID NOs: 182, 183, and 184, respectively, and the LCDR1, LCDR2, and LCDR3 comprise amino acid sequences of SEQ ID NO: 197, KVS, and SEQ ID NO: 199, respectively. In an aspect, the HCDR1, HCDR2, and HCDR3 comprise amino acid sequences of SEQ ID NOs: 185, 186, and 187, respectively, and the LCDR1, LCDR2, and LCDR3 comprise amino acid sequences of SEQ ID NOs: 200, 201, and 202, respectively. In an aspect, the HCDR1, HCDR2, and HCDR3 comprise amino acid sequences of SEQ ID NOs: 205, 206, and 207, respectively, and the LCDR1, LCDR2, and LCDR3 comprise amino acid sequences of SEQ ID NOs: 220, 221, and 222, respectively. In an aspect, the HCDR1, HCDR2, and HCDR3 comprise amino acid sequences of SEQ ID NOs: 208, 209, and 210, respectively, and the LCDR1, LCDR2, and LCDR3 comprise amino acid sequences of SEQ ID NOs: 223, 224, and 225, respectively. In an aspect, the HCDR1, HCDR2, and HCDR3 comprise amino acid sequences of SEQ ID NOs: 211, 212, and 213, respectively, and the LCDR1, LCDR2, and LCDR3 comprise amino acid sequences of SEQ ID NOs: 226, 227, and 228, respectively. In an aspect, the HCDR1, HCDR2, and HCDR3 comprise amino acid sequences of SEQ ID NOs: 214, 215, and 216, respectively, and the LCDR1, LCDR2, and LCDR3 comprise amino acid sequences of SEQ ID NO: 229, KVS, and SEQ ID NO: 231, respectively. In an aspect, the HCDR1, HCDR2, and HCDR3 comprise amino acid sequences of SEQ ID NOs: 217, 218, and 219, respectively, and the LCDR1, LCDR2, and LCDR3 comprise amino acid sequences of SEQ ID NOs: 232, 233, and 234, respectively.

In certain aspects, the HC1 and LC1 form a binding site for a first antigen, and the HC2 and LC2 form a binding site for a second antigen. By way of an example, the binding site for the first antigen can bind to a Vβ17 on a CD8+ or CD4+ T cell, and the binding site for the second antigen can, for example, bind a tumor antigen present on the surface of a cancer cell. The binding of the Vβ17 bispecific antibody to Vβ17 present on the surface of the CD8+ or CD4+ T cell, and the binding of the tumor antigen present on the surface of the cancer cells can, for example, result in the killing of the cancer cell.

In an aspect, the isolated Vβ17 bispecific antibodies or antigen-binding fragments thereof are isolated anti-Vβ17/anti-CD123 bispecific antibodies or antigen-binding fragments thereof. In an aspect, the isolated anti-Vβ17/anti-KLK2 bispecific antibodies or antigen-binding fragments thereof. In an aspect, the isolated anti-Vβ17/anti-PSMA bispecific antibodies or antigen-binding fragments thereof. In an aspect, isolated anti-Vβ17/anti-BCMA bispecific antibodies or antigen-binding fragments thereof. In certain aspects, HC2 and LC2 bind to CD123. In certain aspects, HC2 and LC2 bind to KLK2. In certain aspects, HC2 and LC2 bind to PSMA. In certain aspects, HC2 and LC2 bind to BCMA. In an aspect, the HC2 comprises a heavy chain complementarity determining region 1 (HCDR1-2), a heavy chain complementarity determining region 2 (HCDR2-2), and a heavy chain complementarity determining region 3 (HCDR3-2) comprising the amino acid sequences of SEQ ID NO:34, SEQ ID NO:35, and SEQ ID NO: 36, respectively, and the LC2 comprises a light chain complementarity determining region 1 (LCDR1-2), a light chain complementarity determining region 2 (LCDR2-2), and a light chain complementarity determining region 3 (LCDR3-2) comprising the amino acid sequences of SEQ ID NO:37, SEQ ID NO:38, and SEQ ID NO:39, respectively. In an aspect, the HCDR1-2, HCDR2-2, and HCDR3-2 comprise amino acid sequences of SEQ ID NOs: 237, 238, and 239, respectively, and the LCDR1-2, LCDR2-2, and LCDR3-2 comprise amino acid sequences of SEQ ID NOs: 252, 253, and 254, respectively. In an aspect, the HCDR1-2, HCDR2-2, and HCDR3-2 comprise amino acid sequences of SEQ ID NOs: 240, 241, and 242, respectively, and the LCDR1-2, LCDR2-2, and LCDR3-2 comprise amino acid sequences of SEQ ID NOs: 255, 256, and 257, respectively. In an aspect, the HCDR1-2, HCDR2-2, and HCDR3-2 comprise amino acid sequences of SEQ ID NOs: 243, 244, and 245, respectively, and the LCDR1-2, LCDR2-2, and LCDR3-2 comprise amino acid sequences of SEQ ID NOs: 258, 259, and 260, respectively. In an aspect, the HCDR1-2, HCDR2-2, and HCDR3-2 comprise amino acid sequences of SEQ ID NOs: 246, 247, and 248, respectively, and the LCDR1-2, LCDR2-2, and LCDR3-2 comprise amino acid sequences of SEQ ID NO: 261, AAS, and SEQ ID NO: 263, respectively. In an aspect, the HCDR1-2, HCDR2-2, and HCDR3-2 comprise amino acid sequences of SEQ ID NOs: 249, 250, and 251, respectively, and the LCDR1-2, LCDR2-2, and LCDR3-2 comprise amino acid sequences of SEQ ID NOs: 264, 265, and 266, respectively. In an aspect, the HCDR1-2, HCDR2-2, and HCDR3-2 comprise amino acid sequences of SEQ ID NOs: 269, 270, and 271, respectively, and the LCDR1-2, LCDR2-2, and LCDR3-2 comprise amino acid sequences of SEQ ID NOs: 284, 285, and 286, respectively. In an aspect, the HCDR1-2, HCDR2-2, and HCDR3-2 comprise amino acid sequences of SEQ ID NOs: 272, 273, and 274, respectively, and the LCDR1-2, LCDR2-2, and LCDR3-2 comprise amino acid sequences of SEQ ID NOs: 287, 288, and 289, respectively. In an aspect, the HCDR1-2, HCDR2-2, and HCDR3-2 comprise amino acid sequences of SEQ ID NOs: 275, 276, and 277, respectively, and the LCDR1-2, LCDR2-2, and LCDR3-2 comprise amino acid sequences of SEQ ID NOs: 290, 291, and 292, respectively. In an aspect, the HCDR1-2, HCDR2-2, and HCDR3-2 comprise amino acid sequences of SEQ ID NOs: 278, 279, and 280, respectively, and the LCDR1-2, LCDR2-2, and LCDR3-2 comprise amino acid sequences of SEQ ID NO: 293, DNN, and SEQ ID NO: 295, respectively. In an aspect, the HCDR1-2, HCDR2-2, and HCDR3-2 comprise amino acid sequences of SEQ ID NOs: 281, 282, and 283, respectively, and the LCDR1-2, LCDR2-2, and LCDR3-2 comprise amino acid sequences of SEQ ID NOs: 296, 297, and 298, respectively. In an aspect, the HCDR1-2, HCDR2-2, and HCDR3-2 comprise amino acid sequences of SEQ ID NOs: 301, 302, and 303, respectively, and the LCDR1-2, LCDR2-2, and LCDR3-2 comprise amino acid sequences of SEQ ID NOs: 316, 317, and 318, respectively. In an aspect, the HCDR1-2, HCDR2-2, and HCDR3-2 comprise amino acid sequences of SEQ ID NOs: 304, 305, and 306, respectively, and the LCDR1-2, LCDR2-2, and LCDR3-2 comprise amino acid sequences of SEQ ID NOs: 319, 320, and 321, respectively. In an aspect, the HCDR1-2, HCDR2-2, and HCDR3-2 comprise amino acid sequences of SEQ ID NOs: 307, 308, and 309, respectively, and the LCDR1-2, LCDR2-2, and LCDR3-2 comprise amino acid sequences of SEQ ID NOs: 322, 323, and 324, respectively. In an aspect, the HCDR1-2, HCDR2-2, and HCDR3-2 comprise amino acid sequences of SEQ ID NOs: 310, 311, and 312, respectively, and the LCDR1-2, LCDR2-2, and LCDR3-2 comprise amino acid sequences of SEQ ID NO: 325, GAS, and SEQ ID NO: 327, respectively. In an aspect, the HCDR1-2, HCDR2-2, and HCDR3-2 comprise amino acid sequences of SEQ ID NOs: 313, 314, and 315, respectively, and the LCDR1-2, LCDR2-2, and LCDR3-2 comprise amino acid sequences of SEQ ID NOs: 328, 329, and 330, respectively. In an aspect, the HCDR1-2, HCDR2-2, and HCDR3-2 comprise amino acid sequences of SEQ ID NOs: 333, 334, and 335, respectively, and the LCDR1-2, LCDR2-2, and LCDR3-2 comprise amino acid sequences of SEQ ID NOs: 348, 349, and 350, respectively. In an aspect, the HCDR1-2, HCDR2-2, and HCDR3-2 comprise amino acid sequences of SEQ ID NOs: 336, 337, and 338, respectively, and the LCDR1-2, LCDR2-2, and LCDR3-2 comprise amino acid sequences of SEQ ID NOs: 351, 352, and 353, respectively. In an aspect, the HCDR1-2, HCDR2-2, and HCDR3-2 comprise amino acid sequences of SEQ ID NOs: 339, 340, and 341, respectively, and the LCDR1-2, LCDR2-2, and LCDR3-2 comprise amino acid sequences of SEQ ID NOs: 354, 355, and 356, respectively. In an aspect, the HCDR1-2, HCDR2-2, and HCDR3-2 comprise amino acid sequences of SEQ ID NOs: 342, 343, and 344, respectively, and the LCDR1-2, LCDR2-2, and LCDR3-2 comprise amino acid sequences of SEQ ID NO: 357, DNN, and SEQ ID NO: 359, respectively. In an aspect, the HCDR1-2, HCDR2-2, and HCDR3-2 comprise amino acid sequences of SEQ ID NOs: 345, 346, and 347, respectively, and the LCDR1-2, LCDR2-2, and LCDR3-2 comprise amino acid sequences of SEQ ID NOs: 360, 361, and 362, respectively. In an aspect, the HCDR1-2, HCDR2-2, and HCDR3-2 comprise amino acid sequences of SEQ ID NOs: 365, 366, and 367, respectively, and the LCDR1-2, LCDR2-2, and LCDR3-2 comprise amino acid sequences of SEQ ID NOs: 380, 381, and 382, respectively. In an aspect, the HCDR1-2, HCDR2-2, and HCDR3-2 comprise amino acid sequences of SEQ ID NOs: 368, 369, and 370, respectively, and the LCDR1-2, LCDR2-2, and LCDR3-2 comprise amino acid sequences of SEQ ID NOs: 383, 384, and 385, respectively. In an aspect, the HCDR1-2, HCDR2-2, and HCDR3-2 comprise amino acid sequences of SEQ ID NOs: 371, 372, and 373, respectively, and the LCDR1-2, LCDR2-2, and LCDR3-2 comprise amino acid sequences of SEQ ID NOs: 386, 387, and 388, respectively. In an aspect, the HCDR1-2, HCDR2-2, and HCDR3-2 comprise amino acid sequences of SEQ ID NOs: 374, 375, and 376, respectively, and the LCDR1-2, LCDR2-2, and LCDR3-2 comprise amino acid sequences of SEQ ID NO: 389, DNN, and SEQ ID NO: 391, respectively. In an aspect, the HCDR1-2, HCDR2-2, and HCDR3-2 comprise amino acid sequences of SEQ ID NOs: 377, 378, and 379, respectively, and the LCDR1-2, LCDR2-2, and LCDR3-2 comprise amino acid sequences of SEQ ID NOs: 392, 393, and 394, respectively. In an aspect, the HCDR1-2, HCDR2-2, and HCDR3-2 comprise amino acid sequences of SEQ ID NOs: 397, 398, and 399, respectively, and the LCDR1-2, LCDR2-2, and LCDR3-2 comprise amino acid sequences of SEQ ID NOs: 412, 413, and 414, respectively. In an aspect, the HCDR1-2, HCDR2-2, and HCDR3-2 comprise amino acid sequences of SEQ ID NOs: 400, 401, and 402, respectively, and the LCDR1-2, LCDR2-2, and LCDR3-2 comprise amino acid sequences of SEQ ID NOs: 415, 416, and 417, respectively. In an aspect, the HCDR1-2, HCDR2-2, and HCDR3-2 comprise amino acid sequences of SEQ ID NOs: 403, 404, and 405, respectively, and the LCDR1-2, LCDR2-2, and LCDR3-2 comprise amino acid sequences of SEQ ID NOs: 418, 419, and 420, respectively. In an aspect, the HCDR1-2, HCDR2-2, and HCDR3-2 comprise amino acid sequences of SEQ ID NOs: 404, 405, and 406, respectively, and the LCDR1-2, LCDR2-2, and LCDR3-2 comprise amino acid sequences of SEQ ID NO: 421, KAS, and SEQ ID NO: 423, respectively. In an aspect, the HCDR1-2, HCDR2-2, and HCDR3-2 comprise amino acid sequences of SEQ ID NOs: 407, 408, and 409, respectively, and the LCDR1-2, LCDR2-2, and LCDR3-2 comprise amino acid sequences of SEQ ID NOs: 424, 425, and 426, respectively. In certain embodiments, the V□17 is on the surface of a CD8+ or CD4+ T cell. In certain embodiments, the CD123 is on the surface of a cancer cell. In certain embodiments, the KLK2 is on the surface of a cancer cell. In certain embodiments, the PSMA is on the surface of a cancer cell. In certain embodiments, the BCMA is on the surface of a cancer cell.

In certain embodiments, the Vβ17 is on the surface of a CD8+ or CD4+ T cell. In certain embodiments, the CD123 is on the surface of a cancer cell (e.g., a leukemia cell).

In some embodiments, the bispecific antibodies disclosed herein can take the form of a diabody, a cross-body, or a bispecific antibody obtained via a controlled Fab arm exchange as described herein.

In some embodiments, the bispecific antibodies include IgG-like molecules with complementary CH3 domains that promote heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus) and the DuoBody (Genmab A/S).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (InnClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual (ScFv) 2-Fab (National Research Center for Antibody Medicine—China).

In some embodiments, Fab fusion bispecific antibodies include F (ab) 2 (Medarex/AMGEN), Dual-Action or Bis-Fab (Genentech), Dock-and-Lock (DNL) (ImmunoMedics), Bivalent Bispecific (Biotecnoland Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Full length bispecific antibodies disclosed herein can be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms can be engineered to favor heterodimerization over homodimerization. The resulting product is a bispecific antibody having two Fab arms or half molecules, each binding a distinct epitope, i.e., an epitope on Vβ17 and an epitope on a tumor antigen.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Inti. Publ. No. WO 2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S L368A_Y407V.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y F405AY407V/T394W, T366I_K392M_T394W/F405A_Y407V, T366L K392M_T394W/F405A_Y407V, L351Y_Y407A/T366A_K409F, L351Y_Y407A/T366V K409F Y407A/T366A K409F, or T350V L351Y F405A Y407V/T350V T366L K392L T394W as described in U.S. Pat. Publ. No. US2012/0149876 or U.S. Pat. Publ. No. US2013/0195849.

In addition to methods described above, bispecific antibodies disclosed herein can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in International Pat. Publ. No. WO2011/131746. In the methods, the first monospecific bivalent antibody (e.g., anti-CD33 antibody) and the second monospecific bivalent antibody (e.g., anti-CD3 antibody) are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that may be used are 2-mercaptoethylamine (2-MEA), dithiothreitol (DTT), dithioerythritol (DTE), glutathione, tris(2-carboxyethyl) phosphine (TCEP), L-cysteine and beta-mercaptoethanol, preferably a reducing agent selected from the group consisting of: 2-mercaptoethylamine, dithiothreitol and tris(2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 may be used.

In certain embodiments, the anti-Vβ17 bispecific antibody or antigen-binding fragment thereof comprises:
a. a heavy chain 1 (HC1) having the amino acid sequence of SEQ ID NO: 429, a heavy chain 2 (HC2) having the amino acid sequence of SEQ ID NO: 430, and a light chain 2 (LC2) having the amino acid sequence of SEQ ID NO: 431; or
b. a HC1, a HC2, and a LC2 having the amino acid sequences of SEQ ID NOs: 432, 433, and 434, respectively; or
c. a HC1, a HC2, and a LC2 having the amino acid sequences of SEQ ID NOs: 435, 436, and 437, respectively; or
d. a HC1, a light chain 1 (LC1) and a HC2 having the amino acid sequences of SEQ ID NOs: 438, 439, and 440, respectively; or
e. a HC1, a HC2, and a LC2 having the amino acid sequences of SEQ ID NOs: 441, 442, and 443, respectively; or
f. a HC1, a LC1 and a HC2 having the amino acid sequences of SEQ ID NOs: 444, 445, and 446, respectively; or
g. a HC1, a HC2, and a LC2 having the amino acid sequences of SEQ ID NOs: 447, 448, and 449, respectively; or
   a HC1, a HC2, and a LC2 having the amino acid sequences of SEQ ID NOs: 450, 451, and 452, respectively.

According to another particular aspect, the invention relates to an isolated anti-Vβ17/anti-CD123 bispecific antibody or antigen-binding fragment thereof that induces antibody-dependent cell-mediated cytotoxicity (ADCC). The anti-Vβ17/anti-CD123 bispecific antibody or antigen-binding fragment thereof can, for example, induce ADCC in vitro. The bispecific antibody or antigen-binding fragment thereof can induce ADCC with an EC₅₀ of less than about 1 pM. In certain embodiments, the EC₅₀ is less than about 1 pM, less than about 0.9 pM, less than about 0.8 pM, less than about 0.7 pM, less than about 0.6 pM, less than about 0.5 pM, less than about 0.4 pM, less than about 0.300 pM, less than about 0.2 pM, less than about 0.19 pM, less than about 0.18 pM, less than about 0.17 pM, less than about 0.16 pM, less than about 0.15 pM, less than about 0.14 pM, less than about 0.13 pM, less than about 0.12 pM, less than about 0.11 pM, less than about 0.1 pM, less than about 0.09 pM, less than about 0.08 pM, less than about 0.07 pM, less than about 0.06 pM, less than about 0.05 pM, less than about 0.04 pM, less than about 0.03 pM, less than about 0.02 pM, or less than about 0.01 pM. In certain embodiments, the anti-Vβ17/anti-CD123 bispecific antibody or antigen-binding fragment thereof comprises an IgG1, IgG2, IgG3, or IgG4 backbone. In one such embodiment, the anti-Vβ17/anti-CD123 bispecific antibody or antigen-binding fragment thereof has an antibody backbone of the IgG4 isotype. In another such embodiment, the anti-Vβ17/anti-CD123 bispecific antibody or antigen-binding fragment thereof has an antibody backbone of the IgG1 isotype.

According to another particular aspect, the invention relates to an isolated anti-Vβ17/anti-KLK2 bispecific antibody or antigen-binding fragment thereof that induces antibody-dependent cell-mediated cytotoxicity (ADCC). The anti-Vβ17/anti-KLK2 bispecific antibody or antigen-binding fragment thereof can, for example, induce ADCC in vitro. The bispecific antibody or antigen-binding fragment thereof can induce ADCC with an EC₅₀ of less than about 1 pM. In certain embodiments, the EC₅₀ is less than about 1 pM, less than about 0.9 pM, less than about 0.8 pM, less than about 0.7 pM, less than about 0.6 pM, less than about 0.5 pM, less than about 0.4 pM, less than about 0.300 pM, less than about 0.2 pM, less than about 0.19 pM, less than about 0.18 pM, less than about 0.17 pM, less than about 0.16 pM, less than about 0.15 pM, less than about 0.14 pM, less than about 0.13 pM, less than about 0.12 pM, less than about 0.11 pM, less than about 0.1 pM, less than about 0.09 pM, less than about 0.08 pM, less than about 0.07 pM, less than about 0.06 pM, less than about 0.05 pM, less than about 0.04 pM, less than about 0.03 pM, less than about 0.02 pM, or less than about 0.01 pM. In certain embodiments, the anti-Vβ17/anti-KLK2 bispecific antibody or antigen-binding fragment thereof comprises an IgG1, IgG2, IgG3, or IgG4 backbone. In one such embodiment, the anti-Vβ17/anti-KLK2 bispecific antibody or antigen-binding fragment thereof has an antibody backbone of the IgG4 isotype. In another such embodiment, the anti-Vβ17/anti-KLK2 bispecific antibody or antigen-binding fragment thereof has an antibody backbone of the IgG1 isotype.

According to another particular aspect, the invention relates to an isolated anti-Vβ17/anti-PSMA bispecific antibody or antigen-binding fragment thereof that induces antibody-dependent cell-mediated cytotoxicity (ADCC). The anti-Vβ17/anti-PSMA bispecific antibody or antigen-binding fragment thereof can, for example, induce ADCC in vitro. The bispecific antibody or antigen-binding fragment thereof can induce ADCC with an EC₅₀ of less than about 1 pM. In certain embodiments, the EC₅₀ is less than about 1 pM, less than about 0.9 pM, less than about 0.8 pM, less than about 0.7 pM, less than about 0.6 pM, less than about 0.5 pM, less than about 0.4 pM, less than about 0.300 pM, less than about 0.2 pM, less than about 0.19 pM, less than about 0.18 pM, less than about 0.17 pM, less than about 0.16 pM, less than about 0.15 pM, less than about 0.14 pM, less than about 0.13 pM, less than about 0.12 pM, less than about 0.11 pM, less than about 0.1 pM, less than about 0.09 pM, less than about 0.08 pM, less than about 0.07 pM, less than about 0.06 pM, less than about 0.05 pM, less than about 0.04 pM, less than about 0.03 pM, less than about 0.02 pM, or less than about 0.01 pM. In certain embodiments, the anti-Vβ17/anti-PSMA bispecific antibody or antigen-binding fragment thereof comprises an IgG1, IgG2, IgG3, or IgG4 backbone. In one such embodiment, the anti-Vβ17/anti-PSMA bispecific antibody or antigen-binding fragment thereof has an antibody backbone of the IgG4 isotype. In another such embodiment, the anti-Vβ17/anti-PSMA bispecific antibody or antigen-binding fragment thereof has an antibody backbone of the IgG1 isotype.

According to another particular aspect, the invention relates to an isolated anti-Vβ17/anti-BCMA bispecific antibody or antigen-binding fragment thereof that induces antibody-dependent cell-mediated cytotoxicity (ADCC). The anti-Vβ17/anti-BCMA bispecific antibody or antigen-binding fragment thereof can, for example, induce ADCC in vitro. The bispecific antibody or antigen-binding fragment thereof can induce ADCC with an EC₅₀ of less than about 1 pM. In certain embodiments, the EC₅₀ is less than about 1 pM, less than about 0.9 pM, less than about 0.8 pM, less than about 0.7 pM, less than about 0.6 pM, less than about 0.5 pM, less than about 0.4 pM, less than about 0.300 pM, less than about 0.2 pM, less than about 0.19 pM, less than about 0.18 pM, less than about 0.17 pM, less than about 0.16 pM, less than about 0.15 pM, less than about 0.14 pM, less than about 0.13 pM, less than about 0.12 pM, less than about 0.11 pM, less than about 0.1 pM, less than about 0.09 pM, less than about 0.08 pM, less than about 0.07 pM, less than about 0.06 pM, less than about 0.05 pM, less than about 0.04 pM, less than about 0.03 pM, less than about 0.02 pM, or less than about 0.01 pM. In certain embodiments, the anti-Vβ17/anti-BCMA bispecific antibody or antigen-binding fragment thereof comprises an IgG1, IgG2, IgG3, or IgG4 backbone. In one such embodiment, the anti-Vβ17/anti-BCMA bispecific antibody or antigen-binding fragment thereof has an antibody backbone of the IgG4 isotype. In another such embodiment, the anti-Vβ17/anti-BCMA bispecific antibody or antigen-binding fragment thereof has an antibody backbone of the IgG1 isotype.

In some embodiments described herein, immune effector properties of the anti-Vβ17/anti-CD123 bispecific antibodies provided herein, the anti-Vβ17/anti-KLK2 bispecific antibodies provided herein, the anti-Vβ17/anti-PSMA bispecific antibodies provided herein, and/or the anti-Vβ17/anti-BCMA bispecific antibodies provided herein can be enhanced or silenced through Fc modifications by techniques known to those skilled in the art. For example, Fc effector functions such as C1q binding, complement dependent cytotoxicity (CDC), antibody-dependent cell-mediated cytotoxicity (ADCC), antibody-dependent cell-mediated phagocytosis (ADCP), down regulation of cell surface receptors (e.g., B cell receptor; BCR), etc. may be provided and/or controlled by modifying residues in the Fc responsible for these activities.

“Antibody-dependent cell-mediated cytotoxicity” or “ADCC” refers to a cell-mediated reaction in which non-specific cytotoxic cells that express Fc receptors (FcRs) (e.g., Natural Killer (NK) cells, neutrophils, and macrophages) recognize bound antibody on a target cell and subsequently cause lysis of the target cell.

The ability of antibodies to induce ADCC can be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen-binding or CDC activity. Such Abs can be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2 (4), 2010; Epub ahead of print; PMID: 20562582), application of a rat hybridoma cell line YB2/0 as the host cell line (Shinkawa et al., J Biol Chem 278:3466-3473, 2003), introduction of small interfering RNA specifically against the α-1,6-fucosyltrasferase (FUT8) gene (Mori et al., Biotechnol Bioeng 88:901-908, 2004), or coexpression of β-1,4-N-acetylglucosaminyltransferase III and golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments described herein, ADCC elicited by the anti-Vβ17/anti-CD123 bispecific antibodies provided herein, the anti-Vβ17/anti-KLK2 bispecific antibodies provided herein, the anti-Vβ17/anti-PSMA bispecific antibodies provided herein, and/or the anti-Vβ17/anti-BCMA bispecific antibodies provided herein can also be enhanced by certain substitutions in the antibody Fc. Exemplary substitutions include, for example, substitutions at amino acid positions 256, 290, 298, 312, 356, 330, 333, 334, 360, 378 or 430 (residue numbering according to the EU index) as described in U.S. Pat. No. 6,737,056.

According to another particular aspect, the invention relates to an isolated anti-Vβ17/anti-CD123 bispecific antibody or antigen-binding fragment thereof capable of inducing T-cell dependent cytotoxicity in Vβ17-expressing cells and/or CD123-expressing cells. The bispecific antibody or antigen-binding fragment thereof can, for example, induce T-cell dependent cytotoxicity in Vβ17-expressing cells and/or CD123-expressing cells in vitro with an EC₅₀ value of less than about 2 nM. In certain embodiments, the EC₅₀ is less than about 2.0 nM, less than about 1.9 nM, less than about 1.8 nM, less than about 1.7 nM, less than about 1.6 nM, less than about 1.5 nM, less than about 1.4 nM, less than about 1.3 nM, less than about 1.2 nM, less than about 1.1 nM, less than about 1.0 nM, less than about 0.9 nM, less than about 0.8 nM, less than about 0.7 nM, less than about 0.6 nM, less than about 0.5 nM, less than about 0.4 nM, less than about 0.3 nM, less than about 0.2 nM, and less than about 0.1 nM.

According to another particular aspect, the invention relates to an isolated anti-Vβ17/anti-KLK2 bispecific antibody or antigen-binding fragment thereof capable of inducing T-cell dependent cytotoxicity in Vβ17-expressing cells and/or KLK2-expressing cells. The bispecific antibody or antigen-binding fragment thereof can, for example, induce T-cell dependent cytotoxicity in Vβ17-expressing cells and/or KLK2-expressing cells in vitro with an EC₅₀ value of less than about 2 nM. In certain embodiments, the EC₅₀ is less than about 2.0 nM, less than about 1.9 nM, less than about 1.8 nM, less than about 1.7 nM, less than about 1.6 nM, less than about 1.5 nM, less than about 1.4 nM, less than about 1.3 nM, less than about 1.2 nM, less than about 1.1 nM, less than about 1.0 nM, less than about 0.9 nM, less than about 0.8 nM, less than about 0.7 nM, less than about 0.6 nM, less than about 0.5 nM, less than about 0.4 nM, less than about 0.3 nM, less than about 0.2 nM, and less than about 0.1 nM.

According to another particular aspect, the invention relates to an isolated anti-Vβ17/anti-PSMA bispecific antibody or antigen-binding fragment thereof capable of inducing T-cell dependent cytotoxicity in Vβ17-expressing cells and/or PSMA-expressing cells. The bispecific antibody or antigen-binding fragment thereof can, for example, induce T-cell dependent cytotoxicity in Vβ17-expressing cells and/or PSMA-expressing cells in vitro with an EC₅₀ value of less than about 2 nM. In certain embodiments, the EC₅₀ is less than about 2.0 nM, less than about 1.9 nM, less than about 1.8 nM, less than about 1.7 nM, less than about 1.6 nM, less than about 1.5 nM, less than about 1.4 nM, less than about 1.3 nM, less than about 1.2 nM, less than about 1.1 nM, less than about 1.0 nM, less than about 0.9 nM, less than about 0.8 nM, less than about 0.7 nM, less than about 0.6 nM, less than about 0.5 nM, less than about 0.4 nM, less than about 0.3 nM, less than about 0.2 nM, and less than about 0.1 nM.

According to another particular aspect, the invention relates to an isolated anti-Vβ17/anti-BCMA bispecific antibody or antigen-binding fragment thereof capable of inducing T-cell dependent cytotoxicity in Vβ17-expressing cells and/or BCMA-expressing cells. The bispecific antibody or antigen-binding fragment thereof can, for example, induce T-cell dependent cytotoxicity in Vβ17-expressing cells and/or BCMA-expressing cells in vitro with an EC₅₀ value of less than about 2 nM. In certain embodiments, the EC₅₀ is less than about 2.0 nM, less than about 1.9 nM, less than about 1.8 nM, less than about 1.7 nM, less than about 1.6 nM, less than about 1.5 nM, less than about 1.4 nM, less than about 1.3 nM, less than about 1.2 nM, less than about 1.1 nM, less than about 1.0 nM, less than about 0.9 nM, less than about 0.8 nM, less than about 0.7 nM, less than about 0.6 nM, less than about 0.5 nM, less than about 0.4 nM, less than about 0.3 nM, less than about 0.2 nM, and less than about 0.1 nM.

In an aspect, the invention relates to an isolated anti-Vβ17/anti-CD123 bispecific antibody or antigen-binding fragment thereof, wherein the anti-Vβ17/anti-CD123 bispecific antibody or antigen-binding fragment thereof is chimeric. In an aspect, the invention relates to an isolated anti-Vβ17/anti-KLK2 bispecific antibody or antigen-binding fragment thereof, wherein the anti-Vβ17/anti-KLK2 bispecific antibody or antigen-binding fragment thereof is chimeric. In an aspect, the invention relates to an isolated anti-Vβ17/anti-PSMA bispecific antibody or antigen-binding fragment thereof, wherein the anti-Vβ17/anti-PSMA bispecific antibody or antigen-binding fragment thereof is chimeric. In an aspect, the invention relates to an isolated anti-Vβ17/anti-BCMA bispecific antibody or antigen-binding fragment thereof, wherein the anti-Vβ17/anti-BCMA bispecific antibody or antigen-binding fragment thereof is chimeric.

In an aspect, the invention relates to an isolated anti-Vβ17/anti-CD123 bispecific antibody or antigen-binding fragment thereof, wherein the anti-Vβ17/anti-CD123 bispecific antibody or antigen-binding fragment thereof is human or humanized. In an aspect, the invention relates to an isolated anti-Vβ17/anti-KLK2 bispecific antibody or antigen-binding fragment thereof, wherein the anti-Vβ17/anti-KLK2 bispecific antibody or antigen-binding fragment thereof is humanized. In an aspect, the invention relates to an isolated anti-Vβ17/anti-PSMA bispecific antibody or antigen-binding fragment thereof, wherein the anti-Vβ17/anti-PSMA bispecific antibody or antigen-binding fragment thereof is humanized. In an aspect, the invention relates to an isolated anti-Vβ17/anti-BCMA bispecific antibody or antigen-binding fragment thereof, wherein the anti-Vβ17/anti-BCMA bispecific antibody or antigen-binding fragment thereof is humanized.

In another general aspect, the invention relates to an isolated humanized Vβ17 monoclonal antibody or antigen-binding fragment thereof. In aspects, the isolated humanized Vβ17 monoclonal antibody or antigen-binding fragment thereof comprises an amino acid sequence with at least 85%, preferably 90%, more preferably 95% or more, such as 95%, 96%, 97%, 98%, or 99% identity to the amino acid sequence of SEQ ID NO:28. In a certain aspect, the humanized Vβ17 monoclonal antibody or antigen-binding fragment thereof comprises the amino acid sequence of SEQ ID NO:28. In aspects, the isolated humanized Vβ17 monoclonal antibody or antigen-binding fragment thereof can comprise a heavy chain (HC) having at least 95% identity to an amino acid sequence selected from the group consisting of SEQ ID NOs: 429, 432, 435, 438, 441, 444, 447, and 450. In aspects, the isolated humanized Vβ17 monoclonal antibody or antigen-binding fragment thereof can comprise a heavy chain (HC) having an amino acid sequence selected from the group consisting of SEQ ID NOs: 429, 432, 435, 438, 441, 444, 447, and 450. In aspects, the isolated humanized Vβ17 monoclonal antibody or antigen-binding fragment thereof can comprise a light chain (LC) having at least 95% identity to an amino acid sequence selected from the group consisting of SEQ ID NOs: 431, 434, 437, 439, 443, 445, 449, and 452. In aspects, the isolated humanized Vβ17 monoclonal antibody or antigen-binding fragment thereof can comprise a light chain (LC) having an amino acid sequence selected from the group consisting of SEQ ID NOs: 431, 434, 437, 439, 443, 445, 449, and 452. In aspects, the isolated humanized Vβ17 monoclonal antibody or antigen-binding fragment thereof can comprise a heavy chain (HC) having at least 95% identity to an amino acid sequence selected from the group consisting of SEQ ID NOs: 429, 432, 435, 438, 441, 444, 447, and 450, and a light chain (LC) having at least 95% identity to an amino acid sequence selected from the group consisting of SEQ ID NOs: 431, 434, 437, 439, 443, 445, 449, and 452. In aspects, the isolated humanized Vβ17 monoclonal antibody or antigen-binding fragment thereof can comprise a heavy chain (HC) having an amino acid sequence selected from the group consisting of SEQ ID NOs: 429, 432, 435, 438, 441, 444, 447, and 450, and a light chain (LC) having an amino acid sequence selected from the group consisting of SEQ ID NOs: 431, 434, 437, 439, 443, 445, 449, and 452

In another general aspect, the invention relates to isolated nucleic acids encoding the monoclonal antibodies or antigen-binding fragments thereof disclosed herein. In another general aspect, the invention relates to isolated nucleic acids encoding the bispecific antibodies or antigen-binding fragments thereof disclosed herein. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding monoclonal antibodies and/or bispecific antibodies disclosed herein can be altered without changing the amino acid sequences of the proteins.

In another general aspect, the invention relates to vectors comprising the isolated nucleic acids disclosed herein. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments disclosed herein. Such techniques are well known to those skilled in the art in view of the present disclosure.

In another general aspect, the invention relates to host cells comprising the isolated nucleic acids encoding the monoclonal antibodies and/or bispecific antibodies or antigen-binding fragments thereof disclosed herein. Any host cell known to those skilled in the art in view of the present disclosure can be used for recombinant expression of antibodies or antigen-binding fragments thereof disclosed herein. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a bispecific antibody or antigen-binding fragment thereof disclosed herein. The methods comprise culturing a cell comprising a nucleic acid encoding the bispecific antibody or antigen-binding fragment thereof under conditions to produce a bispecific antibody or antigen-binding fragment thereof disclosed herein, and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

### Methods of Use

In a general aspect, the invention relates to a method of targeting CD123 on the surface of a cancer cell, the method comprising exposing the cancer cell to an anti-Vβ17/anti-CD123 bispecific antibody or antigen-binding fragment thereof. In a general aspect, the invention relates to a method of targeting KLK2 on the surface of a cancer cell, the method comprising exposing the cancer cell to an anti-Vβ17/anti-KLK2 bispecific antibody or antigen-binding fragment thereof. In a general aspect, the invention relates to a method of targeting PSMA on the surface of a cancer cell, the method comprising exposing the cancer cell to an anti-Vβ17/anti-PSMA bispecific antibody or antigen-binding fragment thereof. In a general aspect, the invention relates to a method of targeting BCMA on the surface of a cancer cell, the method comprising exposing the cancer cell to an anti-Vβ17/anti-BCMA bispecific antibody or antigen-binding fragment thereof.

The functional activity of bispecific antibodies and antigen-binding fragments thereof that bind Vβ17 and/or CD123 can be characterized by methods known in the art and as described herein. Methods for characterizing antibodies and antigen-binding fragments thereof that bind Vβ17 and/or CD123 include, but are not limited to, affinity and specificity assays including Biacore, ELISA, and OctetRed analysis; binding assays to detect the binding of antibodies to CD123 on cancer cells by FACS; binding assays to detect the binding of antibodies to Vβ17 on CD8+ or CD4+ T cells. According to particular embodiments, the methods for characterizing antibodies and antigen-binding fragments thereof that bind Vβ17 and/or CD123 include those described below.

The functional activity of bispecific antibodies and antigen-binding fragments thereof that bind Vβ17 and/or KLK2 can be characterized by methods known in the art and as described herein. Methods for characterizing antibodies and antigen-binding fragments thereof that bind Vβ17 and/or KLK2 include, but are not limited to, affinity and specificity assays including Biacore, ELISA, and OctetRed analysis; binding assays to detect the binding of antibodies to KLK2 on cancer cells by FACS; binding assays to detect the binding of antibodies to Vβ17 on CD8+ or CD4+ T cells. According to particular embodiments, the methods for characterizing antibodies and antigen-binding fragments thereof that bind Vβ17 and/or KLK2 include those described below.

The functional activity of bispecific antibodies and antigen-binding fragments thereof that bind Vβ17 and/or PSMA can be characterized by methods known in the art and as described herein. Methods for characterizing antibodies and antigen-binding fragments thereof that bind Vβ17 and/or PSMA include, but are not limited to, affinity and specificity assays including Biacore, ELISA, and OctetRed analysis; binding assays to detect the binding of antibodies to PSMA on cancer cells by FACS; binding assays to detect the binding of antibodies to Vβ17 on CD8+ or CD4+ T cells. According to particular embodiments, the methods for characterizing antibodies and antigen-binding fragments thereof that bind Vβ17 and/or PSMA include those described below.

The functional activity of bispecific antibodies and antigen-binding fragments thereof that bind Vβ17 and/or BCMA can be characterized by methods known in the art and as described herein. Methods for characterizing antibodies and antigen-binding fragments thereof that bind Vβ17 and/or BCMA include, but are not limited to, affinity and specificity assays including Biacore, ELISA, and OctetRed analysis; binding assays to detect the binding of antibodies to BCMA on cancer cells by FACS; binding assays to detect the binding of antibodies to Vβ17 on CD8+ or CD4+ T cells. According to particular embodiments, the methods for characterizing antibodies and antigen-binding fragments thereof that bind Vβ17 and/or BCMA include those described below.

In an aspect, the invention relates to a method of directing Vβ17-expressing CD8+ or CD4+ T cells to a cancer cell. In aspects, the methods comprise contacting the Vβ17-expressing CD8+ or CD4+ T cell with an anti-Vβ17/anti-CD123 bispecific antibody or antigen-binding fragment thereof, wherein the antibody or antibody fragment directs the Vβ17-expressing CD8+ or CD4+ T cell to a cancer cell having CD123 on its surface. In aspects, the methods comprise contacting the Vβ17-expressing CD8+ or CD4+ T cell with an anti-Vβ17/anti-KLK2 bispecific antibody or antigen-binding fragment thereof, wherein the antibody or antibody fragment directs the Vβ17-expressing CD8+ or CD4+ T cell to a cancer cell having KLK2 on its surface. In aspects, the methods comprise contacting the Vβ17-expressing CD8+ or CD4+ T cell with an anti-Vβ17/anti-PSMA bispecific antibody or antigen-binding fragment thereof, wherein the antibody or antibody fragment directs the Vβ17-expressing CD8+ or CD4+ T cell to a cancer cell having PSMA on its surface. In aspects, the methods comprise contacting the Vβ17-expressing CD8+ or CD4+ T cell with an anti-Vβ17/anti-BCMA bispecific antibody or antigen-binding fragment thereof, wherein the antibody or antibody fragment directs the Vβ17-expressing CD8+ or CD4+ T cell to a cancer cell having BCMA on its surface.

In an aspect, the invention relates to a method for inhibiting growth or proliferation of cancer cells. In aspects, the methods comprise contacting the Vβ17-expressing CD8+ T cells with an anti-Vβ17/anti-CD123 bispecific antibody or antigen-binding fragment thereof, wherein contacting the cancer cells with the antibody or antibody fragment inhibits the growth or proliferation of the cancer cells. In aspects, the methods comprise contacting the Vβ17-expressing CD8+ T cells with an anti-Vβ17/anti-KLK2 bispecific antibody or antigen-binding fragment thereof, wherein contacting the cancer cells with the antibody or antibody fragment inhibits the growth or proliferation of the cancer cells. In aspects, the methods comprise contacting the Vβ17-expressing CD8+ T cells with an anti-Vβ17/anti-PSMA bispecific antibody or antigen-binding fragment thereof, wherein contacting the cancer cells with the antibody or antibody fragment inhibits the growth or proliferation of the cancer cells. In aspects, the methods comprise contacting the Vβ17-expressing CD8+ T cells with an anti-Vβ17/anti-BCMA bispecific antibody or antigen-binding fragment thereof, wherein contacting the cancer cells with the antibody or antibody fragment inhibits the growth or proliferation of the cancer cells.

According to embodiments of the invention, the described anti-Vβ17/anti-CD123 bispecific antibody or antigen-binding fragment thereof can be provided in a buffered composition for storage or use. Suitable buffers for the storage of the described anti-Vβ17/anti-CD123 bispecific antibody or antigen-binding fragment thereof would serve to maintain the stability of the antibody or antibody fragment by minimizing deterioration while stored, not promoting aggregation of the antibody or antibody fragment, or minimizing adhesion to the storage vessel.

According to embodiments of the invention, the described anti-Vβ17/anti-KLK2 bispecific antibody or antigen-binding fragment thereof can be provided in a buffered composition for storage or use. Suitable buffers for the storage of the described anti-Vβ17/anti-KLK2 bispecific antibody or antigen-binding fragment thereof would serve to maintain the stability of the antibody or antibody fragment by minimizing deterioration while stored, not promoting aggregation of the antibody or antibody fragment, or minimizing adhesion to the storage vessel.

According to embodiments of the invention, the described anti-Vβ17/anti-PSMA bispecific antibody or antigen-binding fragment thereof can be provided in a buffered composition for storage or use. Suitable buffers for the storage of the described anti-Vβ17/anti-PSMA bispecific antibody or antigen-binding fragment thereof would serve to maintain the stability of the antibody or antibody fragment by minimizing deterioration while stored, not promoting aggregation of the antibody or antibody fragment, or minimizing adhesion to the storage vessel.

According to embodiments of the invention, the described anti-Vβ17/anti-BCMA bispecific antibody or antigen-binding fragment thereof can be provided in a buffered composition for storage or use. Suitable buffers for the storage of the described anti-Vβ17/anti-BCMA bispecific antibody or antigen-binding fragment thereof would serve to maintain the stability of the antibody or antibody fragment by minimizing deterioration while stored, not promoting aggregation of the antibody or antibody fragment, or minimizing adhesion to the storage vessel.

According to embodiments of the invention, the described anti-Vβ17 antibodies or antigen-binding fragment thereof can be provided in a buffered composition for storage or use. Suitable buffers for the storage of the described anti-Vβ17 antibodies or antigen-binding fragment thereof would serve to maintain the stability of the antibody or antibody fragment by minimizing deterioration while stored, not promoting aggregation of the antibody or antibody fragment, or minimizing adhesion to the storage vessel.

### Embodiments

This invention provides the following non-limiting embodiments.

Embodiment 1 is a Vβ17 bispecific antibody or antigen-binding fragment thereof, the Vβ17 bispecific antibody or antigen-binding fragment thereof comprising:
a. a first heavy chain (HC1);
b. a second heavy chain (HC2);
c. a first light chain (LC1); and
d. a second light chain (LC2),
   wherein HC1 is associated with LC1 to form a binding site for a first antigen, and HC2 is associated with LC2 to form a binding site for a second antigen, wherein HC1 comprises a heavy chain complementarity determining region 1 (HCDR1), a heavy chain complementarity determining region 2 (HCDR2), and a heavy chain complementarity determining region 3 (HCDR3), and wherein LC1 comprises a light chain complementarity determining region 1 (LCDR1), a light chain complementarity determining region 2 (LCDR2), and a light chain complementarity determining region 3 (LCDR3), wherein
i. the HCDR1, HCDR2, and HCDR3 comprise amino acid sequences of SEQ ID NOs: 45, 46, and 47, respectively, and the LCDR1, LCDR2, and LCDR3 comprise amino acid sequences of SEQ ID NOs: 60, 61, and 62, respectively; or
ii. the HCDR1, HCDR2, and HCDR3 comprise amino acid sequences of SEQ ID NOs: 48, 49, and 50, respectively, and the LCDR1, LCDR2, and LCDR3 comprise amino acid sequences of SEQ ID NOs: 63, 64, and 65, respectively; or
iii. the HCDR1, HCDR2, and HCDR3 comprise amino acid sequences of SEQ ID NOs: 51, 52, and 53, respectively, and the LCDR1, LCDR2, and LCDR3 comprise amino acid sequences of SEQ ID NOs: 66, 67, and 68, respectively; or
iv. the HCDR1, HCDR2, and HCDR3 comprise amino acid sequences of SEQ ID NOs: 54, 55, and 56, respectively, and the LCDR1, LCDR2, and LCDR3 comprise amino acid sequences of SEQ ID NO: 69, KVS, and SEQ ID NO: 71, respectively; or
v. the HCDR1, HCDR2, and HCDR3 comprise amino acid sequences of SEQ ID NOs: 57, 58, and 59, respectively, and the LCDR1, LCDR2, and LCDR3 comprise amino acid sequences of SEQ ID NOs: 72, 73, and 74, respectively; or
vi. the HCDR1, HCDR2, and HCDR3 comprise amino acid sequences of SEQ ID NOs: 77, 78, and 79, respectively, and the LCDR1, LCDR2, and LCDR3 comprise amino acid sequences of SEQ ID NOs: 92, 93, and 94, respectively; or
vii. the HCDR1, HCDR2, and HCDR3 comprise amino acid sequences of SEQ ID NOs: 80, 81, and 82, respectively, and the LCDR1, LCDR2, and LCDR3 comprise amino acid sequences of SEQ ID NOs: 95, 96, and 97, respectively; or
viii. the HCDR1, HCDR2, and HCDR3 comprise amino acid sequences of SEQ ID NOs: 83, 84, and 85, respectively, and the LCDR1, LCDR2, and LCDR3 comprise amino acid sequences of SEQ ID NOs: 98, 99, and 100, respectively; or
ix. the HCDR1, HCDR2, and HCDR3 comprise amino acid sequences of SEQ ID NOs: 86, 87, and 88, respectively, and the LCDR1, LCDR2, and LCDR3 comprise amino acid sequences of SEQ ID NOs: 101, KVS, and SEQ ID NO: 103, respectively; or
x. the HCDR1, HCDR2, and HCDR3 comprise amino acid sequences of SEQ ID NOs: 89, 90, and 91, respectively, and the LCDR1, LCDR2, and LCDR3 comprise amino acid sequences of SEQ ID NOs: 104, 105, and 106, respectively; or
xi. the HCDR1, HCDR2, and HCDR3 comprise amino acid sequences of SEQ ID NOs: 109, 110, and 111, respectively, and the LCDR1, LCDR2, and LCDR3 comprise amino acid sequences of SEQ ID NOs: 124, 125, and 126, respectively; or
xii. the HCDR1, HCDR2, and HCDR3 comprise amino acid sequences of SEQ ID NOs: 112, 113, and 114, respectively, and the LCDR1, LCDR2, and LCDR3 comprise amino acid sequences of SEQ ID NOs: 127, 128, and 129, respectively; or
xiii. the HCDR1, HCDR2, and HCDR3 comprise amino acid sequences of SEQ ID NOs: 115, 116, and 117, respectively, and the LCDR1, LCDR2, and LCDR3 comprise amino acid sequences of SEQ ID NOs: 130, 131, and 132, respectively; or
xiv. the HCDR1, HCDR2, and HCDR3 comprise amino acid sequences of SEQ ID NOs: 118, 119, and 120, respectively, and the LCDR1, LCDR2, and LCDR3 comprise amino acid sequences of SEQ ID NO: 133, KVS, and SEQ ID NO: 135, respectively; or
xv. the HCDR1, HCDR2, and HCDR3 comprise amino acid sequences of SEQ ID NOs: 121, 122, and 123, respectively, and the LCDR1, LCDR2, and LCDR3 comprise amino acid sequences of SEQ ID NOs: 136, 137, and 138, respectively; or
xvi. the HCDR1, HCDR2, and HCDR3 comprise amino acid sequences of SEQ ID NOs: 141, 142, and 143, respectively, and the LCDR1, LCDR2, and LCDR3 comprise amino acid sequences of SEQ ID NOs: 156, 157, and 158, respectively; or
xvii. the HCDR1, HCDR2, and HCDR3 comprise amino acid sequences of SEQ ID NOs: 144, 145, and 146, respectively, and the LCDR1, LCDR2, and LCDR3 comprise amino acid sequences of SEQ ID NOs: 159, 160, and 161, respectively; or
xviii. the HCDR1, HCDR2, and HCDR3 comprise amino acid sequences of SEQ ID NOs: 147, 148, and 149, respectively, and the LCDR1, LCDR2, and LCDR3 comprise amino acid sequences of SEQ ID NOs: 162, 163, and 164, respectively; or
xix. the HCDR1, HCDR2, and HCDR3 comprise amino acid sequences of SEQ ID NOs: 150, 151, and 152, respectively, and the LCDR1, LCDR2, and LCDR3 comprise amino acid sequences of SEQ ID NOs: 165, KVS, and SEQ ID NO: 167, respectively; or
xx. the HCDR1, HCDR2, and HCDR3 comprise amino acid sequences of SEQ ID NOs: 153, 154, and 155, respectively, and the LCDR1, LCDR2, and LCDR3 comprise amino acid sequences of SEQ ID NOs: 168, 169, and 170, respectively; or
xxi. the HCDR1, HCDR2, and HCDR3 comprise amino acid sequences of SEQ ID NOs: 173, 174, and 175, respectively, and the LCDR1, LCDR2, and LCDR3 comprise amino acid sequences of SEQ ID NOs: 188, 189, and 190, respectively; or
xxii. the HCDR1, HCDR2, and HCDR3 comprise amino acid sequences of SEQ ID NOs: 176, 177, and 178, respectively, and the LCDR1, LCDR2, and LCDR3 comprise amino acid sequences of SEQ ID NOs: 191, 192, and 193, respectively; or
xxiii. the HCDR1, HCDR2, and HCDR3 comprise amino acid sequences of SEQ ID NOs: 179, 180, and 181, respectively, and the LCDR1, LCDR2, and LCDR3 comprise amino acid sequences of SEQ ID NOs: 194, 195, and 196, respectively; or
xxiv. the HCDR1, HCDR2, and HCDR3 comprise amino acid sequences of SEQ ID NOs: 182, 183, and 184, respectively, and the LCDR1, LCDR2, and LCDR3 comprise amino acid sequences of SEQ ID NO: 197, KVS, and SEQ ID NO: 199, respectively; or
xxv. the HCDR1, HCDR2, and HCDR3 comprise amino acid sequences of SEQ ID NOs: 185, 186, and 187, respectively, and the LCDR1, LCDR2, and LCDR3 comprise amino acid sequences of SEQ ID NOs: 200, 201, and 202, respectively; or
xxvi. the HCDR1, HCDR2, and HCDR3 comprise amino acid sequences of SEQ ID NOs: 205, 206, and 207, respectively, and the LCDR1, LCDR2, and LCDR3 comprise amino acid sequences of SEQ ID NOs: 220, 221, and 222, respectively; or
xxvii. the HCDR1, HCDR2, and HCDR3 comprise amino acid sequences of SEQ ID NOs: 208, 209, and 210, respectively, and the LCDR1, LCDR2, and LCDR3 comprise amino acid sequences of SEQ ID NOs: 223, 224, and 225, respectively; or
xxviii. the HCDR1, HCDR2, and HCDR3 comprise amino acid sequences of SEQ ID NOs: 211, 212, and 213, respectively, and the LCDR1, LCDR2, and LCDR3 comprise amino acid sequences of SEQ ID NOs: 226, 227, and 228, respectively; or
xxix. the HCDR1, HCDR2, and HCDR3 comprise amino acid sequences of SEQ ID NOs: 214, 215, and 216, respectively, and the LCDR1, LCDR2, and LCDR3 comprise amino acid sequences of SEQ ID NO: 229, KVS, and SEQ ID NO: 231, respectively; or
xxx. the HCDR1, HCDR2, and HCDR3 comprise amino acid sequences of SEQ ID NOs: 217, 218, and 219, respectively, and the LCDR1, LCDR2, and LCDR3 comprise amino acid sequences of SEQ ID NOs: 232, 233, and 234, respectively.

Embodiment 2 is the Vβ17 bispecific antibody or antigen-binding fragment thereof of embodiment 1, wherein the binding site for the first antigen binds to Vβ17 on a CD8+ or CD4+ T cell.

Embodiment 3 is the Vβ17 bispecific antibody or antigen-binding fragment thereof of embodiment 1 or 2, wherein the binding site for the second antigen binds to a tumor antigen present on the surface of a cancer cell.

Embodiment 4 is the Vβ17 bispecific antibody or antigen-binding fragment of any one of embodiments 1 to 3, wherein HC1 and LC1 are humanized.

Embodiment 5 is the Vβ17 bispecific antibody or antigen-binding fragment thereof of any one of embodiments 1 to 4, wherein the binding site for the second antigen binds to CD123, kallikrein related peptidase 2 (KLK2), prostate-specific membrane antigen (PSMA), or B-cell maturation antigen (BCMA).

Embodiment 6 is the Vβ17 bispecific antibody or antigen-binding fragment thereof of any one of embodiments 1 to 5, wherein HC2 and LC2 bind to CD123, kallikrein related peptidase 2 (KLK2), prostate-specific membrane antigen (PSMA), or B-cell maturation antigen (BCMA).

Embodiment 7 is the Vβ17 bispecific antibody or antigen-binding fragment thereof of any one of embodiments 1 to 6, wherein the HC2 comprises a heavy chain complementarity determining region 1 (HCDR1-2), a heavy chain complementarity determining region 2 (HCDR2-2), and a heavy chain complementarity determining region 3 (HCDR3-2), and wherein LC2 comprises a light chain complementarity determining region 1 (LCDR1-2), a light chain complementarity determining region 2 (LCDR2-2), and a light chain complementarity determining region 3 (LCDR3-2), and wherein
i. the HCDR1-2, HCDR2-2, and HCDR3-2 comprise amino acid sequences of SEQ ID NOs: 34, 35, and 36, respectively, and the LCDR1-2, LCDR2-2, and LCDR3-2 comprise amino acid sequences of SEQ ID NOs: 37, 38, and 39, respectively; or
ii. the HCDR1-2, HCDR2-2, and HCDR3-2 comprise amino acid sequences of SEQ ID NOs: 237, 238, and 239, respectively, and the LCDR1-2, LCDR2-2, and LCDR3-2 comprise amino acid sequences of SEQ ID NOs: 252, 253, and 254, respectively; or
iii. the HCDR1-2, HCDR2-2, and HCDR3-2 comprise amino acid sequences of SEQ ID NOs: 240, 241, and 242, respectively, and the LCDR1-2, LCDR2-2, and LCDR3-2 comprise amino acid sequences of SEQ ID NOs: 255, 256, and 257, respectively; or
iv. the HCDR1-2, HCDR2-2, and HCDR3-2 comprise amino acid sequences of SEQ ID NOs: 243, 244, and 245, respectively, and the LCDR1-2, LCDR2-2, and LCDR3-2 comprise amino acid sequences of SEQ ID NOs: 258, 259, and 260, respectively; or
v. the HCDR1-2, HCDR2-2, and HCDR3-2 comprise amino acid sequences of SEQ ID NOs: 246, 247, and 248, respectively, and the LCDR1-2, LCDR2-2, and LCDR3-2 comprise amino acid sequences of SEQ ID NO: 261, AAS, and SEQ ID NO: 263, respectively; or
vi. the HCDR1-2, HCDR2-2, and HCDR3-2 comprise amino acid sequences of SEQ ID NOs: 249, 250, and 251, respectively, and the LCDR1-2, LCDR2-2, and LCDR3-2 comprise amino acid sequences of SEQ ID NOs: 264, 265, and 266, respectively; or
vii. the HCDR1-2, HCDR2-2, and HCDR3-2 comprise amino acid sequences of SEQ ID NOs: 269, 270, and 271, respectively, and the LCDR1-2, LCDR2-2, and LCDR3-2 comprise amino acid sequences of SEQ ID NOs: 284, 285, and 286, respectively; or
viii. the HCDR1-2, HCDR2-2, and HCDR3-2 comprise amino acid sequences of SEQ ID NOs: 272, 273, and 274, respectively, and the LCDR1-2, LCDR2-2, and LCDR3-2 comprise amino acid sequences of SEQ ID NOs: 287, 288, and 289, respectively; or
ix. the HCDR1-2, HCDR2-2, and HCDR3-2 comprise amino acid sequences of SEQ ID NOs: 275, 276, and 277, respectively, and the LCDR1-2, LCDR2-2, and LCDR3-2 comprise amino acid sequences of SEQ ID NOs: 290, 291, and 292, respectively; or
x. the HCDR1-2, HCDR2-2, and HCDR3-2 comprise amino acid sequences of SEQ ID NOs: 278, 279, and 280, respectively, and the LCDR1-2, LCDR2-2, and LCDR3-2 comprise amino acid sequences of SEQ ID NO: 293, DNN, and SEQ ID NO: 295, respectively; or
xi. the HCDR1-2, HCDR2-2, and HCDR3-2 comprise amino acid sequences of SEQ ID NOs: 281, 282, and 283, respectively, and the LCDR1-2, LCDR2-2, and LCDR3-2 comprise amino acid sequences of SEQ ID NOs: 296, 297, and 298, respectively; or
xii. the HCDR1-2, HCDR2-2, and HCDR3-2 comprise amino acid sequences of SEQ ID NOs: 301, 302, and 303, respectively, and the LCDR1-2, LCDR2-2, and LCDR3-2 comprise amino acid sequences of SEQ ID NOs: 316, 317, and 318, respectively; or
xiii. the HCDR1-2, HCDR2-2, and HCDR3-2 comprise amino acid sequences of SEQ ID NOs: 304, 305, and 306, respectively, and the LCDR1-2, LCDR2-2, and LCDR3-2 comprise amino acid sequences of SEQ ID NOs: 319, 320, and 321, respectively; or
xiv. the HCDR1-2, HCDR2-2, and HCDR3-2 comprise amino acid sequences of SEQ ID NOs: 307, 308, and 309, respectively, and the LCDR1-2, LCDR2-2, and LCDR3-2 comprise amino acid sequences of SEQ ID NOs: 322, 323, and 324, respectively; or
xv. the HCDR1-2, HCDR2-2, and HCDR3-2 comprise amino acid sequences of SEQ ID NOs: 310, 311, and 312, respectively, and the LCDR1-2, LCDR2-2, and LCDR3-2 comprise amino acid sequences of SEQ ID NO: 325, GAS, and SEQ ID NO: 327, respectively; or
xvi. the HCDR1-2, HCDR2-2, and HCDR3-2 comprise amino acid sequences of SEQ ID NOs: 313, 314, and 315, respectively, and the LCDR1-2, LCDR2-2, and LCDR3-2 comprise amino acid sequences of SEQ ID NOs: 328, 329, and 330, respectively; or
xvii. the HCDR1-2, HCDR2-2, and HCDR3-2 comprise amino acid sequences of SEQ ID NOs: 333, 334, and 335, respectively, and the LCDR1-2, LCDR2-2, and LCDR3-2 comprise amino acid sequences of SEQ ID NOs: 348, 349, and 350, respectively; or
xviii. the HCDR1-2, HCDR2-2, and HCDR3-2 comprise amino acid sequences of SEQ ID NOs: 336, 337, and 338, respectively, and the LCDR1-2, LCDR2-2, and LCDR3-2 comprise amino acid sequences of SEQ ID NOs: 351, 352, and 353, respectively; or
xix. the HCDR1-2, HCDR2-2, and HCDR3-2 comprise amino acid sequences of SEQ ID NOs: 339, 340, and 341, respectively, and the LCDR1-2, LCDR2-2, and LCDR3-2 comprise amino acid sequences of SEQ ID NOs: 354, 355, and 356, respectively; or
xx. the HCDR1-2, HCDR2-2, and HCDR3-2 comprise amino acid sequences of SEQ ID NOs: 342, 343, and 344, respectively, and the LCDR1-2, LCDR2-2, and LCDR3-2 comprise amino acid sequences of SEQ ID NO: 357, DNN, and SEQ ID NO: 359, respectively; or
xxi. the HCDR1-2, HCDR2-2, and HCDR3-2 comprise amino acid sequences of SEQ ID NOs: 345, 346, and 347, respectively, and the LCDR1-2, LCDR2-2, and LCDR3-2 comprise amino acid sequences of SEQ ID NOs: 360, 361, and 362, respectively; or
xxii. the HCDR1-2, HCDR2-2, and HCDR3-2 comprise amino acid sequences of SEQ ID NOs: 365, 366, and 367, respectively, and the LCDR1-2, LCDR2-2, and LCDR3-2 comprise amino acid sequences of SEQ ID NOs: 380, 381, and 382, respectively; or
xxiii. the HCDR1-2, HCDR2-2, and HCDR3-2 comprise amino acid sequences of SEQ ID NOs: 368, 369, and 370, respectively, and the LCDR1-2, LCDR2-2, and LCDR3-2 comprise amino acid sequences of SEQ ID NOs: 383, 384, and 385, respectively; or
xxiv. the HCDR1-2, HCDR2-2, and HCDR3-2 comprise amino acid sequences of SEQ ID NOs: 371, 372, and 373, respectively, and the LCDR1-2, LCDR2-2, and LCDR3-2 comprise amino acid sequences of SEQ ID NOs: 386, 387, and 388, respectively; or
xxv. the HCDR1-2, HCDR2-2, and HCDR3-2 comprise amino acid sequences of SEQ ID NOs: 374, 375, and 376, respectively, and the LCDR1-2, LCDR2-2, and LCDR3-2 comprise amino acid sequences of SEQ ID NO: 389, DNN, and SEQ ID NO: 391, respectively; or
xxvi. the HCDR1-2, HCDR2-2, and HCDR3-2 comprise amino acid sequences of SEQ ID NOs: 377, 378, and 379, respectively, and the LCDR1-2, LCDR2-2, and LCDR3-2 comprise amino acid sequences of SEQ ID NOs: 392, 393, and 394, respectively; or
xxvii. the HCDR1-2, HCDR2-2, and HCDR3-2 comprise amino acid sequences of SEQ ID NOs: 397, 398, and 399, respectively, and the LCDR1-2, LCDR2-2, and LCDR3-2 comprise amino acid sequences of SEQ ID NOs: 412, 413, and 414, respectively; or
xxviii. the HCDR1-2, HCDR2-2, and HCDR3-2 comprise amino acid sequences of SEQ ID NOs: 400, 401, and 402, respectively, and the LCDR1-2, LCDR2-2, and LCDR3-2 comprise amino acid sequences of SEQ ID NOs: 415, 416, and 417, respectively; or
xxix. the HCDR1-2, HCDR2-2, and HCDR3-2 comprise amino acid sequences of SEQ ID NOs: 403, 404, and 405, respectively, and the LCDR1-2, LCDR2-2, and LCDR3-2 comprise amino acid sequences of SEQ ID NOs: 418, 419, and 420, respectively; or
xxx. the HCDR1-2, HCDR2-2, and HCDR3-2 comprise amino acid sequences of SEQ ID NOs: 404, 405, and 406, respectively, and the LCDR1-2, LCDR2-2, and LCDR3-2 comprise amino acid sequences of SEQ ID NO: 421, KAS, and SEQ ID NO: 423, respectively; or
xxxi. the HCDR1-2, HCDR2-2, and HCDR3-2 comprise amino acid sequences of SEQ ID NOs: 407, 408, and 409, respectively, and the LCDR1-2, LCDR2-2, and LCDR3-2 comprise amino acid sequences of SEQ ID NOs: 424, 425, and 426, respectively.

Embodiment 8 is the Vβ17 bispecific antibody or antigen-binding fragment thereof of any one of embodiments 1 to 7, wherein the bispecific antibody or antigen-binding fragment thereof is an IgG isotype.

Embodiment 9 is the Vβ17 bispecific antibody or antigen-binding fragment thereof of any one of embodiments 1 to 8, wherein the bispecific antibody or antigen-binding fragment thereof is a IgG4 isotype.

Embodiment 10 is the Vβ17 bispecific antibody or antigen-binding fragment thereof of any one of embodiments 1 to 8, wherein the bispecific antibody or antigen-binding fragment thereof is a IgG1 isotype.

Embodiment 11 is the Vβ17 bispecific antibody or antigen-binding fragment thereof of any one of embodiments 1 to 10, wherein the bispecific antibody or antigen-binding fragment thereof induces CD8+ or CD4+ T-cell dependent cytotoxicity of a cancer cell in vitro with an EC₅₀ of less than about 0.2 picomolar (pM).

Embodiment 12 is an isolated nucleic acid encoding HC1 and LC1 of the Vβ17 bispecific antibody or antigen-binding fragment thereof of any one of embodiments 1 to 11.

Embodiment 13 is an isolated nucleic acid encoding HC2 and LC2 of the Vβ17 bispecific antibody or antigen-binding fragment thereof of any one of embodiments 1 to 11.

Embodiment 14 is a vector comprising the isolated nucleic acid of embodiment 12 or embodiment 13.

Embodiment 15 is a host cell comprising the vector of embodiment 14.

Embodiment 16 is a buffered composition comprising the Vβ17 bispecific antibody or antigen-binding fragment thereof of any one of embodiments 1 to 11 and a buffered solution.

Embodiment 17 is an isolated anti-Vβ17/anti-CD123 bispecific antibody or antigen-binding fragment thereof comprising:
a. a first heavy chain (HC1);
b. a second heavy chain (HC2)
c. a first light chain (LC1); and
d. a second light chain (LC2),
   wherein HC1 is associated with LC1 and HC2 is associated with LC2, and wherein HC1 comprises a heavy chain complementarity determining region 1 (HCDR1), HCDR2, and HCDR3 comprising the amino acid sequences of SEQ ID NO:1, SEQ ID NO:2, and SEQ ID NO:3, respectively, and LC1 comprises a light chain complementarity determining region 1 (LCDR1), LCDR2, and LCDR3 comprising the amino acid sequences of SEQ ID NO:4, SEQ ID NO:5, and SEQ ID NO:6, respectively, to form a binding site for a first antigen that specifically binds Vβ17, and wherein HC2 comprises a heavy chain complementarity determining region 1 (HCDR1), HCDR2, and HCDR3 comprising the amino acid sequences of SEQ ID NO:34, SEQ ID NO: 35, and SEQ ID NO:36, respectively, and LC2 comprises a light chain complementarity determining region 1 (LCDR1), LCDR2, and LCDR3 comprising the amino acid sequences of SEQ ID NO:37, SEQ ID NO:38, and SEQ ID NO:39, respectively, to form a binding site for a second antigen that specifically binds CD123.

Embodiment 18 is the isolated anti-Vβ17/anti-CD123 bispecific antibody or antigen-binding fragment of embodiment 17, wherein HC1 comprises the amino acid sequence of SEQ ID NO: 13 and LC1 comprises the amino acid sequence of SEQ ID NO:14, and wherein HC2 comprises the amino acid sequence of SEQ ID NO: 15 and LC2 comprises the amino acid sequence of SEQ ID NO: 16.

Embodiment 19 is the isolated anti-Vβ17/anti-CD123 bispecific antibody or antigen-binding fragment thereof of embodiment 17 or embodiment 18, wherein the Vβ17 is on the surface of a CD8+ or CD4+ T cell.

Embodiment 20 is the isolated anti-Vβ17/anti-CD123 bispecific antibody or antigen-binding fragment thereof of any one of embodiments 17 to 19, wherein the CD123 is on the surface of a cancer cell.

Embodiment 21 is the isolated anti-Vβ17/anti-CD123 bispecific antibody or antigen-binding fragment thereof of any one of embodiments 17 to 20, wherein bispecific antibody or antigen-binding fragment thereof induces CD8+ or CD4+ T-cell dependent cytotoxicity of a cancer cell in vitro with an EC₅₀ of less than about 0.2 picomolar (pM).

Embodiment 22 is an isolated nucleic acid encoding the HC1 and LC1 of the anti-Vβ17/anti-CD123 bispecific antibody or antigen-binding fragment thereof of any one of embodiments 17 to 21.

Embodiment 23 is an isolated nucleic acid encoding the HC2 and LC2 of the anti-Vβ17/anti-CD123 bispecific antibody or antigen-binding fragment thereof of any one of embodiments 17 to 21.

Embodiment 24 is a vector comprising the isolated nucleic acid of embodiment 22 or embodiment 23.

Embodiment 25 is a host cell comprising the vector of embodiment 24.

Embodiment 26 is a buffered composition comprising the isolated anti-Vβ17/anti-CD123 bispecific antibody or antigen-binding fragment thereof of any one of embodiments 17 to 21 and a buffered solution.

Embodiment 27 is a method of directing a Vβ17-expressing CD8+ or CD4+ T cell to a cancer cell, the method comprising contacting a Vβ17-expressing CD8+ or CD4+ T cell with the Vβ17 bispecific antibody or antigen-binding fragment thereof of any one of embodiments 1 to 9, wherein contacting the Vβ17-expressing CD8+ or CD4+ T cell with the Vβ17 bispecific antibody or antigen-binding fragment thereof directs the Vβ17-expressing CD8+ or CD4+ T cell to a cancer cell having a second antigen on its surface.

Embodiment 28 is the method of embodiment 27, wherein the second antigen is selected from the group consisting of CD123, kallikrein related peptidase 2 (KLK2), prostate-specific membrane antigen (PSMA), or B-cell maturation antigen (BCMA).

Embodiment 29 (a) is the method of embodiment 27 or 28, wherein the Vβ17-expressing CD8+ or CD4+ T cell is contacted with the Vβ17 bispecific antibody of any one of embodiments 1 to 9.

Embodiment 29 (b) is the method of embodiment 27 or 28, wherein the Vβ17-expressing CD8+ or CD4+ T cell is contacted with the Vβ17 bispecific antibody fragment of any one of embodiments 1 to 9.

Embodiment 30 is a method for inhibiting growth or proliferation of cancer cells, the method comprising contacting the cancer cells with the Vβ17 bispecific antibody or fragment thereof of embodiments 1 to 9, wherein contacting the cancer cells with said antibody or antibody fragment inhibits the growth or proliferation of the cancer cells.

Embodiment 31 (a) is the method of embodiment 30, wherein the cancer cells are in the presence of a Vβ17-expressing CD8+ T cell while in contact with the Vβ17 bispecific antibody or fragment thereof.

Embodiment 31 (b) is the method of embodiment 30 or 31 (a), wherein the cancer cells are contacted with the Vβ17 bispecific antibody of any one of embodiments 1 to 9.

Embodiment 32 is a kit comprising the Vβ17 bispecific antibody or antigen-binding fragment thereof of any one of embodiments 1 to 9 and packaging for the same.

Embodiment 33 is a kit comprising the Vβ17 bispecific antibody or antigen-binding fragment thereof of any one of embodiments 1 to 9 and instructions for use.

Embodiment 34 is a method of producing a Vβ17 bispecific antibody or antigen-binding fragment thereof, comprising culturing the host cell of embodiment 15 under conditions to produce the Vβ17 bispecific antibody or antigen-binding fragment thereof, and recovering the Vβ17 bispecific antibody or antigen-binding fragment thereof from the cell or culture.

Embodiment 35 is an isolated humanized Vβ17 monoclonal antibody or antigen-binding fragment thereof, the humanized Vβ17 monoclonal antibody or antigen-binding fragment thereof comprising a heavy chain (HC) having at least 95% identity to an amino acid sequence selected from the group consisting of SEQ ID NOs: 429, 432, 435, 438, 441, 444, 447, and 450.

Embodiment 36 is the isolated humanized Vβ17 monoclonal antibody or antigen-binding fragment thereof of embodiment 35, wherein the heavy chain (HC) sequence is selected from the group consisting of SEQ ID NOs: 429, 432, 435, 438, 441, 444, 447, and 450.

Embodiment 37 is the isolated humanized Vβ17 monoclonal antibody or antigen-binding fragment thereof of embodiment 35 or 36, wherein the humanized Vβ17 monoclonal antibody or antigen-binding fragment thereof further comprises a light chain (LC) having at least 95% identity to an amino acid sequence selected from the group consisting of SEQ ID NOs: 431, 434, 437, 439, 443, 445, 449, and 452.

Embodiment 38 is the isolated humanized Vβ17 monoclonal antibody or antigen-binding fragment thereof of any one of embodiment 35 to 37, wherein the light chain (LC) sequence is selected from the group consisting of SEQ ID NOs: 431, 434, 437, 439, 443, 445, 449, and 452.

Embodiment 39 is an isolated nucleic acid encoding the humanized Vβ17 monoclonal antibody or antigen-binding fragment thereof of any one of embodiments 35 to 38.

Embodiment 40 is a vector comprising the isolated nucleic acid of any one of embodiments 35 to 39.

Embodiment 41 is a host cell comprising the vector of embodiment 40.

Embodiment 42 is a buffered composition comprising the isolated humanized Vβ17 monoclonal antibody or antigen-binding fragment thereof of any one of embodiments 35 to 39.

Embodiment 42 is a Vβ17 antibody or antigen-binding fragment thereof, the Vβ17 antibody or antigen-binding fragment thereof comprising a first heavy chain (HC1) having a heavy chain complementarity determining region 1 (HCDR1), a heavy chain complementarity determining region 2 (HCDR2), and a heavy chain complementarity determining region 3 (HCDR3), and a first light chain (LC1) having a light chain complementarity determining region 1 (LCDR1), a light chain complementarity determining region 2 (LCDR2), and a light chain complementarity determining region 3 (LCDR3),
wherein
   i. the HCDR1, HCDR2, and HCDR3 comprise amino acid sequences of SEQ ID NOs: 45, 46, and 47, respectively, and the LCDR1, LCDR2, and LCDR3 comprise amino acid sequences of SEQ ID NOs: 60, 61, and 62, respectively; or
   ii. the HCDR1, HCDR2, and HCDR3 comprise amino acid sequences of SEQ ID NOs: 48, 49, and 50, respectively, and the LCDR1, LCDR2, and LCDR3 comprise amino acid sequences of SEQ ID NOs: 63, 64, and 65, respectively; or
   iii. the HCDR1, HCDR2, and HCDR3 comprise amino acid sequences of SEQ ID NOs: 51, 52, and 53, respectively, and the LCDR1, LCDR2, and LCDR3 comprise amino acid sequences of SEQ ID NOs: 66, 67, and 68, respectively; or
   iv. the HCDR1, HCDR2, and HCDR3 comprise amino acid sequences of SEQ ID NOs: 54, 55, and 56, respectively, and the LCDR1, LCDR2, and LCDR3 comprise amino acid sequences of SEQ ID NO: 69, KVS, and SEQ ID NO: 71, respectively; or
   v. the HCDR1, HCDR2, and HCDR3 comprise amino acid sequences of SEQ ID NOs: 57, 58, and 59, respectively, and the LCDR1, LCDR2, and LCDR3 comprise amino acid sequences of SEQ ID NOs: 72, 73, and 74, respectively; or
   vi. the HCDR1, HCDR2, and HCDR3 comprise amino acid sequences of SEQ ID NOs: 77, 78, and 79, respectively, and the LCDR1, LCDR2, and LCDR3 comprise amino acid sequences of SEQ ID NOs: 92, 93, and 94, respectively; or
   vii. the HCDR1, HCDR2, and HCDR3 comprise amino acid sequences of SEQ ID NOs: 80, 81, and 82, respectively, and the LCDR1, LCDR2, and LCDR3 comprise amino acid sequences of SEQ ID NOs: 95, 96, and 97, respectively; or the HCDR1, HCDR2, and HCDR3 comprise amino acid sequences of SEQ ID viii. NOs: 83, 84, and 85, respectively, and the LCDR1, LCDR2, and LCDR3 comprise amino acid sequences of SEQ ID NOs: 98, 99, and 100, respectively; or
   ix. the HCDR1, HCDR2, and HCDR3 comprise amino acid sequences of SEQ ID NOs: 86, 87, and 88, respectively, and the LCDR1, LCDR2, and LCDR3 comprise amino acid sequences of SEQ ID NO: 101, KVS, and SEQ ID NO: 103, respectively; or
   x. the HCDR1, HCDR2, and HCDR3 comprise amino acid sequences of SEQ ID NOs: 89, 90, and 91, respectively, and the LCDR1, LCDR2, and LCDR3 comprise amino acid sequences of SEQ ID NOs: 104, 105, and 106, respectively; or
   xi. the HCDR1, HCDR2, and HCDR3 comprise amino acid sequences of SEQ ID NOs: 109, 110, and 111, respectively, and the LCDR1, LCDR2, and LCDR3 comprise amino acid sequences of SEQ ID NOs: 124, 125, and 126, respectively; or
   xii. the HCDR1, HCDR2, and HCDR3 comprise amino acid sequences of SEQ ID NOs: 112, 113, and 114, respectively, and the LCDR1, LCDR2, and LCDR3 comprise amino acid sequences of SEQ ID NOs: 127, 128, and 129, respectively; or
   xiii. the HCDR1, HCDR2, and HCDR3 comprise amino acid sequences of SEQ ID NOs: 115, 116, and 117, respectively, and the LCDR1, LCDR2, and LCDR3 comprise amino acid sequences of SEQ ID NOs: 130, 131, and 132, respectively; or
   xiv. the HCDR1, HCDR2, and HCDR3 comprise an amino acid sequences of SEQ ID NOs: 118, 119, and 120, respectively, and the LCDR1, LCDR2, and LCDR3 comprise amino acid sequences of SEQ ID NO: 133, KVS, and SEQ ID NO: 135, respectively; or
   xv. the HCDR1, HCDR2, and HCDR3 comprise amino acid sequences of SEQ ID NOs: 121, 122, and 123, respectively, and the LCDR1, LCDR2, and LCDR3 comprise amino acid sequences of SEQ ID NOs: 136, 137, and 138, respectively; or
   xvi. the HCDR1, HCDR2, and HCDR3 comprise amino acid sequences of SEQ ID NOs: 141, 142, and 143, respectively, and the LCDR1, LCDR2, and LCDR3 comprise amino acid sequences of SEQ ID NOs: 156, 157, and 158, respectively; or
   xvii. the HCDR1, HCDR2, and HCDR3 comprise amino acid sequences of SEQ ID NOs: 144, 145, and 146, respectively, and the LCDR1, LCDR2, and LCDR3 comprise amino acid sequences of SEQ ID NOs: 159, 160, and 161, respectively; or
   xviii. the HCDR1, HCDR2, and HCDR3 comprise amino acid sequences of SEQ ID NOs: 147, 148, and 149, respectively, and the LCDR1, LCDR2, and LCDR3 comprise amino acid sequences of SEQ ID NOs: 162, 163, and 164, respectively; or
   xix. the HCDR1, HCDR2, and HCDR3 comprise amino acid sequences of SEQ ID NOs: 150, 151, and 152, respectively, and the LCDR1, LCDR2, and LCDR3 comprise amino acid sequences of SEQ ID NO: 165, KVS, and SEQ ID NO: 167, respectively; or
   xx. the HCDR1, HCDR2, and HCDR3 comprise amino acid sequences of SEQ ID NOs: 153, 154, and 155, respectively, and the LCDR1, LCDR2, and LCDR3 comprise amino acid sequences of SEQ ID NOs: 168, 169, and 170, respectively; or
   xxi. the HCDR1, HCDR2, and HCDR3 comprise amino acid sequences of SEQ ID NOs: 173, 174, and 175, respectively, and the LCDR1, LCDR2, and LCDR3 comprise amino acid sequences of SEQ ID NOs: 188, 189, and 190, respectively; or
   xxii. the HCDR1, HCDR2, and HCDR3 comprise amino acid sequences of SEQ ID NOs: 176, 177, and 178, respectively, and the LCDR1, LCDR2, and LCDR3 comprise amino acid sequences of SEQ ID NOs: 191, 192, and 193, respectively; or
   xxiii. the HCDR1, HCDR2, and HCDR3 comprise amino acid sequences of SEQ ID NOs: 179, 180, and 181, respectively, and the LCDR1, LCDR2, and LCDR3 comprise amino acid sequences of SEQ ID NOs: 194, 195, and 196, respectively; or
   xxiv. the HCDR1, HCDR2, and HCDR3 comprise amino acid sequences of SEQ ID NOs: 182, 183, and 184, respectively, and the LCDR1, LCDR2, and LCDR3 comprise amino acid sequences of SEQ ID NO: 197, KVS, and SEQ ID NO: 199, respectively; or
   xxv. the HCDR1, HCDR2, and HCDR3 comprise amino acid sequences of SEQ ID NOs: 185, 186, and 187, respectively, and the LCDR1, LCDR2, and LCDR3 comprise amino acid sequences of SEQ ID NOs: 200, 201, and 202, respectively; or
   xxvi. the HCDR1, HCDR2, and HCDR3 comprise amino acid sequences of SEQ ID NOs: 205, 206, and 207, respectively, and the LCDR1, LCDR2, and LCDR3 comprise amino acid sequences of SEQ ID NOs: 220, 221, and 222, respectively; or
   xxvii. the HCDR1, HCDR2, and HCDR3 comprise amino acid sequences of SEQ ID NOs: 208, 209, and 210, respectively, and the LCDR1, LCDR2, and LCDR3 comprise amino acid sequences of SEQ ID NOs: 223, 224, and 225, respectively; or
   xxviii. the HCDR1, HCDR2, and HCDR3 comprise amino acid sequences of SEQ ID NOs: 211, 212, and 213, respectively, and the LCDR1, LCDR2, and LCDR3 comprise amino acid sequences of SEQ ID NOs: 226, 227, and 228, respectively; or
   xxix. the HCDR1, HCDR2, and HCDR3 comprise amino acid sequences of SEQ ID NOs: 214, 215, and 216, respectively, and the LCDR1, LCDR2, and LCDR3 comprise amino acid sequences of SEQ ID NO: 229, KVS, and SEQ ID NO: 231, respectively; or
   xxx. the HCDR1, HCDR2, and HCDR3 comprise amino acid sequences of SEQ ID NOs: 217, 218, and 219, respectively, and the LCDR1, LCDR2, and LCDR3 comprise amino acid sequences of SEQ ID NOs: 232, 233, and 234, respectively.

Embodiment 43 is an antibody or antigen-binding fragment thereof, the antibody or antigen binding-fragment comprising a second heavy chain (HC2) having a heavy chain complementarity determining region 1 (HCDR1-2), a heavy chain complementarity determining region 2 (HCDR2-2), and a heavy chain complementarity determining region 3 (HCDR3-2), and a second light chain (LC2) having a light chain complementarity determining region 1 (LCDR1-2), a light chain complementarity determining region 2 (LCDR2-2), and a light chain complementarity determining region 3 (LCDR3-2), and
wherein
   i. the HCDR1-2, HCDR2-2, and HCDR3-2 comprise amino acid sequences of SEQ ID NOs: 34, 35, and 36, respectively, and the LCDR1-2, LCDR2-2, and LCDR3-2 comprise amino acid sequences of SEQ ID NOs: 37, 38, and 39, respectively; or
   ii. the HCDR1-2, HCDR2-2, and HCDR3-2 comprise amino acid sequences of SEQ ID NOs: 237, 238, and 239, respectively, and the LCDR1-2, LCDR2-2, and LCDR3-2 comprise amino acid sequences of SEQ ID NOs: 252, 253, and 254, respectively; or
   iii. the HCDR1-2, HCDR2-2, and HCDR3-2 comprise amino acid sequences of SEQ ID NOs: 240, 241, and 242, respectively, and the LCDR1-2, LCDR2-2, and LCDR3-2 comprise amino acid sequences of SEQ ID NOs: 255, 256, and 257, respectively; or
   iv. the HCDR1-2, HCDR2-2, and HCDR3-2 comprise amino acid sequences of SEQ ID NOs: 243, 244, and 245, respectively, and the LCDR1-2, LCDR2-2, and LCDR3-2 comprise amino acid sequences of SEQ ID NOs: 258, 259, and 260, respectively; or
   v. the HCDR1-2, HCDR2-2, and HCDR3-2 comprise amino acid sequences of SEQ ID NOs: 246, 247, and 248, respectively, and the LCDR1-2, LCDR2-2, and LCDR3-2 comprise amino acid sequences of SEQ ID NO: 261, AAS, and SEQ ID NO: 263, respectively; or
   vi. the HCDR1-2, HCDR2-2, and HCDR3-2 comprise amino acid sequences of SEQ ID NOs: 249, 250, and 251, respectively, and the LCDR1-2, LCDR2-2, and LCDR3-2 comprise amino acid sequences of SEQ ID NOs: 264, 265, and 266, respectively; or
   vii. the HCDR1-2, HCDR2-2, and HCDR3-2 comprise amino acid sequences of SEQ ID NOs: 269, 270, and 271, respectively, and the LCDR1-2, LCDR2-2, and LCDR3-2 comprise amino acid sequences of SEQ ID NOs: 284, 285, and 286, respectively; or
   viii. the HCDR1-2, HCDR2-2, and HCDR3-2 comprise amino acid sequences of SEQ ID NOs: 272, 273, and 274, respectively, and the LCDR1-2, LCDR2-2, and LCDR3-2 comprise amino acid sequences of SEQ ID NOs: 287, 288, and 289, respectively; or
   ix. the HCDR1-2, HCDR2-2, and HCDR3-2 comprise amino acid sequences of SEQ ID NOs: 275, 276, and 277, respectively, and the LCDR1-2, LCDR2-2, and LCDR3-2 comprise amino acid sequences of SEQ ID NOs: 290, 291, and 292, respectively; or
   x. the HCDR1-2, HCDR2-2, and HCDR3-2 comprise amino acid sequences of SEQ ID NOs: 278, 279, and 280, respectively, and the LCDR1-2, LCDR2-2, and LCDR3-2 comprise amino acid sequences of SEQ ID NO: 293, DNN, and SEQ ID NO: 295, respectively; or
   xi. the HCDR1-2, HCDR2-2, and HCDR3-2 comprise amino acid sequences of SEQ ID NOs: 281, 282, and 283, respectively, and the LCDR1-2, LCDR2-2, and LCDR3-2 comprise amino acid sequences of SEQ ID NOs: 296, 297, and 298, respectively; or
   xii. the HCDR1-2, HCDR2-2, and HCDR3-2 comprise amino acid sequences of SEQ ID NOs: 301, 302, and 303, respectively, and the LCDR1-2, LCDR2-2, and LCDR3-2 comprise amino acid sequences of SEQ ID NOs: 316, 317, and 318, respectively; or
   xiii. the HCDR1-2, HCDR2-2, and HCDR3-2 comprise amino acid sequences of SEQ ID NOs: 304, 305, and 306, respectively, and the LCDR1-2, LCDR2-2, and LCDR3-2 comprise amino acid sequences of SEQ ID NOs: 319, 320, and 321, respectively; or
   xiv. the HCDR1-2, HCDR2-2, and HCDR3-2 comprise amino acid sequences of SEQ ID NOs: 307, 308, and 309, respectively, and the LCDR1-2, LCDR2-2, and LCDR3-2 comprise amino acid sequences of SEQ ID NOs: 322, 323, and 324, respectively; or
   xv. the HCDR1-2, HCDR2-2, and HCDR3-2 comprise amino acid sequences of SEQ ID NOs: 310, 311, and 312, respectively, and the LCDR1-2, LCDR2-2, and LCDR3-2 comprise amino acid sequences of SEQ ID NO: 325, GAS, and SEQ ID NO: 327, respectively; or
   xvi. the HCDR1-2, HCDR2-2, and HCDR3-2 comprise amino acid sequences of SEQ ID NOs: 313, 314, and 315, respectively, and the LCDR1-2, LCDR2-2, and LCDR3-2 comprise amino acid sequences of SEQ ID NOs: 328, 329, and 330, respectively; or
   xvii. the HCDR1-2, HCDR2-2, and HCDR3-2 comprise amino acid sequences of SEQ ID NOs: 333, 334, and 335, respectively, and the LCDR1-2, LCDR2-2, and LCDR3-2 comprise amino acid sequences of SEQ ID NOs: 348, 349, and 350, respectively; or
   xviii. the HCDR1-2, HCDR2-2, and HCDR3-2 comprise amino acid sequences of SEQ ID NOs: 336, 337, and 338, respectively, and the LCDR1-2, LCDR2-2, and LCDR3-2 comprise amino acid sequences of SEQ ID NOs: 351, 352, and 353, respectively; or
   xix. the HCDR1-2, HCDR2-2, and HCDR3-2 comprise amino acid sequences of SEQ ID NOs: 339, 340, and 341, respectively, and the LCDR1-2, LCDR2-2, and LCDR3-2 comprise amino acid sequences of SEQ ID NOs: 354, 355, and 356, respectively; or
   xx. the HCDR1-2, HCDR2-2, and HCDR3-2 comprise amino acid sequences of SEQ ID NOs: 342, 343, and 344, respectively, and the LCDR1-2, LCDR2-2, and LCDR3-2 comprise amino acid sequences of SEQ ID NO: 357, DNN, and SEQ ID NO: 359, respectively; or
   xxi. the HCDR1-2, HCDR2-2, and HCDR3-2 comprise amino acid sequences of SEQ ID NOs: 345, 346, and 347, respectively, and the LCDR1-2, LCDR2-2, and LCDR3-2 comprise amino acid sequences of SEQ ID NOs: 360, 361, and 362, respectively; or
   xxii. the HCDR1-2, HCDR2-2, and HCDR3-2 comprise amino acid sequences of SEQ ID NOs: 365, 366, and 367, respectively, and the LCDR1-2, LCDR2-2, and LCDR3-2 comprise amino acid sequences of SEQ ID NOs: 380, 381, and 382, respectively; or
   xxiii. the HCDR1-2, HCDR2-2, and HCDR3-2 comprise amino acid sequences of SEQ ID NOs: 368, 369, and 370, respectively, and the LCDR1-2, LCDR2-2, and LCDR3-2 comprise amino acid sequences of SEQ ID NOs: 383, 384, and 385, respectively; or
   xxiv. the HCDR1-2, HCDR2-2, and HCDR3-2 comprise amino acid sequences of SEQ ID NOs: 371, 372, and 373, respectively, and the LCDR1-2, LCDR2-2, and LCDR3-2 comprise amino acid sequences of SEQ ID NOs: 386, 387, and 388, respectively; or
   xxv. the HCDR1-2, HCDR2-2, and HCDR3-2 comprise amino acid sequences of SEQ ID NOs: 374, 375, and 376, respectively, and the LCDR1-2, LCDR2-2, and LCDR3-2 comprise amino acid sequences of SEQ ID NO: 389, DNN, and SEQ ID NO: 391, respectively; or
   xxvi. the HCDR1-2, HCDR2-2, and HCDR3-2 comprise amino acid sequences of SEQ ID NOs: 377, 378, and 379, respectively, and the LCDR1-2, LCDR2-2, and LCDR3-2 comprise amino acid sequences of SEQ ID NOs: 392, 393, and 394, respectively; or
   xxvii. the HCDR1-2, HCDR2-2, and HCDR3-2 comprise amino acid sequences of SEQ ID NOs: 397, 398, and 399, respectively, and the LCDR1-2, LCDR2-2, and LCDR3-2 comprise amino acid sequences of SEQ ID NOs: 412, 413, and 414, respectively; or
   xxviii. the HCDR1-2, HCDR2-2, and HCDR3-2 comprise amino acid sequences of SEQ ID NOs: 400, 401, and 402, respectively, and the LCDR1-2, LCDR2-2, and LCDR3-2 comprise amino acid sequences of SEQ ID NOs: 415, 416, and 417, respectively; or
   xxix. the HCDR1-2, HCDR2-2, and HCDR3-2 comprise amino acid sequences of SEQ ID NOs: 403, 404, and 405, respectively, and the LCDR1-2, LCDR2-2, and LCDR3-2 comprise amino acid sequences of SEQ ID NOs: 418, 419, and 420, respectively; or
   xxx. the HCDR1-2, HCDR2-2, and HCDR3-2 comprise amino acid sequences of SEQ ID NOs: 404, 405, and 406, respectively, and the LCDR1-2, LCDR2-2, and LCDR3-2 comprise amino acid sequences of SEQ ID NO: 421, KAS, and SEQ ID NO: 423, respectively; or
   xxxi. the HCDR1-2, HCDR2-2, and HCDR3-2 comprise amino acid sequences of SEQ ID NOs: 407, 408, and 409, respectively, and the LCDR1-2, LCDR2-2, and LCDR3-2 comprise amino acid sequences of SEQ ID NOs: 424, 425, and 426, respectively.

### EXAMPLES

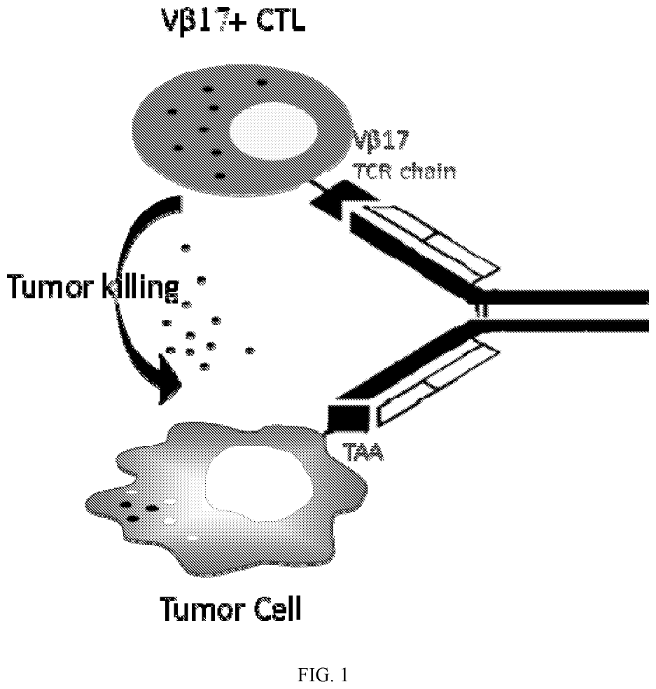

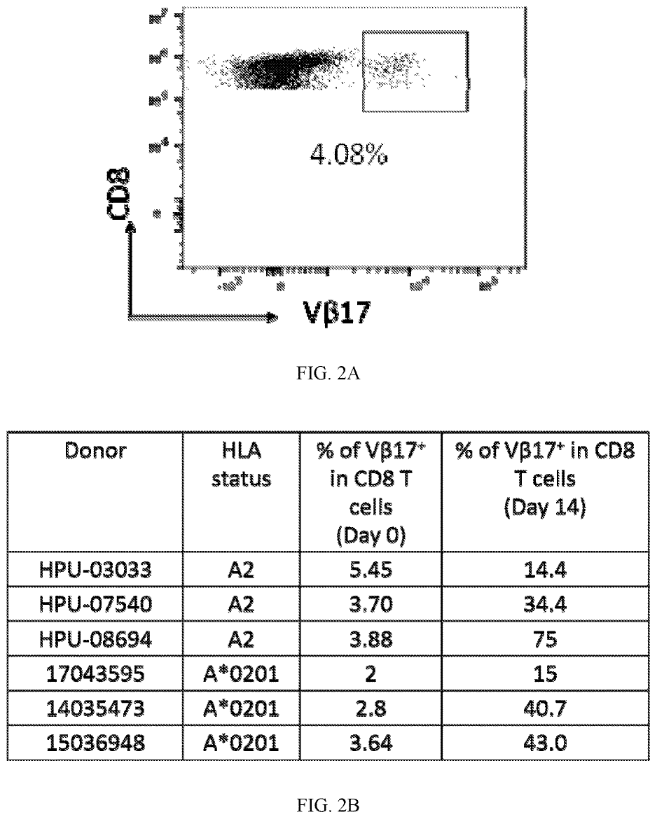

The following examples are based on the premise that influenza virus derived peptide M1 is capable of expanding a select set of T cells. These cells express TCR-haplotype-Vβ17 and majority of these cells exhibit efficient cytotoxicity of tumor target cells. This ability is then harnessed using bispecific antibodies constructed such that one arm binds to the Vβ17 structure and the other arm binds to an antigen expressed by the cancer cells. Thus, the bispecific antibody bridges the effector and target cells together-resulting in cancer cell killing. This mechanism of action is described in the schematic outlined in FIG. 1.

The subsequent examples can be divided into the following categories: (1) Generation of bispecific antibodies capable of binding to the Vβ17 arm of T-cell receptors (TCR) on CTL (Examples 1 and 2); and (2) Evidence for bispecific antibody-enabled target cell killing by CTL expanded in vitro (Example 3).

### Example 1: Human Framework Adaptation of Anti-Vβ17 mAb E17.5F

The mouse IgG1 anti-human T cell receptor Vβ17 clone E17.5F was obtained from BeckmanCoulter, Inc. (Brea, CA). Sample preparation and LC/MSMS analysis were performed at Protea Bioscience Inc. (Morgantown, WV). The sample was reduced and alkylated, divided into seven aliquots, and proteolytically digested with Trypsin/LysC, Chymotrypsin, LysC, Pepsin, and AspN, Elastase, and Proteinase K enzymes. Resulting peptides were desalted using a ZipTip C18 Pipette Tips and separated on-line using reverse phase chromatography. Mass spectrometry was performed on Thermo Q-Exactive spectrometer using HCD fragmentation. MS data sets were analyzed using PEAKS software by matching de novo sequence tags to an IMGT-based antibody sequences database. Gaps in the sequence were assigned using Contig sequence assembly of de novo identified peptides. All CDRs and hyper-mutations were confirmed by inspecting the MS/MS spectra.

The sequences obtained are shown in Tables 1 and 2.

TABLE 1
CDR Sequences of TCR Vβ17 clone E17.5F.

| | | SEQ | | SEQ | | SEQ |
|---|---|---|---|---|---|---|
| Antibody | HCDR1 | ID NO: | HCDR2 | ID NO: | HCDR3 | ID NO: |
| E17.5F | GYSITSGYFWN | 1 | YISYDGSNN | 2 | PSPGTGYAVDY | 3 |
| | | SEQ | | SEQ | | SEQ |
| Antibody | LCDR1 | ID NO: | LCDR2 | ID NO: | LCDR3 | ID NO: |
| E17.5F | RSSQSLVHSNGNTYLH | 4 | KVSNRFS | 5 | SQSTHVPFT | 6 |

TABLE 2
Heavy chain and light chain sequences of TCR Vβ17 clone E17.5F.

| mAb ID | Heavy Chain Amino Acid Sequence | SEQ ID NO: |
|---|---|---|
| B17B01 | NVQLQESGPGLVKPSQSLSLTCSVAGYSITSGYFWNWIRQFPGNKLEWMGYIS | 7 |
| | YDGSNNYNPSLKNRISITRDTSKNQFFLKLNSVTTEDTATYYCASPSPGTGYAV | |
| | DYWGQGTSVTVSSAKTTPPSVYPLAPGSAAQTNSMVTLGCLVKGYFPEPVTV | |
| | TWNSGSLSSGVHTFPAVLQSDLYTLSSSVTVPSSTWPSQTVTCNVAHPASSTKV | |
| | DKKIVPRDCGCKPCICTVPEVSSVFIFPPKPKDVLTITLTPKVTCVVVDISKDDPE | |
| | VQFSWFVDDVEVHTAQTKPREEQINSTFRSVSELPIMHQDWLNGKEFKCRVNS | |
| | AAFPAPIEKTISKTYGRPKAPQVYTIPPPKEQMAKDKVSLTCMITNFFPEDITVE | |
| | WQWNGQPAENYKNTQPIMDTDGSYFVYSKLNVQKSNWEAGNTFTCSVLHEG | |
| | LHNHHTEKSLSHSPGK | |
| | Light Chain Amino Acid Sequence | SEQ ID NO: |
| B17B01 | NVVMTQTPLSLPVSLGDQASISCRSSQSLVHSNGNTYLHWYLQKPGQSPKFLIY | 8 |
| | KVSNRFSGVPDRFSGGGSGTEFTLKISRVEAEDLGVYFCSQSTHVPFTFGSGTK | |
| | LEIKRADAAPTVSIFPPSSEQLTSGGASVVCFLNNFYPKDINVKWKIDGSERQN | |
| | GVLNSWTDQDSKDSTYSMSSTLTLTKDEYERHNSYTCEATHKTSTSPIVKSFN | |
| | RNEC | |

Changes were made in the sequences for the preparation of bispecific antibodies (Table 3). The changes include the following: (1) a framework mutation Asn1 of the heavy chain was not conserved, so the sequence has been modified to have the DVQLW sequence; (2) another mutation identified in the Fc, K337Y, was deemed uncharacteristic, and, thus, a construct without this mutation was synthesized; and (3) a potential secondary glycosylation site on the heavy chain was observed, and, thus, two versions of this mAb with and without the N-linked site (N82a, based on Chothia numbering) were synthesized.

TABLE 3
Heavy and Light Chain sequences for Vβ17 clone E17.5F antibody variants

| mAb ID | Heavy Chain Amino Acid Sequence | SEQ ID NO: |
|---|---|---|
| B17B1 | NVQLQESGPGLVKPSQSLSLTCSVAGYSITSGYFWNWIRQFPGNKLEWMGYIS | 9 |
| | YDGSNNYNPSLKNRISITRDTSKNQFFLKLNSVTTEDTATYYCASPSPGTGYAV | |
| | DYWGQGTSVTVSSASTKGPSVFPLAPCSRSTSESTAALGCLVKDYFPEPVTVS | |
| | WNSGALTSGVHTFPAVLQSSGLYSLSSVVTVPSSSLGTKTYTCNVDHKPSNTK | |
| | VDKRVESKYGPPCPPCPAPEAAGGPSVFLFPPKPKDTLMISRTPEVTCVVVDVS | |
| | QEDPEVQFNWYVDGVEVHNAKTKPREEQFNSTYRVVSVLTVLHQDWLNGKE | |
| | YKCKVSNKGLPSSIEKTISKAKGQPREPQVYTLPPSQEEMTKNQVSLTCLVKGF | |
| | YPSDIAVEWESNGQPENNYKTTPPVLDSDGSFLLYSKLTVDKSRWQEGNVFSC | |
| | SVMHEALHNHYTQKSLSLSLGK | |
| B17B2 | DVQLKESGPGLVKPSQSLSVTCSVTGYSITSGYYWNWYRQFPGNKLEWMGYI | 11 |
| | SYDGSNNYNPSLKNRISITRDTSKNQILLKLTYVTTEDTATYYCTRPSPGTGYA | |
| | VDYWGQGTLVTVSSASTKGPSVFPLAPCSRSTSESTAALGCLVKDYFPEPVTVS | |
| | WNSGALTSGVHTFPAVLQSSGLYSLSSVVTVPSSSLGTKTYTCNVDHKPSNTK | |
| | VDKRVESKYGPPCPPCPAPEAAGGPSVFLFPPKPKDTLMISRTPEVTCVVVDVS | |
| | QEDPEVQFNWYVDGVEVHNAKTKPREEQFNSTYRVVSVLTVLHQDWLNGKE | |
| | YKCKVSNKGLPSSIEKTISKAKGQPREPQVYTLPPSQEEMTKNQVSLTCLVKGF | |
| | YPSDIAVEWESNGQPENNYKTTPPVLDSDGSFLLYSKLTVDKSRWQEGNVFSC | |
| | SVMHEALHNHYTQKSLSLSLGK | |
| | Light Chain Amino Acid Sequence | SEQ ID NO: |
| B17B1 | NVVMTQTPLSLPVSLGDQASISCRSSQSLVHSNGNTYLHWYLQKPGQSPKFLIY | 10 |
| | KVSNRFSGVPDRFSGGGSGTEFTLKISRVEAEDLGVYFCSQSTHVPFTFGSGTK | |
| | LEIKRADAAPTVSIFPPSSEQLTSGGASVVCFLNNFYPKDINVKWKIDGSERQN | |
| | GVLNSWTDQDSKDSTYSMSSTLTLTKDEYERHNSYTCEATHKTSTSPIVKSFN | |
| | RNEC | |
| B17B2 | DIVMTQSPDSLAVSLGERATINCRSSQSLVHSNGNTYLHWYQQKPGQPPKLLI | 12 |
| | YKVSNRFSGVPDRFSGSGSGTDFTLTISSLQAEDVAVYYCSQSTHVPFTFGQGT | |
| | KVEIKRADAAPTVSIFPPSSEQLTSGGASVVCFLNNFYPKDINVKWKIDGSERQ | |
| | NGVLNSWTDQDSKDSTYSMSSTLTLTKDEYERHNSYTCEATHKTSTSPIVKSF | |
| | NRNEC | |

The two antibodies (B17B1 and B17B2) were expressed in HEK293Expi cells. The supernatants were tested for Vβ17 binding (B17B1 and B17B2) and only B17B1 demonstrated binding. Thus, B17B1 was expressed having an IgG4 constant region with Fc substitutions.

The anti-human TCR Vβ17 mouse mAb B17B1 was humanized using the Human Framework Adaptation (HFA) method (Fransson J, et al. J. Mol. Biol. 2010; 398:214-231). To find the best combination of humanized heavy and light chains, several human V-region sequences were selected for testing (Table 4). Selection of human germlines was based solely on the overall sequence similarity to the mouse antibody in the framework (FR) region. Neither the CDR sequences, nor their length or canonical structures, were considered in this selection.

The CDR definition used in HFA is described in (Fransson J, et al. J. Mol. Biol. 2010; 398:214-231) and corresponds to the Martin's definition (Abhinandan K R and Martin A C. Mol. Immunol. 2008; 45:3832-3839). The CDRs (Table 1) were defined as described below (using the Chothia numbering scheme [Chothia C, and Lesk A. J. Mol. Biol. 1987; 196:901-917]):
HCDR1 (SEQ ID NO: 1) 26-35
HCDR2 (SEQ ID NO: 2) 50-58
HCDR3 (SEQ ID NO: 3) 95-102
LCDR1 (SEQ ID NO: 4) 24-34
LCDR2 (SEQ ID NO: 5) 50-56
LCDR3 (SEQ ID NO: 6) 89-97

The selected human germlines are provided in Table 4 (in the IMGT notation).

TABLE 4
VH and VL variants

| Ab VH | Sequence | SEQ ID NO: |
|---|---|---|
| B17H1 | NVQLQESGPGLVKPSQSLSLTCSVAGYSITSGYFWNWIRQFPGNKLEWMG | 25 |
| | YISYDGSNNYNPSLKNRISITRDTSKNQFFLKLNSVTTEDTATYYCASPSPG | |
| | TGYAVDYWGQGTSVTVSS | |
| B17H3 | EVQLLESGGGLVQPGGSLRLSCAASGYSITSGYFWNWVRQAPGKGLEWVS | 19 |
| | YISYDGSNNYADSVKGRFTISRDNSKNTLYLQMNSLRAEDTAVYYCAKPSP | |
| | GTGYAVDYWGQGTLVTVSS | |
| B17H4 | EVQLLESGGGLVQPGGSLRLSCAASGYSITSGYFWNWVRQAPGKGLEWVS | 20 |
| | YISYDGSNNYADSVKGRFTISRDNSKNTLYLQMNSLRAEDTAVYYCASPSPG | |
| | TGYAVDYWGQGTLVTVSS | |
| B17H5 | QVQLQESGPGLVKPSETLSLTCTVSGYSITSGYFWNWIRQPPGKGLEWIGYIS | 21 |
| | YDGSNNYNPSLKSRVTISRDTSKNQFSLKLSSVTAADTAVYYCASPSPGTGY | |
| | AVDYWGQGTLVTVSS | |
| Ab VL | Sequence | SEQ ID NO: |
| B17L1 | NVVMTQTPLSLPVSLGDQASISCRSSQSLVHSNGNTYLHWYLQKPGQSPKFLIY | 26 |
| | KVSNRFSGVPDRFSGGGSGTEFTLKISRVEAEDLGVYFCSQSTHVPFTFGSGTKL | |
| | EIK | |
| B17L3 | DIQMTQSPSSLSASVGDRVTITCRSSQSLVHSNGNTYLHWYQQKPGKAPKLLIY | 22 |
| | KVSNRFSGVPSRFSGSGSGTDFTLTISSLQPEDFATYYCSQSTHVPFTFGQGTKL | |
| | EIK | |
| B17L4 | DIQMTQSPSSLSASVGDRVTITCRSSQSLVHSNGNTYLHWYQQKPGKAPKFLIY | 23 |
| | KVSNRFSGVPSRFSGSGSGTDFTLTISSLQPEDFATYYCSQSTHVPFTFGQGTKL | |
| | EIK | |
| B17L5 | DVVMTQSPLSLPVTLGQPASISCRSSQSLVHSNGNTYLHWFQQRPGQSPRFLIY | 24 |
| | KVSNRFSGVPDRFSGSGSGTDFTLKISRVEAEDVGVYYCSQSTHVPFTFGQGTKL | |
| | EIK | |

CDRs1-3 are underlined

“Back mutations” in several variants were introduced at FR positions that are known to be important for VL/VH pairing and CDR conformation. The selected human germlines are provided in Table 5 (in the IMGT notation), with the back mutations noted.

TABLE 5
The selected J-regions

| J-region | Sequence | SEQ ID NO: |
|---|---|---|
| IGHJ1*01 HC | WGQGTLVTVSS | 42 |
| IGKJ2*01 LC | FGQGTKLEIK | 43 |

Amino acid sequences of all nine pairwise combinations of three heavy chains and three light chains were back-translated to DNA, and cDNA was prepared using gene synthesis techniques (U.S. Pat. Nos. 6,670,127; 6,521,427). Heavy chain (HC) variable regions were subcloned onto human IgG4 constant region using an in-house expression vector with the CMV promoter using standard molecular biology techniques. Light chain (LC) variable regions were subcloned onto a human Lambda (2) constant regions using an in-house expression vector with the CMV promoter using standard molecular biology techniques. Resulting plasmids were transfected into HEK EXPI cells (LifeTechnologies; Carlsbad, CA) and mAbs were expressed. Purification was by standard methods using a Protein A column (hiTrap MAbSelect SuRe column). After elution, the pools were dialyzed into D-PBS, pH 7.2.

TABLE 6
Heavy and Light chains of nine humanized Vβ17 antibodies

| | | | | | Concentration |
|---|---|---|---|---|---|
| mAb | Hc | SEQ ID NO: | Lc | SEQ ID NO: | (μg/mL) |
| B17B14 | B17H3 | 19 | B17L3 | 22 | 686.3 |
| B17B15 | B17H3 | 19 | B17L4 | 23 | 13.8 |
| B17B16 | B17H3 | 19 | B17L5 | 24 | 14.6 |
| B17B17 | B17H4 | 20 | B17L3 | 22 | 335.1 |
| B17B18 | B17H4 | 20 | B17L4 | 23 | 45.2 |
| B17B19 | B17H4 | 20 | B17L5 | 24 | 27.5 |
| B17B20 | B17H5 | 21 | B17L3 | 22 | 602.1 |
| B17B21 | B17H5 | 21 | B17L4 | 23 | 570.9 |
| B17B22 | B17H5 | 21 | B17L5 | 24 | 320.5 |

The humanized antibodies were screened for binding to a TCRVβ17 (SEQID NO: 27)/Va10.2-Fc (SEQ ID NO:44) fusion protein by ELISA. Biotinylated TCRVβ17/Va10.2-Fc fusion protein was added to a streptavidin-coated ELISA plate. Unbound protein was washed away and mAb was added at a range of concentrations (0.01-10 μg/mL). Plates were washed and anti-kappa: HRP detection antibody was added. Plates were washed, chemiluminescent detection reagent was added, and the plates were read on a Perkin Elmer EnVision plate reader for luminescence. B17B20 and B17B21 showed positive binding to the TCR-Vβ17 protein. B17B22 showed weak binding to this protein. These antibodies were then purified as described above for further studies. B17B21 demonstrated the best binding to recombinant TCR-Vβ17 protein and to M1-stimulated T-cells and was thus chosen as the molecule for further functional studies, specifically T-cell re-directed cancer cell killing as a bispecific antibody.

Thus, the variable region sequence of B17B21 (anti-Vβ17) and I3RB217 (anti-CD123 antibody) was used to generate a bispecific antibody to be tested for T-cell re-directed killing of acute myeloid leukemia (AML) cells.

### Example 2. Preparation of Anti-Vβ17/Anti-CD123 Bispecific Antibodies

VB11 (anti-Vβ17/anti-CD123) and VB13 (Vβ17×Null) bispecific antibodies were produced as full-length antibodies in the knob-into-hole format as human IgG4, as previously described (Atwell et al. J. Mol. Biol. 270:26-35, 1997). Nucleic acid sequences encoding variable regions were subcloned into a custom mammalian expression vectors containing constant region of IgG4 expression cassettes using standard PCR restriction enzyme based cloning techniques. The bispecific antibodies were expressed by transient transfection in Chinese hamster ovary cell line. The antibodies were initially purified by Mab Select SuRe Protein A column (GE healthcare, Piscataway, New Jersey) (Brown, Bottomley et al. 1998). The column was equilibrated with Phosphate Buffer Saline (PBS), pH 7.2 and loaded with fermentation supernatant at a flow rate of 2 mL/min. After loading, the column was washed with PBS (4 CV) followed by elution in 30 mM sodium acetate, pH 3.5. Fractions containing protein peaks as monitored by Absorbance at 280 nm in Akta Explorer (GE healthcare) were pooled together and were neutralized to pH 5.0 by adding 1% of 3M sodium acetate, pH 9.0. As a polishing step, the antibodies were purified on a preparative size exclusion chromatography (SEC) using a Superdex 200 column (GE healthcare). The integrity of the sample was assessed by endotoxin measurement and SDS polyacrylamide gel electrophoresis under reducing and non-reducing conditions. The final protein concentrations were 0.48 mg/ml for anti-Vβ17/anti-CD123 and 0.24 mg/mL for Vβ17×Null. The final EU levels of anti-Vβ17/anti-CD123 and Vβ17×Null based on these protein concentrations were 2.053 EU/mg and 4.219 EU/mg, respectively.

TABLE 7
Sequences of half antibodies expressed in CHO cells

| mAb ID | | SEQ ID NO: |
|---|---|---|
| 9 | ‘Knob’ arm and ‘hole’ arm amino acid sequence | |
| B17B21 | MAWVWTLLFLMAAAQSIQADIQMTQSPSSLSASVGDRVTITCRSSQSLVHS | 28 |
| (VB17 | NGNTYLHWYQQKPGKAPKFLIYKVSNRFSGVPSRFSGSGSGTDFTLTISSLQ | |
| half Ab) | PEDFATYYCSQSTHVPFTFGQGTKLEIKRTVAAPSVFIFPPSDEQLKSGTASV | |
| | VCLLNNFYPREAKVQWKVDNALQSGNSQESVTEQDSKDSTYSLSSTLTLSK | |
| | ADYEKHKVYACEVTHQGLSSPVTKSFNRGECGGSEGKSSGSGSESKSTEGK | |
| | SSGSGSESKSTGGSQVQLQESGPGLVKPSETLSLTCTVSGYSITSGYFWNWIR | |
| | QPPGKGLEWIGYISYDGSNNYNPSLKSRVTISRDTSKNQFSLKLSSVTAADT | |
| | AVYYCASPSPGTGYAVDYWGQGTLVTVSSASTKGPSVFPLAPCSRSTSESTA | |
| | ALGCLVKDYFPEPVTVSWNSGALTSGVHTFPAVLQSSGLYSLSSVVTVPSSS | |
| | LGTKTYTCNVDHKPSNTKVDKRVESKYGPPCPPCPAPEAAGGPSVFLFPPKP | |
| | KDTLMISRTPEVTCVVVDVSQEDPEVQFNWYVDGVEVHNAKTKPREEQFN | |
| | STYRVVSVLTVLHQDWLNGKEYKCKVSNKGLPSSIEKTISKAKGQPREPQV | |
| | YTLPPSQEEMTKNQVSLSCAVKGFYPSDIAVEWESNGQPENNYKTTPPVLDS | |
| | DGSFFLVSRLTVDKSRWQEGNVFSCSVMHEALHNRFTQKSLSLSLGK | |
| I3RB217 | MAWVWTLLFLMAAAQSIQAEIVLTQSPGTLSLSPGERATLSCRASQSVSSSY | 30 |
| (CD123 | LAWYQQKPGQAPRLLIYGASSRATGIPDRFSGSGSGTDFTLTISRLEPEDFAV | |
| half Ab) | YYCQQDYGFPWTFGQGTKVEIKRTVAAPSVFIFPPSDEQLKSGTASVVCLLN | |
| | NFYPREAKVQWKVDNALQSGNSQESVTEQDSKDSTYSLSSTLTLSKADYEK | |
| | HKVYACEVTHQGLSSPVTKSFNRGECGGSEGKSSGSGSESKSTEGKSSGSGS | |
| | ESKSTGGSEVQLVQSGAEVKKPGESLKISCKGSGYSFTSYWISWVRQMPGK | |
| | GLEWMGIIDPSDSDTRYSPSFQGQVTISADKSISTAYLQWSSLKASDTAMYY | |
| | CARGDGSTDLDYWGQGTLVTVSSASTKGPSVFPLAPCSRSTSESTAALGCLV | |
| | KDYFPEPVTVSWNSGALTSGVHTFPAVLQSSGLYSLSSVVTVPSSSLGTKTY | |
| | TCNVDHKPSNTKVDKRVESKYGPPCPPCPAPEAAGGPSVFLFPPKPKDTLMI | |
| | SRTPEVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVYTLPPSQ | |
| | EEMTKNQVSLWCLVKGFYPSDIAVEWESNGQPENNYKTTPPVLDSDGSFFL | |
| | YSRLTVDKSRWQEGNVFSCSVMHEALHNHYTQKSLSLSLGK | |
| B23B49 | MAWVWTLLFLMAAAQSIQAEIVLTQSPGTLSLSPGERATLSCRASQSVSSSY | |
| (Null half | LAWYQQKPGQAPRLLIYGASSRATGIPDRFSGSGSGTDFTLTISRLEPEDFAV | |
| Ab) | YYCQQDYGFPWTFGQGTKVEIKRTVAAPSVFIFPPSDEQLKSGTASVVCLLN | |
| | NFYPREAKVQWKVDNALQSGNSQESVTEQDSKDSTYSLSSTLTLSKADYEK | |
| | HKVYACEVTHQGLSSPVTKSFNRGECGGSEGKSSGSGSESKSTEGKSSGSGS | |
| | ESKSTGGSEVQLVQSGAEVKKPGESLKISCKGSGYSFTSYWISWVRQMPGK | |
| | GLEWMGIIDPSDSDTRYSPSFQGQVTISADKSISTAYLQWSSLKASDTAMYY | |
| | CARGDGSTDLDYWGQGTLVTVSSASTKGPSVFPLAPCSRSTSESTAALGCLV | |
| | KDYFPEPVTVSWNSGALTSGVHTFPAVLQSSGLYSLSSVVTVPSSSLGTKTY | |
| | TCNVDHKPSNTKVDKRVESKYGPPCPPCPAPEAAGGPSVFLFPPKPKDTLMI | |
| | SRTPEVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVYTLPPSQ | |
| | EEMTKNQVSLWCLVKGFYPSDIAVEWESNGQPENNYKTTPPVLDSDGSFFL | |
| | YSRLTVDKSRWQEGNVFSCSVMHEALHNHYTQKSLSLSLGK | |
| | Half Antibody DNA sequence | |
| B17B21 | ATGGCCTGGGTGTGGACCCTGCTGTTCCTGATGGCCGCCGCCCAGAGCAT | 29 |
| (VB17 | CCAGGCCGACATCCAGATGACCCAGAGCCCAAGCAGCCTGAGCGCCAGC | |
| half Ab) | GTGGGCGACCGCGTGACCATCACCTGCCGCAGCAGCCAGAGCCTGGTGC | |
| | ACAGCAACGGCAACACCTACCTGCACTGGTACCAGCAGAAGCCAGGCAA | |
| | GGCCCCAAAGTTCCTGATCTACAAGGTGAGCAACCGCTTCAGCGGCGTG | |
| | CCAAGCCGCTTCAGCGGCAGCGGCAGCGGCACCGACTTCACCCTGACCA | |
| | TCAGCAGCCTGCAGCCAGAGGACTTCGCCACCTACTACTGCAGCCAGAG | |
| | CACCCACGTGCCATTCACCTTCGGCCAGGGCACCAAGCTGGAGATCAAG | |
| | CGCACCGTGGCCGCCCCAAGCGTGTTCATCTTCCCACCAAGCGACGAGC | |
| | AGCTGAAGAGCGGCACCGCCAGCGTGGTGTGCCTGCTGAACAACTTCTA | |
| | CCCACGCGAGGCCAAGGTGCAGTGGAAGGTGGACAACGCCCTGCAGAG | |
| | CGGCAACAGCCAGGAGAGCGTGACCGAGCAGGACAGCAAGGACAGCAC | |
| | CTACAGCCTGAGCAGCACCCTGACCCTGAGCAAGGCCGACTACGAGAAG | |
| | CACAAGGTGTACGCCTGCGAGGTGACCCACCAGGGCCTGAGCAGCCCAG | |
| | TGACCAAGAGCTTCAACCGCGGCGAGTGCGGCGGCAGCGAGGGCAAGA | |
| | GCAGCGGCAGCGGCAGCGAGAGCAAGAGCACCGAGGGCAAGAGCAGCG | |
| | GCAGCGGCAGCGAGAGCAAGAGCACCGGCGGCAGCCAGGTGCAGCTGC | |
| | AGGAGAGCGGCCCAGGCCTGGTGAAGCCAAGCGAGACCCTGAGCCTGA | |
| | CCTGCACCGTGAGCGGCTACAGCATCACCAGCGGCTACTTCTGGAACTG | |
| | GATCCGCCAGCCACCAGGCAAGGGCCTGGAGTGGATCGGCTACATCAGC | |
| | TACGACGGCAGCAACAACTACAACCCAAGCCTGAAGAGCCGCGTGACCA | |
| | TCAGCCGCGACACCAGCAAGAACCAGTTCAGCCTGAAGCTGAGCAGCGT | |
| | GACCGCCGCCGACACCGCCGTGTACTACTGCGCCAGCCCAAGCCCAGGC | |
| | ACCGGCTACGCCGTGGACTACTGGGGCCAGGGCACCCTGGTGACCGTGA | |
| | GCAGCGCCAGCACCAAGGGCCCAAGCGTGTTCCCACTGGCCCCATGCAG | |
| | CCGCAGCACCAGCGAGAGCACCGCCGCCCTGGGCTGCCTGGTGAAGGAC | |
| | TACTTCCCAGAGCCAGTGACCGTGAGCTGGAACAGCGGCGCCCTGACCA | |
| | GCGGCGTGCACACCTTCCCAGCCGTGCTGCAGAGCAGCGGCCTGTACAG | |
| | CCTGAGCAGCGTGGTGACCGTGCCAAGCAGCAGCCTGGGCACCAAGACC | |
| | TACACCTGCAACGTGGACCACAAGCCAAGCAACACCAAGGTGGACAAG | |
| | CGCGTGGAGAGCAAGTACGGCCCACCATGCCCACCATGCCCAGCCCCAG | |
| | AGGCCGCCGGCGGCCCAAGCGTGTTCCTGTTCCCACCAAAGCCAAAGGA | |
| | CACCCTGATGATCAGCCGCACCCCAGAGGTGACCTGCGTGGTGGTGGAC | |
| | GTGAGCCAGGAGGACCCAGAGGTGCAGTTCAACTGGTACGTGGACGGCG | |
| | TGGAGGTGCACAACGCCAAGACCAAGCCACGCGAGGAGCAGTTCAACA | |
| | GCACCTACCGCGTGGTGAGCGTGCTGACCGTGCTGCACCAGGACTGGCT | |
| | GAACGGCAAGGAGTACAAGTGCAAGGTGAGCAACAAGGGCCTGCCAAG | |
| | CAGCATCGAGAAGACCATCAGCAAGGCCAAGGGCCAGCCACGCGAGCC | |
| | ACAGGTGTACACCCTGCCACCAAGCCAGGAGGAGATGACCAAGAACCA | |
| | GGTGAGCCTGAGCTGCGCCGTGAAGGGCTTCTACCCAAGCGACATCGCC | |
| | GTGGAGTGGGAGAGCAACGGCCAGCCAGAGAACAACTACAAGACCACC | |
| | CCACCAGTGCTGGACAGCGACGGCAGCTTCTTCCTGGTGAGCCGCCTGA | |
| | CCGTGGACAAGAGCCGCTGGCAGGAGGGCAACGTGTTCAGCTGCAGCGT | |
| | GATGCACGAGGCCCTGCACAACCGCTTCACCCAGAAGAGCCTGAGCCTG | |
| | AGCCTGGGCAAGATGGCCTGGGTGTGGACCCTGCTGTTCCTGATGGCCG | |
| | CCGCCCAGAGCATCCAGGCCGACATCCAGATGACCCAGAGCCCAAGCAG | |
| | CCTGAGCGCCAGCGTGGGCGACCGCGTGACCATCACCTGCCGCAGCAGC | |
| | CAGAGCCTGGTGCACAGCAACGGCAACACCTACCTGCACTGGTACCAGC | |
| | AGAAGCCAGGCAAGGCCCCAAAGTTCCTGATCTACAAGGTGAGCAACCG | |
| | CTTCAGCGGCGTGCCAAGCCGCTTCAGCGGCAGCGGCAGCGGCACCGAC | |
| | TTCACCCTGACCATCAGCAGCCTGCAGCCAGAGGACTTCGCCACCTACTA | |
| | CTGCAGCCAGAGCACCCACGTGCCATTCACCTTCGGCCAGGGCACCAAG | |
| | CTGGAGATCAAGCGCACCGTGGCCGCCCCAAGCGTGTTCATCTTCCCACC | |
| | AAGCGACGAGCAGCTGAAGAGCGGCACCGCCAGCGTGGTGTGCCTGCTG | |
| | AACAACTTCTACCCACGCGAGGCCAAGGTGCAGTGGAAGGTGGACAACG | |
| | CCCTGCAGAGCGGCAACAGCCAGGAGAGCGTGACCGAGCAGGACAGCA | |
| | AGGACAGCACCTACAGCCTGAGCAGCACCCTGACCCTGAGCAAGGCCGA | |
| | CTACGAGAAGCACAAGGTGTACGCCTGCGAGGTGACCCACCAGGGCCTG | |
| | AGCAGCCCAGTGACCAAGAGCTTCAACCGCGGCGAGTGCGGCGGCAGC | |
| | GAGGGCAAGAGCAGCGGCAGCGGCAGCGAGAGCAAGAGCACCGAGGGC | |
| | AAGAGCAGCGGCAGCGGCAGCGAGAGCAAGAGCACCGGCGGCAGCCAG | |
| | GTGCAGCTGCAGGAGAGCGGCCCAGGCCTGGTGAAGCCAAGCGAGACC | |
| | CTGAGCCTGACCTGCACCGTGAGCGGCTACAGCATCACCAGCGGCTACT | |
| | TCTGGAACTGGATCCGCCAGCCACCAGGCAAGGGCCTGGAGTGGATCGG | |
| | CTACATCAGCTACGACGGCAGCAACAACTACAACCCAAGCCTGAAGAGC | |
| | CGCGTGACCATCAGCCGCGACACCAGCAAGAACCAGTTCAGCCTGAAGC | |
| | TGAGCAGCGTGACCGCCGCCGACACCGCCGTGTACTACTGCGCCAGCCC | |
| | AAGCCCAGGCACCGGCTACGCCGTGGACTACTGGGGCCAGGGCACCCTG | |
| | GTGACCGTGAGCAGCGCCAGCACCAAGGGCCCAAGCGTGTTCCCACTGG | |
| | CCCCATGCAGCCGCAGCACCAGCGAGAGCACCGCCGCCCTGGGCTGCCT | |
| | GGTGAAGGACTACTTCCCAGAGCCAGTGACCGTGAGCTGGAACAGCGGC | |
| | GCCCTGACCAGCGGCGTGCACACCTTCCCAGCCGTGCTGCAGAGCAGCG | |
| | GCCTGTACAGCCTGAGCAGCGTGGTGACCGTGCCAAGCAGCAGCCTGGG | |
| | CACCAAGACCTACACCTGCAACGTGGACCACAAGCCAAGCAACACCAAG | |
| | GTGGACAAGCGCGTGGAGAGCAAGTACGGCCCACCATGCCCACCATGCC | |
| | CAGCCCCAGAGGCCGCCGGCGGCCCAAGCGTGTTCCTGTTCCCACCAAA | |
| | GCCAAAGGACACCCTGATGATCAGCCGCACCCCAGAGGTGACCTGCGTG | |
| | GTGGTGGACGTGAGCCAGGAGGACCCAGAGGTGCAGTTCAACTGGTACG | |
| | TGGACGGCGTGGAGGTGCACAACGCCAAGACCAAGCCACGCGAGGAGC | |
| | AGTTCAACAGCACCTACCGCGTGGTGAGCGTGCTGACCGTGCTGCACCA | |
| | GGACTGGCTGAACGGCAAGGAGTACAAGTGCAAGGTGAGCAACAAGGG | |
| | CCTGCCAAGCAGCATCGAGAAGACCATCAGCAAGGCCAAGGGCCAGCC | |
| | ACGCGAGCCACAGGTGTACACCCTGCCACCAAGCCAGGAGGAGATGACC | |
| | AAGAACCAGGTGAGCCTGAGCTGCGCCGTGAAGGGCTTCTACCCAAGCG | |
| | ACATCGCCGTGGAGTGGGAGAGCAACGGCCAGCCAGAGAACAACTACA | |
| | AGACCACCCCACCAGTGCTGGACAGCGACGGCAGCTTCTTCCTGGTGAG | |
| | CCGCCTGACCGTGGACAAGAGCCGCTGGCAGGAGGGCAACGTGTTCAGC | |
| | TGCAGCGTGATGCACGAGGCCCTGCACAACCGCTTCACCCAGAAGAGCC | |
| | TGAGCCTGAGCCTGGGCAAGTGATAG | |
| I3RB217 | ATGGCCTGGGTGTGGACCCTGCTGTTCCTGATGGCCGCCGCCCAGAGCAT | 31 |
| (CD123 | CCAGGCCGAGATCGTGCTGACCCAGAGCCCAGGCACCCTGAGCCTGAGC | |
| half Ab) | CCAGGCGAGCGCGCCACCCTGAGCTGCCGCGCCAGCCAGAGCGTGAGCA | |
| | GCAGCTACCTGGCCTGGTACCAGCAGAAGCCAGGCCAGGCCCCACGCCT | |
| | GCTGATCTACGGCGCCAGCAGCCGCGCCACCGGCATCCCAGACCGCTTC | |
| | AGCGGCAGCGGCAGCGGCACCGACTTCACCCTGACCATCAGCCGCCTGG | |
| | AGCCAGAGGACTTCGCCGTGTACTACTGCCAGCAGGACTACGGCTTCCC | |
| | ATGGACCTTCGGCCAGGGCACCAAGGTGGAGATCAAGCGCACCGTGGCC | |
| | GCCCCAAGCGTGTTCATCTTCCCACCAAGCGACGAGCAGCTGAAGAGCG | |
| | GCACCGCCAGCGTGGTGTGCCTGCTGAACAACTTCTACCCACGCGAGGC | |
| | CAAGGTGCAGTGGAAGGTGGACAACGCCCTGCAGAGCGGCAACAGCCA | |
| | GGAGAGCGTGACCGAGCAGGACAGCAAGGACAGCACCTACAGCCTGAG | |
| | CAGCACCCTGACCCTGAGCAAGGCCGACTACGAGAAGCACAAGGTGTAC | |
| | GCCTGCGAGGTGACCCACCAGGGCCTGAGCAGCCCAGTGACCAAGAGCT | |
| | TCAACCGCGGCGAGTGCggcggcagcgagggcaagagcagcggcagcggcagcgagagcaagag | |
| | caccgagggcaagagcagcggcagcggcagcgagagcaagagcaccggcggcagcGAGGTGCAGCT | |
| | GGTGCAGAGCGGCGCCGAGGTGAAGAAGCCAGGCGAGAGCCTGAAGAT | |
| | CAGCTGCAAGGGCAGCGGCTACAGCTTCACCAGCTACTGGATCAGCTGG | |
| | GTGCGCCAGATGCCAGGCAAGGGCCTGGAGTGGATGGGCATCATCGACC | |
| | CAAGCGACAGCGACACCCGCTACAGCCCAAGCTTCCAGGGCCAGGTGAC | |
| | CATCAGCGCCGACAAGAGCATCAGCACCGCCTACCTGCAGTGGAGCAGC | |
| | CTGAAGGCCAGCGACACCGCCATGTACTACTGCGCCCGCGGCGACGGCA | |
| | GCACCGACCTGGACTACTGGGGCCAGGGCACCCTGGTGACCGTGAGCAG | |
| | CGCCAGCACCAAGGGCCCAAGCGTGTTCCCACTGGCCCCATGCAGCCGC | |
| | AGCACCAGCGAGAGCACCGCCGCCCTGGGCTGCCTGGTGAAGGACTACT | |
| | TCCCAGAGCCAGTGACCGTGAGCTGGAACAGCGGCGCCCTGACCAGCGG | |
| | CGTGCACACCTTCCCAGCCGTGCTGCAGAGCAGCGGCCTGTACAGCCTG | |
| | AGCAGCGTGGTGACCGTGCCAAGCAGCAGCCTGGGCACCAAGACCTACA | |
| | CCTGCAACGTGGACCACAAGCCAAGCAACACCAAGGTGGACAAGCGCG | |
| | TGGAGAGCAAGTACGGCCCACCATGCCCACCATGCCCAGCCCCAGAGGC | |
| | CGCCGGCGGCCCAAGCGTGTTCCTGTTCCCACCAAAGCCAAAGGACACC | |
| | CTGATGATCAGCCGCACCCCAGAGGTGACCTGCGTGGTGGTGGACGTGA | |
| | GCCAGGAGGACCCAGAGGTGCAGTTCAACTGGTACGTGGACGGCGTGGA | |
| | GGTGCACAACGCCAAGACCAAGCCACGCGAGGAGCAGTTCAACAGCAC | |
| | CTACCGCGTGGTGAGCGTGCTGACCGTGCTGCACCAGGACTGGCTGAAC | |
| | GGCAAGGAGTACAAGTGCAAGGTGAGCAACAAGGGCCTGCCAAGCAGC | |
| | ATCGAGAAGACCATCAGCAAGGCCAAGGGCCAGCCACGCGAGCCACAG | |
| | GTGTACACCCTGCCACCAAGCCAGGAGGAGATGACCAAGAACCAGGTG | |
| | AGCCTGTGGTGCCTGGTGAAGGGCTTCTACCCAAGCGACATCGCCGTGG | |
| | AGTGGGAGAGCAACGGCCAGCCAGAGAACAACTACAAGACCACCCCAC | |
| | CAGTGCTGGACAGCGACGGCAGCTTCTTCCTGTACAGCCGCCTGACCGT | |
| | GGACAAGAGCCGCTGGCAGGAGGGCAACGTGTTCAGCTGCAGCGTGATG | |
| | CACGAGGCCCTGCACAACCACTACACCCAGAAGAGCCTGAGCCTGAGCC | |
| | TGGGCAAG | |
| B23B49 | ATGGCCTGGGTGTGGACCCTGCTGTTCCTGATGGCCGCCGCCCAGAGCAT | |
| (Null half | CCAGGCCGACATCGTGATGACCCAGAGCCCAGACAGCCTGGCCGTGAGC | |
| Ab) | CTGGGCGAGCGCGCCACCATCAACTGCCGCGCCAGCCAGAGCGTGGACT | |
| | ACAACGGCATCAGCTACATGCACTGGTACCAGCAGAAGCCAGGCCAGCC | |
| | ACCAAAGCTGCTGATCTACGCCGCCAGCAACCCAGAGAGCGGCGTGCCA | |
| | GACCGCTTCAGCGGCAGCGGCAGCGGCACCGACTTCACCCTGACCATCA | |
| | GCAGCCTGCAGGCCGAGGACGTGGCCGTGTACTACTGCCAGCAGATCAT | |
| | CGAGGACCCATGGACCTTCGGCCAGGGCACCAAGGTGGAGATCAAGCGC | |
| | ACCGTGGCCGCCCCAAGCGTGTTCATCTTCCCACCAAGCGACGAGCAGC | |
| | TGAAGAGCGGCACCGCCAGCGTGGTGTGCCTGCTGAACAACTTCTACCC | |
| | ACGCGAGGCCAAGGTGCAGTGGAAGGTGGACAACGCCCTGCAGAGCGG | |
| | CAACAGCCAGGAGAGCGTGACCGAGCAGGACAGCAAGGACAGCACCTA | |
| | CAGCCTGAGCAGCACCCTGACCCTGAGCAAGGCCGACTACGAGAAGCAC | |
| | AAGGTGTACGCCTGCGAGGTGACCCACCAGGGCCTGAGCAGCCCAGTGA | |
| | CCAAGAGCTTCAACCGCGGCGAGTGCGGCGGCAGCGAGGGCAAGAGCA | |
| | GCGGCAGCGGCAGCGAGAGCAAGAGCACCGAGGGCAAGAGCAGCGGCA | |
| | GCGGCAGCGAGAGCAAGAGCACCGGCGGCAGCCAGATCACCCTGAAGG | |
| | AGAGCGGCCCAACCCTGGTGAAGCCAACCCAGACCCTGACCCTGACCTG | |
| | CACCTTCAGCGGCTTCAGCCTGAGCACCAGCGGCATGGGCGTGAGCTGG | |
| | ATCCGCCAGCCACCAGGCAAGGCCCTGGAGTGGCTGGCCCACATCTACT | |
| | GGGACGACGACAAGCGCTACAACCCAAGCCTGAAGAGCCGCCTGACCAT | |
| | CACCAAGGACACCAGCAAGAACCAGGTGGTGCTGACCATGACCAACATG | |
| | GACCCAGTGGACACCGCCACCTACTACTGCGCCCGCCTGTACGGCTTCAC | |
| | CTACGGCTTCGCCTACTGGGGCCAGGGCACCCTGGTGACCGTGAGCAGC | |
| | GCCAGCACCAAGGGCCCAAGCGTGTTCCCACTGGCCCCATGCAGCCGCA | |
| | GCACCAGCGAGAGCACCGCCGCCCTGGGCTGCCTGGTGAAGGACTACTT | |
| | CCCAGAGCCAGTGACCGTGAGCTGGAACAGCGGCGCCCTGACCAGCGGC | |
| | GTGCACACCTTCCCAGCCGTGCTGCAGAGCAGCGGCCTGTACAGCCTGA | |
| | GCAGCGTGGTGACCGTGCCAAGCAGCAGCCTGGGCACCAAGACCTACAC | |
| | CTGCAACGTGGACCACAAGCCAAGCAACACCAAGGTGGACAAGCGCGT | |
| | GGAGAGCAAGTACGGCCCACCATGCCCACCATGCCCAGCCCCAGAGGCC | |
| | GCCGGCGGCCCAAGCGTGTTCCTGTTCCCACCAAAGCCAAAGGACACCC | |
| | TGATGATCAGCCGCACCCCAGAGGTGACCTGCGTGGTGGTGGACGTGAG | |
| | CCAGGAGGACCCAGAGGTGCAGTTCAACTGGTACGTGGACGGCGTGGAG | |
| | GTGCACAACGCCAAGACCAAGCCACGCGAGGAGCAGTTCAACAGCACCT | |
| | ACCGCGTGGTGAGCGTGCTGACCGTGCTGCACCAGGACTGGCTGAACGG | |
| | CAAGGAGTACAAGTGCAAGGTGAGCAACAAGGGCCTGCCAAGCAGCAT | |
| | CGAGAAGACCATCAGCAAGGCCAAGGGCCAGCCACGCGAGCCACAGGT | |
| | GTACACCCTGCCACCAAGCCAGGAGGAGATGACCAAGAACCAGGTGAG | |
| | CCTGTGGTGCCTGGTGAAGGGCTTCTACCCAAGCGACATCGCCGTGGAG | |
| | TGGGAGAGCAACGGCCAGCCAGAGAACAACTACAAGACCACCCCACCA | |
| | GTGCTGGACAGCGACGGCAGCTTCTTCCTGTACAGCCGCCTGACCGTGG | |
| | ACAAGAGCCGCTGGCAGGAGGGCAACGTGTTCAGCTGCAGCGTGATGCA | |
| | CGAGGCCCTGCACAACCACTACACCCAGAAGAGCCTGAGCCTGAGCCTG | |
| | GGCAAG | |

TABLE 8
Heavy and Light Chain Sequences for Vβ17 bispecific antibodies

| Bispecific | | |
|---|---|---|
| Antibody | | Amino Acid Sequence |
| Anti-Vβ17/ | Heavy chain 1 | QVQLQESGPGLVKPSETLSLTCTVSGYSITSGYFWNWIRQPP |
| anti-CD123 | B17B21 | GKGLEWIGYISYDGSNNYNPSLKSRVTISRDTSKNQFSLKLS |
| | (SEQ ID NO: 13) | SVTAADTAVYYCASPSPGTGYAVDYWGQGTLVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEAAGGPSVFLFPPKPKDT |
| | | LMISRTPEVTCVVVDVSQEDPEVQFNWYVDGVEVHNAKTK |
| | | PREEQFNSTYRVVSVLTVLHQDWLNGKEYKCKVSNKGLPS |
| | | SIEKTISKAKGQPREPQVYTLPPSQEEMTKNQVSLSCAVKGF |
| | | YPSDIAVEWESNGQPENNYKTTPPVLDSDGSFFLVSRLTVD |
| | | KSRWQEGNVFSCSVMHEALHNRFTQKSLSLSLGK |
| | Light Chain 1 | DIQMTQSPSSLSASVGDRVTITCRSSQSLVHSNGNTYLHWY |
| | B17B21 | QQKPGKAPKFLIYKVSNRFSGVPSRFSGSGSGTDFTLTISSLQ |
| | (SEQ ID NO: 14) | PEDFATYYCSQSTHVPFTFGQGTKLEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |
| | Heavy chain 2 | EVQLVQSGAEVKKPGESLKISCKGSGYSFTSYWISWVRQMP |
| | I3RB217 | GKGLEWMGIIDPSDSDTRYSPSFQGQVTISADKSISTAYLQW |
| | (SEQ ID NO: 15) | SSLKASDTAMYYCARGDGSTDLDYWGQGTLVTVSSASTKG |
| | | PSVFPLAPCSRSTSESTAALGCLVKDYFPEPVTVSWNSGALT |
| | | SGVHTFPAVLQSSGLYSLSSVVTVPSSSLGTKTYTCNVDHKP |
| | | SNTKVDKRVESKYGPPCPPCPAPEAAGGPSVFLFPPKPKDTL |
| | | MISRTPEVTCVVVDVSQEDPEVQFNWYVDGVEVHNAKTKP |
| | | REEQFNSTYRVVSVLTVLHQDWLNGKEYKCKVSNKGLPSSI |
| | | EKTISKAKGQPREPQVYTLPPSQEEMTKNQVSLWCLVKGFY |
| | | PSDIAVEWESNGQPENNYKTTPPVLDSDGSFFLYSRLTVDKS |
| | | RWQEGNVFSCSVMHEALHNHYTQKSLSLSLGK |
| | Light Chain 2 | EIVLTQSPGTLSLSPGERATLSCRASQSVSSSYLAWYQQKPG |
| | I3RB217 | QAPRLLIYGASSRATGIPDRFSGSGSGTDFTLTISRLEPEDFA |
| | (SEQ ID NO: 16) | VYYCQQDYGFPWTFGQGTKVEIKRTVAAPSVFIFPPSDEQL |
| | | KSGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC |
| Vβ17 x Null | Heavy chain 1 | QVQLQESGPGLVKPSETLSLTCTVSGYSITSGYFWNWIRQPP |
| | B17B21 | GKGLEWIGYISYDGSNNYNPSLKSRVTISRDTSKNQFSLKLS |
| | (SEQ ID NO: 13) | SVTAADTAVYYCASPSPGTGYAVDYWGQGTLVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEAAGGPSVFLFPPKPKDT |
| | | LMISRTPEVTCVVVDVSQEDPEVQFNWYVDGVEVHNAKTK |
| | | PREEQFNSTYRVVSVLTVLHQDWLNGKEYKCKVSNKGLPS |
| | | SIEKTISKAKGQPREPQVYTLPPSQEEMTKNQVSLSCAVKGF |
| | | YPSDIAVEWESNGQPENNYKTTPPVLDSDGSFFLVSRLTVD |
| | | KSRWQEGNVFSCSVMHEALHNRFTQKSLSLSLGK |
| | Light Chain 1 | DIQMTQSPSSLSASVGDRVTITCRSSQSLVHSNGNTYLHWY |
| | B17B21 | QQKPGKAPKFLIYKVSNRFSGVPSRFSGSGSGTDFTLTISSLQ |
| | (SEQ ID NO: 14) | PEDFATYYCSQSTHVPFTFGQGTKLEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |
| | Heavy chain 2 | QITLKESGPTLVKPTQTLTLTCTFSGFSLSTSGMGVSWIRQPP |
| | Null | GKALEWLAHIYWDDDKRYNPSLKSRLTITKDTSKNQVVLT |
| | (SEQ ID NO: 17) | MTNMDPVDTATYYCARLYGFTYGFAYWGQGTLVTVSSAS |
| | | TKGPSVFPLAPCSRSTSESTAALGCLVKDYFPEPVTVSWNSG |
| | | ALTSGVHTFPAVLQSSGLYSLSSVVTVPSSSLGTKTYTCNVD |
| | | HKPSNTKVDKRVESKYGPPCPPCPAPEAAGGPSVFLFPPKPK |
| | | DTLMISRTPEVTCVVVDVSQEDPEVQFNWYVDGVEVHNAK |
| | | TKPREEQFNSTYRVVSVLTVLHQDWLNGKEYKCKVSNKGL |
| | | PSSIEKTISKAKGQPREPQVYTLPPSQEEMTKNQVSLWCLVK |
| | | GFYPSDIAVEWESNGQPENNYKTTPPVLDSDGSFFLYSRLTV |
| | | DKSRWQEGNVFSCSVMHEALHNHYTQKSLSLSLGK |
| | Light Chain 2 | DIVMTQSPDSLAVSLGERATINCRASQSVDYNGISYMHWYQ |
| | Null | QKPGQPPKLLIYAASNPESGVPDRFSGSGSGTDFTLTISSLQA |
| | (SEQ ID NO: 18) | EDVAVYYCQQIIEDPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |

### Example 3. Evaluation of Binding and Cytotoxic Properties of Anti-Vβ17/Anti-CD123 Bispecific Antibody Using Kasumi-3 Cells and Human CD8⁺ T Cells

Stimulation and expansion of Vβ17⁺ CD8⁺ T cells from total PBMCs

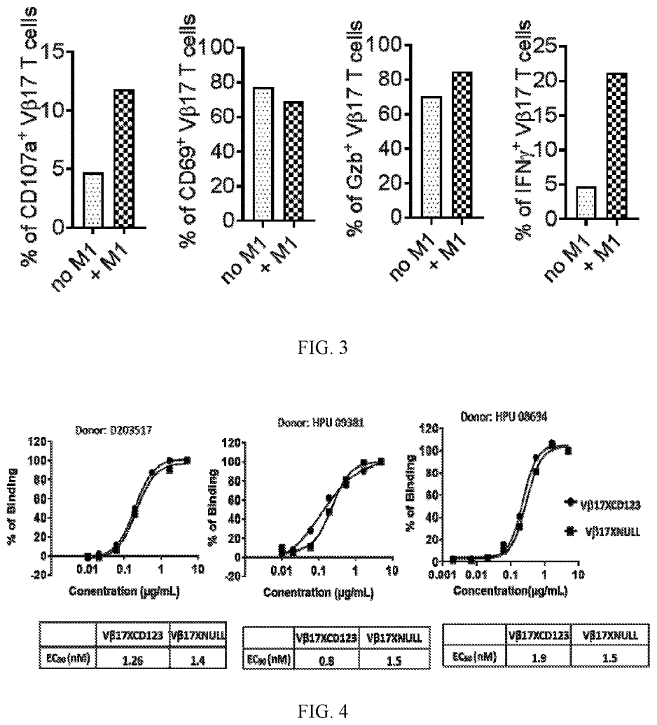

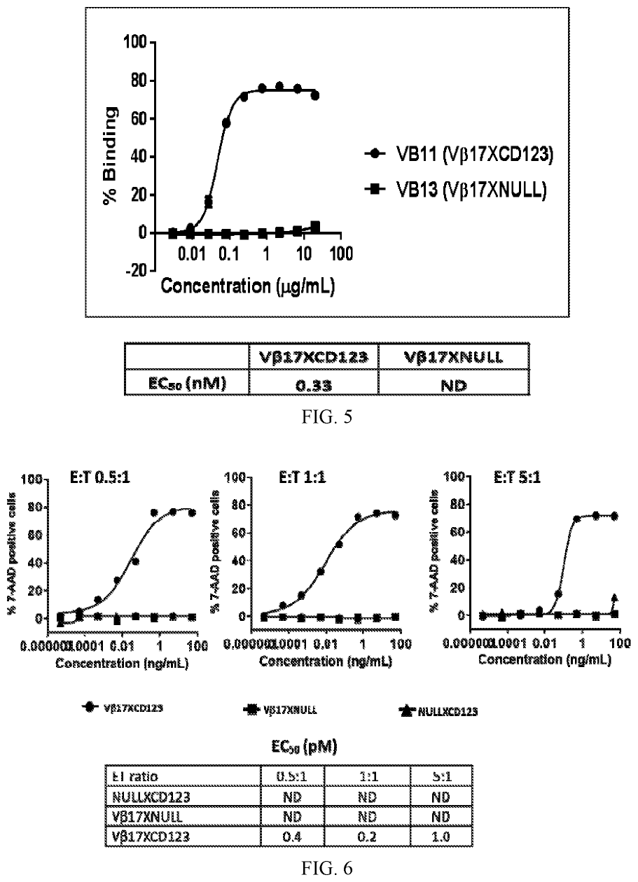

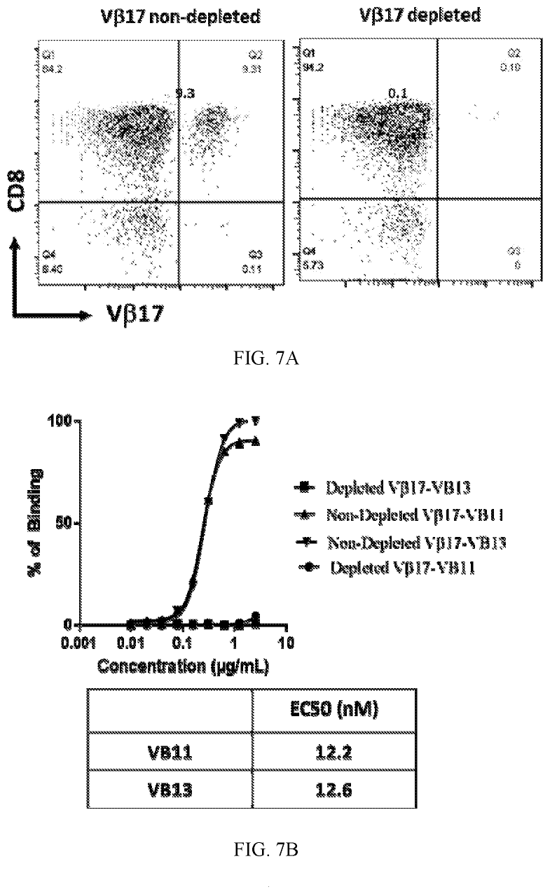

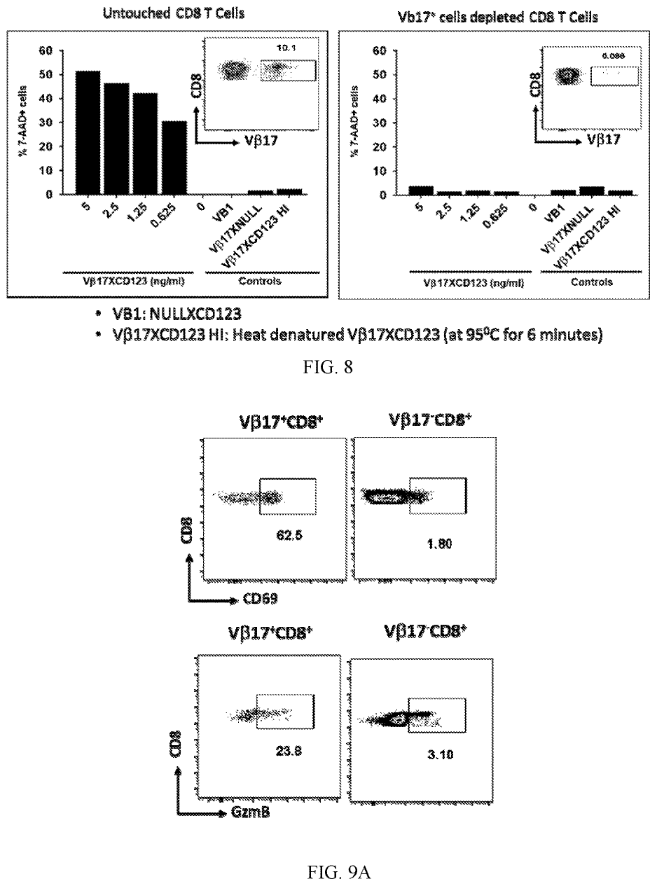

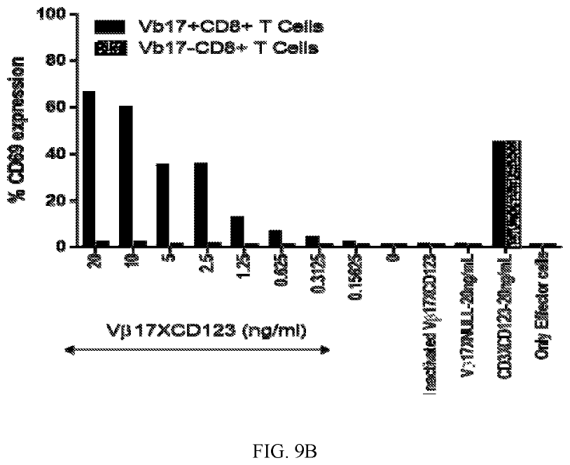

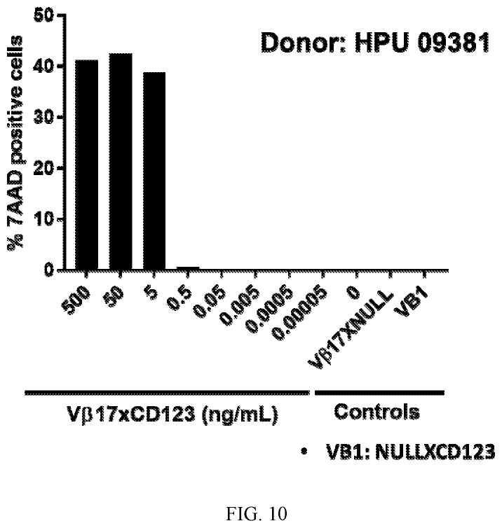

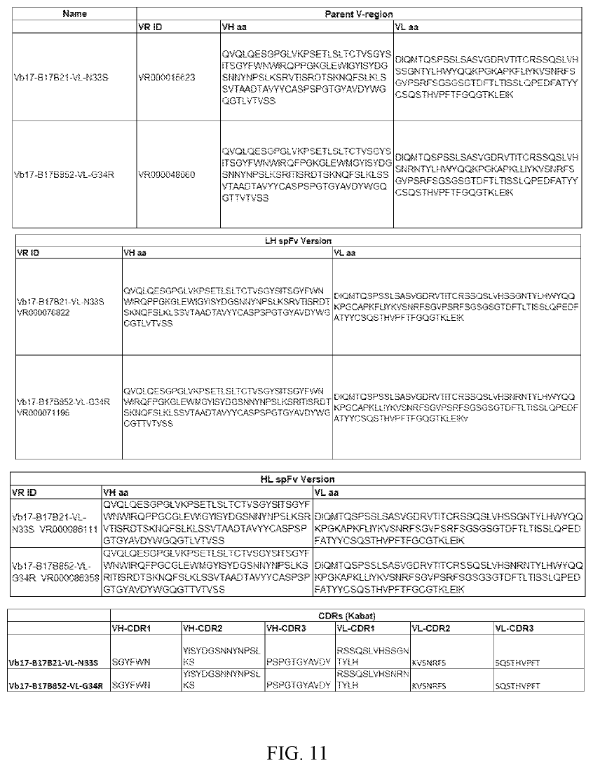

To expand Vβ17⁺ CD8⁺ T cells, whole PBMCs from HLA-A2 donor (HPU-08694) were stimulated with 1 μg/mL FLU MP 58 peptide (in DMSO). Frequency of Vβ17⁺ cells among total CD8⁺ T cells was determined on day 8 and 14 of the culture period. To enumerate the frequency of Vβ17⁺ cells among total CD8⁺ T cells, total live PBMCs were initially gated, doublets were excluded, total CD8⁺ T cells were gated and then Vβ17′ cells were gated (FIG. 3A). Compared to the frequency of Vβ17+ cells among total CD8⁺ T cells on day 0, a substantial expansion of these cells were observed at day 8 of the culture period (FIG. 3B). A larger fraction of CD8⁺ T cells on day 8 were Vβ17⁺ cells (FIG. 3B) in this donor.

### Anti-Vβ17/Anti-CD123 Bispecific Antibody Binding Assay

### On Kasumi-3 Cells

To understand the binding kinetics of the anti-Vβ17/anti-CD123 bispecific antibody, Kasumi-3 cells were incubated with the anti-Vβ17/anti-CD123 bispecific antibody and Vβ17×NULL arm control at various concentrations (concentration range from 5 μg/mL to 0 μg/mL). Cell bound bispecific antibody was detected with mouse anti-human IgG4 Fc-PE secondary antibody. Table 9 shows the frequency of Kasumi-3 cells positive for PE (secondary antibody) when treated with different concentration of bispecific antibodies. The EC₅₀ for anti-Vβ17/anti-CD123 and NULL×CD123 was determined as 6 and 42.7 nM respectively (Table 9).

TABLE 9
Binding affinity of Kasumi-3 cells for bispecific antibodies.

| | anti-Vβ17/ | | |
|---|---|---|---|
| Conc.(ug/mL) | anti-CD123 | Vβ17XNULL | NULLXCD123 |
| 5 | 87.37 | −0.02 | 47.47 |
| 1.667 | 74.37 | 0.09 | 16.37 |
| 0.556 | 24.07 | 0.15 | 1.2 |
| 0.185 | 1.65 | 0.35 | 0.31 |
| 0.062 | 0.32 | 0.12 | 0.04 |
| 0.021 | 0.16 | 0.14 | 0.05 |
| 0.007 | 0.23 | 0.19 | −0.06 |
| 0.002 | 0.04 | −0.09 | −0.04 |
| 0.001 | 0.09 | 0 | −0.18 |
| EC₅₀ (μg/mL) | 0.9 | ND | 6.4 |
| EC₅₀ (nM) | 6 | ND | 42.7 |

Bispecific antibody binding affinities to Kasumi-3 cells were determined by flow cytometry. Half maximal effective concentration (EC₅₀) values were calculated as the bispecific concentration that generates 50% of maximal Binding (PE positive cells). ND: Not determined.

### On Enriched CD8⁺ T Cells

Enriched FLU MP 58 peptide stimulated CD8+T (from day 14 culture) cells were incubated with various concentrations of anti-Vβ17/anti-CD123 bispecific and Vβ17×NULL arm control antibodies. Mouse anti-human IgG4 Fc-PE secondary antibody was used to detect the bispecific antibody. Table 10 shows the frequency of CD8⁺ T cells positive for PE (secondary antibody) when treated with different concentration of bispecific antibodies. The EC₅₀ for anti-Vβ17/anti-CD123, Vβ17×NULL, was determined as 9.0 nm, 18.7 nm respectively (Table 10).

TABLE 10
Binding activity of CD8⁺ T cell for bispecific antibodies.

| | anti-Vβ17/ | | |
|---|---|---|---|
| Conc.(μg/mL) | anti-CD123 | Vβ17XNULL | NULLXCD123 |
| 20 | 74.7 | 76.2 | 0.1 |
| 10 | 72.4 | 75.4 | 0.4 |
| 5 | 70.8 | 64.6 | 0.3 |
| 2.5 | 64.8 | 42.5 | 0.5 |
| 1.25 | 38.0 | 32.4 | 0.4 |
| 0.625 | 41.4 | 21.7 | −0.1 |
| 0.3125 | 26.5 | 11.8 | 0.8 |
| 0.15625 | 19.9 | 3.4 | 0.6 |
| 0.078125 | 10.8 | 1.8 | 0.7 |
| EC₅₀ (μg/mL) | 1.35 | 2.80 | ND |
| EC₅₀ (nM) | 9 | 18.7 | ND |

Bispecific antibody binding affinities to CD8+ T cell were determined by flow cytometry. Half maximal effective concentration (EC₅₀) values were calculated as the antibody concentration that generates 50% of the maximal binding (PE positive cells). ND: Not Determined

### Bispecific Mediated Cytotoxicity Assay

In order to analyze the potency of the anti-Vβ17/anti-CD123 bispecific antibody mediated cytotoxicity, CFSE labelled target (Kasumi-3) cells were co-cultured with stimulated CD8+ T cells (effectors) from day 14 of culture at an effector to target (ET) ratio 0.5:1, 1:1, 5:1 for 14 and 24 hours with various concentrations of anti-Vβ17/anti-CD123 bispecific and Vβ17×NULL arm control antibody. CD123 expression on target Kasumi-3 cells were checked by using a commercially available anti-CD123 antibody. Target cells (Kasumi-3) were labelled with CFSE to identify them as CFSE⁺ during flow cytometry analysis. Post co-culture period, 7-AAD was added to analyze the percentage of 7-AAD⁺ CFSE⁺ cells as a measure of cytotoxicity. Basal cytotoxicity observed in the absence of bispecific antibody was subtracted to obtain specific cytotoxicity in response to bispecific antibody. The assay was performed once with a single donor (HPU-08694). The EC₅₀ for the anti-Vβ17/anti-CD123 bispecific antibody at 0.5:1, 1:1 and 5:1 ET ratios for 14-hour time point were 3.7, 0.1 and 0.133 pM respectively (Table 11).

TABLE 11
Summary of EC₅₀ values for various bispecific antibodies upon
co-culturing FLU MP 58 peptide stimulated CD8⁺ T cell
with Kasumi-3 cells at ET ratios 0.5:1, 1:1 and 5:1 for 14 hours.

| | EC₅₀ (ng/mL) | | |
|---|---|---|---|
| | E:T Ratio | E:T Ratio | E:T Ratio |
| Bispecific Ab | (0.5:1) | (1:1) | (5:1) |
| NULLXCD123 | UD | UD | UD |
| Vβ17XNULL | UD | UD | UD |
| Anti-Vβ17/anti-CD123 | 0.55 | 0.015 | 0.02 |
| | EC₅₀ (pM) | | |
| | E:T Ratio | E:T Ratio | E:T Ratio |
| Bispecific Ab | (0.5:1) | (1:1) | (5:1) |
| NULLXCD123 | UD | UD | UD |
| Vβ17XNULL | UD | UD | UD |
| anti-Vβ17/anti-CD123 | 3.7 | 0.1 | 0.133 |

UD: Undetectable, as the activity was too low for proper curve fitting.

The EC₅₀ for the anti-Vβ17/anti-CD123 bispecific at 0.5:1, 1:1 and 5:1 ET ratio for 24-hour time point were 0.4, 0.2 and 1.0 pM respectively (Table 12).

TABLE 12
Summary of EC₅₀ values for various bispecific antibodies upon
co-culturing FLU MP 58 peptide stimulated CD8⁺ T cells
with Kasumi-3 cells at ET ratios 0.5:1, 1:1 and 5:1 for 24 hours.

| | EC₅₀ (ng/mL) | | |
|---|---|---|---|
| | E:T Ratio | E:T Ratio | E:T Ratio |
| Bispecific Ab | (0.5:1) | (1:1) | (5:1) |
| NULLXCD123 | UD | UD | UD |
| Vβ17XNULL | UD | UD | UD |
| anti-Vβ17/anti-CD123 | 0.06 | 0.03 | 0.15 |
| | EC₅₀ (pM) | | |
| | E:T Ratio | E:T Ratio | E:T Ratio |
| Bispecific Ab | (0.5:1) | (1:1) | (5:1) |
| NULLXCD123 | UD | UD | UD |
| Vβ17XNULL | UD | UD | UD |
| anti-Vβ17/anti-CD123 | 0.4 | 0.2 | 1.0 |

UD: Undetectable

Similarly, anti-Vβ17/anti-CD123 bispecific mediated unstimulated CD8⁺ T cell cytotoxicity was tested at ET ratio 0.5:1, 1:1, 5:1 for 14 (Table 13) and 24 (Table 14) hours. At 5 ng/ml anti-Vβ17/anti-CD123 bispecific concentration and 14-hour time point, unstimulated CD8⁺ T cells at 0.5:1 and 1:1 ET ratio showed 2.8% and 9.8% target cell cytotoxicity respectively (Table 13), compared to 77% and 73% cytotoxicity by stimulated CD8⁺ T cells. At 5:1 ET ratio, unstimulated CD8⁺ T cells exhibited 31.65% target cytotoxicity, compared to 70.9% by stimulated CD8⁺ T cells. Similar results were obtained from 24-hour time point (Table 12, 15, 16, and 17). At highest concentration (5 ng/ml) of anti-Vβ17/anti-CD123 bispecific tested, unstimulated CD8⁺ T cells exhibited higher cytotoxicity towards target cells at a higher ET ratio.

TABLE 13
Cytotoxicity assay with unstimulated CD8⁺ T cells at various ET ratios
for 14 hours. Frequency of CFSE and 7-AAD positive cells when treated
with different concentrations of bispecific antibodies.

| | Conc | E:T Ratio | E:T Ratio | E:T Ratio |
|---|---|---|---|---|
| Bispecific Ab | (ng/mL) | (0.5:1) | (1:1) | (5:1) |
| NULLXCD123 | 5 | 1.3 | −0.6 | −0.45 |
| | 0.005 | 1.3 | −0.5 | 0.45 |
| Vβ17XNULL | 5 | −0.6 | −0.8 | 3.25 |
| | 0.005 | −0.3 | 1 | −1.35 |
| anti-Vβ17/anti- | 5 | 2.8 | 9.8 | 31.65 |
| CD123 | | | | |
| | 0.005 | 0.1 | 3.2 | 12.25 |

TABLE 14
Cytotoxicity assay with unstimulated CD8⁺ T cells at various ET ratios
for 24 hours. Frequency of CFSE and 7-AAD positive cells when treated
with different concentrations of bispecific antibodies.
Unstimulated CD8⁺ T cells

| | Conc | E:T Ratio | E:T Ratio | E:T Ratio |
|---|---|---|---|---|
| | (ng/mL) | (0.5:1) | (1:1) | (5:1) |
| NULLXCD123 | 5 | −1.3 | −0.55 | −4.85 |
| | 0.005 | −2 | −0.85 | −2.95 |
| Vβ17XNULL | 5 | −0.8 | −1.35 | 10.85 |
| | 0.005 | −1 | −1.05 | −1.95 |
| anti-Vβ17/anti-CD123 | 5 | 4.8 | 11.55 | 30.65 |
| | 0.005 | 1.5 | 1.75 | 10.95 |

TABLE 15
Cytotoxicity assay at 0.5:1 ET ratio (stimulated CD8⁺ T cell:
Kasumi-3 cells) upon incubation for 14 hours. Frequency of CFSE
and 7-AAD positive cells when treated with different
concentrations of bispecific antibodies at 0.5:1 ET ratio for 14 hrs.

| | | | anti-Vβ17/ |
|---|---|---|---|
| Conc.(ng/mL) | NULLXCD123 | Vβ17XNULL | anti-CD123 |
| 50 | 3.47 | 2.57 | 77.07 |
| 5 | 2.67 | 2.17 | 77.47 |
| 0.5 | −0.73 | 1.87 | 46.77 |
| 0.05 | 0.77 | 1.67 | 3.77 |
| 0.005 | −0.03 | 1.47 | 1.97 |
| 0.0005 | 0.67 | 1.07 | 2.17 |
| 0.00005 | 1.67 | 0.17 | 0.17 |
| 0.000005 | −0.43 | 3.27 | 0.87 |
| EC₅₀ (ng/mL) | ND | ND | 0.55 |
| EC₅₀ (pM) | ND | ND | 3.7 |

Half maximal effective concentration (EC₅₀) values were calculated as the antibody concentration that generates 50% of maximal cytotoxicity (CFSE⁺ 7AAD⁺) cells. ND: Not Determined.

TABLE 16
Cytotoxicity assay at 1:1 ET ratio (stimulated CD8⁺ T cell:
Kasumi-3 cells) upon incubation for 14 hrs. Frequency of CFSE
and 7-AAD positive cells when treated with different
concentrations of bispecific antibodies at 11 ET ratio for 14 hrs.

| | | | anti-Vβ17/ |
|---|---|---|---|
| Conc.(ng/mL) | NULLXCD123 | Vβ17XNULL | anti-CD123 |
| 50 | 0.6 | -0.5 | 76.0 |
| 5 | 0.1 | 0.9 | 73.0 |
| 0.5 | 1.0 | 0.9 | 77.9 |
| 0.05 | 0.4 | 1.8 | 59.4 |
| 0.005 | 1.8 | 0.9 | 36.8 |
| 0.0005 | 0.9 | 1.1 | 18.0 |
| 0.00005 | 0.7 | 0.9 | 6.1 |
| 0.000005 | 1.6 | 0.8 | 1.9 |
| EC₅₀ (ng/mL) | ND | ND | 0.015 |
| EC₅₀ (pM) | ND | ND | 0.1 |

Half maximal effective concentration (EC₅₀) values were calculated as the antibody concentration that generates 50% of maximal cytotoxicity (CFSE⁺ 7AAD⁺) cells. ND: Not Determined

TABLE 17
Cytotoxicity assay at 5:1 E:T ratio (stimulated CD8⁺ T cell:
Kasumi-3 cells) upon incubation for 14 hrs. Frequency of CFSE
and 7-AAD positive cells when treated with different
concentrations of bispecific antibodies at 5:1 ET ratio for 14 hrs.

| | | | anti-Vβ17/ |
|---|---|---|---|
| Conc.(ng/mL) | NULLXCD123 | Vβ17XNULL | anti-CD123 |
| 50 | 2.3 | 0.0 | 70.2 |
| 5 | 3.1 | 2.7 | 70.9 |
| 0.5 | 1.8 | 4.4 | 74.5 |
| 0.05 | 3.0 | 1.5 | 73.2 |
| 0.005 | 2.1 | 1.6 | 2.5 |
| 0.0005 | 2.9 | 3.5 | 1.4 |
| 0.00005 | 3.2 | 5.5 | 2.5 |
| 0.000005 | 4.1 | 4.4 | 4.8 |
| EC₅₀ (ng/mL) | UD | UD | 0.02 |
| EC₅₀ (pM) | UD | UD | 0.13 |

Half maximal effective concentration (EC₅₀) values were calculated as the antibody concentration that generates 50% of maximal cytotoxicity (CFSE⁺ 7AAD⁺) cells. UD: Undetectable.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

| SEQ | | | | |
|---|---|---|---|---|
| ID | | | | |
| NO: | Type | Species | Description | Sequence |
| 1 | PRT | mouse | B17B01- | GYSITSGYFWN |
| | | | HCDR1 | |
| 2 | PRT | mouse | B12B01- | YISYDGSNN |
| | | | HCDR2 | |
| 3 | PRT | mouse | B12B01- | PSPGTGYAVDY |
| | | | HCDR2 | |
| 4 | PRT | mouse | B17B01- | RSSQSLVHSNGNTYLH |
| | | | LCDR1 | |
| 5 | PRT | mouse | B12B01- | KVSNRFS |
| | | | LCDR2 | |
| 6 | PRT | mouse | B12B01- | SQSTHVPFT |
| | | | LCDR2 | |
| 7 | PRT | mouse | B17B01-HC | NVQLQESGPGLVKPSQSLSLTCSVAGYSITSG |
| | | | | YFWNWIRQFPGNKLEWMGYISYDGSNNYNP |
| | | | | SLKNRISITRDTSKNQFFLKLNSVTTEDTATY |
| | | | | YCASPSPGTGYAVDYWGQGTSVTVSSAKTTP |
| | | | | PSVYPLAPGSAAQTNSMVTLGCLVKGYFPEP |
| | | | | VTVTWNSGSLSSGVHTFPAVLQSDLYTLSSS |
| | | | | VTVPSSTWPSQTVTCNVAHPASSTKVDKKIV |
| | | | | PRDCGCKPCICTVPEVSSVFIFPPKPKDVLTIT |
| | | | | LTPKVTCVVVDISKDDPEVQFSWFVDDVEVH |
| | | | | TAQTKPREEQINSTFRSVSELPIMHQDWLNGK |
| | | | | EFKCRVNSAAFPAPIEKTISKTYGRPKAPQVY |
| | | | | TIPPPKEQMAKDKVSLTCMITNFFPEDITVEW |
| | | | | QWNGQPAENYKNTQPIMDTDGSYFVYSKLN |
| | | | | VQKSNWEAGNTFTCSVLHEGLHNHHTEKSLS |
| | | | | HSPGK |
| 8 | PRT | mouse | B17B01-LC | NVVMTQTPLSLPVSLGDQASISCRSSQSLVHS |
| | | | | NGNTYLHWYLQKPGQSPKFLIYKVSNRFSGV |
| | | | | PDRFSGGGSGTEFTLKISRVEAEDLGVYFCSQ |
| | | | | STHVPFTFGSGTKLEIKRADAAPTVSIFPPSSE |
| | | | | QLTSGGASVVCFLNNFYPKDINVKWKIDGSE |
| | | | | RQNGVLNSWTDQDSKDSTYSMSSTLTLTKDE |
| | | | | YERHNSYTCEATHKTSTSPIVKSFNRNEC |
| 9 | PRT | mouse | B17B1-HC | NVQLQESGPGLVKPSQSLSLTCSVAGYSITSG |
| | | | | YFWNWIRQFPGNKLEWMGYISYDGSNNYNP |
| | | | | SLKNRISITRDTSKNQFFLKLNSVTTEDTATY |
| | | | | YCASPSPGTGYAVDYWGQGTSVTVSSASTK |
| | | | | GPSVFPLAPCSRSTSESTAALGCLVKDYFPEP |
| | | | | VTVSWNSGALTSGVHTFPAVLQSSGLYSLSS |
| | | | | VVTVPSSSLGTKTYTCNVDHKPSNTKVDKRV |
| | | | | ESKYGPPCPPCPAPEAAGGPSVFLFPPKPKDT |
| | | | | LMISRTPEVTCVVVDVSQEDPEVQFNWYVD |
| | | | | GVEVHNAKTKPREEQFNSTYRVVSVLTVLHQ |
| | | | | DWLNGKEYKCKVSNKGLPSSIEKTISKAKGQ |
| | | | | PREPQVYTLPPSQEEMTKNQVSLTCLVKGFY |
| | | | | PSDIAVEWESNGQPENNYKTTPPVLDSDGSFL |
| | | | | LYSKLTVDKSRWQEGNVFSCSVMHEALHNH |
| | | | | YTQKSLSLSLGK |
| 10 | PRT | mouse | B17B1-LC | NVVMTQTPLSLPVSLGDQASISCRSSQSLVHS |
| | | | | NGNTYLHWYLQKPGQSPKFLIYKVSNRFSGV |
| | | | | PDRFSGGGSGTEFTLKISRVEAEDLGVYFCSQ |
| | | | | STHVPFTFGSGTKLEIKRADAAPTVSIFPPSSE |
| | | | | QLTSGGASVVCFLNNFYPKDINVKWKIDGSE |
| | | | | RQNGVLNSWTDQDSKDSTYSMSSTLTLTKDE |
| | | | | YERHNSYTCEATHKTSTSPIVKSFNRNEC |
| 11 | PRT | mouse | B17B2-HC | DVQLKESGPGLVKPSQSLSVTCSVTGYSITSG |
| | | | | YYWNWYRQFPGNKLEWMGYISYDGSNNYN |
| | | | | PSLKNRISITRDTSKNQILLKLTYVTTEDTATY |
| | | | | YCTRPSPGTGYAVDYWGQGTLVTVSSASTK |
| | | | | GPSVFPLAPCSRSTSESTAALGCLVKDYFPEP |
| | | | | VTVSWNSGALTSGVHTFPAVLQSSGLYSLSS |
| | | | | VVTVPSSSLGTKTYTCNVDHKPSNTKVDKRV |
| | | | | ESKYGPPCPPCPAPEAAGGPSVFLFPPKPKDT |
| | | | | LMISRTPEVTCVVVDVSQEDPEVQFNWYVD |
| | | | | GVEVHNAKTKPREEQFNSTYRVVSVLTVLHQ |
| | | | | DWLNGKEYKCKVSNKGLPSSIEKTISKAKGQ |
| | | | | PREPQVYTLPPSQEEMTKNQVSLTCLVKGFY |
| | | | | PSDIAVEWESNGQPENNYKTTPPVLDSDGSFL |
| | | | | LYSKLTVDKSRWQEGNVFSCSVMHEALHNH |
| | | | | YTQKSLSLSLGK |
| 12 | PRT | mouse | B17B2-LC | DIVMTQSPDSLAVSLGERATINCRSSQSLVHS |
| | | | | NGNTYLHWYQQKPGQPPKLLIYKVSNRFSGV |
| | | | | PDRFSGSGSGTDFTLTISSLQAEDVAVYYCSQ |
| | | | | STHVPFTFGQGTKVEIKRADAAPTVSIFPPSSE |
| | | | | QLTSGGASVVCFLNNFYPKDINVKWKIDGSE |
| | | | | RQNGVLNSWTDQDSKDSTYSMSSTLTLTKDE |
| | | | | YERHNSYTCEATHKTSTSPIVKSFNRNEC |
| 13 | PRT | artificial | B17B21-HC | QVQLQESGPGLVKPSETLSLTCTVSGYSITSG |
| | | | | YFWNWIRQPPGKGLEWIGYISYDGSNNYNPS |
| | | | | LKSRVTISRDTSKNQFSLKLSSVTAADTAVYY |
| | | | | CASPSPGTGYAVDYWGQGTLVTVSSASTKGP |
| | | | | SVFPLAPCSRSTSESTAALGCLVKDYFPEPVT |
| | | | | VSWNSGALTSGVHTFPAVLQSSGLYSLSSVV |
| | | | | TVPSSSLGTKTYTCNVDHKPSNTKVDKRVES |
| | | | | KYGPPCPPCPAPEAAGGPSVFLFPPKPKDTLM |
| | | | | ISRTPEVTCVVVDVSQEDPEVQFNWYVDGVE |
| | | | | VHNAKTKPREEQFNSTYRVVSVLTVLHQDW |
| | | | | LNGKEYKCKVSNKGLPSSIEKTISKAKGQPRE |
| | | | | PQVYTLPPSQEEMTKNQVSLSCAVKGFYPSDI |
| | | | | AVEWESNGQPENNYKTTPPVLDSDGSFFLVS |
| | | | | RLTVDKSRWQEGNVFSCSVMHEALHNRFTQ |
| | | | | KSLSLSLGK |
| 14 | PRT | artificial | B17B21-LC | DIQMTQSPSSLSASVGDRVTITCRSSQSLVHS |
| | | | | NGNTYLHWYQQKPGKAPKFLIYKVSNRFSG |
| | | | | VPSRFSGSGSGTDFTLTISSLQPEDFATYYCSQ |
| | | | | STHVPFTFGQGTKLEIKRTVAAPSVFIFPPSDE |
| | | | | QLKSGTASVVCLLNNFYPREAKVQWKVDNA |
| | | | | LQSGNSQESVTEQDSKDSTYSLSSTLTLSKAD |
| | | | | YEKHKVYACEVTHQGLSSPVTKSFNRGEC |
| 15 | PRT | human | I3RB217-HC | EVQLVQSGAEVKKPGESLKISCKGSGYSFTSY |
| | | | | WISWVRQMPGKGLEWMGIIDPSDSDTRYSPS |
| | | | | FQGQVTISADKSISTAYLQWSSLKASDTAMY |
| | | | | YCARGDGSTDLDYWGQGTLVTVSSASTKGP |
| | | | | SVFPLAPCSRSTSESTAALGCLVKDYFPEPVT |
| | | | | VSWNSGALTSGVHTFPAVLQSSGLYSLSSVV |
| | | | | TVPSSSLGTKTYTCNVDHKPSNTKVDKRVES |
| | | | | KYGPPCPPCPAPEAAGGPSVFLFPPKPKDTLM |
| | | | | ISRTPEVTCVVVDVSQEDPEVQFNWYVDGVE |
| | | | | VHNAKTKPREEQFNSTYRVVSVLTVLHQDW |
| | | | | LNGKEYKCKVSNKGLPSSIEKTISKAKGQPRE |
| | | | | PQVYTLPPSQEEMTKNQVSLWCLVKGFYPSD |
| | | | | IAVEWESNGQPENNYKTTPPVLDSDGSFFLYS |
| | | | | RLTVDKSRWQEGNVFSCSVMHEALHNHYTQ |
| | | | | KSLSLSLGK |
| 16 | PRT | human | I3RB217-LC | EIVLTQSPGTLSLSPGERATLSCRASQSVSSSY |
| | | | | LAWYQQKPGQAPRLLIYGASSRATGIPDRFS |
| | | | | GSGSGTDFTLTISRLEPEDFAVYYCQQDYGFP |
| | | | | WTFGQGTKVEIKRTVAAPSVFIFPPSDEQLKS |
| | | | | GTASVVCLLNNFYPREAKVQWKVDNALQSG |
| | | | | NSQESVTEQDSKDSTYSLSSTLTLSKADYEKH |
| | | | | KVYACEVTHQGLSSPVTKSFNRGEC |
| 17 | PRT | human | Null-HC | QITLKESGPTLVKPTQTLTLTCTFSGFSLSTSG |
| | | | | MGVSWIRQPPGKALEWLAHIYWDDDKRYNP |
| | | | | SLKSRLTITKDTSKNQVVLTMTNMDPVDTAT |
| | | | | YYCARLYGFTYGFAYWGQGTLVTVSSASTK |
| | | | | GPSVFPLAPCSRSTSESTAALGCLVKDYFPEP |
| | | | | VTVSWNSGALTSGVHTFPAVLQSSGLYSLSS |
| | | | | VVTVPSSSLGTKTYTCNVDHKPSNTKVDKRV |
| | | | | ESKYGPPCPPCPAPEAAGGPSVFLFPPKPKDT |
| | | | | LMISRTPEVTCVVVDVSQEDPEVQFNWYVD |
| | | | | GVEVHNAKTKPREEQFNSTYRVVSVLTVLHQ |
| | | | | DWLNGKEYKCKVSNKGLPSSIEKTISKAKGQ |
| | | | | PREPQVYTLPPSQEEMTKNQVSLWCLVKGFY |
| | | | | PSDIAVEWESNGQPENNYKTTPPVLDSDGSFF |
| | | | | LYSRLTVDKSRWQEGNVFSCSVMHEALHNH |
| | | | | YTQKSLSLSLGK |
| 18 | PRT | human | Null-LC | DIVMTQSPDSLAVSLGERATINCRASQSVDY |
| | | | | NGISYMHWYQQKPGQPPKLLIYAASNPESGV |
| | | | | PDRFSGSGSGTDFTLTISSLQAEDVAVYYCQQ |
| | | | | IIEDPWTFGQGTKVEIKRTVAAPSVFIFPPSDE |
| | | | | QLKSGTASVVCLLNNFYPREAKVQWKVDNA |
| | | | | LQSGNSQESVTEQDSKDSTYSLSSTLTLSKAD |
| | | | | YEKHKVYACEVTHQGLSSPVTKSFNRGEC |
| 19 | PRT | artificial | B17H3 | EVQLLESGGGLVQPGGSLRLSCAASGYSITSG |
| | | | | YFWNWVRQAPGKGLEWVSYISYDGSNNYA |
| | | | | DSVKGRFTISRDNSKNTLYLQMNSLRAEDTA |
| | | | | VYYCAKPSPGTGYAVDYWGQGTLVTVS |
| 20 | PRT | artificial | B17H4 | EVQLLESGGGLVQPGGSLRLSCAASGYSITSG |
| | | | | YFWNWVRQAPGKGLEWVSYISYDGSNNYA |
| | | | | DSVKGRFTISRDNSKNTLYLQMNSLRAEDTA |
| | | | | VYYCASPSPGTGYAVDYWGQGTLVTVSS |
| 21 | PRT | artificial | B17H5 | QVQLQESGPGLVKPSETLSLTCTVSGYSITSG |
| | | | | YFWNWIRQPPGKGLEWIGYISYDGSNNYNPS |
| | | | | LKSRVTISRDTSKNQFSLKLSSVTAADTAVYY |
| | | | | CASPSPGTGYAVDYWGQGTLVTVSS |
| 22 | PRT | artificial | B17L3 | DIQMTQSPSSLSASVGDRVTITCRSSQSLVHS |
| | | | | NGNTYLHWYQQKPGKAPKLLIYKVSNRFSG |
| | | | | VPSRFSGSGSGTDFTLTISSLQPEDFATYYCSQ |
| | | | | STHVPFTFGQGTKLEIK |
| 23 | PRT | artificial | B17L4 | DIQMTQSPSSLSASVGDRVTITCRSSQSLVHS |
| | | | | NGNTYLHWYQQKPGKAPKFLIYKVSNRFSG |
| | | | | VPSRFSGSGSGTDFTLTISSLQPEDFATYYCSQ |
| | | | | STHVPFTFGQGTKLEIK |
| 24 | PRT | artificial | B17L5 | DVVMTQSPLSLPVTLGQPASISCRSSQSLVHS |
| | | | | NGNTYLHWFQQRPGQSPRFLIYKVSNRFSGV |
| | | | | PDRFSGSGSGTDFTLKISRVEAEDVGVYYCSQ |
| | | | | STHVPFTFGQGTKLEIK |
| 25 | PRT | artificial | B17H1 | NVQLQESGPGLVKPSQSLSLTCSVAGYSITSG |
| | | | | YFWNWIRQFPGNKLEWMGYISYDGSNNYNP |
| | | | | SLKNRISITRDTSKNQFFLKLNSVTTEDTATY |
| | | | | YCASPSPGTGYAVDYWGQGTSVTVSS |
| 26 | PRT | artificial | B17L1 | NVVMTQTPLSLPVSLGDQASISCRSSQSLVHS |
| | | | | NGNTYLHWYLQKPGQSPKFLIYKVSNRFSGV |
| | | | | PDRFSGGGSGTEFTLKISRVEAEDLGVYFCSQ |
| | | | | STHVPFTFGSGTKLEIK |
| 27 | PRT | human | TCR-VB17 | MAWVWTLLFLMAAAQSIQAVDGGITQSPKY |
| | | | | LFRKEGQNVTLSCEQNLNHDAMYWYRQDPG |
| | | | | QGLRLIYYSQIVNDFQKGDIAEGYSVSREKKE |
| | | | | SFPLTVTSAQKNPTAFYLCASSSRSSYEQYFG |
| | | | | PGTRLTVTEDLKNVFPPEVAVFEPSEAEISHT |
| | | | | QKATLVCLATGFYPDHVELSWWVNGKEVHS |
| | | | | GVSTDPQPLKEQPALNDSRYSLSSRLRVSATF |
| | | | | WQNPRNHFRCQVQFYGLSENDEWTQDRAKP |
| | | | | VTQIVSAEAWGRADepkscdkthtcppcpapeLLggps |
| | | | | vflfppkpkdtlmisrtpevtcvvvDvshedpevkfnwyvdgvev |
| | | | | hnaktkpreeqynstyrvvsvltvlhqdwlngkeykckvsnkalpa |
| | | | | piektiskakgqprepqvyVlppsreemtknqvslLclvkgfyps |
| | | | | diavewesngqpennyLtWppvldsdgsfflyskltvdksrwqq |
| | | | | gnvfscsvmhealhnhytqkslslspg |
| 28 | PRT | artificial | B17B21 half | MAWVWTLLFLMAAAQSIQADIQMTQSPSSLS |
| | | | antibody | ASVGDRVTITCRSSQSLVHSNGNTYLHWYQQ |
| | | | | KPGKAPKFLIYKVSNRFSGVPSRFSGSGSGTD |
| | | | | FTLTISSLQPEDFATYYCSQSTHVPFTFGQGTK |
| | | | | LEIKRTVAAPSVFIFPPSDEQLKSGTASVVCLL |
| | | | | NNFYPREAKVQWKVDNALQSGNSQESVTEQ |
| | | | | DSKDSTYSLSSTLTLSKADYEKHKVYACEVT |
| | | | | HQGLSSPVTKSFNRGECGGSEGKSSGSGSESK |
| | | | | STEGKSSGSGSESKSTGGSQVQLQESGPGLVK |
| | | | | PSETLSLTCTVSGYSITSGYFWNWIRQPPGKG |
| | | | | LEWIGYISYDGSNNYNPSLKSRVTISRDTSKN |
| | | | | QFSLKLSSVTAADTAVYYCASPSPGTGYAVD |
| | | | | YWGQGTLVTVSSASTKGPSVFPLAPCSRSTSE |
| | | | | STAALGCLVKDYFPEPVTVSWNSGALTSGVH |
| | | | | TFPAVLQSSGLYSLSSVVTVPSSSLGTKTYTC |
| | | | | NVDHKPSNTKVDKRVESKYGPPCPPCPAPEA |
| | | | | AGGPSVFLFPPKPKDTLMISRTPEVTCVVVDV |
| | | | | SQEDPEVQFNWYVDGVEVHNAKTKPREEQF |
| | | | | NSTYRVVSVLTVLHQDWLNGKEYKCKVSNK |
| | | | | GLPSSIEKTISKAKGQPREPQVYTLPPSQEEMT |
| | | | | KNQVSLSCAVKGFYPSDIAVEWESNGQPENN |
| | | | | YKTTPPVLDSDGSFFLVSRLTVDKSRWQEGN |
| | | | | VFSCSVMHEALHNRFTQKSLSLSLGK |
| | | | | ATGGCCTGGGTGTGGACCCTGCTGTTCCTG |
| 29 | DNA | artificial | B17B21 half | ATGGCCGCCGCCCAGAGCATCCAGGCCGAC |
| | | | antibody | ATCCAGATGACCCAGAGCCCAAGCAGCCTG |
| | | | | AGCGCCAGCGTGGGCGACCGCGTGACCATC |
| | | | | ACCTGCCGCAGCAGCCAGAGCCTGGTGCAC |
| | | | | AGCAACGGCAACACCTACCTGCACTGGTAC |
| | | | | CAGCAGAAGCCAGGCAAGGCCCCAAAGTT |
| | | | | CCTGATCTACAAGGTGAGCAACCGCTTCAG |
| | | | | CGGCGTGCCAAGCCGCTTCAGCGGCAGCGG |
| | | | | CAGCGGCACCGACTTCACCCTGACCATCAG |
| | | | | CAGCCTGCAGCCAGAGGACTTCGCCACCTA |
| | | | | CTACTGCAGCCAGAGCACCCACGTGCCATT |
| | | | | CACCTTCGGCCAGGGCACCAAGCTGGAGAT |
| | | | | CAAGCGCACCGTGGCCGCCCCAAGCGTGTT |
| | | | | CATCTTCCCACCAAGCGACGAGCAGCTGAA |
| | | | | GAGCGGCACCGCCAGCGTGGTGTGCCTGCT |
| | | | | GAACAACTTCTACCCACGCGAGGCCAAGGT |
| | | | | GCAGTGGAAGGTGGACAACGCCCTGCAGA |
| | | | | GCGGCAACAGCCAGGAGAGCGTGACCGAG |
| | | | | CAGGACAGCAAGGACAGCACCTACAGCCT |
| | | | | GAGCAGCACCCTGACCCTGAGCAAGGCCG |
| | | | | ACTACGAGAAGCACAAGGTGTACGCCTGC |
| | | | | GAGGTGACCCACCAGGGCCTGAGCAGCCC |
| | | | | AGTGACCAAGAGCTTCAACCGCGGCGAGT |
| | | | | GCGGCGGCAGCGAGGGCAAGAGCAGCGGC |
| | | | | AGCGGCAGCGAGAGCAAGAGCACCGAGGG |
| | | | | CAAGAGCAGCGGCAGCGGCAGCGAGAGCA |
| | | | | AGAGCACCGGCGGCAGCCAGGTGCAGCTG |
| | | | | CAGGAGAGCGGCCCAGGCCTGGTGAAGCC |
| | | | | AAGCGAGACCCTGAGCCTGACCTGCACCGT |
| | | | | GAGCGGCTACAGCATCACCAGCGGCTACTT |
| | | | | CTGGAACTGGATCCGCCAGCCACCAGGCAA |
| | | | | GGGCCTGGAGTGGATCGGCTACATCAGCTA |
| | | | | CGACGGCAGCAACAACTACAACCCAAGCC |
| | | | | TGAAGAGCCGCGTGACCATCAGCCGCGAC |
| | | | | ACCAGCAAGAACCAGTTCAGCCTGAAGCTG |
| | | | | AGCAGCGTGACCGCCGCCGACACCGCCGTG |
| | | | | TACTACTGCGCCAGCCCAAGCCCAGGCACC |
| | | | | GGCTACGCCGTGGACTACTGGGGCCAGGGC |
| | | | | ACCCTGGTGACCGTGAGCAGCGCCAGCACC |
| | | | | AAGGGCCCAAGCGTGTTCCCACTGGCCCCA |
| | | | | TGCAGCCGCAGCACCAGCGAGAGCACCGC |
| | | | | CGCCCTGGGCTGCCTGGTGAAGGACTACTT |
| | | | | CCCAGAGCCAGTGACCGTGAGCTGGAACA |
| | | | | GCGGCGCCCTGACCAGCGGCGTGCACACCT |
| | | | | TCCCAGCCGTGCTGCAGAGCAGCGGCCTGT |
| | | | | ACAGCCTGAGCAGCGTGGTGACCGTGCCAA |
| | | | | GCAGCAGCCTGGGCACCAAGACCTACACCT |
| | | | | GCAACGTGGACCACAAGCCAAGCAACACC |
| | | | | AAGGTGGACAAGCGCGTGGAGAGCAAGTA |
| | | | | CGGCCCACCATGCCCACCATGCCCAGCCCC |
| | | | | AGAGGCCGCCGGCGGCCCAAGCGTGTTCCT |
| | | | | GTTCCCACCAAAGCCAAAGGACACCCTGAT |
| | | | | GATCAGCCGCACCCCAGAGGTGACCTGCGT |
| | | | | GGTGGTGGACGTGAGCCAGGAGGACCCAG |
| | | | | AGGTGCAGTTCAACTGGTACGTGGACGGCG |
| | | | | TGGAGGTGCACAACGCCAAGACCAAGCCA |
| | | | | CGCGAGGAGCAGTTCAACAGCACCTACCGC |
| | | | | GTGGTGAGCGTGCTGACCGTGCTGCACCAG |
| | | | | GACTGGCTGAACGGCAAGGAGTACAAGTG |
| | | | | CAAGGTGAGCAACAAGGGCCTGCCAAGCA |
| | | | | GCATCGAGAAGACCATCAGCAAGGCCAAG |
| | | | | GGCCAGCCACGCGAGCCACAGGTGTACAC |
| | | | | CCTGCCACCAAGCCAGGAGGAGATGACCA |
| | | | | AGAACCAGGTGAGCCTGAGCTGCGCCGTG |
| | | | | AAGGGCTTCTACCCAAGCGACATCGCCGTG |
| | | | | GAGTGGGAGAGCAACGGCCAGCCAGAGAA |
| | | | | CAACTACAAGACCACCCCACCAGTGCTGGA |
| | | | | CAGCGACGGCAGCTTCTTCCTGGTGAGCCG |
| | | | | CCTGACCGTGGACAAGAGCCGCTGGCAGG |
| | | | | AGGGCAACGTGTTCAGCTGCAGCGTGATGC |
| | | | | ACGAGGCCCTGCACAACCGCTTCACCCAGA |
| | | | | AGAGCCTGAGCCTGAGCCTGGGCAAGATG |
| | | | | GCCTGGGTGTGGACCCTGCTGTTCCTGATG |
| | | | | GCCGCCGCCCAGAGCATCCAGGCCGACATC |
| | | | | CAGATGACCCAGAGCCCAAGCAGCCTGAG |
| | | | | CGCCAGCGTGGGCGACCGCGTGACCATCAC |
| | | | | CTGCCGCAGCAGCCAGAGCCTGGTGCACAG |
| | | | | CAACGGCAACACCTACCTGCACTGGTACCA |
| | | | | GCAGAAGCCAGGCAAGGCCCCAAAGTTCC |
| | | | | TGATCTACAAGGTGAGCAACCGCTTCAGCG |
| | | | | GCGTGCCAAGCCGCTTCAGCGGCAGCGGCA |
| | | | | GCGGCACCGACTTCACCCTGACCATCAGCA |
| | | | | GCCTGCAGCCAGAGGACTTCGCCACCTACT |
| | | | | ACTGCAGCCAGAGCACCCACGTGCCATTCA |
| | | | | CCTTCGGCCAGGGCACCAAGCTGGAGATCA |
| | | | | AGCGCACCGTGGCCGCCCCAAGCGTGTTCA |
| | | | | TCTTCCCACCAAGCGACGAGCAGCTGAAGA |
| | | | | GCGGCACCGCCAGCGTGGTGTGCCTGCTGA |
| | | | | ACAACTTCTACCCACGCGAGGCCAAGGTGC |
| | | | | AGTGGAAGGTGGACAACGCCCTGCAGAGC |
| | | | | GGCAACAGCCAGGAGAGCGTGACCGAGCA |
| | | | | GGACAGCAAGGACAGCACCTACAGCCTGA |
| | | | | GCAGCACCCTGACCCTGAGCAAGGCCGACT |
| | | | | ACGAGAAGCACAAGGTGTACGCCTGCGAG |
| | | | | GTGACCCACCAGGGCCTGAGCAGCCCAGTG |
| | | | | ACCAAGAGCTTCAACCGCGGCGAGTGCGG |
| | | | | CGGCAGCGAGGGCAAGAGCAGCGGCAGCG |
| | | | | GCAGCGAGAGCAAGAGCACCGAGGGCAAG |
| | | | | AGCAGCGGCAGCGGCAGCGAGAGCAAGAG |
| | | | | CACCGGCGGCAGCCAGGTGCAGCTGCAGG |
| | | | | AGAGCGGCCCAGGCCTGGTGAAGCCAAGC |
| | | | | GAGACCCTGAGCCTGACCTGCACCGTGAGC |
| | | | | GGCTACAGCATCACCAGCGGCTACTTCTGG |
| | | | | AACTGGATCCGCCAGCCACCAGGCAAGGG |
| | | | | CCTGGAGTGGATCGGCTACATCAGCTACGA |
| | | | | CGGCAGCAACAACTACAACCCAAGCCTGA |
| | | | | AGAGCCGCGTGACCATCAGCCGCGACACC |
| | | | | AGCAAGAACCAGTTCAGCCTGAAGCTGAG |
| | | | | CAGCGTGACCGCCGCCGACACCGCCGTGTA |
| | | | | CTACTGCGCCAGCCCAAGCCCAGGCACCGG |
| | | | | CTACGCCGTGGACTACTGGGGCCAGGGCAC |
| | | | | CCTGGTGACCGTGAGCAGCGCCAGCACCAA |
| | | | | GGGCCCAAGCGTGTTCCCACTGGCCCCATG |
| | | | | CAGCCGCAGCACCAGCGAGAGCACCGCCG |
| | | | | CCCTGGGCTGCCTGGTGAAGGACTACTTCC |
| | | | | CAGAGCCAGTGACCGTGAGCTGGAACAGC |
| | | | | GGCGCCCTGACCAGCGGCGTGCACACCTTC |
| | | | | CCAGCCGTGCTGCAGAGCAGCGGCCTGTAC |
| | | | | AGCCTGAGCAGCGTGGTGACCGTGCCAAGC |
| | | | | AGCAGCCTGGGCACCAAGACCTACACCTGC |
| | | | | AACGTGGACCACAAGCCAAGCAACACCAA |
| | | | | GGTGGACAAGCGCGTGGAGAGCAAGTACG |
| | | | | GCCCACCATGCCCACCATGCCCAGCCCCAG |
| | | | | AGGCCGCCGGCGGCCCAAGCGTGTTCCTGT |
| | | | | TCCCACCAAAGCCAAAGGACACCCTGATGA |
| | | | | TCAGCCGCACCCCAGAGGTGACCTGCGTGG |
| | | | | TGGTGGACGTGAGCCAGGAGGACCCAGAG |
| | | | | GTGCAGTTCAACTGGTACGTGGACGGCGTG |
| | | | | GAGGTGCACAACGCCAAGACCAAGCCACG |
| | | | | CGAGGAGCAGTTCAACAGCACCTACCGCGT |
| | | | | GGTGAGCGTGCTGACCGTGCTGCACCAGGA |
| | | | | CTGGCTGAACGGCAAGGAGTACAAGTGCA |
| | | | | AGGTGAGCAACAAGGGCCTGCCAAGCAGC |
| | | | | ATCGAGAAGACCATCAGCAAGGCCAAGGG |
| | | | | CCAGCCACGCGAGCCACAGGTGTACACCCT |
| | | | | GCCACCAAGCCAGGAGGAGATGACCAAGA |
| | | | | ACCAGGTGAGCCTGAGCTGCGCCGTGAAG |
| | | | | GGCTTCTACCCAAGCGACATCGCCGTGGAG |
| | | | | TGGGAGAGCAACGGCCAGCCAGAGAACAA |
| | | | | CTACAAGACCACCCCACCAGTGCTGGACAG |
| | | | | CGACGGCAGCTTCTTCCTGGTGAGCCGCCT |
| | | | | GACCGTGGACAAGAGCCGCTGGCAGGAGG |
| | | | | GCAACGTGTTCAGCTGCAGCGTGATGCACG |
| | | | | AGGCCCTGCACAACCGCTTCACCCAGAAGA |
| | | | | GCCTGAGCCTGAGCCTGGGCAAGTGATAG |
| 30 | PRT | artificial | I3RB217 half | MAWVWTLLFLMAAAQSIQAEIVLTQSPGTLS |
| | | | antibody | LSPGERATLSCRASQSVSSSYLAWYQQKPGQ |
| | | | | APRLLIYGASSRATGIPDRFSGSGSGTDFTLTI |
| | | | | SRLEPEDFAVYYCQQDYGFPWTFGQGTKVEI |
| | | | | KRTVAAPSVFIFPPSDEQLKSGTASVVCLLNN |
| | | | | FYPREAKVQWKVDNALQSGNSQESVTEQDS |
| | | | | KDSTYSLSSTLTLSKADYEKHKVYACEVTHQ |
| | | | | GLSSPVTKSFNRGECGGSEGKSSGSGSESKST |
| | | | | EGKSSGSGSESKSTGGSEVQLVQSGAEVKKP |
| | | | | GESLKISCKGSGYSFTSYWISWVRQMPGKGL |
| | | | | EWMGIIDPSDSDTRYSPSFQGQVTISADKSIST |
| | | | | AYLQWSSLKASDTAMYYCARGDGSTDLDY |
| | | | | WGQGTLVTVSSASTKGPSVFPLAPCSRSTSES |
| | | | | TAALGCLVKDYFPEPVTVSWNSGALTSGVHT |
| | | | | FPAVLQSSGLYSLSSVVTVPSSSLGTKTYTCN |
| | | | | VDHKPSNTKVDKRVESKYGPPCPPCPAPEAA |
| | | | | GGPSVFLFPPKPKDTLMISRTPEVTCVVVDVS |
| | | | | QEDPEVQFNWYVDGVEVHNAKTKPREEQFN |
| | | | | STYRVVSVLTVLHQDWLNGKEYKCKVSNKG |
| | | | | LPSSIEKTISKAKGQPREPQVYTLPPSQEEMTK |
| | | | | NQVSLWCLVKGFYPSDIAVEWESNGQPENN |
| | | | | YKTTPPVLDSDGSFFLYSRLTVDKSRWQEGN |
| | | | | VFSCSVMHEALHNHYTQKSLSLSLGK |
| 31 | DNA | artificial | I3RB217 half | ATGGCCTGGGTGTGGACCCTGCTGTTCCTG |
| | | | | GATCGTGCTGACCCAGAGCCCAGGCACCCT |
| | | | | GAGCCTGAGCCCAGGCGAGCGCGCCACCCT |
| | | | | GAGCTGCCGCGCCAGCCAGAGCGTGAGCA |
| | | | | GCAGCTACCTGGCCTGGTACCAGCAGAAGC |
| | | | | CAGGCCAGGCCCCACGCCTGCTGATCTACG |
| | | | | GCGCCAGCAGCCGCGCCACCGGCATCCCAG |
| | | | | ACCGCTTCAGCGGCAGCGGCAGCGGCACC |
| | | | | GACTTCACCCTGACCATCAGCCGCCTGGAG |
| | | | | CCAGAGGACTTCGCCGTGTACTACTGCCAG |
| | | | | CAGGACTACGGCTTCCCATGGACCTTCGGC |
| | | | | CAGGGCACCAAGGTGGAGATCAAGCGCAC |
| | | | | CGTGGCCGCCCCAAGCGTGTTCATCTTCCC |
| | | | | ACCAAGCGACGAGCAGCTGAAGAGCGGCA |
| | | | | CCGCCAGCGTGGTGTGCCTGCTGAACAACT |
| | | | | TCTACCCACGCGAGGCCAAGGTGCAGTGGA |
| | | | | AGGTGGACAACGCCCTGCAGAGCGGCAAC |
| | | | | AGCCAGGAGAGCGTGACCGAGCAGGACAG |
| | | | | CAAGGACAGCACCTACAGCCTGAGCAGCA |
| | | | | CCCTGACCCTGAGCAAGGCCGACTACGAGA |
| | | | | AGCACAAGGTGTACGCCTGCGAGGTGACCC |
| | | | | ACCAGGGCCTGAGCAGCCCAGTGACCAAG |
| | | | | AGCTTCAACCGCGGCGAGTGCggcggcagcgagg |
| | | | | gcaagagcagcggcagcggcagcgagagcaagagcaccgagggc |
| | | | | aagagcagcggcagcggcagcgagagcaagagcaccggcggcag |
| | | | | cGAGGTGCAGCTGGTGCAGAGCGGCGCCGA |
| | | | | GGTGAAGAAGCCAGGCGAGAGCCTGAAGA |
| | | | | TCAGCTGCAAGGGCAGCGGCTACAGCTTCA |
| | | | | CCAGCTACTGGATCAGCTGGGTGCGCCAGA |
| | | | | TGCCAGGCAAGGGCCTGGAGTGGATGGGC |
| | | | | ATCATCGACCCAAGCGACAGCGACACCCGC |
| | | | | TACAGCCCAAGCTTCCAGGGCCAGGTGACC |
| | | | | ATCAGCGCCGACAAGAGCATCAGCACCGC |
| | | | | CTACCTGCAGTGGAGCAGCCTGAAGGCCAG |
| | | | | CGACACCGCCATGTACTACTGCGCCCGCGG |
| | | | | CGACGGCAGCACCGACCTGGACTACTGGG |
| | | | | GCCAGGGCACCCTGGTGACCGTGAGCAGC |
| | | | | GCCAGCACCAAGGGCCCAAGCGTGTTCCCA |
| | | | | CTGGCCCCATGCAGCCGCAGCACCAGCGAG |
| | | | | AGCACCGCCGCCCTGGGCTGCCTGGTGAAG |
| | | | | GACTACTTCCCAGAGCCAGTGACCGTGAGC |
| | | | | TGGAACAGCGGCGCCCTGACCAGCGGCGT |
| | | | | GCACACCTTCCCAGCCGTGCTGCAGAGCAG |
| | | | | CGGCCTGTACAGCCTGAGCAGCGTGGTGAC |
| | | | | CGTGCCAAGCAGCAGCCTGGGCACCAAGA |
| | | | | CCTACACCTGCAACGTGGACCACAAGCCAA |
| | | | | GCAACACCAAGGTGGACAAGCGCGTGGAG |
| | | | | AGCAAGTACGGCCCACCATGCCCACCATGC |
| | | | | CCAGCCCCAGAGGCCGCCGGCGGCCCAAG |
| | | | | CGTGTTCCTGTTCCCACCAAAGCCAAAGGA |
| | | | | CACCCTGATGATCAGCCGCACCCCAGAGGT |
| | | | | GACCTGCGTGGTGGTGGACGTGAGCCAGG |
| | | | | AGGACCCAGAGGTGCAGTTCAACTGGTACG |
| | | | | TGGACGGCGTGGAGGTGCACAACGCCAAG |
| | | | | ACCAAGCCACGCGAGGAGCAGTTCAACAG |
| | | | | CACCTACCGCGTGGTGAGCGTGCTGACCGT |
| | | | | GCTGCACCAGGACTGGCTGAACGGCAAGG |
| | | | | AGTACAAGTGCAAGGTGAGCAACAAGGGC |
| | | | | CTGCCAAGCAGCATCGAGAAGACCATCAG |
| | | | | CAAGGCCAAGGGCCAGCCACGCGAGCCAC |
| | | | | AGGTGTACACCCTGCCACCAAGCCAGGAG |
| | | | | GAGATGACCAAGAACCAGGTGAGCCTGTG |
| | | | | GTGCCTGGTGAAGGGCTTCTACCCAAGCGA |
| | | | | CATCGCCGTGGAGTGGGAGAGCAACGGCC |
| | | | | AGCCAGAGAACAACTACAAGACCACCCCA |
| | | | | CCAGTGCTGGACAGCGACGGCAGCTTCTTC |
| | | | | CTGTACAGCCGCCTGACCGTGGACAAGAGC |
| | | | | CGCTGGCAGGAGGGCAACGTGTTCAGCTGC |
| | | | | AGCGTGATGCACGAGGCCCTGCACAACCAC |
| | | | | TACACCCAGAAGAGCCTGAGCCTGAGCCTG |
| | | | | GGCAAG |
| 32 | PRT | artificial | B23B49 half | MAWVWTLLFLMAAAQSIQAEIVLTQSPGTLS |
| | | | antibody | LSPGERATLSCRASQSVSSSYLAWYQQKPGQ |
| | | | | APRLLIYGASSRATGIPDRFSGSGSGTDFTLTI |
| | | | | SRLEPEDFAVYYCQQDYGFPWTFGQGTKVEI |
| | | | | KRTVAAPSVFIFPPSDEQLKSGTASVVCLLNN |
| | | | | FYPREAKVQWKVDNALQSGNSQESVTEQDS |
| | | | | KDSTYSLSSTLTLSKADYEKHKVYACEVTHQ |
| | | | | GLSSPVTKSFNRGECGGSEGKSSGSGSESKST |
| | | | | EGKSSGSGSESKSTGGSEVQLVQSGAEVKKP |
| | | | | GESLKISCKGSGYSFTSYWISWVRQMPGKGL |
| | | | | EWMGIIDPSDSDTRYSPSFQGQVTISADKSIST |
| | | | | AYLQWSSLKASDTAMYYCARGDGSTDLDY |
| | | | | WGQGTLVTVSSASTKGPSVFPLAPCSRSTSES |
| | | | | TAALGCLVKDYFPEPVTVSWNSGALTSGVHT |
| | | | | FPAVLQSSGLYSLSSVVTVPSSSLGTKTYTCN |
| | | | | VDHKPSNTKVDKRVESKYGPPCPPCPAPEAA |
| | | | | GGPSVFLFPPKPKDTLMISRTPEVTCVVVDVS |
| | | | | QEDPEVQFNWYVDGVEVHNAKTKPREEQFN |
| | | | | STYRVVSVLTVLHQDWLNGKEYKCKVSNKG |
| | | | | LPSSIEKTISKAKGQPREPQVYTLPPSQEEMTK |
| | | | | NQVSLWCLVKGFYPSDIAVEWESNGQPENN |
| | | | | YKTTPPVLDSDGSFFLYSRLTVDKSRWQEGN |
| | | | | VFSCSVMHEALHNHYTQKSLSLSLGK |
| 33 | DNA | artificial | B23B49 half | ATGGCCTGGGTGTGGACCCTGCTGTTCCTG |
| | | | antibody | ATGGCCGCCGCCCAGAGCATCCAGGCCGAC |
| | | | | ATCGTGATGACCCAGAGCCCAGACAGCCTG |
| | | | | GCCGTGAGCCTGGGCGAGCGCGCCACCATC |
| | | | | AACTGCCGCGCCAGCCAGAGCGTGGACTAC |
| | | | | AACGGCATCAGCTACATGCACTGGTACCAG |
| | | | | CAGAAGCCAGGCCAGCCACCAAAGCTGCT |
| | | | | GATCTACGCCGCCAGCAACCCAGAGAGCG |
| | | | | GCGTGCCAGACCGCTTCAGCGGCAGCGGCA |
| | | | | GCGGCACCGACTTCACCCTGACCATCAGCA |
| | | | | GCCTGCAGGCCGAGGACGTGGCCGTGTACT |
| | | | | ACTGCCAGCAGATCATCGAGGACCCATGGA |
| | | | | CCTTCGGCCAGGGCACCAAGGTGGAGATCA |
| | | | | AGCGCACCGTGGCCGCCCCAAGCGTGTTCA |
| | | | | TCTTCCCACCAAGCGACGAGCAGCTGAAGA |
| | | | | GCGGCACCGCCAGCGTGGTGTGCCTGCTGA |
| | | | | ACAACTTCTACCCACGCGAGGCCAAGGTGC |
| | | | | AGTGGAAGGTGGACAACGCCCTGCAGAGC |
| | | | | GGCAACAGCCAGGAGAGCGTGACCGAGCA |
| | | | | GGACAGCAAGGACAGCACCTACAGCCTGA |
| | | | | GCAGCACCCTGACCCTGAGCAAGGCCGACT |
| | | | | ACGAGAAGCACAAGGTGTACGCCTGCGAG |
| | | | | GTGACCCACCAGGGCCTGAGCAGCCCAGTG |
| | | | | ACCAAGAGCTTCAACCGCGGCGAGTGCGG |
| | | | | CGGCAGCGAGGGCAAGAGCAGCGGCAGCG |
| | | | | GCAGCGAGAGCAAGAGCACCGAGGGCAAG |
| | | | | AGCAGCGGCAGCGGCAGCGAGAGCAAGAG |
| | | | | CACCGGCGGCAGCCAGATCACCCTGAAGG |
| | | | | AGAGCGGCCCAACCCTGGTGAAGCCAACC |
| | | | | CAGACCCTGACCCTGACCTGCACCTTCAGC |
| | | | | GGCTTCAGCCTGAGCACCAGCGGCATGGGC |
| | | | | GTGAGCTGGATCCGCCAGCCACCAGGCAA |
| | | | | GGCCCTGGAGTGGCTGGCCCACATCTACTG |
| | | | | GGACGACGACAAGCGCTACAACCCAAGCC |
| | | | | TGAAGAGCCGCCTGACCATCACCAAGGAC |
| | | | | ACCAGCAAGAACCAGGTGGTGCTGACCAT |
| | | | | GACCAACATGGACCCAGTGGACACCGCCA |
| | | | | CCTACTACTGCGCCCGCCTGTACGGCTTCA |
| | | | | CCTACGGCTTCGCCTACTGGGGCCAGGGCA |
| | | | | CCCTGGTGACCGTGAGCAGCGCCAGCACCA |
| | | | | AGGGCCCAAGCGTGTTCCCACTGGCCCCAT |
| | | | | GCAGCCGCAGCACCAGCGAGAGCACCGCC |
| | | | | GCCCTGGGCTGCCTGGTGAAGGACTACTTC |
| | | | | CCAGAGCCAGTGACCGTGAGCTGGAACAG |
| | | | | CGGCGCCCTGACCAGCGGCGTGCACACCTT |
| | | | | CCCAGCCGTGCTGCAGAGCAGCGGCCTGTA |
| | | | | CAGCCTGAGCAGCGTGGTGACCGTGCCAAG |
| | | | | CAGCAGCCTGGGCACCAAGACCTACACCTG |
| | | | | CAACGTGGACCACAAGCCAAGCAACACCA |
| | | | | AGGTGGACAAGCGCGTGGAGAGCAAGTAC |
| | | | | GGCCCACCATGCCCACCATGCCCAGCCCCA |
| | | | | GAGGCCGCCGGCGGCCCAAGCGTGTTCCTG |
| | | | | TTCCCACCAAAGCCAAAGGACACCCTGATG |
| | | | | ATCAGCCGCACCCCAGAGGTGACCTGCGTG |
| | | | | GTGGTGGACGTGAGCCAGGAGGACCCAGA |
| | | | | GGTGCAGTTCAACTGGTACGTGGACGGCGT |
| | | | | GGAGGTGCACAACGCCAAGACCAAGCCAC |
| | | | | GCGAGGAGCAGTTCAACAGCACCTACCGC |
| | | | | GTGGTGAGCGTGCTGACCGTGCTGCACCAG |
| | | | | GACTGGCTGAACGGCAAGGAGTACAAGTG |
| | | | | CAAGGTGAGCAACAAGGGCCTGCCAAGCA |
| | | | | GCATCGAGAAGACCATCAGCAAGGCCAAG |
| | | | | GGCCAGCCACGCGAGCCACAGGTGTACAC |
| | | | | CCTGCCACCAAGCCAGGAGGAGATGACCA |
| | | | | AGAACCAGGTGAGCCTGTGGTGCCTGGTGA |
| | | | | AGGGCTTCTACCCAAGCGACATCGCCGTGG |
| | | | | AGTGGGAGAGCAACGGCCAGCCAGAGAAC |
| | | | | AACTACAAGACCACCCCACCAGTGCTGGAC |
| | | | | AGCGACGGCAGCTTCTTCCTGTACAGCCGC |
| | | | | CTGACCGTGGACAAGAGCCGCTGGCAGGA |
| | | | | GGGCAACGTGTTCAGCTGCAGCGTGATGCA |
| | | | | CGAGGCCCTGCACAACCACTACACCCAGAA |
| | | | | GAGCCTGAGCCTGAGCCTGGGCAAG |
| 34 | PRT | Artificial | HCDR1 | SYWIS |
| 35 | PRT | Artificial | HCDR2 | IIDPSDSDTRYSPSFQG |
| 36 | PRT | Artificia | HCDR3 | GDGSTDLDY |
| 37 | PRT | Artificial | LCDR1 | RASQSVSSSYL |
| 38 | PRT | Artificial | LCDR2 | GASSRAT |
| 39 | PRT | Artificial | LCDR3 | QQDYGFPWT |
| 40 | PRT | Artificial | HC | EVQLVQSGAEVKKPGESLKISCKGSGYSFTSY |
| | | | | WISWVRQMPGKGLEWMGIIDPSDSDTRYSPS |
| | | | | FQGQVTISADKSISTAYLQWSSLKASDTAMY |
| | | | | YCARGDGSTDLDYWGQGTLVTVSS |
| 41 | PRT | Artificial | LC | EIVLTQSPGTLSLSPGERATLSCRASQSVSSSY |
| | | | | LAWYQQKPGQAPRLLIYGASSRATGIPDRFS |
| | | | | GSGSGTDFTLTISRLEPEDFAVYYCQQDYGFP |
| | | | | WTFGQGTKVEIK |
| 42 | PRT | Artificial | IGHJ1*01 HC | WGQGTLVTVSS |
| 43 | PRT | Artificial | IGKJ2*01 LC | FGQGTKLEIK |
| 44 | PRT | Artificial | Va10.2 Fc | MAWVWTLLFLMAAAQSIQAQLLEQSPQFLSI |
| | | | | QEGENLTVYCNSSSVFSSLQWYRQEPGEGPV |
| | | | | LLVTVVTGGEVKKLKRLTFQFGDARKDSSLH |
| | | | | ITAAQPGDTGLYLCAGAGSQGNLIFGKGTKL |
| | | | | SVKPNIQNPDPAVYQLRDSKSSDKSVCLFTDF |
| | | | | DSQTNVSQSKDSDVYITDKTVLDMRSMDFKS |
| | | | | NSAVAWSNKSDFACANAFNNSIIPEDTFFPSep |
| | | | | kscdkthtcppcpapeLLggpsvflfppkpkdtlmisrtpevtcvv |
| | | | | vDvshedpevkfnwyvdgvevhnaktkpreeqynstyrvvsvlt |
| | | | | vlhqdwlngkeykckvsnkalpapiektiskakgqprepqvyVY |
| | | | | ppsreemtknqvsltclvkgfypsdiavewesngqpennykttpp |
| | | | | vldsdgsfAlVskltvdksrwqqgnvfscsvmhealhnhytqksl |
| | | | | slspg |
| 45 | PRT | Mouse- | B17B21-N33S- | GYSITSGYFWN |
| | | humanized | HL-spFv VH | |
| | | | AbM CDR1 | |
| 46 | PRT | Mouse- | B17B21-N33S- | YISYDGSNN |
| | | humanized | HL-spFv VH | |
| | | | AbM CDR2 | |
| 47 | PRT | Mouse- | B17B21-N33S- | PSPGTGYAVDY |
| | | humanized | HL-spFv VH | |
| | | | AbM CDR3 | |
| 48 | PRT | Mouse- | B17B21-N33S- | SGYFWN |
| | | humanized | HL-spFv VH | |
| | | | KABAT CDR1 | |
| 49 | PRT | Mouse- | B17B21-N33S- | YISYDGSNNYNPSLKS |
| | | humanized | HL-spFv VH | |
| | | | KABAT CDR2 | |
| 50 | PRT | Mouse- | B17B21-N33S- | PSPGTGYAVDY |
| | | humanized | HL-spFv VH | |
| | | | KABAT CDR3 | |
| 51 | PRT | Mouse- | B17B21-N33S- | GYSITSGY |
| | | humanized | HL-spFv VH | |
| | | | CHOTHIA | |
| | | | CDR1 | |
| 52 | PRT | Mouse- | B17B21-N33S- | SYDGS |
| | | humanized | HL-spFv VH | |
| | | | CHOTHIA | |
| | | | CDR2 | |
| 53 | PRT | Mouse- | B17B21-N33S- | PSPGTGYAVDY |
| | | humanized | HL-spFv VH | |
| | | | CHOTHIA | |
| | | | CDR3 | |
| 54 | PRT | Mouse- | B17B21-N33S- | GYSITSGYF |
| | | humanized | HL-spFv VH | |
| | | | IMGT CDR1 | |
| 55 | PRT | Mouse- | B17B21-N33S- | ISYDGSN |
| | | humanized | HL-spFv VH | |
| | | | IMGT CDR2 | |
| 56 | PRT | Mouse- | B17B21-N33S- | ASPSPGTGYAVDY |
| | | humanized | HL-spFv VH | |
| | | | IMGT CDR3 | |
| 57 | PRT | Mouse- | B17B21-N33S- | TSGYFWN |
| | | humanized | HL-spFv VH | |
| | | | CONTACT | |
| | | | CDR1 | |
| 58 | PRT | Mouse- | B17B21-N33S- | WIGYISYDGSNN |
| | | humanized | HL-spFv VH | |
| | | | CONTACT | |
| | | | CDR2 | |
| 59 | PRT | Mouse- | B17B21-N33S- | ASPSPGTGYAVD |
| | | humanized | HL-spFv VH | |
| | | | CONTACT | |
| | | | CDR3 | |
| 60 | PRT | Mouse- | B17B21-N33S- | RSSQSLVHSSGNTYLH |
| | | humanized | HL-spFv VL | |
| | | | AbM CDR1 | |
| 61 | PRT | Mouse- | B17B21-N33S- | KVSNRFS |
| | | humanized | HL-spFv VL | |
| | | | AbM CDR2 | |
| 62 | PRT | Mouse- | B17B21-N33S- | SQSTHVPFT |
| | | humanized | HL-spFv VL | |
| | | | AbM CDR3 | |
| 63 | PRT | Mouse- | B17B21-N33S- | RSSQSLVHSSGNTYLH |
| | | humanized | HL-spFv VL | |
| | | | KABAT CDR1 | |
| 64 | PRT | Mouse- | B17B21-N33S- | KVSNRFS |
| | | humanized | HL-spFv VL | |
| | | | KABAT CDR2 | |
| 65 | PRT | Mouse- | B17B21-N33S- | SQSTHVPFT |
| | | humanized | HL-spFv VL | |
| | | | KABAT CDR3 | |
| 66 | PRT | Mouse- | B17B21-N33S- | RSSQSLVHSSGNTYLH |
| | | humanized | HL-spFv VL | |
| | | | CHOTHIA | |
| | | | CDR1 | |
| 67 | PRT | Mouse- | B17B21-N33S- | KVSNRFS |
| | | humanized | HL-spFv VL | |
| | | | CHOTHIA | |
| | | | CDR2 | |
| 68 | PRT | Mouse- | B17B21-N33S- | SQSTHVPFT |
| | | humanized | HL-spFv VL | |
| | | | CHOTHIA | |
| | | | CDR3 | |
| 69 | PRT | Mouse- | B17B21-N33S- | QSLVHSSGNTY |
| | | humanized | HL-spFv VL | |
| | | | IMGT CDR1 | |
| 71 | PRT | Mouse- | B17B21-N33S- | SQSTHVPFT |
| | | humanized | HL-spFv VL | |
| | | | IMGT CDR3 | |
| 72 | PRT | Mouse- | B17B21-N33S- | VHSSGNTYLHWY |
| | | humanized | HL-spFv VL | |
| | | | CONTACT | |
| | | | CDR1 | |
| 73 | PRT | Mouse- | B17B21-N33S- | FLIYKVSNRF |
| | | humanized | HL-spFv VL | |
| | | | CONTACT | |
| | | | CDR2 | |
| 74 | PRT | Mouse- | B17B21-N33S- | SQSTHVPF |
| | | humanized | HL-spFv VL | |
| | | | CONTACT | |
| | | | CDR3 | |
| 75 | PRT | Mouse- | B17B21-N33S- | QVQLQESGPGLVKPSETLSLTCTVSGYSITSG |
| | | humanized | HL-spFv VH | YFWNWIRQPPGCGLEWIGYISYDGSNNYNPS |
| | | | | LKSRVTISRDTSKNQFSLKLSSVTAADTAVYY |
| | | | | CASPSPGTGYAVDYWGQGTLVTVSS |
| 76 | PRT | Mouse- | B17B21-N33S- | DIQMTQSPSSLSASVGDRVTITCRSSQSLVHSS |
| | | humanized | HL-spFv VL | GNTYLHWYQQKPGKAPKFLIYKVSNRFSGVP |
| | | | | SRFSGSGSGTDFTLTISSLQPEDFATYYCSQST |
| | | | | HVPFTFGCGTKLEIK |
| 77 | PRT | Mouse- | B17B21-VL- | GYSITSGYFWN |
| | | humanized | N33S VH AbM | |
| | | | CDR1 | |
| 78 | PRT | Mouse- | B17B21-VL- | YISYDGSNN |
| | | humanized | N33S VH AbM | |
| | | | CDR2 | |
| 79 | PRT | Mouse- | B17B21-VL- | PSPGTGYAVDY |
| | | humanized | N33S VH AbM | |
| | | | CDR3 | |
| 80 | PRT | Mouse- | B17B21-VL- | SGYFWN |
| | | humanized | N33S VH | |
| | | | KABAT CDR1 | |
| 81 | PRT | Mouse- | B17B21-VL- | YISYDGSNNYNPSLKS |
| | | humanized | N33S VH | |
| | | | KABAT CDR2 | |
| 82 | PRT | Mouse- | B17B21-VL- | PSPGTGYAVDY |
| | | humanized | N33S VH | |
| | | | KABAT CDR3 | |
| 83 | PRT | Mouse- | B17B21-VL- | GYSITSGY |
| | | humanized | N33S VH | |
| | | | CHOTHIA | |
| | | | CDR1 | |
| 84 | PRT | Mouse- | B17B21-VL- | SYDGS |
| | | humanized | N33S VH | |
| | | | CHOTHIA | |
| | | | CDR2 | |
| 85 | PRT | Mouse- | B17B21-VL- | PSPGTGYAVDY |
| | | humanized | N33S VH | |
| | | | CHOTHIA | |
| | | | CDR3 | |
| 86 | PRT | Mouse- | B17B21-VL- | GYSITSGYF |
| | | humanized | N33S VH | |
| | | | IMGT CDR1 | |
| 87 | PRT | Mouse- | B17B21-VL- | ISYDGSN |
| | | humanized | N33S VH | |
| | | | IMGT CDR2 | |
| 88 | PRT | Mouse- | B17B21-VL- | ASPSPGTGYAVDY |
| | | humanized | N33S VH | |
| | | | IMGT CDR3 | |
| 89 | PRT | Mouse- | B17B21-VL- | TSGYFWN |
| | | humanized | N33S VH | |
| | | | CONTACT | |
| | | | CDR1 | |
| 90 | PRT | Mouse- | B17B21-VL- | WIGYISYDGSNN |
| | | humanized | N33S VH | |
| | | | CONTACT | |
| | | | CDR2 | |
| 91 | PRT | Mouse- | B17B21-VL- | ASPSPGTGYAVD |
| | | humanized | N33S VH | |
| | | | CONTACT | |
| | | | CDR3 | |
| 92 | PRT | Mouse- | B17B21-VL- | RSSQSLVHSSGNTYLH |
| | | humanized | N33S VL AbM | |
| | | | CDR1 | |
| 93 | PRT | Mouse- | B17B21-VL- | KVSNRFS |
| | | humanized | N33S VL AbM | |
| | | | CDR2 | |
| 94 | PRT | Mouse- | B17B21-VL- | SQSTHVPFT |
| | | humanized | N33S VL AbM | |
| | | | CDR3 | |
| 95 | PRT | Mouse- | B17B21-VL- | RSSQSLVHSSGNTYLH |
| | | humanized | N33S VL | |
| | | | KABAT CDR1 | |
| 96 | PRT | Mouse- | B17B21-VL- | KVSNRFS |
| | | humanized | N33S VL | |
| | | | KABAT CDR2 | |
| 97 | PRT | Mouse- | B17B21-VL- | SQSTHVPFT |
| | | humanized | N33S VL | |
| | | | KABAT CDR3 | |
| 98 | PRT | Mouse- | B17B21-VL- | RSSQSLVHSSGNTYLH |
| | | humanized | N33S VL | |
| | | | CHOTHIA | |
| | | | CDR1 | |
| 99 | PRT | Mouse- | B17B21-VL- | KVSNRFS |
| | | humanized | N33S VL | |
| | | | CHOTHIA | |
| | | | CDR2 | |
| 100 | PRT | Mouse- | B17B21-VL- | SQSTHVPFT |
| | | humanized | N33S VL | |
| | | | CHOTHIA | |
| | | | CDR3 | |
| 101 | PRT | Mouse- | B17B21-VL- | QSLVHSSGNTY |
| | | humanized | N33S VL | |
| | | | IMGT CDR1 | |
| 103 | PRT | Mouse- | B17B21-VL- | SQSTHVPFT |
| | | humanized | N33S VL | |
| | | | IMGT CDR3 | |
| 104 | PRT | Mouse- | B17B21-VL- | VHSSGNTYLHWY |
| | | humanized | N33S VL | |
| | | | CONTACT | |
| | | | CDR1 | |
| 105 | PRT | Mouse- | B17B21-VL- | FLIYKVSNRF |
| | | humanized | N33S VL | |
| | | | CONTACT | |
| | | | CDR2 | |
| 106 | PRT | Mouse- | B17B21-VL- | SQSTHVPF |
| | | humanized | N33S VL | |
| | | | CONTACT | |
| | | | CDR3 | |
| 107 | PRT | Mouse- | B17B21-VL- | QVQLQESGPGLVKPSETLSLTCTVSGYSITSG |
| | | humanized | N33S VH | YFWNWIRQPPGKGLEWIGYISYDGSNNYNPS |
| | | | | LKSRVTISRDTSKNQFSLKLSSVTAADTAVYY |
| | | | | CASPSPGTGYAVDYWGQGTLVTVSS |
| 108 | PRT | Mouse- | B17B21-VL- | DIQMTQSPSSLSASVGDRVTITCRSSQSLVHSS |
| | | humanized | N33S VL | GNTYLHWYQQKPGKAPKFLIYKVSNRFSGVP |
| | | | | SRFSGSGSGTDFTLTISSLQPEDFATYYCSQST |
| | | | | HVPFTFGQGTKLEIK |
| 109 | PRT | Mouse- | B17B21-N33S- | GYSITSGYFWN |
| | | humanized | LH-spFv VH | |
| | | | AbM CDR1 | |
| 110 | PRT | Mouse- | B17B21-N33S- | YISYDGSNN |
| | | humanized | LH-spFv VH | |
| | | | AbM CDR2 | |
| 111 | PRT | Mouse- | B17B21-N33S- | PSPGTGYAVDY |
| | | humanized | LH-spFv VH | |
| | | | AbM CDR3 | |
| 112 | PRT | Mouse- | B17B21-N33S- | SGYFWN |
| | | humanized | LH-spFv VH | |
| | | | KABAT CDR1 | |
| 113 | PRT | Mouse- | B17B21-N33S- | YISYDGSNNYNPSLKS |
| | | humanized | LH-spFv VH | |
| | | | KABAT CDR2 | |
| 114 | PRT | Mouse- | B17B21-N33S- | PSPGTGYAVDY |
| | | humanized | LH-spFv VH | |
| | | | KABAT CDR3 | |
| 115 | PRT | Mouse- | B17B21-N33S- | GYSITSGY |
| | | humanized | LH-spFv VH | |
| | | | CHOTHIA | |
| | | | CDR1 | |
| 116 | PRT | Mouse- | B17B21-N33S- | SYDGS |
| | | humanized | LH-spFv VH | |
| | | | CHOTHIA | |
| | | | CDR2 | |
| 117 | PRT | Mouse- | B17B21-N33S- | PSPGTGYAVDY |
| | | humanized | LH-spFv VH | |
| | | | CHOTHIA | |
| | | | CDR3 | |
| 118 | PRT | Mouse- | B17B21-N33S- | GYSITSGYF |
| | | humanized | LH-spFv VH | |
| | | | IMGT CDR1 | |
| 119 | PRT | Mouse- | B17B21-N33S- | ISYDGSN |
| | | humanized | LH-spFv VH | |
| | | | IMGT CDR2 | |
| 120 | PRT | Mouse- | B17B21-N33S- | ASPSPGTGYAVDY |
| | | humanized | LH-spFv VH | |
| | | | IMGT CDR3 | |
| 121 | PRT | Mouse- | B17B21-N33S- | TSGYFWN |
| | | humanized | LH-spFv VH | |
| | | | CONTACT | |
| | | | CDR1 | |
| 122 | PRT | Mouse- | B17B21-N33S- | WIGYISYDGSNN |
| | | humanized | LH-spFv VH | |
| | | | CONTACT | |
| | | | CDR2 | |
| 123 | PRT | Mouse- | B17B21-N33S- | ASPSPGTGYAVD |
| | | humanized | LH-spFv VH | |
| | | | CONTACT | |
| | | | CDR3 | |
| 124 | PRT | Mouse- | B17B21-N33S- | RSSQSLVHSSGNTYLH |
| | | humanized | LH-spFv VL | |
| | | | AbM CDR1 | |
| 125 | PRT | Mouse- | B17B21-N33S- | KVSNRFS |
| | | humanized | LH-spFv VL | |
| | | | AbM CDR2 | |
| 126 | PRT | Mouse- | B17B21-N33S- | SQSTHVPFT |
| | | humanized | LH-spFv VL | |
| | | | AbM CDR3 | |
| 127 | PRT | Mouse- | B17B21-N33S- | RSSQSLVHSSGNTYLH |
| | | humanized | LH-spFv VL | |
| | | | KABAT CDR1 | |
| 128 | PRT | Mouse- | B17B21-N33S- | KVSNRFS |
| | | humanized | LH-spFv VL | |
| | | | KABAT CDR2 | |
| 129 | PRT | Mouse- | B17B21-N33S- | SQSTHVPFT |
| | | humanized | LH-spFv VL | |
| | | | KABAT CDR3 | |
| 130 | PRT | Mouse- | B17B21-N33S- | RSSQSLVHSSGNTYLH |
| | | humanized | LH-spFv VL | |
| | | | CHOTHIA | |
| | | | CDR1 | |
| 131 | PRT | Mouse- | B17B21-N33S- | KVSNRFS |
| | | humanized | LH-spFv VL | |
| | | | CHOTHIA | |
| | | | CDR2 | |
| 132 | PRT | Mouse- | B17B21-N33S- | SQSTHVPFT |
| | | humanized | LH-spFv VL | |
| | | | CHOTHIA | |
| | | | CDR3 | |
| 133 | PRT | Mouse- | B17B21-N33S- | QSLVHSSGNTY |
| | | humanized | LH-spFv VL | |
| | | | IMGT CDR1 | |
| 135 | PRT | Mouse- | B17B21-N33S- | SQSTHVPFT |
| | | humanized | LH-spFv VL | |
| | | | IMGT CDR3 | |
| 136 | PRT | Mouse- | B17B21-N33S- | VHSSGNTYLHWY |
| | | humanized | LH-spFv VL | |
| | | | CONTACT | |
| | | | CDR1 | |
| 137 | PRT | Mouse- | B17B21-N33S- | FLIYKVSNRF |
| | | humanized | LH-spFv VL | |
| | | | CONTACT | |
| | | | CDR2 | |
| 138 | PRT | Mouse- | B17B21-N33S- | SQSTHVPF |
| | | humanized | LH-spFv VL | |
| | | | CONTACT | |
| | | | CDR3 | |
| 139 | PRT | Mouse- | B17B21-N33S- | QVQLQESGPGLVKPSETLSLTCTVSGYSITSG |
| | | humanized | LH-spFv VH | YFWNWIRQPPGKGLEWIGYISYDGSNNYNPS |
| | | | | LKSRVTISRDTSKNQFSLKLSSVTAADTAVYY |
| | | | | CASPSPGTGYAVDYWGCGTLVTVSS |
| 140 | PRT | Mouse- | B17B21-N33S- | DIQMTQSPSSLSASVGDRVTITCRSSQSLVHSS |
| | | humanized | LH-spFv VL | GNTYLHWYQQKPGCAPKFLIYKVSNRFSGVP |
| | | | | SRFSGSGSGTDFTLTISSLQPEDFATYYCSQST |
| | | | | HVPFTFGQGTKLEIK |
| 141 | PRT | Mouse- | B17B852- | GYSITSGYFWN |
| | | humanized | G34R VH | |
| | | | AbM CDR1 | |
| 142 | PRT | Mouse- | B17B852- | YISYDGSNN |
| | | humanized | G34R VH | |
| | | | AbM CDR2 | |
| 143 | PRT | Mouse- | B17B852- | PSPGTGYAVDY |
| | | humanized | G34R VH | |
| | | | AbM CDR3 | |
| 144 | PRT | Mouse- | B17B852- | SGYFWN |
| | | humanized | G34R VH | |
| | | | KABAT CDR1 | |
| 145 | PRT | Mouse- | B17B852- | YISYDGSNNYNPSLKS |
| | | humanized | G34R VH | |
| | | | KABAT CDR2 | |
| 146 | PRT | Mouse- | B17B852- | PSPGTGYAVDY |
| | | humanized | G34R VH | |
| | | | KABAT CDR3 | |
| 147 | PRT | Mouse- | B17B852- | GYSITSGY |
| | | humanized | G34R VH | |
| | | | CHOTHIA | |
| | | | CDR1 | |
| 148 | PRT | Mouse- | B17B852- | SYDGS |
| | | humanized | G34R VH | |
| | | | CHOTHIA | |
| | | | CDR2 | |
| 149 | PRT | Mouse- | B17B852- | PSPGTGYAVDY |
| | | humanized | G34R VH | |
| | | | CHOTHIA | |
| | | | CDR3 | |
| 150 | PRT | Mouse- | B17B852- | GYSITSGYF |
| | | humanized | G34R VH | |
| | | | IMGT CDR1 | |
| 151 | PRT | Mouse- | B17B852- | ISYDGSN |
| | | humanized | G34R VH | |
| | | | IMGT CDR2 | |
| 152 | PRT | Mouse- | B17B852- | ASPSPGTGYAVDY |
| | | humanized | G34R VH | |
| | | | IMGT CDR3 | |
| 153 | PRT | Mouse- | B17B852- | TSGYFWN |
| | | humanized | G34R VH | |
| | | | CONTACT | |
| | | | CDR1 | |
| 154 | PRT | Mouse- | B17B852- | WMGYISYDGSNN |
| | | humanized | G34R VH | |
| | | | CONTACT | |
| | | | CDR2 | |
| 155 | PRT | Mouse- | B17B852- | ASPSPGTGYAVD |
| | | humanized | G34R VH | |
| | | | CONTACT | |
| | | | CDR3 | |
| 156 | PRT | Mouse- | B17B852- | RSSQSLVHSNRNTYLH |
| | | humanized | G34R VL AbM | |
| | | | CDR1 | |
| 157 | PRT | Mouse- | B17B852- | KVSNRFS |
| | | humanized | G34R VL AbM | |
| | | | CDR2 | |
| 158 | PRT | Mouse- | B17B852- | SQSTHVPFT |
| | | humanized | G34R VL AbM | |
| | | | CDR3 | |
| 159 | PRT | Mouse- | B17B852- | RSSQSLVHSNRNTYLH |
| | | humanized | G34R VL | |
| | | | KABAT CDR1 | |
| 160 | PRT | Mouse- | B17B852- | KVSNRFS |
| | | humanized | G34R VL | |
| | | | KABAT CDR2 | |
| 161 | PRT | Mouse- | B17B852- | SQSTHVPFT |
| | | humanized | G34R VL | |
| | | | KABAT CDR3 | |
| 162 | PRT | Mouse- | B17B852- | RSSQSLVHSNRNTYLH |
| | | humanized | G34R VL | |
| | | | CHOTHIA | |
| | | | CDR1 | |
| 163 | PRT | Mouse- | B17B852- | KVSNRFS |
| | | humanized | G34R VL | |
| | | | CHOTHIA | |
| | | | CDR2 | |
| 164 | PRT | Mouse- | B17B852- | SQSTHVPFT |
| | | humanized | G34R VL | |
| | | | CHOTHIA | |
| | | | CDR3 | |
| 165 | PRT | Mouse- | B17B852- | QSLVHSNRNTY |
| | | humanized | G34R VL | |
| | | | IMGT CDR1 | |
| 167 | PRT | Mouse- | B17B852- | SQSTHVPFT |
| | | humanized | G34R VL | |
| | | | IMGT CDR3 | |
| 168 | PRT | Mouse- | B17B852- | VHSNRNTYLHWY |
| | | humanized | G34R VL | |
| | | | CONTACT | |
| | | | CDR1 | |
| 169 | PRT | Mouse- | B17B852- | LLIYKVSNRF |
| | | humanized | G34R VL | |
| | | | CONTACT | |
| | | | CDR2 | |
| 170 | PRT | Mouse- | B17B852- | SQSTHVPF |
| | | humanized | G34R VL | |
| | | | CONTACT | |
| | | | CDR3 | |
| 171 | PRT | Mouse- | B17B852- | QVQLQESGPGLVKPSETLSLTCTVSGYSITSG |
| | | humanized | G34R VH | YFWNWIRQFPGKGLEWMGYISYDGSNNYNP |
| | | | | SLKSRITISRDTSKNQFSLKLSSVTAADTAVY |
| | | | | YCASPSPGTGYAVDYWGQGTTVTVSS |
| 172 | PRT | Mouse- | B17B852- | DIQMTQSPSSLSASVGDRVTITCRSSQSLVHS |
| | | humanized | G34R VL | NRNTYLHWYQQKPGKAPKLLIYKVSNRFSG |
| | | | | VPSRFSGSGSGTDFTLTISSLQPEDFATYYCSQ |
| | | | | STHVPFTFGQGTKLEIK |
| 173 | PRT | Mouse- | B17B852- | GYSITSGYFWN |
| | | humanized | G34R-LH-spFv | |
| | | | VH AbM | |
| | | | CDR1 | |
| 174 | PRT | Mouse- | B17B852- | YISYDGSNN |
| | | humanized | G34R-LH-spFv | |
| | | | VH AbM | |
| | | | CDR2 | |
| 175 | PRT | Mouse- | B17B852- | PSPGTGYAVDY |
| | | humanized | G34R-LH-spFv | |
| | | | VH AbM | |
| | | | CDR3 | |
| 176 | PRT | Mouse- | B17B852- | SGYFWN |
| | | humanized | G34R-LH-spFv | |
| | | | VH KABAT | |
| | | | CDR1 | |
| 177 | PRT | Mouse- | B17B852- | YISYDGSNNYNPSLKS |
| | | humanized | G34R-LH-spFv | |
| | | | VH KABAT | |
| | | | CDR2 | |
| 178 | PRT | Mouse- | B17B852- | PSPGTGYAVDY |
| | | humanized | G34R-LH-spFv | |
| | | | VH KABAT | |
| | | | CDR3 | |
| 179 | PRT | Mouse- | B17B852- | GYSITSGY |
| | | humanized | G34R-LH-spFv | |
| | | | VH CHOTHIA | |
| | | | CDR1 | |
| 180 | PRT | Mouse- | B17B852- | SYDGS |
| | | humanized | G34R-LH-spFv | |
| | | | VH CHOTHIA | |
| | | | CDR2 | |
| 181 | PRT | Mouse- | B17B852- | PSPGTGYAVDY |
| | | humanized | G34R-LH-spFv | |
| | | | VH CHOTHIA | |
| | | | CDR3 | |
| 182 | PRT | Mouse- | B17B852- | GYSITSGYF |
| | | humanized | G34R-LH-spFv | |
| | | | VH IMGT | |
| | | | CDR1 | |
| 183 | PRT | Mouse- | B17B852- | ISYDGSN |
| | | humanized | G34R-LH-spFv | |
| | | | VH IMGT | |
| | | | CDR2 | |
| 184 | PRT | Mouse- | B17B852- | ASPSPGTGYAVDY |
| | | humanized | G34R-LH-spFv | |
| | | | VH IMGT | |
| | | | CDR3 | |
| 185 | PRT | Mouse- | B17B852- | TSGYFWN |
| | | humanized | G34R-LH-spFv | |
| | | | VH | |
| | | | CONTACT | |
| | | | CDR1 | |
| 186 | PRT | Mouse- | B17B852- | WMGYISYDGSNN |
| | | humanized | G34R-LH-spFv | |
| | | | VH | |
| | | | CONTACT | |
| | | | CDR2 | |
| 187 | PRT | Mouse- | B17B852- | ASPSPGTGYAVD |
| | | humanized | G34R-LH-spFv | |
| | | | VH | |
| | | | CONTACT | |
| | | | CDR3 | |
| 188 | PRT | Mouse- | B17B852- | RSSQSLVHSNRNTYLH |
| | | humanized | G34R-LH-spFv | |
| | | | VL AbM | |
| | | | CDR1 | |
| 189 | PRT | Mouse- | B17B852- | KVSNRFS |
| | | humanized | G34R-LH-spFv | |
| | | | VL AbM | |
| | | | CDR2 | |
| 190 | PRT | Mouse- | B17B852- | SQSTHVPFT |
| | | humanized | G34R-LH-spFv | |
| | | | VL AbM | |
| | | | CDR3 | |
| 191 | PRT | Mouse- | B17B852- | RSSQSLVHSNRNTYLH |
| | | humanized | G34R-LH-spFv | |
| | | | VL KABAT | |
| | | | CDR1 | |
| 192 | PRT | Mouse- | B17B852- | KVSNRFS |
| | | humanized | G34R-LH-spFv | |
| | | | VL KABAT | |
| | | | CDR2 | |
| 193 | PRT | Mouse- | B17B852- | SQSTHVPFT |
| | | humanized | G34R-LH-spFv | |
| | | | VL KABAT | |
| | | | CDR3 | |
| 194 | PRT | Mouse- | B17B852- | RSSQSLVHSNRNTYLH |
| | | humanized | G34R-LH-spFv | |
| | | | VL CHOTHIA | |
| | | | CDR1 | |
| 195 | PRT | Mouse- | B17B852- | KVSNRFS |
| | | humanized | G34R-LH-spFv | |
| | | | VL CHOTHIA | |
| | | | CDR2 | |
| 196 | PRT | Mouse- | B17B852- | SQSTHVPFT |
| | | humanized | G34R-LH-spFv | |
| | | | VL CHOTHIA | |
| | | | CDR3 | |
| 197 | PRT | Mouse- | B17B852- | QSLVHSNRNTY |
| | | humanized | G34R-LH-spFv | |
| | | | VL IMGT | |
| | | | CDR1 | |
| 199 | PRT | Mouse- | B17B852- | SQSTHVPFT |
| | | humanized | G34R-LH-spFv | |
| | | | VL IMGT | |
| | | | CDR3 | |
| 200 | PRT | Mouse- | B17B852- | VHSNRNTYLHWY |
| | | humanized | G34R-LH-spFv | |
| | | | VL CONTACT | |
| | | | CDR1 | |
| 201 | PRT | Mouse- | B17B852- | LLIYKVSNRF |
| | | humanized | G34R-LH-spFv | |
| | | | VL CONTACT | |
| | | | CDR2 | |
| 202 | PRT | Mouse- | B17B852- | SQSTHVPF |
| | | humanized | G34R-LH-spFv | |
| | | | VL CONTACT | |
| | | | CDR3 | |
| 203 | PRT | Mouse- | B17B852- | QVQLQESGPGLVKPSETLSLTCTVSGYSITSG |
| | | humanized | G34R-LH-spFv | YFWNWIRQFPGKGLEWMGYISYDGSNNYNP |
| | | | VH | SLKSRITISRDTSKNQFSLKLSSVTAADTAVY |
| | | | | YCASPSPGTGYAVDYWGCGTTVTVSS |
| 204 | PRT | Mouse- | B17B852- | DIQMTQSPSSLSASVGDRVTITCRSSQSLVHS |
| | | humanized | G34R-LH-spFv | NRNTYLHWYQQKPGCAPKLLIYKVSNRFSG |
| | | | VL | VPSRFSGSGSGTDFTLTISSLQPEDFATYYCSQ |
| | | | | STHVPFTFGQGTKLEIK |
| 205 | PRT | Mouse- | B17B852- | GYSITSGYFWN |
| | | humanized | G34R-HL-spFv | |
| | | | VH AbM | |
| | | | CDR1 | |
| 206 | PRT | Mouse- | B17B852- | YISYDGSNN |
| | | humanized | G34R- HL- | |
| | | | spFv VH AbM | |
| | | | CDR2 | |
| 207 | PRT | Mouse- | B17B852- | PSPGTGYAVDY |
| | | humanized | G34R- HL- | |
| | | | spFv VH AbM | |
| | | | CDR3 | |
| 208 | PRT | Mouse- | B17B852- | SGYFWN |
| | | humanized | G34R- HL- | |
| | | | spFv VH | |
| | | | KABAT CDR1 | |
| 209 | PRT | Mouse- | B17B852- | YISYDGSNNYNPSLKS |
| | | humanized | G34R- HL- | |
| | | | spFv VH | |
| | | | KABAT CDR2 | |
| 210 | PRT | Mouse- | B17B852- | PSPGTGYAVDY |
| | | humanized | G34R- HL- | |
| | | | spFv VH | |
| | | | KABAT CDR3 | |
| 211 | PRT | Mouse- | B17B852- | GYSITSGY |
| | | humanized | G34R- HL- | |
| | | | spFv VH | |
| | | | CHOTHIA | |
| | | | CDR1 | |
| 212 | PRT | Mouse- | B17B852- | SYDGS |
| | | humanized | G34R- HL- | |
| | | | spFv VH | |
| | | | CHOTHIA | |
| | | | CDR2 | |
| 213 | PRT | Mouse- | B17B852- | PSPGTGYAVDY |
| | | humanized | G34R- HL- | |
| | | | spFv VH | |
| | | | CHOTHIA | |
| | | | CDR3 | |
| 214 | PRT | Mouse- | B17B852- | GYSITSGYF |
| | | humanized | G34R- HL- | |
| | | | spFv VH | |
| | | | IMGT CDR1 | |
| 215 | PRT | Mouse- | B17B852- | ISYDGSN |
| | | humanized | G34R-HL-spFv | |
| | | | VH IMGT | |
| | | | CDR2 | |
| 216 | PRT | Mouse- | B17B852- | ASPSPGTGYAVDY |
| | | humanized | G34R-HL-spFv | |
| | | | VH IMGT | |
| | | | CDR3 | |
| 217 | PRT | Mouse- | B17B852- | TSGYFWN |
| | | humanized | G34R-HL-spFv | |
| | | | VH | |
| | | | CONTACT | |
| | | | CDR1 | |
| 218 | PRT | Mouse- | B17B852- | WMGYISYDGSNN |
| | | humanized | G34R-HL-spFv | |
| | | | VH | |
| | | | CONTACT | |
| | | | CDR2 | |
| 219 | PRT | Mouse- | B17B852- | ASPSPGTGYAVD |
| | | humanized | G34R-HL-spFv | |
| | | | VH | |
| | | | CONTACT | |
| | | | CDR3 | |
| 220 | PRT | Mouse- | B17B852- | RSSQSLVHSNRNTYLH |
| | | humanized | G34R-HL-spFv | |
| | | | VL AbM | |
| | | | CDR1 | |
| 221 | PRT | Mouse- | B17B852- | KVSNRFS |
| | | humanized | G34R-HL-spFv | |
| | | | VL AbM | |
| | | | CDR2 | |
| 222 | PRT | Mouse- | B17B852- | SQSTHVPFT |
| | | humanized | G34R-HL-spFv | |
| | | | VL AbM | |
| | | | CDR3 | |
| 223 | PRT | Mouse- | B17B852- | RSSQSLVHSNRNTYLH |
| | | humanized | G34R-HL-spFv | |
| | | | VL KABAT | |
| | | | CDR1 | |
| 224 | PRT | Mouse- | B17B852- | KVSNRFS |
| | | humanized | G34R-HL-spFv | |
| | | | VL KABAT | |
| | | | CDR2 | |
| 225 | PRT | Mouse- | B17B852- | SQSTHVPFT |
| | | humanized | G34R-HL-spFv | |
| | | | VL KABAT | |
| | | | CDR3 | |
| 226 | PRT | Mouse- | B17B852- | RSSQSLVHSNRNTYLH |
| | | humanized | G34R-HL-spFv | |
| | | | VL CHOTHIA | |
| | | | CDR1 | |
| 227 | PRT | Mouse- | B17B852- | KVSNRFS |
| | | humanized | G34R-HL-spFv | |
| | | | VL CHOTHIA | |
| | | | CDR2 | |
| 228 | PRT | Mouse- | B17B852- | SQSTHVPFT |
| | | humanized | G34R-HL-spFv | |
| | | | VL CHOTHIA | |
| | | | CDR3 | |
| 229 | PRT | Mouse- | B17B852- | QSLVHSNRNTY |
| | | humanized | G34R-HL-spFv | |
| | | | VL IMGT | |
| | | | CDR1 | |
| 231 | PRT | Mouse- | B17B852- | SQSTHVPFT |
| | | humanized | G34R-HL-spFv | |
| | | | VL IMGT | |
| | | | CDR3 | |
| 232 | PRT | Mouse- | B17B852- | VHSNRNTYLHWY |
| | | humanized | G34R-HL-spFv | |
| | | | VL CONTACT | |
| | | | CDR1 | |
| 233 | PRT | Mouse- | B17B852- | LLIYKVSNRF |
| | | humanized | G34R-HL-spFv | |
| | | | VL CONTACT | |
| | | | CDR2 | |
| 234 | PRT | Mouse- | B17B852- | SQSTHVPF |
| | | humanized | G34R-HL-spFv | |
| | | | VL CONTACT | |
| | | | CDR3 | |
| 235 | PRT | Mouse- | B17B852- | QVQLQESGPGLVKPSETLSLTCTVSGYSITSG |
| | | humanized | G34R-HL-spFv | YFWNWIRQFPGCGLEWMGYISYDGSNNYNP |
| | | | VH | SLKSRITISRDTSKNQFSLKLSSVTAADTAVY |
| | | | | YCASPSPGTGYAVDYWGQGTTVTVSS |
| 236 | PRT | Mouse- | B17B852- | DIQMTQSPSSLSASVGDRVTITCRSSQSLVHS |
| | | humanized | G34R-HL-spFv | NRNTYLHWYQQKPGKAPKLLIYKVSNRFSG |
| | | | VL | VPSRFSGSGSGTDFTLTISSLQPEDFATYYCSQ |
| | | | | STHVPFTFGCGTKLEIK |
| 237 | PRT | Mouse- | KL2B30 VH | GGSISSYYWS |
| | | humanized | AbM CDR1 | |
| 238 | PRT | Mouse- | KL2B30 VH | YIYYSGSTN |
| | | humanized | AbM CDR2 | |
| 239 | PRT | Mouse- | KL2B30 VH | TTIFGVVTPNFYYGMDV |
| | | humanized | AbM CDR3 | |
| 240 | PRT | Mouse- | KL2B30 VH | SYYWS |
| | | humanized | KABAT CDR1 | |
| 241 | PRT | Mouse- | KL2B30 VH | YIYYSGSTNYNPSLKS |
| | | humanized | KABAT CDR2 | |
| 242 | PRT | Mouse- | KL2B30 VH | TTIFGVVTPNFYYGMDV |
| | | humanized | KABAT CDR3 | |
| 243 | PRT | Mouse- | KL2B30 VH | GGSISSY |
| | | humanized | CHOTHIA | |
| | | | CDR1 | |
| 244 | PRT | Mouse- | KL2B30 VH | YYSGS |
| | | humanized | CHOTHIA | |
| | | | CDR2 | |
| 245 | PRT | Mouse- | KL2B30 VH | TTIFGVVTPNFYYGMDV |
| | | humanized | CHOTHIA | |
| | | | CDR3 | |
| 246 | PRT | Mouse- | KL2B30 VH | GGSISSYY |
| | | humanized | IMGT CDR1 | |
| 247 | PRT | Mouse- | KL2B30 VH | IYYSGST |
| | | humanized | IMGT CDR2 | |
| 248 | PRT | Mouse- | KL2B30 VH | AGTTIFGVVTPNFYYGMDV |
| | | humanized | IMGT CDR3 | |
| 249 | PRT | Mouse- | KL2B30 VH | SSYYWS |
| | | humanized | CONTACT | |
| | | | CDR1 | |
| 250 | PRT | Mouse- | KL2B30 VH | WIGYIYYSGSTN |
| | | humanized | CONTACT | |
| | | | CDR2 | |
| 251 | PRT | Mouse- | KL2B30 VH | AGTTIFGVVTPNFYYGMD |
| | | humanized | CONTACT | |
| | | | CDR3 | |
| 252 | PRT | Mouse- | KL2B30 VL | RASQGISSYLA |
| | | humanized | AbM CDR1 | |
| 253 | PRT | Mouse- | KL2B30 VL | AASTLQS |
| | | humanized | AbM CDR2 | |
| 254 | PRT | Mouse- | KL2B30 VL | QQLNSYPLT |
| | | humanized | AbM CDR3 | |
| 255 | PRT | Mouse- | KL2B30 VL | RASQGISSYLA |
| | | humanized | KABAT CDR1 | |
| 256 | PRT | Mouse- | KL2B30 VL | AASTLQS |
| | | humanized | KABAT CDR2 | |
| 257 | PRT | Mouse- | KL2B30 VL | QQLNSYPLT |
| | | humanized | KABAT CDR3 | |
| 258 | PRT | Mouse- | KL2B30 VL | RASQGISSYLA |
| | | humanized | CHOTHIA | |
| | | | CDR1 | |
| 259 | PRT | Mouse- | KL2B30 VL | AASTLQS |
| | | humanized | CHOTHIA | |
| | | | CDR2 | |
| 260 | PRT | Mouse- | KL2B30 VL | QQLNSYPLT |
| | | humanized | CHOTHIA | |
| | | | CDR3 | |
| 261 | PRT | Mouse- | KL2B30 VL | QGISSY |
| | | humanized | IMGT CDR1 | |
| 263 | PRT | Mouse- | KL2B30 VL | QQLNSYPLT |
| | | humanized | IMGT CDR3 | |
| 264 | PRT | Mouse- | KL2B30 VL | SSYLAWY |
| | | humanized | CONTACT | |
| | | | CDR1 | |
| 265 | PRT | Mouse- | KL2B30 VL | FLIYAASTLQ |
| | | humanized | CONTACT | |
| | | | CDR2 | |
| 266 | PRT | Mouse- | KL2B30 VL | QQLNSYPL |
| | | humanized | CONTACT | |
| | | | CDR3 | |
| 267 | PRT | Mouse- | KL2B30 VH | QVQLQESGPGLVKPSETLSLTCTVSGGSISSY |
| | | humanized | | YWSWIRQPPGKGLEWIGYIYYSGSTNYNPSL |
| | | | | KSRVTISVDTSKNQFSLKLSSVTAADTAVYY |
| | | | | CAGTTIFGVVTPNFYYGMDVWGQGTTVTVS |
| | | | | S |
| 268 | PRT | Mouse- | KL2B30 VL | DIQMTQSPSFLSASVGDRVTITCRASQGISSYL |
| | | humanized | | AWYQQKPGKAPKFLIYAASTLQSGVPSRFSG |
| | | | | SGSGTEFTLTISSLQPEDFATYYCQQLNSYPLT |
| | | | | FGGGTKVEIK |
| 269 | PRT | Mouse- | PSMB896- | GFTFSSYAMS |
| | | humanized | G100A VH | |
| | | | AbM CDR1 | |
| 270 | PRT | Mouse- | PSMB896- | AISGGIGSTY |
| | | humanized | G100A VH | |
| | | | AbM CDR2 | |
| 271 | PRT | Mouse- | PSMB896- | DAVGATPYYFDY |
| | | humanized | G100A VH | |
| | | | AbM CDR3 | |
| 272 | PRT | Mouse- | PSMB896- | SYAMS |
| | | humanized | G100A VH | |
| | | | KABAT CDR1 | |
| 273 | PRT | Mouse- | PSMB896- | AISGGIGSTYYADSVKG |
| | | humanized | G100A VH | |
| | | | KABAT CDR2 | |
| 274 | PRT | Mouse- | PSMB896- | DAVGATPYYFDY |
| | | humanized | G100A VH | |
| | | | KABAT CDR3 | |
| 275 | PRT | Mouse- | PSMB896- | GFTFSSY |
| | | humanized | G100A VH | |
| | | | CHOTHIA | |
| | | | CDR1 | |
| 276 | PRT | Mouse- | PSMB896- | SGGIGS |
| | | humanized | G100A VH | |
| | | | CHOTHIA | |
| | | | CDR2 | |
| 277 | PRT | Mouse- | PSMB896- | DAVGATPYYFDY |
| | | humanized | G100A VH | |
| | | | CHOTHIA | |
| | | | CDR3 | |
| 278 | PRT | Mouse- | PSMB896- | GFTFSSYA |
| | | humanized | G100A VH | |
| | | | IMGT CDR1 | |
| 279 | PRT | Mouse- | PSMB896- | ISGGIGST |
| | | humanized | G100A VH | |
| | | | IMGT CDR2 | |
| 280 | PRT | Mouse- | PSMB896- | AKDAVGATPYYFDY |
| | | humanized | G100A VH | |
| | | | IMGT CDR3 | |
| 281 | PRT | Mouse- | PSMB896- | SSYAMS |
| | | humanized | G100A VH | |
| | | | CONTACT | |
| | | | CDR1 | |
| 282 | PRT | Mouse- | PSMB896- | WVSAISGGIGSTY |
| | | humanized | G100A VH | |
| | | | CONTACT | |
| | | | CDR2 | |
| 283 | PRT | Mouse- | PSMB896- | AKDAVGATPYYFD |
| | | humanized | G100A VH | |
| | | | CONTACT | |
| | | | CDR3 | |
| 284 | PRT | Mouse- | PSMB896- | SGSSSNIGINYVS |
| | | humanized | G100A VL | |
| | | | AbM CDR1 | |
| 285 | PRT | Mouse- | PSMB896- | DNNKRPS |
| | | humanized | G100A VL | |
| | | | AbM CDR2 | |
| 286 | PRT | Mouse- | PSMB896- | GTWDSSLSAVV |
| | | humanized | G100A VL | |
| | | | AbM CDR3 | |
| 287 | PRT | Mouse- | PSMB896- | SGSSSNIGINYVS |
| | | humanized | G100A VL | |
| | | | KABAT CDR1 | |
| 288 | PRT | Mouse- | PSMB896- | DNNKRPS |
| | | humanized | G100A VL | |
| | | | KABAT CDR2 | |
| 289 | PRT | Mouse- | PSMB896- | GTWDSSLSAVV |
| | | humanized | G100A VL | |
| | | | KABAT CDR3 | |
| 290 | PRT | Mouse- | PSMB896- | SGSSSNIGINYVS |
| | | humanized | G100A VL | |
| | | | CHOTHIA | |
| | | | CDR1 | |
| 291 | PRT | Mouse- | PSMB896- | DNNKRPS |
| | | humanized | G100A VL | |
| | | | CHOTHIA | |
| | | | CDR2 | |
| 292 | PRT | Mouse- | PSMB896- | GTWDSSLSAVV |
| | | humanized | G100A VL | |
| | | | CHOTHIA | |
| | | | CDR3 | |
| 293 | PRT | Mouse- | PSMB896- | SSNIGINY |
| | | humanized | G100A VL | |
| | | | IMGT CDR1 | |
| 295 | PRT | Mouse- | PSMB896- | GTWDSSLSAVV |
| | | humanized | G100A VL | |
| | | | IMGT CDR3 | |
| 296 | PRT | Mouse- | PSMB896- | IGINYVSWY |
| | | humanized | G100A VL | |
| | | | CONTACT | |
| | | | CDR1 | |
| 297 | PRT | Mouse- | PSMB896- | LLIYDNNKRP |
| | | humanized | G100A VL | |
| | | | CONTACT | |
| | | | CDR2 | |
| 298 | PRT | Mouse- | PSMB896- | GTWDSSLSAV |
| | | humanized | G100A VL | |
| | | | CONTACT | |
| | | | CDR3 | |
| 299 | PRT | Mouse- | PSMB896- | EVQLVESGGGLVQPGGSLRLSCAASGFTFSSY |
| | | humanized | G100A VH | AMSWVRQAPGKGLEWVSAISGGIGSTYYAD |
| | | | | SVKGRFTISRDNSKNTLWLQMNSLRAEDTAV |
| | | | | YYCAKDAVGATPYYFDYWGQGTLVTVSS |
| 300 | PRT | Mouse- | PSMB896- | QSVLTQPPSVSAAPGQKVTISCSGSSSNIGINY |
| | | humanized | G100A VL | VSWYQQLPGTAPKLLIYDNNKRPSGIPDRFSG |
| | | | | SKSGTSATLGITGLQTGDEADYYCGTWDSSL |
| | | | | SAVVFGGGTKLTVL |
| 301 | PRT | Mouse- | BCMB519 VH | GFTFSSYAMS |
| | | humanized | AbM CDR1 | |
| 302 | PRT | Mouse- | BCMB519 VH | AISGSGGSTY |
| | | humanized | AbM CDR2 | |
| 303 | PRT | Mouse- | BCMB519 VH | DEGYSSGHYYGMDV |
| | | humanized | AbM CDR3 | |
| 304 | PRT | Mouse- | BCMB519 VH | SYAMS |
| | | humanized | KABAT CDR1 | |
| 305 | PRT | Mouse- | BCMB519 VH | AISGSGGSTYYADSVKG |
| | | humanized | KABAT CDR2 | |
| 306 | PRT | Mouse- | BCMB519 VH | DEGYSSGHYYGMDV |
| | | humanized | KABAT CDR3 | |
| 307 | PRT | Mouse- | BCMB519 VH | GFTFSSY |
| | | humanized | CHOTHIA | |
| | | | CDR1 | |
| 308 | PRT | Mouse- | BCMB519 VH | SGSGGS |
| | | humanized | CHOTHIA | |
| | | | CDR2 | |
| 309 | PRT | Mouse- | BCMB519 VH | DEGYSSGHYYGMDV |
| | | humanized | CHOTHIA | |
| | | | CDR3 | |
| 310 | PRT | Mouse- | BCMB519 VH | GFTFSSYA |
| | | humanized | IMGT CDR1 | |
| 311 | PRT | Mouse- | BCMB519 VH | ISGSGGST |
| | | humanized | IMGT CDR2 | |
| 312 | PRT | Mouse- | BCMB519 VH | AKDEGYSSGHYYGMDV |
| | | humanized | IMGT CDR3 | |
| 313 | PRT | Mouse- | BCMB519 VH | SSYAMS |
| | | humanized | CONTACT | |
| | | | CDR1 | |
| 314 | PRT | Mouse- | BCMB519 VH | WVSAISGSGGSTY |
| | | humanized | CONTACT | |
| | | | CDR2 | |
| 315 | PRT | Mouse- | BCMB519 VH | AKDEGYSSGHYYGMD |
| | | humanized | CONTACT | |
| | | | CDR3 | |
| 316 | PRT | Mouse- | BCMB519 VL | RASQSISSSFLT |
| | | humanized | AbM CDR1 | |
| 317 | PRT | Mouse- | BCMB519 VL | GASSRAT |
| | | humanized | AbM CDR2 | |
| 318 | PRT | Mouse- | BCMB519 VL | QHYGSSPMYT |
| | | humanized | AbM CDR3 | |
| 319 | PRT | Mouse- | BCMB519 VL | RASQSISSSFLT |
| | | humanized | KABAT CDR1 | |
| 320 | PRT | Mouse- | BCMB519 VL | GASSRAT |
| | | humanized | KABAT CDR2 | |
| 321 | PRT | Mouse- | BCMB519 VL | QHYGSSPMYT |
| | | humanized | KABAT CDR3 | |
| 322 | PRT | Mouse- | BCMB519 VL | RASQSISSSFLT |
| | | humanized | CHOTHIA | |
| | | | CDR1 | |
| 323 | PRT | Mouse- | BCMB519 VL | GASSRAT |
| | | humanized | CHOTHIA | |
| | | | CDR2 | |
| 324 | PRT | Mouse- | BCMB519 VL | QHYGSSPMYT |
| | | humanized | CHOTHIA | |
| | | | CDR3 | |
| 325 | PRT | Mouse- | BCMB519 VL | QSISSSF |
| | | humanized | IMGT CDR1 | |
| 327 | PRT | Mouse- | BCMB519 VL | QHYGSSPMYT |
| | | humanized | IMGT CDR3 | |
| 328 | PRT | Mouse- | BCMB519 VL | SSSFLTWY |
| | | humanized | CONTACT | |
| | | | CDR1 | |
| 329 | PRT | Mouse- | BCMB519 VL | LLIYGASSRA |
| | | humanized | CONTACT | |
| | | | CDR2 | |
| 330 | PRT | Mouse- | BCMB519 VL | QHYGSSPMY |
| | | humanized | CONTACT | |
| | | | CDR3 | |
| 331 | PRT | Mouse- | BCMB519 VH | EVQLLESGGGLVQPGGSLRLSCAASGFTFSSY |
| | | humanized | | AMSWVRQAPGKGLEWVSAISGSGGSTYYAD |
| | | | | SVKGRFTISRDNSKNTLYLQMNSLRAEDTAV |
| | | | | YYCAKDEGYSSGHYYGMDVWGQGTTVTVS |
| | | | | S |
| 332 | PRT | Mouse- | BCMB519 VL | EIVLTQSPGTLSLSPGERATLSCRASQSISSSFL |
| | | humanized | | TWYQQKPGQAPRLLIYGASSRATGIPDRFSG |
| | | | | GGSGTDFTLTISRLEPEDFAVYYCQHYGSSPM |
| | | | | YTFGQGTKLEIK |
| 333 | PRT | Mouse- | PSMB896 VH | GFTFSSYAMS |
| | | humanized | AbM CDR1 | |
| 334 | PRT | Mouse- | PSMB896 VH | AISGGIGSTY |
| | | humanized | AbM CDR2 | |
| 335 | PRT | Mouse- | PSMB896 VH | DGVGATPYYFDY |
| | | humanized | AbM CDR3 | |
| 336 | PRT | Mouse- | PSMB896 VH | SYAMS |
| | | humanized | KABAT CDR1 | |
| 337 | PRT | Mouse- | PSMB896 VH | AISGGIGSTYYADSVKG |
| | | humanized | KABAT CDR2 | |
| 338 | PRT | Mouse- | PSMB896 VH | DGVGATPYYFDY |
| | | humanized | KABAT CDR3 | |
| 339 | PRT | Mouse- | PSMB896 VH | GFTFSSY |
| | | humanized | CHOTHIA | |
| | | | CDR1 | |
| 340 | PRT | Mouse- | PSMB896 VH | SGGIGS |
| | | humanized | CHOTHIA | |
| | | | CDR2 | |
| 341 | PRT | Mouse- | PSMB896 VH | DGVGATPYYFDY |
| | | humanized | CHOTHIA | |
| | | | CDR3 | |
| 342 | PRT | Mouse- | PSMB896 VH | GFTFSSYA |
| | | humanized | IMGT CDR1 | |
| 343 | PRT | Mouse- | PSMB896 VH | ISGGIGST |
| | | humanized | IMGT CDR2 | |
| 344 | PRT | Mouse- | PSMB896 VH | AKDGVGATPYYFDY |
| | | humanized | IMGT CDR3 | |
| 345 | PRT | Mouse- | PSMB896 VH | SSYAMS |
| | | humanized | CONTACT | |
| | | | CDR1 | |
| 346 | PRT | Mouse- | PSMB896 VH | WVSAISGGIGSTY |
| | | humanized | CONTACT | |
| | | | CDR2 | |
| 347 | PRT | Mouse- | PSMB896 VH | AKDGVGATPYYFD |
| | | humanized | CONTACT | |
| | | | CDR3 | |
| 348 | PRT | Mouse- | PSMB896 VL | SGSSSNIGINYVS |
| | | humanized | AbM CDR1 | |
| 349 | PRT | Mouse- | PSMB896 VL | DNNKRPS |
| | | humanized | AbM CDR2 | |
| 350 | PRT | Mouse- | PSMB896 VL | GTWDSSLSAVV |
| | | humanized | AbM CDR3 | |
| 351 | PRT | Mouse- | PSMB896 VL | SGSSSNIGINYVS |
| | | humanized | KABAT CDR1 | |
| 352 | PRT | Mouse- | PSMB896 VL | DNNKRPS |
| | | humanized | KABAT CDR2 | |
| 353 | PRT | Mouse- | PSMB896 VL | GTWDSSLSAVV |
| | | humanized | KABAT CDR3 | |
| 354 | PRT | Mouse- | PSMB896 VL | SGSSSNIGINYVS |
| | | humanized | CHOTHIA | |
| | | | CDR1 | |
| 355 | PRT | Mouse- | PSMB896 VL | DNNKRPS |
| | | humanized | CHOTHIA | |
| | | | CDR2 | |
| 356 | PRT | Mouse- | PSMB896 VL | GTWDSSLSAVV |
| | | humanized | CHOTHIA | |
| | | | CDR3 | |
| 357 | PRT | Mouse- | PSMB896 VL | SSNIGINY |
| | | humanized | IMGT CDR1 | |
| 359 | PRT | Mouse- | PSMB896 VL | GTWDSSLSAVV |
| | | humanized | IMGT CDR3 | |
| 360 | PRT | Mouse- | PSMB896 VL | IGINYVSWY |
| | | humanized | CONTACT | |
| | | | CDR1 | |
| 361 | PRT | Mouse- | PSMB896 VL | LLIYDNNKRP |
| | | humanized | CONTACT | |
| | | | CDR2 | |
| 362 | PRT | Mouse- | PSMB896 VL | GTWDSSLSAV |
| | | humanized | CONTACT | |
| | | | CDR3 | |
| 363 | PRT | Mouse- | PSMB896 VH | EVQLVESGGGLVQPGGSLRLSCAASGFTFSSY |
| | | humanized | | AMSWVRQAPGKGLEWVSAISGGIGSTYYAD |
| | | | | SVKGRFTISRDNSKNTLWLQMNSLRAEDTAV |
| | | | | YYCAKDGVGATPYYFDYWGQGTLVTVSS |
| 364 | PRT | Mouse- | PSMB896 VL | QSVLTQPPSVSAAPGQKVTISCSGSSSNIGINY |
| | | humanized | | VSWYQQLPGTAPKLLIYDNNKRPSGIPDRFSG |
| | | | | SKSGTSATLGITGLQTGDEADYYCGTWDSSL |
| | | | | SAVVFGGGTKLTVL |
| 365 | PRT | Mouse- | PSMB896- | GFTFSSYAMS |
| | | humanized | G100A-LH- | |
| | | | spFv VH AbM | |
| | | | CDR1 | |
| 366 | PRT | Mouse- | PSMB896- | AISGGIGSTY |
| | | humanized | G100A-LH- | |
| | | | spFv VH AbM | |
| | | | CDR2 | |
| 367 | PRT | Mouse- | PSMB896- | DAVGATPYYFDY |
| | | humanized | G100A-LH- | |
| | | | spFv VH AbM | |
| | | | CDR3 | |
| 368 | PRT | Mouse- | PSMB896- | SYAMS |
| | | humanized | G100A-LH- | |
| | | | spFv VH | |
| | | | KABAT CDR1 | |
| 369 | PRT | Mouse- | PSMB896- | AISGGIGSTYYADSVKG |
| | | humanized | G100A-LH- | |
| | | | spFv VH | |
| | | | KABAT CDR2 | |
| 370 | PRT | Mouse- | PSMB896- | DAVGATPYYFDY |
| | | humanized | G100A-LH- | |
| | | | spFv VH | |
| | | | KABAT CDR3 | |
| 371 | PRT | Mouse- | PSMB896- | GFTFSSY |
| | | humanized | G100A-LH- | |
| | | | spFv VH | |
| | | | CHOTHIA | |
| | | | CDR1 | |
| 372 | PRT | Mouse- | PSMB896- | SGGIGS |
| | | humanized | G100A-LH- | |
| | | | spFv VH | |
| | | | CHOTHIA | |
| | | | CDR2 | |
| 373 | PRT | Mouse- | PSMB896- | DAVGATPYYFDY |
| | | humanized | G100A-LH- | |
| | | | spFv VH | |
| | | | CHOTHIA | |
| | | | CDR3 | |
| 374 | PRT | Mouse- | PSMB896- | GFTFSSYA |
| | | humanized | G100A-LH- | |
| | | | spFv VH | |
| | | | IMGT CDR1 | |
| 375 | PRT | Mouse- | PSMB896- | ISGGIGST |
| | | humanized | G100A-LH- | |
| | | | spFv VH | |
| | | | IMGT CDR2 | |
| 376 | PRT | Mouse- | PSMB896- | AKDAVGATPYYFDY |
| | | humanized | G100A-LH- | |
| | | | spFv VH | |
| | | | IMGT CDR3 | |
| 377 | PRT | Mouse- | PSMB896- | SSYAMS |
| | | humanized | G100A-LH- | |
| | | | spFv VH | |
| | | | CONTACT | |
| | | | CDR1 | |
| 378 | PRT | Mouse- | PSMB896- | WVSAISGGIGSTY |
| | | humanized | G100A-LH- | |
| | | | spFv VH | |
| | | | CONTACT | |
| | | | CDR2 | |
| 379 | PRT | Mouse- | PSMB896- | AKDAVGATPYYFD |
| | | humanized | G100A-LH- | |
| | | | spFv VH | |
| | | | CONTACT | |
| | | | CDR3 | |
| 380 | PRT | Mouse- | PSMB896- | SGSSSNIGINYVS |
| | | humanized | G100A-LH- | |
| | | | spFv VL AbM | |
| | | | CDR1 | |
| 381 | PRT | Mouse- | PSMB896- | DNNKRPS |
| | | humanized | G100A-LH- | |
| | | | spFv VL AbM | |
| | | | CDR2 | |
| 382 | PRT | Mouse- | PSMB896- | GTWDSSLSAVV |
| | | humanized | G100A-LH- | |
| | | | spFv VL AbM | |
| | | | CDR3 | |
| 383 | PRT | Mouse- | PSMB896- | SGSSSNIGINYVS |
| | | humanized | G100A-LH- | |
| | | | spFv VL | |
| | | | KABAT CDR1 | |
| 384 | PRT | Mouse- | PSMB896- | DNNKRPS |
| | | humanized | G100A-LH- | |
| | | | spFv VL | |
| | | | KABAT CDR2 | |
| 385 | PRT | Mouse- | PSMB896- | GTWDSSLSAVV |
| | | humanized | G100A-LH- | |
| | | | spFv VL | |
| | | | KABAT CDR3 | |
| 386 | PRT | Mouse- | PSMB896- | SGSSSNIGINYVS |
| | | humanized | G100A-LH- | |
| | | | spFv VL | |
| | | | CHOTHIA | |
| | | | CDR1 | |
| 387 | PRT | Mouse- | PSMB896- | DNNKRPS |
| | | humanized | G100A-LH- | |
| | | | spFv VL | |
| | | | CHOTHIA | |
| | | | CDR2 | |
| 388 | PRT | Mouse- | PSMB896- | GTWDSSLSAVV |
| | | humanized | G100A-LH- | |
| | | | spFv VL | |
| | | | CHOTHIA | |
| | | | CDR3 | |
| 389 | PRT | Mouse- | PSMB896- | SSNIGINY |
| | | humanized | G100A-LH- | |
| | | | spFv VL IMGT | |
| | | | CDR1 | |
| 391 | PRT | Mouse- | PSMB896- | GTWDSSLSAVV |
| | | humanized | G100A-LH- | |
| | | | spFv VL IMGT | |
| | | | CDR3 | |
| 392 | PRT | Mouse- | PSMB896- | IGINYVSWY |
| | | humanized | G100A-LH- | |
| | | | spFv VL | |
| | | | CONTACT | |
| | | | CDR1 | |
| 393 | PRT | Mouse- | PSMB896- | LLIYDNNKRP |
| | | humanized | G100A-LH- | |
| | | | spFv VL | |
| | | | CONTACT | |
| | | | CDR2 | |
| 394 | PRT | Mouse- | PSMB896- | GTWDSSLSAV |
| | | humanized | G100A-LH- | |
| | | | spFv VL | |
| | | | CONTACT | |
| | | | CDR3 | |
| 395 | PRT | Mouse- | PSMB896- | EVQLVESGGGLVQPGGSLRLSCAASGFTFSSY |
| | | humanized | G100A-LH- | AMSWVRQAPGKGLEWVSAISGGIGSTYYAD |
| | | | spFv VH | SVKGRFTISRDNSKNTLWLQMNSLRAEDTAV |
| | | | | YYCAKDAVGATPYYFDYWGCGTLVTVSS |
| 396 | PRT | Mouse- | PSMB896- | QSVLTQPPSVSAAPGQKVTISCSGSSSNIGINY |
| | | humanized | G100A-LH- | VSWYQQLPGCAPKLLIYDNNKRPSGIPDRFSG |
| | | | spFv VL | SKSGTSATLGITGLQTGDEADYYCGTWDSSL |
| | | | | SAVVFGGGTKLTVL |
| 397 | PRT | Mouse- | PSMB410-LH- | GASISSPSYYWG |
| | | humanized | spFv VH AbM | |
| | | | CDR1 | |
| 398 | PRT | Mouse- | PSMB410-LH- | SIFYSGSSY |
| | | humanized | spFv VH AbM | |
| | | | CDR2 | |
| 399 | PRT | Mouse- | PSMB410-LH- | QSGVSGWYGAEYFQH |
| | | humanized | spFv VH AbM | |
| | | | CDR3 | |
| 400 | PRT | Mouse- | PSMB410-LH- | SPSYYWG |
| | | humanized | spFv VH | |
| | | | KABAT CDR1 | |
| 401 | PRT | Mouse- | PSMB410-LH- | SIFYSGSSYYNPSLKS |
| | | humanized | spFv VH | |
| | | | KABAT CDR2 | |
| 402 | PRT | Mouse- | PSMB410-LH- | QSGVSGWYGAEYFQH |
| | | humanized | spFv VH | |
| | | | KABAT CDR3 | |
| 403 | PRT | Mouse- | PSMB410-LH- | GASISSPSY |
| | | humanized | spFv VH | |
| | | | CHOTHIA | |
| | | | CDR1 | |
| 404 | PRT | Mouse- | PSMB410-LH- | FYSGS |
| | | humanized | spFv VH | |
| | | | CHOTHIA | |
| | | | CDR2 | |
| 405 | PRT | Mouse- | PSMB410-LH- | QSGVSGWYGAEYFQH |
| | | humanized | spFv VH | |
| | | | CHOTHIA | |
| | | | CDR3 | |
| 406 | PRT | Mouse- | PSMB410-LH- | GASISSPSYY |
| | | humanized | spFv VH | |
| | | | IMGT CDR1 | |
| 407 | PRT | Mouse- | PSMB410-LH- | IFYSGSS |
| | | humanized | spFv VH | |
| | | | IMGT CDR2 | |
| 408 | PRT | Mouse- | PSMB410-LH- | ASQSGVSGWYGAEYFQH |
| | | humanized | spFv VH | |
| | | | IMGT CDR3 | |
| 409 | PRT | Mouse- | PSMB410-LH- | SSPSYYWG |
| | | humanized | spFv VH | |
| | | | CONTACT | |
| | | | CDR1 | |
| 410 | PRT | Mouse- | PSMB410-LH- | WIGSIFYSGSSY |
| | | humanized | spFv VH | |
| | | | CONTACT | |
| | | | CDR2 | |
| 411 | PRT | Mouse- | PSMB410-LH- | ASQSGVSGWYGAEYFQ |
| | | humanized | spFv VH | |
| | | | CONTACT | |
| | | | CDR3 | |
| 412 | PRT | Mouse- | PSMB410-LH- | RASQSISSWLA |
| | | humanized | spFv VL AbM | |
| | | | CDR1 | |
| 413 | PRT | Mouse- | PSMB410-LH- | KASSLES |
| | | humanized | spFv VL AbM | |
| | | | CDR2 | |
| 414 | PRT | Mouse- | PSMB410-LH- | QQYNSYSRT |
| | | humanized | spFv VL AbM | |
| | | | CDR3 | |
| 415 | PRT | Mouse- | PSMB410-LH- | RASQSISSWLA |
| | | humanized | spFv VL | |
| | | | KABAT CDR1 | |
| 416 | PRT | Mouse- | PSMB410-LH- | KASSLES |
| | | humanized | spFv VL | |
| | | | KABAT CDR2 | |
| 417 | PRT | Mouse- | PSMB410-LH- | QQYNSYSRT |
| | | humanized | spFv VL | |
| | | | KABAT CDR3 | |
| 418 | PRT | Mouse- | PSMB410-LH- | RASQSISSWLA |
| | | humanized | spFv VL | |
| | | | CHOTHIA | |
| | | | CDR1 | |
| 419 | PRT | Mouse- | PSMB410-LH- | KASSLES |
| | | humanized | spFv VL | |
| | | | CHOTHIA | |
| | | | CDR2 | |
| 420 | PRT | Mouse- | PSMB410-LH- | QQYNSYSRT |
| | | humanized | spFv VL | |
| | | | CHOTHIA | |
| | | | CDR3 | |
| 421 | PRT | Mouse- | PSMB410-LH- | QSISSW |
| | | humanized | spFv VL IMGT | |
| | | | CDR1 | |
| 423 | PRT | Mouse- | PSMB410-LH- | QQYNSYSRT |
| | | humanized | spFv VL IMGT | |
| | | | CDR3 | |
| 424 | PRT | Mouse- | PSMB410-LH- | SSWLAWY |
| | | humanized | spFv VL | |
| | | | CONTACT | |
| | | | CDR1 | |
| 425 | PRT | Mouse- | PSMB410-LH- | LLIYKASSLE |
| | | humanized | spFv VL | |
| | | | CONTACT | |
| | | | CDR2 | |
| 426 | PRT | Mouse- | PSMB410-LH- | QQYNSYSR |
| | | humanized | spFv VL | |
| | | | CONTACT | |
| | | | CDR3 | |
| 427 | PRT | Mouse- | PSMB410-LH- | QLQLQESGPGLVKPSETLSLTCTVSGASISSPS |
| | | humanized | spFv VH | YYWGWIRQPPGKGLEWIGSIFYSGSSYYNPSL |
| | | | | KSRVIMSVDTSKNQFSLKLSSVTAADTALYY |
| | | | | CASQSGVSGWYGAEYFQHWGCGTLVTVSS |
| 428 | PRT | Mouse- | PSMB410-LH- | DIQMTQSPSTLSASVGDRVTITCRASQSISSWL |
| | | humanized | spFv VL | AWYQQKPGCAPKLLIYKASSLESGVPSRFSGS |
| | | | | GSGTEFTLTISSLQPDDFATYYCQQYNSYSRT |
| | | | | FGQGTKVEIK |
| 429 | PRT | Mouse- | PS3B1376- | DIQMTQSPSSLSASVGDRVTITCRSSQSLVHSS |
| | | humanized | HC1 (B17B21- | GNTYLHWYQQKPGKAPKFLIYKVSNRFSGVP |
| | | | N33S-LH- | SRFSGSGSGTDFTLTISSLQPEDFATYYCSQST |
| | | | scFv) | HVPFTFGQGTKLEIKGGSEGKSSGSGSESKST |
| | | | | GGSQVQLQESGPGLVKPSETLSLTCTVSGYSI |
| | | | | TSGYFWNWIRQPPGKGLEWIGYISYDGSNNY |
| | | | | NPSLKSRVTISRDTSKNQFSLKLSSVTAADTA |
| | | | | VYYCASPSPGTGYAVDYWGQGTLVTVSSEP |
| | | | | KSSDKTHTCPPCPAPEAAGGPSVFLFPPKPKD |
| | | | | TLMISRTPEVTCVVVSVSHEDPEVKFNWYVD |
| | | | | GVEVHNAKTKPREEQYNSTYRVVSVLTVLH |
| | | | | QDWLNGKEYKCKVSNKALPAPIEKTISKAKG |
| | | | | QPREPQVYTLPPSREEMTKNQVSLWCLVKGF |
| | | | | YPSDIAVEWESNGQPENNYKTTPPVLDSDGS |
| | | | | FFLYSKLTVDKSRWQQGNVFSCSVMHEALH |
| | | | | NHYTQKSLSLSPGK |
| 430 | PRT | Mouse- | PS3B1376- | EVQLVESGGGLVQPGGSLRLSCAASGFTFSSY |
| | | humanized | HC2 | AMSWVRQAPGKGLEWVSAISGGIGSTYYAD |
| | | | (PSMB896- | SVKGRFTISRDNSKNTLWLQMNSLRAEDTAV |
| | | | Fab) | YYCAKDGVGATPYYFDYWGQGTLVTVSSAS |
| | | | | TKGPSVFPLAPSSKSTSGGTAALGCLVKDYFP |
| | | | | EPVTVSWNSGALTSGVHTFPAVLQSSGLYSL |
| | | | | SSVVTVPSSSLGTQTYICNVNHKPSNTKVDK |
| | | | | KVEPKSCDKTHTCPPCPAPEAAGGPSVFLFPP |
| | | | | KPKDTLMISRTPEVTCVVVSVSHEDPEVKFN |
| | | | | WYVDGVEVHNAKTKPREEQYNSTYRVVSVL |
| | | | | TVLHQDWLNGKEYKCKVSNKALPAPIEKTIS |
| | | | | KAKGQPREPQVYTLPPSREEMTKNQVSLSCA |
| | | | | VKGFYPSDIAVEWESNGQPENNYKTTPPVLD |
| | | | | SDGSFFLVSKLTVDKSRWQQGNVFSCSVMHE |
| | | | | ALHNRFTQKSLSLSPGK |
| 431 | PRT | Mouse- | PS3B1376- | QSVLTQPPSVSAAPGQKVTISCSGSSSNIGINY |
| | | humanized | LC2 | VSWYQQLPGTAPKLLIYDNNKRPSGIPDRFSG |
| | | | | SKSGTSATLGITGLQTGDEADYYCGTWDSSL |
| | | | | SAVVFGGGTKLTVLGQPKAAPSVTLFPPSSEE |
| | | | | LQANKATLVCLISDFYPGAVTVAWKADSSPV |
| | | | | KAGVETTTPSKQSNNKYAASSYLSLTPEQWK |
| | | | | SHRSYSCQVTHEGSTVEKTVAPTECS |
| 432 | PRT | Mouse- | V7BCB37- | DIQMTQSPSSLSASVGDRVTITCRSSQSLVHS |
| | | humanized | HC1 | NRNTYLHWYQQKPGKAPKLLIYKVSNRFSG |
| | | | (B17B852- | VPSRFSGSGSGTDFTLTISSLQPEDFATYYCSQ |
| | | | G34R-LH- | STHVPFTFGQGTKLEIKGGSEGKSSGSGSESK |
| | | | scFv) | STGGSQVQLQESGPGLVKPSETLSLTCTVSGY |
| | | | | SITSGYFWNWIRQFPGKGLEWMGYISYDGSN |
| | | | | NYNPSLKSRITISRDTSKNQFSLKLSSVTAADT |
| | | | | AVYYCASPSPGTGYAVDYWGQGTTVTVSSE |
| | | | | PKSSDKTHTCPPCPAPEAAGGPSVFLFPPKPK |
| | | | | DTLMISRTPEVTCVVVSVSHEDPEVKFNWYV |
| | | | | DGVEVHNAKTKPREEQYNSTYRVVSVLTVL |
| | | | | HQDWLNGKEYKCKVSNKALPAPIEKTISKAK |
| | | | | GQPREPQVYVYPPSREEMTKNQVSLTCLVKG |
| | | | | FYPSDIAVEWESNGQPENNYKTTPPVLDSDG |
| | | | | SFALVSKLTVDKSRWQQGNVFSCSVMHEAL |
| | | | | HNHYTQKSLSLSPGK |
| 433 | PRT | Mouse- | V7BCB37- | EVQLLESGGGLVQPGGSLRLSCAASGFTFSSY |
| | | humanized | HC2 | AMSWVRQAPGKGLEWVSAISGSGGSTYYAD |
| | | | (BCMB519- | SVKGRFTISRDNSKNTLYLQMNSLRAEDTAV |
| | | | Fab) | YYCAKDEGYSSGHYYGMDVWGQGTTVTVS |
| | | | | SASTKGPSVFPLAPSSKSTSGGTAALGCLVKD |
| | | | | YFPEPVTVSWNSGALTSGVHTFPAVLQSSGL |
| | | | | YSLSSVVTVPSSSLGTQTYICNVNHKPSNTKV |
| | | | | DKKVEPKSCDKTHTCPPCPAPEAAGGPSVFLF |
| | | | | PPKPKDTLMISRTPEVTCVVVSVSHEDPEVKF |
| | | | | NWYVDGVEVHNAKTKPREEQYNSTYRVVSV |
| | | | | LTVLHQDWLNGKEYKCKVSNKALPAPIEKTI |
| | | | | SKAKGQPREPQVYVLPPSREEMTKNQVSLLC |
| | | | | LVKGFYPSDIAVEWESNGQPENNYLTWPPVL |
| | | | | DSDGSFFLYSKLTVDKSRWQQGNVFSCSVM |
| | | | | HEALHNRFTQKSLSLSPGK |
| 434 | PRT | Mouse- | V7BCB37-LC2 | EIVLTQSPGTLSLSPGERATLSCRASQSISSSFL |
| | | humanized | | TWYQQKPGQAPRLLIYGASSRATGIPDRFSG |
| | | | | GGSGTDFTLTISRLEPEDFAVYYCQHYGSSPM |
| | | | | YTFGQGTKLEIKRTVAAPSVFIFPPSDEQLKSG |
| | | | | TASVVCLLNNFYPREAKVQWKVDNALQSGN |
| | | | | SQESVTEQDSKDSTYSLSSTLTLSKADYEKHK |
| | | | | VYACEVTHQGLSSPVTKSFNRGEC |
| 435 | PRT | Mouse- | KLCB293- | DIQMTQSPSSLSASVGDRVTITCRSSQSLVHSS |
| | | humanized | HC1 (B17B21- | GNTYLHWYQQKPGCAPKFLIYKVSNRFSGVP |
| | | | N33S-LH- | SRFSGSGSGTDFTLTISSLQPEDFATYYCSQST |
| | | | spFv) | HVPFTFGQGTKLEIKGGGSGGSGGCPPCGGS |
| | | | | GGQVQLQESGPGLVKPSETLSLTCTVSGYSIT |
| | | | | SGYFWNWIRQPPGKGLEWIGYISYDGSNNYN |
| | | | | PSLKSRVTISRDTSKNQFSLKLSSVTAADTAV |
| | | | | YYCASPSPGTGYAVDYWGCGTLVTVSSEPKS |
| | | | | SDKTHTCPPCPAPEAAGGPSVFLFPPKPKDTL |
| | | | | MISRTPEVTCVVVSVSHEDPEVKFNWYVDGV |
| | | | | EVHNAKTKPREEQYNSTYRVVSVLTVLHQD |
| | | | | WLNGKEYKCKVSNKALPAPIEKTISKAKGQP |
| | | | | REPQVYTLPPSREEMTKNQVSLWCLVKGFYP |
| | | | | SDIAVEWESNGQPENNYKTTPPVLDSDGSFFL |
| | | | | YSKLTVDKSRWQQGNVFSCSVMHEALHNHY |
| | | | | TQKSLSLSPGK |
| 436 | PRT | Mouse- | KLCB293- | QVQLQESGPGLVKPSETLSLTCTVSGGSISSY |
| | | humanized | HC2 (KL2B30- | YWSWIRQPPGKGLEWIGYIYYSGSTNYNPSL |
| | | | Fab) | KSRVTISVDTSKNQFSLKLSSVTAADTAVYY |
| | | | | CAGTTIFGVVTPNFYYGMDVWGQGTTVTVS |
| | | | | SASTKGPSVFPLAPSSKSTSGGTAALGCLVKD |
| | | | | YFPEPVTVSWNSGALTSGVHTFPAVLQSSGL |
| | | | | YSLSSVVTVPSSSLGTQTYICNVNHKPSNTKV |
| | | | | DKKVEPKSCDKTHTCPPCPAPEAAGGPSVFLF |
| | | | | PPKPKDTLMISRTPEVTCVVVSVSHEDPEVKF |
| | | | | NWYVDGVEVHNAKTKPREEQYNSTYRVVSV |
| | | | | LTVLHQDWLNGKEYKCKVSNKALPAPIEKTI |
| | | | | SKAKGQPREPQVYTLPPSREEMTKNQVSLSC |
| | | | | AVKGFYPSDIAVEWESNGQPENNYKTTPPVL |
| | | | | DSDGSFFLVSKLTVDKSRWQQGNVFSCSVM |
| | | | | HEALHNRFTQKSLSLSPGK |
| 437 | PRT | Mouse- | KLCB293-LC2 | DIQMTQSPSFLSASVGDRVTITCRASQGISSYL |
| | | humanized | | AWYQQKPGKAPKFLIYAASTLQSGVPSRFSG |
| | | | | SGSGTEFTLTISSLQPEDFATYYCQQLNSYPLT |
| | | | | FGGGTKVEIKRTVAAPSVFIFPPSDEQLKSGT |
| | | | | ASVVCLLNNFYPREAKVQWKVDNALQSGNS |
| | | | | QESVTEQDSKDSTYSLSSTLTLSKADYEKHK |
| | | | | VYACEVTHQGLSSPVTKSFNRGEC |
| 438 | PRT | Mouse- | VP28B21-HC1 | QVQLQESGPGLVKPSETLSLTCTVSGYSITSG |
| | | humanized | (B17B852- | YFWNWIRQFPGKGLEWMGYISYDGSNNYNP |
| | | | G34R-Fab) | SLKSRITISRDTSKNQFSLKLSSVTAADTAVY |
| | | | | YCASPSPGTGYAVDYWGQGTTVTVSSASTK |
| | | | | GPSVFPLAPSSKSTSGGTAALGCLVKDYFPEP |
| | | | | VTVSWNSGALTSGVHTFPAVLQSSGLYSLSS |
| | | | | VVTVPSSSLGTQTYICNVNHKPSNTKVDKKV |
| | | | | EPKSCDKTHTCPPCPAPEAAGGPSVFLFPPKP |
| | | | | KDTLMISRTPEVTCVVVSVSHEDPEVKFNWY |
| | | | | VDGVEVHNAKTKPREEQYNSTYRVVSVLTV |
| | | | | LHQDWLNGKEYKCKVSNKALPAPIEKTISKA |
| | | | | KGQPREPQVYTLPPSREEMTKNQVSLSCAVK |
| | | | | GFYPSDIAVEWESNGQPENNYKTTPPVLDSD |
| | | | | GSFFLVSKLTVDKSRWQQGNVFSCSVMHEA |
| | | | | LHNRFTQKSLSLSPGK |
| 439 | PRT | Mouse- | VP28B21-LC1 | DIQMTQSPSSLSASVGDRVTITCRSSQSLVHS |
| | | humanized | | NRNTYLHWYQQKPGKAPKLLIYKVSNRFSG |
| | | | | VPSRFSGSGSGTDFTLTISSLQPEDFATYYCSQ |
| | | | | STHVPFTFGQGTKLEIKRTVAAPSVFIFPPSDE |
| | | | | QLKSGTASVVCLLNNFYPREAKVQWKVDNA |
| | | | | LQSGNSQESVTEQDSKDSTYSLSSTLTLSKAD |
| | | | | YEKHKVYACEVTHQGLSSPVTKSFNRGEC |
| 440 | PRT | Mouse- | VP28B21-HC2 | DIQMTQSPSTLSASVGDRVTITCRASQSISSWL |
| | | humanized | (PSMB410- | AWYQQKPGCAPKLLIYKASSLESGVPSRFSGS |
| | | | LH-spFv) | GSGTEFTLTISSLQPDDFATYYCQQYNSYSRT |
| | | | | FGQGTKVEIKGGGSGGSGGCPPCGGSGGQLQ |
| | | | | LQESGPGLVKPSETLSLTCTVSGASISSPSYY |
| | | | | WGWIRQPPGKGLEWIGSIFYSGSSYYNPSLKS |
| | | | | RVIMSVDTSKNQFSLKLSSVTAADTALYYCA |
| | | | | SQSGVSGWYGAEYFQHWGCGTLVTVSSEPK |
| | | | | SSDKTHTCPPCPAPEAAGGPSVFLFPPKPKDT |
| | | | | LMISRTPEVTCVVVSVSHEDPEVKFNWYVDG |
| | | | | VEVHNAKTKPREEQYNSTYRVVSVLTVLHQ |
| | | | | DWLNGKEYKCKVSNKALPAPIEKTISKAKGQ |
| | | | | PREPQVYTLPPSREEMTKNQVSLWCLVKGFY |
| | | | | PSDIAVEWESNGQPENNYKTTPPVLDSDGSFF |
| | | | | LYSKLTVDKSRWQQGNVFSCSVMHEALHNH |
| | | | | YTQKSLSLSPGK |
| 441 | PRT | Mouse- | VB17B27-HC1 | DIQMTQSPSSLSASVGDRVTITCRSSQSLVHS |
| | | humanized | (B17B852- | NRNTYLHWYQQKPGCAPKLLIYKVSNRFSG |
| | | | G34R-LH- | VPSRFSGSGSGTDFTLTISSLQPEDFATYYCSQ |
| | | | spFv) | STHVPFTFGQGTKLEIKGGGSGGSGGCPPCGG |
| | | | | SGGQVQLQESGPGLVKPSETLSLTCTVSGYSI |
| | | | | TSGYFWNWIRQFPGKGLEWMGYISYDGSNN |
| | | | | YNPSLKSRITISRDTSKNQFSLKLSSVTAADTA |
| | | | | VYYCASPSPGTGYAVDYWGCGTTVTVSSEP |
| | | | | KSSDKTHTCPPCPAPEAAGGPSVFLFPPKPKD |
| | | | | TLMISRTPEVTCVVVSVSHEDPEVKFNWYVD |
| | | | | GVEVHNAKTKPREEQYNSTYRVVSVLTVLH |
| | | | | QDWLNGKEYKCKVSNKALPAPIEKTISKAKG |
| | | | | QPREPQVYTLPPSREEMTKNQVSLWCLVKGF |
| | | | | YPSDIAVEWESNGQPENNYKTTPPVLDSDGS |
| | | | | FFLYSKLTVDKSRWQQGNVFSCSVMHEALH |
| | | | | NHYTQKSLSLSPGK |
| 442 | PRT | Mouse- | VB17B27-HC2 | EVQLVESGGGLVQPGGSLRLSCAASGFTFSSY |
| | | humanized | (PSMB896- | AMSWVRQAPGKGLEWVSAISGGIGSTYYAD |
| | | | G100A-Fab) | SVKGRFTISRDNSKNTLWLQMNSLRAEDTAV |
| | | | | YYCAKDAVGATPYYFDYWGQGTLVTVSSAS |
| | | | | TKGPSVFPLAPSSKSTSGGTAALGCLVKDYFP |
| | | | | EPVTVSWNSGALTSGVHTFPAVLQSSGLYSL |
| | | | | SSVVTVPSSSLGTQTYICNVNHKPSNTKVDK |
| | | | | KVEPKSCDKTHTCPPCPAPEAAGGPSVFLFPP |
| | | | | KPKDTLMISRTPEVTCVVVSVSHEDPEVKFN |
| | | | | WYVDGVEVHNAKTKPREEQYNSTYRVVSVL |
| | | | | TVLHQDWLNGKEYKCKVSNKALPAPIEKTIS |
| | | | | KAKGQPREPQVYTLPPSREEMTKNQVSLSCA |
| | | | | VKGFYPSDIAVEWESNGQPENNYKTTPPVLD |
| | | | | SDGSFFLVSKLTVDKSRWQQGNVFSCSVMHE |
| | | | | ALHNRFTQKSLSLSPGK |
| 443 | PRT | Mouse- | VB17B27-LC2 | QSVLTQPPSVSAAPGQKVTISCSGSSSNIGINY |
| | | humanized | | VSWYQQLPGTAPKLLIYDNNKRPSGIPDRFSG |
| | | | | SKSGTSATLGITGLQTGDEADYYCGTWDSSL |
| | | | | SAVVFGGGTKLTVLGQPKAAPSVTLFPPSSEE |
| | | | | LQANKATLVCLISDFYPGAVTVAWKADSSPV |
| | | | | KAGVETTTPSKQSNNKYAASSYLSLTPEQWK |
| | | | | SHRSYSCQVTHEGSTVEKTVAPTECS |
| 444 | PRT | Mouse- | VB17B29-HC1 | QVQLQESGPGLVKPSETLSLTCTVSGYSITSG |
| | | humanized | (B17B21- | YFWNWIRQPPGKGLEWIGYISYDGSNNYNPS |
| | | | N33S-Fab) | LKSRVTISRDTSKNQFSLKLSSVTAADTAVYY |
| | | | | CASPSPGTGYAVDYWGQGTLVTVSSASTKGP |
| | | | | SVFPLAPSSKSTSGGTAALGCLVKDYFPEPVT |
| | | | | VSWNSGALTSGVHTFPAVLQSSGLYSLSSVV |
| | | | | TVPSSSLGTQTYICNVNHKPSNTKVDKKVEP |
| | | | | KSCDKTHTCPPCPAPEAAGGPSVFLFPPKPKD |
| | | | | TLMISRTPEVTCVVVSVSHEDPEVKFNWYVD |
| | | | | GVEVHNAKTKPREEQYNSTYRVVSVLTVLH |
| | | | | QDWLNGKEYKCKVSNKALPAPIEKTISKAKG |
| | | | | QPREPQVYTLPPSREEMTKNQVSLSCAVKGF |
| | | | | YPSDIAVEWESNGQPENNYKTTPPVLDSDGS |
| | | | | FFLVSKLTVDKSRWQQGNVFSCSVMHEALH |
| | | | | NRFTQKSLSLSPGK |
| 445 | PRT | Mouse- | VB17B29-LC1 | DIQMTQSPSSLSASVGDRVTITCRSSQSLVHSS |
| | | humanized | | GNTYLHWYQQKPGKAPKFLIYKVSNRFSGVP |
| | | | | SRFSGSGSGTDFTLTISSLQPEDFATYYCSQST |
| | | | | HVPFTFGQGTKLEIKRTVAAPSVFIFPPSDEQL |
| | | | | KSGTASVVCLLNNFYPREAKVQWKVDNALQ |
| | | | | SGNSQESVTEQDSKDSTYSLSSTLTLSKADYE |
| | | | | KHKVYACEVTHQGLSSPVTKSFNRGEC |
| 446 | PRT | Mouse- | VB17B29-HC2 | QSVLTQPPSVSAAPGQKVTISCSGSSSNIGINY |
| | | humanized | (PSMB896- | VSWYQQLPGCAPKLLIYDNNKRPSGIPDRFSG |
| | | | G100A-LH- | SKSGTSATLGITGLQTGDEADYYCGTWDSSL |
| | | | spFv) | SAVVFGGGTKLTVLGGGSGGSGGCPPCGGSG |
| | | | | GEVQLVESGGGLVQPGGSLRLSCAASGFTFSS |
| | | | | YAMSWVRQAPGKGLEWVSAISGGIGSTYYA |
| | | | | DSVKGRFTISRDNSKNTLWLQMNSLRAEDTA |
| | | | | VYYCAKDAVGATPYYFDYWGCGTLVTVSSE |
| | | | | PKSSDKTHTCPPCPAPEAAGGPSVFLFPPKPK |
| | | | | DTLMISRTPEVTCVVVSVSHEDPEVKFNWYV |
| | | | | DGVEVHNAKTKPREEQYNSTYRVVSVLTVL |
| | | | | HQDWLNGKEYKCKVSNKALPAPIEKTISKAK |
| | | | | GQPREPQVYTLPPSREEMTKNQVSLWCLVKG |
| | | | | FYPSDIAVEWESNGQPENNYKTTPPVLDSDG |
| | | | | SFFLYSKLTVDKSRWQQGNVFSCSVMHEAL |
| | | | | HNHYTQKSLSLSPGK |
| 447 | PRT | Mouse- | VB17B31-HC1 | QVQLQESGPGLVKPSETLSLTCTVSGYSITSG |
| | | humanized | (B17B21- | YFWNWIRQPPGCGLEWIGYISYDGSNNYNPS |
| | | | N33S-HL- | LKSRVTISRDTSKNQFSLKLSSVTAADTAVYY |
| | | | spFv) | CASPSPGTGYAVDYWGQGTLVTVSSGGGSG |
| | | | | GSGGCPPCGGSGGDIQMTQSPSSLSASVGDR |
| | | | | VTITCRSSQSLVHSSGNTYLHWYQQKPGKAP |
| | | | | KFLIYKVSNRFSGVPSRFSGSGSGTDFTLTISS |
| | | | | LQPEDFATYYCSQSTHVPFTFGCGTKLEIKEP |
| | | | | KSSDKTHTCPPCPAPEAAGGPSVFLFPPKPKD |
| | | | | TLMISRTPEVTCVVVSVSHEDPEVKFNWYVD |
| | | | | GVEVHNAKTKPREEQYNSTYRVVSVLTVLH |
| | | | | QDWLNGKEYKCKVSNKALPAPIEKTISKAKG |
| | | | | QPREPQVYTLPPSREEMTKNQVSLWCLVKGF |
| | | | | YPSDIAVEWESNGQPENNYKTTPPVLDSDGS |
| | | | | FFLYSKLTVDKSRWQQGNVFSCSVMHEALH |
| | | | | NHYTQKSLSLSPGK |
| 448 | PRT | Mouse- | VB17B31-HC2 | EVQLVESGGGLVQPGGSLRLSCAASGFTFSSY |
| | | humanized | (PSMB896- | AMSWVRQAPGKGLEWVSAISGGIGSTYYAD |
| | | | G100A-Fab) | SVKGRFTISRDNSKNTLWLQMNSLRAEDTAV |
| | | | | YYCAKDAVGATPYYFDYWGQGTLVTVSSAS |
| | | | | TKGPSVFPLAPSSKSTSGGTAALGCLVKDYFP |
| | | | | EPVTVSWNSGALTSGVHTFPAVLQSSGLYSL |
| | | | | SSVVTVPSSSLGTQTYICNVNHKPSNTKVDK |
| | | | | KVEPKSCDKTHTCPPCPAPEAAGGPSVFLFPP |
| | | | | KPKDTLMISRTPEVTCVVVSVSHEDPEVKFN |
| | | | | WYVDGVEVHNAKTKPREEQYNSTYRVVSVL |
| | | | | TVLHQDWLNGKEYKCKVSNKALPAPIEKTIS |
| | | | | KAKGQPREPQVYTLPPSREEMTKNQVSLSCA |
| | | | | VKGFYPSDIAVEWESNGQPENNYKTTPPVLD |
| | | | | SDGSFFLVSKLTVDKSRWQQGNVFSCSVMHE |
| | | | | ALHNRFTQKSLSLSPGK |
| 449 | PRT | Mouse- | VB17B31-LC2 | QSVLTQPPSVSAAPGQKVTISCSGSSSNIGINY |
| | | humanized | | VSWYQQLPGTAPKLLIYDNNKRPSGIPDRFSG |
| | | | | SKSGTSATLGITGLQTGDEADYYCGTWDSSL |
| | | | | SAVVFGGGTKLTVLGQPKAAPSVTLFPPSSEE |
| | | | | LQANKATLVCLISDFYPGAVTVAWKADSSPV |
| | | | | KAGVETTTPSKQSNNKYAASSYLSLTPEQWK |
| | | | | SHRSYSCQVTHEGSTVEKTVAPTECS |
| 450 | PRT | Mouse- | VB17B32-HC1 | QVQLQESGPGLVKPSETLSLTCTVSGYSITSG |
| | | humanized | (B17B852- | YFWNWIRQPPGCGLEWIGYISYDGSNNYNPS |
| | | | G34R-HL- | LKSRVTISRDTSKNQFSLKLSSVTAADTAVYY |
| | | | spFv) | CASPSPGTGYAVDYWGQGTLVTVSSGGGSG |
| | | | | GSGGCPPCGGSGGDIQMTQSPSSLSASVGDR |
| | | | | VTITCRSSQSLVHSSGNTYLHWYQQKPGKAP |
| | | | | KFLIYKVSNRFSGVPSRFSGSGSGTDFTLTISS |
| | | | | LQPEDFATYYCSQSTHVPFTFGCGTKLEIKEP |
| | | | | KSSDKTHTCPPCPAPEAAGGPSVFLFPPKPKD |
| | | | | TLMISRTPEVTCVVVSVSHEDPEVKFNWYVD |
| | | | | GVEVHNAKTKPREEQYNSTYRVVSVLTVLH |
| | | | | QDWLNGKEYKCKVSNKALPAPIEKTISKAKG |
| | | | | QPREPQVYTLPPSREEMTKNQVSLWCLVKGF |
| | | | | YPSDIAVEWESNGQPENNYKTTPPVLDSDGS |
| | | | | FFLYSKLTVDKSRWQQGNVFSCSVMHEALH |
| | | | | NHYTQKSLSLSPGK |
| 451 | PRT | Mouse- | VB17B32-HC2 | EVQLVESGGGLVQPGGSLRLSCAASGFTFSSY |
| | | humanized | (PSMB896- | AMSWVRQAPGKGLEWVSAISGGIGSTYYAD |
| | | | G100A-Fab) | SVKGRFTISRDNSKNTLWLQMNSLRAEDTAV |
| | | | | YYCAKDAVGATPYYFDYWGQGTLVTVSSAS |
| | | | | TKGPSVFPLAPSSKSTSGGTAALGCLVKDYFP |
| | | | | EPVTVSWNSGALTSGVHTFPAVLQSSGLYSL |
| | | | | SSVVTVPSSSLGTQTYICNVNHKPSNTKVDK |
| | | | | KVEPKSCDKTHTCPPCPAPEAAGGPSVFLFPP |
| | | | | KPKDTLMISRTPEVTCVVVSVSHEDPEVKFN |
| | | | | WYVDGVEVHNAKTKPREEQYNSTYRVVSVL |
| | | | | TVLHQDWLNGKEYKCKVSNKALPAPIEKTIS |
| | | | | KAKGQPREPQVYTLPPSREEMTKNQVSLSCA |
| | | | | VKGFYPSDIAVEWESNGQPENNYKTTPPVLD |
| | | | | SDGSFFLVSKLTVDKSRWQQGNVFSCSVMHE |
| | | | | ALHNRFTQKSLSLSPGK |
| 452 | PRT | Mouse- | VB17B32-LC2 | QSVLTQPPSVSAAPGQKVTISCSGSSSNIGINY |
| | | humanized | | VSWYQQLPGTAPKLLIYDNNKRPSGIPDRFSG |
| | | | | SKSGTSATLGITGLQTGDEADYYCGTWDSSL |
| | | | | SAVVFGGGTKLTVLGQPKAAPSVTLFPPSSEE |
| | | | | LQANKATLVCLISDFYPGAVTVAWKADSSPV |
| | | | | KAGVETTTPSKQSNNKYAASSYLSLTPEQWK |
| | | | | SHRSYSCQVTHEGSTVEKTVAPTECS |

1. A Vβ17 bispecific antibody or antigen-binding fragment thereof, the Vβ17 bispecific antibody or antigen-binding fragment thereof comprising:
a. a first heavy chain (HC1),
b. a first light chain (LC1),
c. a second heavy chain (HC2), and
d. a second light chain (LC2),
wherein HC1 is associated with LC1 to form a binding site for a first antigen, and HC2 is associated with LC2 to form a binding site for a second antigen,
wherein HC1 comprises a heavy chain complementarity determining region 1 (HCDR1), a heavy chain complementarity determining region 2 (HCDR2), and a heavy chain complementarity determining region 3 (HCDR3), and
wherein LC1 comprises a light chain complementarity determining region 1 (LCDR1), a light chain complementarity determining region 2 (LCDR2), and a light chain complementarity determining region 3 (LCDR3),
wherein
i. the HCDR1, HCDR2, and HCDR3 comprise amino acid sequences of SEQ ID NOs: 45, 46, and 47, respectively, and the LCDR1, LCDR2, and LCDR3 comprise amino acid sequences of SEQ ID NOs: 60, 61, and 62, respectively; or
ii. the HCDR1, HCDR2, and HCDR3 comprise amino acid sequences of SEQ ID NOs: 48, 49, and 50, respectively, and the LCDR1, LCDR2, and LCDR3 comprise amino acid sequences of SEQ ID NOs: 63, 64, and 65, respectively; or
iii. the HCDR1, HCDR2, and HCDR3 comprise amino acid sequences of SEQ ID NOs: 51, 52, and 53, respectively, and the LCDR1, LCDR2, and LCDR3 comprise amino acid sequences of SEQ ID NOs: 66, 67, and 68, respectively; or
iv. the HCDR1, HCDR2, and HCDR3 comprise amino acid sequences of SEQ ID NOs: 54, 55, and 56, respectively, and the LCDR1, LCDR2, and LCDR3 comprise amino acid sequences of SEQ ID NO: 69, KVS, and SEQ ID NO: 71, respectively; or
v. the HCDR1, HCDR2, and HCDR3 comprise amino acid sequences of SEQ ID NOs: 57, 58, and 59, respectively, and the LCDR1, LCDR2, and LCDR3 comprise amino acid sequences of SEQ ID NOs: 72, 73, and 74, respectively; or
vi. the HCDR1, HCDR2, and HCDR3 comprise amino acid sequences of SEQ ID NOs: 77, 78, and 79, respectively, and the LCDR1, LCDR2, and LCDR3 comprise amino acid sequences of SEQ ID NOs: 92, 93, and 94, respectively; or
vii. the HCDR1, HCDR2, and HCDR3 comprise amino acid sequences of SEQ ID NOs: 80, 81, and 82, respectively, and the LCDR1, LCDR2, and LCDR3 comprise amino acid sequences of SEQ ID NOs: 95, 96, and 97, respectively; or
viii. the HCDR1, HCDR2, and HCDR3 comprise amino acid sequences of SEQ ID NOs: 83, 84, and 85, respectively, and the LCDR1, LCDR2, and LCDR3 comprise amino acid sequences of SEQ ID NOs: 98, 99, and 100, respectively; or
ix. the HCDR1, HCDR2, and HCDR3 comprise amino acid sequences of SEQ ID NOs: 86, 87, and 88, respectively, and the LCDR1, LCDR2, and LCDR3 comprise amino acid sequences of SEQ ID NO: 101, KVS, and SEQ ID NO: 103, respectively; or
x. the HCDR1, HCDR2, and HCDR3 comprise amino acid sequences of SEQ ID NOs: 89, 90, and 91, respectively, and the LCDR1, LCDR2, and LCDR3 comprise amino acid sequences of SEQ ID NOs: 104, 105, and 106, respectively; or
xi. the HCDR1, HCDR2, and HCDR3 comprise amino acid sequences of SEQ ID NOs: 109, 110, and 111, respectively, and the LCDR1, LCDR2, and LCDR3 comprise amino acid sequences of SEQ ID NOs: 124, 125, and 126, respectively; or
xii. the HCDR1, HCDR2, and HCDR3 comprise amino acid sequences of SEQ ID NOs: 112, 113, and 114, respectively, and the LCDR1, LCDR2, and LCDR3 comprise amino acid sequences of SEQ ID NOs: 127, 128, and 129, respectively; or
xiii. the HCDR1, HCDR2, and HCDR3 comprise amino acid sequences of SEQ ID NOs: 115, 116, and 117, respectively, and the LCDR1, LCDR2, and LCDR3 comprise amino acid sequences of SEQ ID NOs: 130, 131, and 132, respectively; or
xiv. the HCDR1, HCDR2, and HCDR3 comprise an amino acid sequences of SEQ ID NOs: 118, 119, and 120, respectively, and the LCDR1, LCDR2, and LCDR3 comprise amino acid sequences of SEQ ID NO: 133, KVS, and SEQ ID NO: 135, respectively; or
xv. the HCDR1, HCDR2, and HCDR3 comprise amino acid sequences of SEQ ID NOs: 121, 122, and 123, respectively, and the LCDR1, LCDR2, and LCDR3 comprise amino acid sequences of SEQ ID NOs: 136, 137, and 138, respectively; or
xvi. the HCDR1, HCDR2, and HCDR3 comprise amino acid sequences of SEQ ID NOs: 141, 142, and 143, respectively, and the LCDR1, LCDR2, and LCDR3 comprise amino acid sequences of SEQ ID NOs: 156, 157, and 158, respectively; or
xvii. the HCDR1, HCDR2, and HCDR3 comprise amino acid sequences of SEQ ID NOs: 144, 145, and 146, respectively, and the LCDR1, LCDR2, and LCDR3 comprise amino acid sequences of SEQ ID NOs: 159, 160, and 161, respectively; or
xviii. the HCDR1, HCDR2, and HCDR3 comprise amino acid sequences of SEQ ID NOs: 147, 148, and 149, respectively, and the LCDR1, LCDR2, and LCDR3 comprise amino acid sequences of SEQ ID NOs: 162, 163, and 164, respectively; or
xix. the HCDR1, HCDR2, and HCDR3 comprise amino acid sequences of SEQ ID NOs: 150, 151, and 152, respectively, and the LCDR1, LCDR2, and LCDR3 comprise amino acid sequences of SEQ ID NO: 165, KVS, and SEQ ID NO: 167, respectively; or
xx. the HCDR1, HCDR2, and HCDR3 comprise amino acid sequences of SEQ ID NOs: 153, 154, and 155, respectively, and the LCDR1, LCDR2, and LCDR3 comprise amino acid sequences of SEQ ID NOs: 168, 169, and 170, respectively; or
xxi. the HCDR1, HCDR2, and HCDR3 comprise amino acid sequences of SEQ ID NOs: 173, 174, and 175, respectively, and the LCDR1, LCDR2, and LCDR3 comprise amino acid sequences of SEQ ID NOs: 188, 189, and 190, respectively; or
xxii. the HCDR1, HCDR2, and HCDR3 comprise amino acid sequences of SEQ ID NOs: 176, 177, and 178, respectively, and the LCDR1, LCDR2, and LCDR3 comprise amino acid sequences of SEQ ID NOs: 191, 192, and 193, respectively; or
xxiii. the HCDR1, HCDR2, and HCDR3 comprise amino acid sequences of SEQ ID NOs: 179, 180, and 181, respectively, and the LCDR1, LCDR2, and LCDR3 comprise amino acid sequences of SEQ ID NOs: 194, 195, and 196, respectively; or
xxiv. the HCDR1, HCDR2, and HCDR3 comprise amino acid sequences of SEQ ID NOs: 182, 183, and 184, respectively, and the LCDR1, LCDR2, and LCDR3 comprise amino acid sequences of SEQ ID NO: 197, KVS, and SEQ ID NO: 199, respectively; or
xxv. the HCDR1, HCDR2, and HCDR3 comprise amino acid sequences of SEQ ID NOs: 185, 186, and 187, respectively, and the LCDR1, LCDR2, and LCDR3 comprise amino acid sequences of SEQ ID NOs: 200, 201, and 202, respectively; or
xxvi. the HCDR1, HCDR2, and HCDR3 comprise amino acid sequences of SEQ ID NOs: 205, 206, and 207, respectively, and the LCDR1, LCDR2, and LCDR3 comprise amino acid sequences of SEQ ID NOs: 220, 221, and 222, respectively; or
xxvii. the HCDR1, HCDR2, and HCDR3 comprise amino acid sequences of SEQ ID NOs: 208, 209, and 210, respectively, and the LCDR1, LCDR2, and LCDR3 comprise amino acid sequences of SEQ ID NOs: 223, 224, and 225, respectively; or
xxviii. the HCDR1, HCDR2, and HCDR3 comprise amino acid sequences of SEQ ID NOs: 211, 212, and 213, respectively, and the LCDR1, LCDR2, and LCDR3 comprise amino acid sequences of SEQ ID NOs: 226, 227, and 228, respectively; or
xxix. the HCDR1, HCDR2, and HCDR3 comprise amino acid sequences of SEQ ID NOs: 214, 215, and 216, respectively, and the LCDR1, LCDR2, and LCDR3 comprise amino acid sequences of SEQ ID NO: 229, KVS, and SEQ ID NO: 231, respectively; or
xxx. the HCDR1, HCDR2, and HCDR3 comprise amino acid sequences of SEQ ID NOs: 217, 218, and 219, respectively, and the LCDR1, LCDR2, and LCDR3 comprise amino acid sequences of SEQ ID NOs: 232, 233, and 234, respectively.

2. The Vβ17 bispecific antibody or antigen-binding fragment thereof of claim 1, wherein the binding site for the first antigen binds to Vβ17 on a CD8+ or CD4+ T cell.

3. The Vβ17 bispecific antibody or antigen-binding fragment thereof of claim 1 or 2, wherein the binding site for the second antigen binds to a tumor antigen present on the surface of a cancer cell.

4. The Vβ17 bispecific antibody or antigen-binding fragment of any one of claims 1 to 3, wherein HC1 and LC1 are humanized.

5. The Vβ17 bispecific antibody or antigen-binding fragment thereof of any one of claims 1 to 4, wherein the binding site for the second antigen binds to CD123, kallikrein related peptidase 2 (KLK2), prostate-specific membrane antigen (PSMA), or B-cell maturation antigen (BCMA).

6. The Vβ17 bispecific antibody or antigen-binding fragment thereof of any one of claims 1 to 5, wherein the HC2 comprises a heavy chain complementarity determining region 1 (HCDR1-2), a heavy chain complementarity determining region 2 (HCDR2-2), and a heavy chain complementarity determining region 3 (HCDR3-2), and wherein LC2 comprises a light chain complementarity determining region 1 (LCDR1-2), a light chain complementarity determining region 2 (LCDR2-2), and a light chain complementarity determining region 3 (LCDR3-2), and
wherein
i. the HCDR1-2, HCDR2-2, and HCDR3-2 comprise amino acid sequences of SEQ ID NOs: 34, 35, and 36, respectively, and the LCDR1-2, LCDR2-2, and LCDR3-2 comprise amino acid sequences of SEQ ID NOs: 37, 38, and 39, respectively; or
ii. the HCDR1-2, HCDR2-2, and HCDR3-2 comprise amino acid sequences of SEQ ID NOs: 237, 238, and 239, respectively, and the LCDR1-2, LCDR2-2, and LCDR3-2 comprise amino acid sequences of SEQ ID NOs: 252, 253, and 254, respectively; or
iii. the HCDR1-2, HCDR2-2, and HCDR3-2 comprise amino acid sequences of SEQ ID NOs: 240, 241, and 242, respectively, and the LCDR1-2, LCDR2-2, and LCDR3-2 comprise amino acid sequences of SEQ ID NOs: 255, 256, and 257, respectively; or
iv. the HCDR1-2, HCDR2-2, and HCDR3-2 comprise amino acid sequences of SEQ ID NOs: 243, 244, and 245, respectively, and the LCDR1-2, LCDR2-2, and LCDR3-2 comprise amino acid sequences of SEQ ID NOs: 258, 259, and 260, respectively; or
v. the HCDR1-2, HCDR2-2, and HCDR3-2 comprise amino acid sequences of SEQ ID NOs: 246, 247, and 248, respectively, and the LCDR1-2, LCDR2-2, and LCDR3-2 comprise amino acid sequences of SEQ ID NO: 261, AAS, and SEQ ID NO: 263, respectively; or
vi. the HCDR1-2, HCDR2-2, and HCDR3-2 comprise amino acid sequences of SEQ ID NOs: 249, 250, and 251, respectively, and the LCDR1-2, LCDR2-2, and LCDR3-2 comprise amino acid sequences of SEQ ID NOs: 264, 265, and 266, respectively; or
vii. the HCDR1-2, HCDR2-2, and HCDR3-2 comprise amino acid sequences of SEQ ID NOs: 269, 270, and 271, respectively, and the LCDR1-2, LCDR2-2, and LCDR3-2 comprise amino acid sequences of SEQ ID NOs: 284, 285, and 286, respectively; or
viii. the HCDR1-2, HCDR2-2, and HCDR3-2 comprise amino acid sequences of SEQ ID NOs: 272, 273, and 274, respectively, and the LCDR1-2, LCDR2-2, and LCDR3-2 comprise amino acid sequences of SEQ ID NOs: 287, 288, and 289, respectively; or
ix. the HCDR1-2, HCDR2-2, and HCDR3-2 comprise amino acid sequences of SEQ ID NOs: 275, 276, and 277, respectively, and the LCDR1-2, LCDR2-2, and LCDR3-2 comprise amino acid sequences of SEQ ID NOs: 290, 291, and 292, respectively; or
x. the HCDR1-2, HCDR2-2, and HCDR3-2 comprise amino acid sequences of SEQ ID NOs: 278, 279, and 280, respectively, and the LCDR1-2, LCDR2-2, and LCDR3-2 comprise amino acid sequences of SEQ ID NO: 293, DNN, and SEQ ID NO: 295, respectively; or
xi. the HCDR1-2, HCDR2-2, and HCDR3-2 comprise amino acid sequences of SEQ ID NOs: 281, 282, and 283, respectively, and the LCDR1-2, LCDR2-2, and LCDR3-2 comprise amino acid sequences of SEQ ID NOs: 296, 297, and 298, respectively; or
xii. the HCDR1-2, HCDR2-2, and HCDR3-2 comprise amino acid sequences of SEQ ID NOs: 301, 302, and 303, respectively, and the LCDR1-2, LCDR2-2, and LCDR3-2 comprise amino acid sequences of SEQ ID NOs: 316, 317, and 318, respectively; or
xiii. the HCDR1-2, HCDR2-2, and HCDR3-2 comprise amino acid sequences of SEQ ID NOs: 304, 305, and 306, respectively, and the LCDR1-2, LCDR2-2, and LCDR3-2 comprise amino acid sequences of SEQ ID NOs: 319, 320, and 321, respectively; or
xiv. the HCDR1-2, HCDR2-2, and HCDR3-2 comprise amino acid sequences of SEQ ID NOs: 307, 308, and 309, respectively, and the LCDR1-2, LCDR2-2, and LCDR3-2 comprise amino acid sequences of SEQ ID NOs: 322, 323, and 324, respectively; or
xv. the HCDR1-2, HCDR2-2, and HCDR3-2 comprise amino acid sequences of SEQ ID NOs: 310, 311, and 312, respectively, and the LCDR1-2, LCDR2-2, and LCDR3-2 comprise amino acid sequences of SEQ ID NO: 325, GAS, and SEQ ID NO: 327, respectively; or
xvi. the HCDR1-2, HCDR2-2, and HCDR3-2 comprise amino acid sequences of SEQ ID NOs: 313, 314, and 315, respectively, and the LCDR1-2, LCDR2-2, and LCDR3-2 comprise amino acid sequences of SEQ ID NOs: 328, 329, and 330, respectively; or
xvii. the HCDR1-2, HCDR2-2, and HCDR3-2 comprise amino acid sequences of SEQ ID NOs: 333, 334, and 335, respectively, and the LCDR1-2, LCDR2-2, and LCDR3-2 comprise amino acid sequences of SEQ ID NOs: 348, 349, and 350, respectively; or
xviii. the HCDR1-2, HCDR2-2, and HCDR3-2 comprise amino acid sequences of SEQ ID NOs: 336, 337, and 338, respectively, and the LCDR1-2, LCDR2-2, and LCDR3-2 comprise amino acid sequences of SEQ ID NOs: 351, 352, and 353, respectively; or
xix. the HCDR1-2, HCDR2-2, and HCDR3-2 comprise amino acid sequences of SEQ ID NOs: 339, 340, and 341, respectively, and the LCDR1-2, LCDR2-2, and LCDR3-2 comprise amino acid sequences of SEQ ID NOs: 354, 355, and 356, respectively; or
xx. the HCDR1-2, HCDR2-2, and HCDR3-2 comprise amino acid sequences of SEQ ID NOs: 342, 343, and 344, respectively, and the LCDR1-2, LCDR2-2, and LCDR3-2 comprise amino acid sequences of SEQ ID NO: 357, DNN, and SEQ ID NO: 359, respectively; or
xxi. the HCDR1-2, HCDR2-2, and HCDR3-2 comprise amino acid sequences of SEQ ID NOs: 345, 346, and 347, respectively, and the LCDR1-2, LCDR2-2, and LCDR3-2 comprise amino acid sequences of SEQ ID NOs: 360, 361, and 362, respectively; or
xxii. the HCDR1-2, HCDR2-2, and HCDR3-2 comprise amino acid sequences of SEQ ID NOs: 365, 366, and 367, respectively, and the LCDR1-2, LCDR2-2, and LCDR3-2 comprise amino acid sequences of SEQ ID NOs: 380, 381, and 382, respectively; or
xxiii. the HCDR1-2, HCDR2-2, and HCDR3-2 comprise amino acid sequences of SEQ ID NOs: 368, 369, and 370, respectively, and the LCDR1-2, LCDR2-2, and LCDR3-2 comprise amino acid sequences of SEQ ID NOs: 383, 384, and 385, respectively; or
xxiv. the HCDR1-2, HCDR2-2, and HCDR3-2 comprise amino acid sequences of SEQ ID NOs: 371, 372, and 373, respectively, and the LCDR1-2, LCDR2-2, and LCDR3-2 comprise amino acid sequences of SEQ ID NOs: 386, 387, and 388, respectively; or
xxv. the HCDR1-2, HCDR2-2, and HCDR3-2 comprise amino acid sequences of SEQ ID NOs: 374, 375, and 376, respectively, and the LCDR1-2, LCDR2-2, and LCDR3-2 comprise amino acid sequences of SEQ ID NO: 389, DNN, and SEQ ID NO: 391, respectively; or
xxvi. the HCDR1-2, HCDR2-2, and HCDR3-2 comprise amino acid sequences of SEQ ID NOs: 377, 378, and 379, respectively, and the LCDR1-2, LCDR2-2, and LCDR3-2 comprise amino acid sequences of SEQ ID NOs: 392, 393, and 394, respectively; or
xxvii. the HCDR1-2, HCDR2-2, and HCDR3-2 comprise amino acid sequences of SEQ ID NOs: 397, 398, and 399, respectively, and the LCDR1-2, LCDR2-2, and LCDR3-2 comprise amino acid sequences of SEQ ID NOs: 412, 413, and 414, respectively; or
xxviii. the HCDR1-2, HCDR2-2, and HCDR3-2 comprise amino acid sequences of SEQ ID NOs: 400, 401, and 402, respectively, and the LCDR1-2, LCDR2-2, and LCDR3-2 comprise amino acid sequences of SEQ ID NOs: 415, 416, and 417, respectively; or
xxix. the HCDR1-2, HCDR2-2, and HCDR3-2 comprise amino acid sequences of SEQ ID NOs: 403, 404, and 405, respectively, and the LCDR1-2, LCDR2-2, and LCDR3-2 comprise amino acid sequences of SEQ ID NOs: 418, 419, and 420, respectively; or
xxx. the HCDR1-2, HCDR2-2, and HCDR3-2 comprise amino acid sequences of SEQ ID NOs: 404, 405, and 406, respectively, and the LCDR1-2, LCDR2-2, and LCDR3-2 comprise amino acid sequences of SEQ ID NO: 421, KAS, and SEQ ID NO: 423, respectively; or
xxxi the HCDR1-2, HCDR2-2, and HCDR3-2 comprise amino acid sequences of SEQ ID NOs: 407, 408, and 409, respectively, and the LCDR1-2, LCDR2-2, and LCDR3-2 comprise amino acid sequences of SEQ ID NOs: 424, 425, and 426, respectively.

7. The Vβ17 bispecific antibody or antigen-binding fragment thereof of any one of claims 1 to 6, wherein the bispecific antibody or antigen-binding fragment thereof is an IgG isotype, for example, IgG4 or IgG1.

8. An isolated polynucleotide comprising a polynucleotide encoding the HC1 and the LC1 of the Vβ17 bispecific antibody or antigen-binding fragment thereof of any one of claims 1 to 7.

9. An isolated polynucleotide comprising a polynucleotide encoding the HC2 and the LC2 of the Vβ17 bispecific antibody or antigen-binding fragment thereof of any one of claims 1 to 7.

10. A vector comprising the isolated polynucleotide of claim 8 and/or claim 9.

11. A host cell comprising the vector of claim 10.

12. A method for inhibiting growth or proliferation of cancer cells, comprising contacting the cancer cells with the Vβ17 bispecific antibody or antigen-binding fragment thereof of any one of claims 1 to 7, wherein the contacting the cancer cells with the Vβ17 bispecific antibody or antigen-binding fragment thereof inhibits the growth or proliferation of the cancer cells.

13. The method of claim 12, wherein the cancer cells are CD123-expressing cancer cells, kallikrein related peptidase 2 (KLK2)-expressing cancer cells, prostate-specific membrane antigen (PSMA)-expressing cancer cells, or B-cell maturation antigen (BCMA)-expressing cancer cells.

14. A kit comprising the Vβ17 bispecific antibody or antigen-binding fragment thereof of any one of claims 1 to 7 and instructions for use.

15. A method of producing a Vβ17 bispecific antibody or antigen-binding fragment thereof, comprising
(a) culturing the host cell of claim 11 to produce the Vβ17 bispecific antibody or antigen-binding fragment thereof, and
(b) recovering the Vβ17 bispecific antibody or antigen-binding fragment thereof from the cell or culture.

16. An isolated humanized Vβ17 monoclonal antibody or antigen-binding fragment thereof, the humanized Vβ17 monoclonal antibody or antigen-binding fragment thereof comprising a heavy chain (HC) having at least 95% identity to an amino acid sequence selected from the group consisting of SEQ ID NOs: 429, 432, 435, 438, 441, 444, 447, and 450.

17. The isolated humanized Vβ17 monoclonal antibody or antigen-binding fragment of claim 16, wherein the heavy chain (HC) sequence is selected from the group consisting of SEQ ID NOs: 429, 432, 435, 438, 441, 444, 447, and 450.

18. The isolated humanized Vβ17 monoclonal antibody or antigen-binding fragment thereof of claim 16 or 17, wherein the humanized Vβ17 monoclonal antibody or antigen-binding fragment thereof further comprises a light chain (LC) having at least 95% identity to an amino acid sequence selected from the group consisting of SEQ ID NOs: 431, 434, 437, 439, 443, 445, 449, and 452.

19. The isolated humanized Vβ17 monoclonal antibody or antigen-binding fragment thereof of any one of claims 16 to 18, wherein the light chain (LC) sequence is selected from the group consisting of SEQ ID NOs: 431, 434, 437, 439, 443, 445, 449, and 452.

20. An isolated nucleic acid encoding the isolated humanized Vβ17 monoclonal antibody or antigen-binding fragment thereof of any one of claims 16 to 19.

21. A vector comprising the isolated nucleic acid of claim 20.

22. A host cell comprising the vector of claim 21.

23. A Vβ17 antibody or antigen-binding fragment thereof, the Vβ17 antibody or antigen-binding fragment thereof comprising a first heavy chain (HC1) having a heavy chain complementarity determining region 1 (HCDR1), a heavy chain complementarity determining region 2 (HCDR2), and a heavy chain complementarity determining region 3 (HCDR3), and a first light chain (LC1) having a light chain complementarity determining region 1 (LCDR1), a light chain complementarity determining region 2 (LCDR2), and a light chain complementarity determining region 3 (LCDR3),
wherein
i. the HCDR1, HCDR2, and HCDR3 comprise amino acid sequences of SEQ ID NOs: 45, 46, and 47, respectively, and the LCDR1, LCDR2, and LCDR3 comprise amino acid sequences of SEQ ID NOs: 60, 61, and 62, respectively; or
ii. the HCDR1, HCDR2, and HCDR3 comprise amino acid sequences of SEQ ID NOs: 48, 49, and 50, respectively, and the LCDR1, LCDR2, and LCDR3 comprise amino acid sequences of SEQ ID NOs: 63, 64, and 65, respectively; or
iii. the HCDR1, HCDR2, and HCDR3 comprise amino acid sequences of SEQ ID NOs: 51, 52, and 53, respectively, and the LCDR1, LCDR2, and LCDR3 comprise amino acid sequences of SEQ ID NOs: 66, 67, and 68, respectively; or
iv. the HCDR1, HCDR2, and HCDR3 comprise amino acid sequences of SEQ ID NOs: 54, 55, and 56, respectively, and the LCDR1, LCDR2, and LCDR3 comprise amino acid sequences of SEQ ID NO: 69, KVS, and SEQ ID NO: 71, respectively; or
v. the HCDR1, HCDR2, and HCDR3 comprise amino acid sequences of SEQ ID NOs: 57, 58, and 59, respectively, and the LCDR1, LCDR2, and LCDR3 comprise amino acid sequences of SEQ ID NOs: 72, 73, and 74, respectively; or
vi. the HCDR1, HCDR2, and HCDR3 comprise amino acid sequences of SEQ ID NOs: 77, 78, and 79, respectively, and the LCDR1, LCDR2, and LCDR3 comprise amino acid sequences of SEQ ID NOs: 92, 93, and 94, respectively; or
vii. the HCDR1, HCDR2, and HCDR3 comprise amino acid sequences of SEQ ID NOs: 80, 81, and 82, respectively, and the LCDR1, LCDR2, and LCDR3 comprise amino acid sequences of SEQ ID NOs: 95, 96, and 97, respectively; or
viii. the HCDR1, HCDR2, and HCDR3 comprise amino acid sequences of SEQ ID NOs: 83, 84, and 85, respectively, and the LCDR1, LCDR2, and LCDR3 comprise amino acid sequences of SEQ ID NOs: 98, 99, and 100, respectively; or
ix. the HCDR1, HCDR2, and HCDR3 comprise amino acid sequences of SEQ ID NOs: 86, 87, and 88, respectively, and the LCDR1, LCDR2, and LCDR3 comprise amino acid sequences of SEQ ID NO: 101, KVS, and SEQ ID NO: 103, respectively; or
x. the HCDR1, HCDR2, and HCDR3 comprise amino acid sequences of SEQ ID NOs: 89, 90, and 91, respectively, and the LCDR1, LCDR2, and LCDR3 comprise amino acid sequences of SEQ ID NOs: 104, 105, and 106, respectively; or
xi. the HCDR1, HCDR2, and HCDR3 comprise amino acid sequences of SEQ ID NOs: 109, 110, and 111, respectively, and the LCDR1, LCDR2, and LCDR3 comprise amino acid sequences of SEQ ID NOs: 124, 125, and 126, respectively; or
xii. the HCDR1, HCDR2, and HCDR3 comprise amino acid sequences of SEQ ID NOs: 112, 113, and 114, respectively, and the LCDR1, LCDR2, and LCDR3 comprise amino acid sequences of SEQ ID NOs: 127, 128, and 129, respectively; or
xiii. the HCDR1, HCDR2, and HCDR3 comprise amino acid sequences of SEQ ID NOs: 115, 116, and 117, respectively, and the LCDR1, LCDR2, and LCDR3 comprise amino acid sequences of SEQ ID NOs: 130, 131, and 132, respectively; or
xiv. the HCDR1, HCDR2, and HCDR3 comprise an amino acid sequences of SEQ ID NOs: 118, 119, and 120, respectively, and the LCDR1, LCDR2, and LCDR3 comprise amino acid sequences of SEQ ID NO: 133, KVS, and SEQ ID NO: 135, respectively; or
xv. the HCDR1, HCDR2, and HCDR3 comprise amino acid sequences of SEQ ID NOs: 121, 122, and 123, respectively, and the LCDR1, LCDR2, and LCDR3 comprise amino acid sequences of SEQ ID NOs: 136, 137, and 138, respectively; or
xvi. the HCDR1, HCDR2, and HCDR3 comprise amino acid sequences of SEQ ID NOs: 141, 142, and 143, respectively, and the LCDR1, LCDR2, and LCDR3 comprise amino acid sequences of SEQ ID NOs: 156, 157, and 158, respectively; or
xvii. the HCDR1, HCDR2, and HCDR3 comprise amino acid sequences of SEQ ID NOs: 144, 145, and 146, respectively, and the LCDR1, LCDR2, and LCDR3 comprise amino acid sequences of SEQ ID NOs: 159, 160, and 161, respectively; or
xviii. the HCDR1, HCDR2, and HCDR3 comprise amino acid sequences of SEQ ID NOs: 147, 148, and 149, respectively, and the LCDR1, LCDR2, and LCDR3 comprise amino acid sequences of SEQ ID NOs: 162, 163, and 164, respectively; or
xix. the HCDR1, HCDR2, and HCDR3 comprise amino acid sequences of SEQ ID NOs: 150, 151, and 152, respectively, and the LCDR1, LCDR2, and LCDR3 comprise amino acid sequences of SEQ ID NO: 165, KVS, and SEQ ID NO: 167, respectively; or
xx. the HCDR1, HCDR2, and HCDR3 comprise amino acid sequences of SEQ ID NOs: 153, 154, and 155, respectively, and the LCDR1, LCDR2, and LCDR3 comprise amino acid sequences of SEQ ID NOs: 168, 169, and 170, respectively; or
xxi. the HCDR1, HCDR2, and HCDR3 comprise amino acid sequences of SEQ ID NOs: 173, 174, and 175, respectively, and the LCDR1, LCDR2, and LCDR3 comprise amino acid sequences of SEQ ID NOs: 188, 189, and 190, respectively; or
xxii. the HCDR1, HCDR2, and HCDR3 comprise amino acid sequences of SEQ ID NOs: 176, 177, and 178, respectively, and the LCDR1, LCDR2, and LCDR3 comprise amino acid sequences of SEQ ID NOs: 191, 192, and 193, respectively; or
xxiii. the HCDR1, HCDR2, and HCDR3 comprise amino acid sequences of SEQ ID NOs: 179, 180, and 181, respectively, and the LCDR1, LCDR2, and LCDR3 comprise amino acid sequences of SEQ ID NOs: 194, 195, and 196, respectively; or
xxiv. the HCDR1, HCDR2, and HCDR3 comprise amino acid sequences of SEQ ID NOs: 182, 183, and 184, respectively, and the LCDR1, LCDR2, and LCDR3 comprise amino acid sequences of SEQ ID NO: 197, KVS, and SEQ ID NO: 199, respectively; or
xxv. the HCDR1, HCDR2, and HCDR3 comprise amino acid sequences of SEQ ID NOs: 185, 186, and 187, respectively, and the LCDR1, LCDR2, and LCDR3 comprise amino acid sequences of SEQ ID NOs: 200, 201, and 202, respectively; or
xxvi. the HCDR1, HCDR2, and HCDR3 comprise amino acid sequences of SEQ ID NOs: 205, 206, and 207, respectively, and the LCDR1, LCDR2, and LCDR3 comprise amino acid sequences of SEQ ID NOs: 220, 221, and 222, respectively; or
xxvii. the HCDR1, HCDR2, and HCDR3 comprise amino acid sequences of SEQ ID NOs: 208, 209, and 210, respectively, and the LCDR1, LCDR2, and LCDR3 comprise amino acid sequences of SEQ ID NOs: 223, 224, and 225, respectively; or
xxviii. the HCDR1, HCDR2, and HCDR3 comprise amino acid sequences of SEQ ID NOs: 211, 212, and 213, respectively, and the LCDR1, LCDR2, and LCDR3 comprise amino acid sequences of SEQ ID NOs: 226, 227, and 228, respectively; or
xxix. the HCDR1, HCDR2, and HCDR3 comprise amino acid sequences of SEQ ID NOs: 214, 215, and 216, respectively, and the LCDR1, LCDR2, and LCDR3 comprise amino acid sequences of SEQ ID NO: 229, KVS, and SEQ ID NO: 231, respectively; or
xxx. the HCDR1, HCDR2, and HCDR3 comprise amino acid sequences of SEQ ID NOs: 217, 218, and 219, respectively, and the LCDR1, LCDR2, and LCDR3 comprise amino acid sequences of SEQ ID NOs: 232, 233, and 234, respectively.

24. An antibody or antigen-binding fragment thereof, the antibody or antigen binding-fragment comprising a second heavy chain (HC2) having a heavy chain complementarity determining region 1 (HCDR1-2), a heavy chain complementarity determining region 2 (HCDR2-2), and a heavy chain complementarity determining region 3 (HCDR3-2), and a second light chain (LC2) having a light chain complementarity determining region 1 (LCDR1-2), a light chain complementarity determining region 2 (LCDR2-2), and a light chain complementarity determining region 3 (LCDR3-2), and
wherein
i. the HCDR1-2, HCDR2-2, and HCDR3-2 comprise amino acid sequences of SEQ ID NOs: 34, 35, and 36, respectively, and the LCDR1-2, LCDR2-2, and LCDR3-2 comprise amino acid sequences of SEQ ID NOs: 37, 38, and 39, respectively; or
ii. the HCDR1-2, HCDR2-2, and HCDR3-2 comprise amino acid sequences of SEQ ID NOs: 237, 238, and 239, respectively, and the LCDR1-2, LCDR2-2, and LCDR3-2 comprise amino acid sequences of SEQ ID NOs: 252, 253, and 254, respectively; or
iii. the HCDR1-2, HCDR2-2, and HCDR3-2 comprise amino acid sequences of SEQ ID NOs: 240, 241, and 242, respectively, and the LCDR1-2, LCDR2-2, and LCDR3-2 comprise amino acid sequences of SEQ ID NOs: 255, 256, and 257, respectively; or
iv. the HCDR1-2, HCDR2-2, and HCDR3-2 comprise amino acid sequences of SEQ ID NOs: 243, 244, and 245, respectively, and the LCDR1-2, LCDR2-2, and LCDR3-2 comprise amino acid sequences of SEQ ID NOs: 258, 259, and 260, respectively; or
v. the HCDR1-2, HCDR2-2, and HCDR3-2 comprise amino acid sequences of SEQ ID NOs: 246, 247, and 248, respectively, and the LCDR1-2, LCDR2-2, and LCDR3-2 comprise amino acid sequences of SEQ ID NO: 261, AAS, and SEQ ID NO: 263, respectively; or
vi. the HCDR1-2, HCDR2-2, and HCDR3-2 comprise amino acid sequences of SEQ ID NOs: 249, 250, and 251, respectively, and the LCDR1-2, LCDR2-2, and LCDR3-2 comprise amino acid sequences of SEQ ID NOs: 264, 265, and 266, respectively; or
vii. the HCDR1-2, HCDR2-2, and HCDR3-2 comprise amino acid sequences of SEQ ID NOs: 269, 270, and 271, respectively, and the LCDR1-2, LCDR2-2, and LCDR3-2 comprise amino acid sequences of SEQ ID NOs: 284, 285, and 286, respectively; or
viii. the HCDR1-2, HCDR2-2, and HCDR3-2 comprise amino acid sequences of SEQ ID NOs: 272, 273, and 274, respectively, and the LCDR1-2, LCDR2-2, and LCDR3-2 comprise amino acid sequences of SEQ ID NOs: 287, 288, and 289, respectively; or
ix. the HCDR1-2, HCDR2-2, and HCDR3-2 comprise amino acid sequences of SEQ ID NOs: 275, 276, and 277, respectively, and the LCDR1-2, LCDR2-2, and LCDR3-2 comprise amino acid sequences of SEQ ID NOs: 290, 291, and 292, respectively; or
x. the HCDR1-2, HCDR2-2, and HCDR3-2 comprise amino acid sequences of SEQ ID NOs: 278, 279, and 280, respectively, and the LCDR1-2, LCDR2-2, and LCDR3-2 comprise amino acid sequences of SEQ ID NO: 293, DNN, and SEQ ID NO: 295, respectively; or
xi. the HCDR1-2, HCDR2-2, and HCDR3-2 comprise amino acid sequences of SEQ ID NOs: 281, 282, and 283, respectively, and the LCDR1-2, LCDR2-2, and LCDR3-2 comprise amino acid sequences of SEQ ID NOs: 296, 297, and 298, respectively; or
xii. the HCDR1-2, HCDR2-2, and HCDR3-2 comprise amino acid sequences of SEQ ID NOs: 301, 302, and 303, respectively, and the LCDR1-2, LCDR2-2, and LCDR3-2 comprise amino acid sequences of SEQ ID NOs: 316, 317, and 318, respectively; or
xiii. the HCDR1-2, HCDR2-2, and HCDR3-2 comprise amino acid sequences of SEQ ID NOs: 304, 305, and 306, respectively, and the LCDR1-2, LCDR2-2, and LCDR3-2 comprise amino acid sequences of SEQ ID NOs: 319, 320, and 321, respectively; or
xiv. the HCDR1-2, HCDR2-2, and HCDR3-2 comprise amino acid sequences of SEQ ID NOs: 307, 308, and 309, respectively, and the LCDR1-2, LCDR2-2, and LCDR3-2 comprise amino acid sequences of SEQ ID NOs: 322, 323, and 324, respectively; or
xv. the HCDR1-2, HCDR2-2, and HCDR3-2 comprise amino acid sequences of SEQ ID NOs: 310, 311, and 312, respectively, and the LCDR1-2, LCDR2-2, and LCDR3-2 comprise amino acid sequences of SEQ ID NO: 325, GAS, and SEQ ID NO: 327, respectively; or
xvi. the HCDR1-2, HCDR2-2, and HCDR3-2 comprise amino acid sequences of SEQ ID NOs: 313, 314, and 315, respectively, and the LCDR1-2, LCDR2-2, and LCDR3-2 comprise amino acid sequences of SEQ ID NOs: 328, 329, and 330, respectively; or
xvii. the HCDR1-2, HCDR2-2, and HCDR3-2 comprise amino acid sequences of SEQ ID NOs: 333, 334, and 335, respectively, and the LCDR1-2, LCDR2-2, and LCDR3-2 comprise amino acid sequences of SEQ ID NOs: 348, 349, and 350, respectively; or
xviii. the HCDR1-2, HCDR2-2, and HCDR3-2 comprise amino acid sequences of SEQ ID NOs: 336, 337, and 338, respectively, and the LCDR1-2, LCDR2-2, and LCDR3-2 comprise amino acid sequences of SEQ ID NOs: 351, 352, and 353, respectively; or
xix. the HCDR1-2, HCDR2-2, and HCDR3-2 comprise amino acid sequences of SEQ ID NOs: 339, 340, and 341, respectively, and the LCDR1-2, LCDR2-2, and LCDR3-2 comprise amino acid sequences of SEQ ID NOs: 354, 355, and 356, respectively; or
xx. the HCDR1-2, HCDR2-2, and HCDR3-2 comprise amino acid sequences of SEQ ID NOs: 342, 343, and 344, respectively, and the LCDR1-2, LCDR2-2, and LCDR3-2 comprise amino acid sequences of SEQ ID NO: 357, DNN, and SEQ ID NO: 359, respectively; or
xxi. the HCDR1-2, HCDR2-2, and HCDR3-2 comprise amino acid sequences of SEQ ID NOs: 345, 346, and 347, respectively, and the LCDR1-2, LCDR2-2, and LCDR3-2 comprise amino acid sequences of SEQ ID NOs: 360, 361, and 362, respectively; or
xxii. the HCDR1-2, HCDR2-2, and HCDR3-2 comprise amino acid sequences of SEQ ID NOs: 365, 366, and 367, respectively, and the LCDR1-2, LCDR2-2, and LCDR3-2 comprise amino acid sequences of SEQ ID NOs: 380, 381, and 382, respectively; or
xxiii. the HCDR1-2, HCDR2-2, and HCDR3-2 comprise amino acid sequences of SEQ ID NOs: 368, 369, and 370, respectively, and the LCDR1-2, LCDR2-2, and LCDR3-2 comprise amino acid sequences of SEQ ID NOs: 383, 384, and 385, respectively; or
xxiv. the HCDR1-2, HCDR2-2, and HCDR3-2 comprise amino acid sequences of SEQ ID NOs: 371, 372, and 373, respectively, and the LCDR1-2, LCDR2-2, and LCDR3-2 comprise amino acid sequences of SEQ ID NOs: 386, 387, and 388, respectively; or
xxv. the HCDR1-2, HCDR2-2, and HCDR3-2 comprise amino acid sequences of SEQ ID NOs: 374, 375, and 376, respectively, and the LCDR1-2, LCDR2-2, and LCDR3-2 comprise amino acid sequences of SEQ ID NO: 389, DNN, and SEQ ID NO: 391, respectively; or
xxvi. the HCDR1-2, HCDR2-2, and HCDR3-2 comprise amino acid sequences of SEQ ID NOs: 377, 378, and 379, respectively, and the LCDR1-2, LCDR2-2, and LCDR3-2 comprise amino acid sequences of SEQ ID NOs: 392, 393, and 394, respectively; or
xxvii. the HCDR1-2, HCDR2-2, and HCDR3-2 comprise amino acid sequences of SEQ ID NOs: 397, 398, and 399, respectively, and the LCDR1-2, LCDR2-2, and LCDR3-2 comprise amino acid sequences of SEQ ID NOs: 412, 413, and 414, respectively; or
xxviii. the HCDR1-2, HCDR2-2, and HCDR3-2 comprise amino acid sequences of SEQ ID NOs: 400, 401, and 402, respectively, and the LCDR1-2, LCDR2-2, and LCDR3-2 comprise amino acid sequences of SEQ ID NOs: 415, 416, and 417, respectively; or
xxix. the HCDR1-2, HCDR2-2, and HCDR3-2 comprise amino acid sequences of SEQ ID NOs: 403, 404, and 405, respectively, and the LCDR1-2, LCDR2-2, and LCDR3-2 comprise amino acid sequences of SEQ ID NOs: 418, 419, and 420, respectively; or
xxx. the HCDR1-2, HCDR2-2, and HCDR3-2 comprise amino acid sequences of SEQ ID NOs: 404, 405, and 406, respectively, and the LCDR1-2, LCDR2-2, and LCDR3-2 comprise amino acid sequences of SEQ ID NO: 421, KAS, and SEQ ID NO: 423, respectively; or
xxxi. the HCDR1-2, HCDR2-2, and HCDR3-2 comprise amino acid sequences of SEQ ID NOs: 407, 408, and 409, respectively, and the LCDR1-2, LCDR2-2, and LCDR3-2 comprise amino acid sequences of SEQ ID NOs: 424, 425, and 426, respectively.
